(19) United States
(12) Patent Application Publication
(10) Pub. No.: US 2023/0048356 A1
(43) Pub. Date: Feb. 16, 2023

(54) CELL BARCODING COMPOSITIONS AND METHODS

(71) Applicant: Factorial Diagnostics, Inc., San Carlos, CA (US)

(72) Inventors: Katie Leigh Zobeck, Sunnyvale, CA (US); Jerry Szejay Chen, San Jose, CA (US); John Daniel Wells, San Francisco, CA (US)

(21) Appl. No.: 17/692,100

(22) Filed: Mar. 10, 2022

Related U.S. Application Data
(60) Provisional application No. 63/159,395, filed on Mar. 10, 2021.
(60) Provisional application No. 63/311,002, filed on Feb. 16, 2022.

(51) Int. Cl. C12N 15/10; C12Q 1/6806; C12Q 1/6855

(52) U.S. Cl. CPC C12N 15/1065; C12Q 1/6806; C12Q 1/6855

(57) ABSTRACT

Aspects of the present disclosure relate generally to methods, compositions, and kits for in situ whole cell barcoding. Aspects of the present disclosure also include a computer readable-medium and a processor to carry out the steps of the method described herein. In some embodiments, the disclosure relates to whole cell barcoding performed in situ.

### CROSS-REFERENCE TO RELATED APPLICATIONS

This application claims the benefit of and priority to U.S. Provisional Application Nos.: 63/159,395, filed Mar. 10, 2021; and 63/311,002, filed Feb. 16, 2022, the disclosures of which are hereby incorporated by reference in their entireties.

### SEQUENCE LISTING

The instant application contains a Sequence Listing with 15 sequences which has been submitted via USPTO Patent Center and is hereby incorporated by reference in its entirety. Said ASCII copy, created on Jul. 1, 2022, is named “36792-51903 US Sequence Listing”, and is 15 kilobytes in size.

### INTRODUCTION

Current diagnostics for detecting and analyzing genetic alterations in heterogeneous cell populations include library preparation, whole genome sequencing or Next Generation Sequencing (NGS).

Traditional library preparation techniques performed on genomic DNA or RNA require lysing the cell to extract the genomic DNA from the cell in order to perform the library preparation steps before sequencing the DNA to identify regions of DNA that represent variant changes, including insertions or deletions in a specific DNA sequence or array of sequences. Many of the emerging technologies for performing NGS library preparation on single cells, also rely on cell lysis early in the NGS library preparation process. Furthermore, these single cell technologies rely on physical isolation of cells either through droplet formation, cell sorting, or portioning methods. And methods that can support pools of cells require splitting and pooling the cells multiple times to provide unique barcoding of each cell. The development of a method that can prepare NGS libraries in situ and identify individual cells without the need for physical isolation of cells or split pooling is important.

### SUMMARY

Detailed understanding of complex cell ecosystems, such as tumor ecosystems, at single-cell resolution has been limited for technological reasons. Conventional genomic, transcriptomic, and epigenomic sequencing protocols require microgram-level input materials, and so cancer-related genomic studies are largely limited to bulk tumor sequencing, which does not address intratumor heterogeneity and complexity. Additionally, conventional techniques of bulk tumor sequencing fail to provide phenotypic insight of tumor heterogeneity. Heterogeneity of cancer cells and tumor-infiltrating immune cells can provide insight into regulatory mechanisms within tumors and new drug targets to modulate tumor progression.

Aspects of the present disclosure relate generally to methods, compositions, and kits for barcoding, including for in situ combinatorial cell barcoding. This in situ combinatorial cell barcoding may be used to determine the heterogeneity of cell populations in a sample and for identifying disease-associated genetic alterations of distinct cell populations within the sample. Aspects of the present disclosure further relate generally to algorithms for tagging reads for each barcoded population, such as for barcoded cellular populations within an in situ single-cell sequencing sample within a cell identifier and quantifying structural variants from these reads. Aspects of the present disclosure also include a computer readable-medium and a processor to carry out the steps of the method described herein.

Aspects of the present disclosure also relate to methods, compositions, and kits for amplifying primers from oligonucleotides using linear amplification. The amplified primers can then be used in downstream applications, including, but not limited to amplification of a nucleic acid sequence.

In one aspect, this disclosure features a method of performing whole cell barcoding, the method including:

(a) contacting nucleic acid fragments within a cell suspension or tissue slices with:

(i) a first set of barcoding oligonucleotides, each barcoding oligonucleotide including:

a first barcode;

two consensus regions, where the two consensus regions of each barcoding primer includes:

one of the two consensus regions includes a nucleotide sequence that is complementary to a 5′ read region of a first strand of one of the DNA or RNA fragments, and

the second of the two consensus regions includes a first adapter sequence;

(ii) a second set of barcoding oligonucleotides, each barcoding oligonucleotides including:

a second barcode;

two consensus regions, where the two consensus regions of each barcoding primer includes:

one of the two consensus regions includes a nucleotide sequence that is complementary to a 5′ read region of a second strand of one of the DNA or RNA fragments, and

the second of the two consensus regions includes a second adapter sequence;

(b) amplifying:

the first set of barcoding oligonucleotides to produce a first set of barcoding primers; and

the second set of barcoding oligonucleotides to produce a second set of barcoding primers;

(c) amplifying the nucleic acid fragments with first and second set of barcoding primers to produce a set of amplicon products, where the set of amplicon products include the first barcoding primer bridging from the 5′ end of the 5′ strand of the nucleic acid fragments and the second barcoding primer bridging from the 5′ end of the opposite strand (3′ strand) of the nucleic acid fragments.

In some embodiments, the first set of barcoding oligonucleotides, second set of barcoding oligonucleotides, or both contain additional sequence for a primer binding site. In some embodiments, the primer binding site is an amplification sequence.

In some embodiments, step (i) further includes contacting the first barcoding oligonucleotide with a first primer set including nucleotide sequences that is complementary to the amplification sequence.

In some embodiments, step (ii) further includes contacting the second barcoding oligonucleotides with a second primer set including a nucleotide sequence that is complementary to the amplification sequence.

In some embodiments, the first set of barcoding oligonucleotides and the first primer set are annealed prior to said contacting to produce a first set of annealed barcoding oligonucleotides.

In some embodiments, the said amplifying in step (b) includes amplifying via polymerase chain reaction, the first and second set of barcoding oligonucleotides with the first and second set of primers to produce the first and second barcoding primers.

In some embodiments, the said amplifying in step (b) includes amplifying via isothermal amplification, the first and second set of barcoding oligonucleotides with the first and second set of primers to produce the first and second barcoding primers.

In some embodiments, the first set of barcoding oligonucleotides and the first primer set are not annealed prior to said contacting.

In some embodiments, step (i) further includes contacting the first barcoding oligonucleotide with a first primer set including nucleotide sequences that are complementary to the adapter sequence of the first barcoding oligonucleotides.

In some embodiments, step (ii) further includes contacting the second barcoding oligonucleotides with a second primer set including a nucleotide sequence that is complementary to the second adapter sequence of the second set of barcoding oligonucleotides.

In some embodiments, the nucleic acid fragments are not amplified during step (b).

In some embodiments, the first and second barcoding oligonucleotides include hairpin barcoding oligonucleotides.

In some embodiments, the DNA is a double-stranded DNA (dsDNA) fragment.

In some embodiments, the first and second barcodes each includes a degenerate nucleotide sequence.

In some embodiments, the first and second barcodes each includes a partially degenerative nucleotide sequence.

In some embodiments, the degenerate sequence includes 8-50 nucleotides. In some embodiments, the degenerate sequence includes 8-20 nucleotides.

In some embodiments, the set of first and set of second barcoding oligonucleotides consist of pooled barcoding oligos with multiple different defined sequences.

In some embodiments, the set of first and set of second barcoding oligonucleotides consist of pooled barcoding oligos with multiple different defined sequences

In some embodiments, the first and second barcodes each includes 8-50 nucleotides.

In some embodiments, the two consensus regions of the first barcoding oligonucleotides flank the first barcode.

In some embodiments, the two consensus regions of the second barcoding oligonucleotides flank the second barcode.

In some embodiments, the nucleotide sequence of the first or second barcode is positioned between the nucleotide sequences of the two consensus regions.

In some embodiments, the degenerate sequence of each first and second barcode is distinguishable from one another.

In some embodiments, the first barcode of the barcoding oligonucleotides within the first set of barcoding oligonucleotides is distinguishable from other first barcodes of the first set of barcoding oligonucleotides by its nucleotide sequence.

In some embodiments, the second barcode of the barcoding oligonucleotides within the second set of barcoding oligonucleotides is distinguishable from other second barcode of the second set of barcoding oligonucleotides by its nucleotide sequence.

In some embodiments, said contacting includes contacting the cell suspension or tissue slices with the first and second set of barcoding oligonucleotides at a concentration such that each cell within the cell suspension or tissue slice includes a first and second barcoding oligonucleotide that is distinguishable from a first and second barcoding oligonucleotide of a different cell. In some embodiments, the concentration ranges from 100 fM to 1 μM. In some embodiments, the concentration ranges from 1 pM to 10 pM.

In some embodiments, said contacting includes contacting the cell suspension or tissue slices with the first and second set of barcoding oligonucleotides at a concentration such that each cell within the cell suspension or tissue slice includes 2-1000 barcoding oligonucleotides. In some embodiments, a cell within the cell suspension or tissue slice includes less than 5% of barcoding oligonucleotides with the same first and second barcode as a different cell within the cell suspension. In some embodiments, a cell within the cell suspension or tissue slice does not include the first and second barcode that is the same first and second barcode of a second cell within the cell suspension or tissue slice.

In some embodiments, the nucleic acid fragment is a DNA amplicon product.

In some embodiments, the nucleic acid fragment is a DNA product of ligation.

In some embodiments, the method includes ligating a consensus read region including a first 5′ read region and a consensus read region including a second 5′ read region to a DNA fragment using a Y-adapter, a hairpin adapter, or a duplex adapter.

In some embodiments, the nucleic acid fragment is a DNA product of tagmentation.

In some embodiments, the DNA fragment includes genomic DNA (gDNA) modified to contain a first consensus read region at the 5′ end of the DNA sequence and a second consensus read region at the 5′ end of the DNA sequence.

In some embodiments, the nucleic acid fragments in step (a) include: a 5′ consensus read region; a 3′ consensus read region; and a target region. In some embodiments, (i) the 5′ consensus read region is a read1 sequence or a reverse complement thereof and the 3′ consensus read region is a read2 sequence or a reverse complement thereof or (ii) the 5′ consensus read region is a read2 sequence or a reverse complement thereof and the 3′ consensus read region is a read1 sequence or a reverse complement thereof.

In some embodiments, (i) the adapter sequence of the first set of oligonucleotides includes a P5 adapter sequences or a reverse complement thereof, and the adapter sequence of the second set of oligonucleotides includes a P7 adapter sequences or a reverse complement thereof, or (ii) the adapter sequence of the first set of oligonucleotides includes a P7 adapter sequences or a reverse complement thereof, and the adapter sequence of the second set of oligonucleotides includes a P5 adapter sequences or a reverse complement thereof.

In some embodiments, the method further includes, after step (c) contacting the amplicon product with a set of indexing primers, and performing an amplification reaction to produce a second set of amplicon products.

In some embodiments, the method includes lysing the cells containing the set of amplicon products. In some embodiments, the method includes lysing the cells containing the second set of amplicon products. In some embodiments, the method further includes contacting the second set of amplicon products with a third primer set including amplification primers, and performing an amplification reaction to produce a third set of amplicon products.

In some embodiments, the method further includes, after step (c), sequencing the DNA or RNA amplicon product to produce a barcoded sequenced library.

In some embodiments, the cell suspension includes 1000 cells or less. In some embodiments, the cell suspension includes 50 cells or less. In some embodiments, the cell suspension includes 5 cells or less. In some embodiments, the cell suspension includes a single cell. In some embodiments, the cell suspension is a single pool of cells. In some embodiments, the single pool is not divided into multiple pools of cells.

In some embodiments, the method is performed within individual cells of the single pool of the cells.

In some embodiments, the method further including: fragmenting nucleic acid within the permeabilized cell suspension or tissue slices to form the nucleic acid fragments; and ligating a consensus read region to one or both ends of the nucleic acid fragments.

In some embodiments, the consensus read region includes a 5′ read region. In some embodiments, the 5′ read region includes a read1 sequence or a read2 sequence. In some embodiments, the fragmenting and ligating steps are performed in a first buffer and the introducing step (a) and the amplifying steps (b) and (c) are performed in a second buffer.

In some embodiments, the method includes conducting a buffer exchange and cell washing step, where the first buffer is removed and replaced with a second buffer.

In some embodiments, the fragmenting and ligating steps are performed in a first set of reagents and the introducing step (a) and the amplifying steps (b) and (c) are performed in a second set of reagents.

In some embodiments, conducting a cell washing step, where the first set of reagents is removed and replaced with the second set of reagents.

In some embodiments, the method further includes, sequencing the amplicon products to produce a sequenced barcoded library including barcoding sequences for each cell within the cell suspension or tissue slices.

In another aspect, this disclosure features a method of generating primers from oligonucleotides using linear amplification, the method including:

(a) introducing to a reaction container:

(i) an oligonucleotide, where the oligonucleotide includes:

an amplification sequence, and

a consensus region that is complementary to a target sequence of a nucleic acid fragment; and

(b) amplifying, in the reaction container, the oligonucleotides to produce a primer including the reverse complement of the consensus region.

In some embodiments, the introducing step (a) further includes introducing an amplification primer including a consensus region that is complementary to the amplification sequence on the oligonucleotide.

In some embodiments, the introducing step (a) further includes introducing a second oligonucleotide, where the second oligonucleotide includes: a second amplification sequence, and a second consensus region that is complementary to a second target sequence of a nucleic acid fragment.

In some embodiments, the introducing step (a) further includes introducing a second amplification primer including a consensus region that is complementary to the second amplification sequence on the second oligonucleotide.

In some embodiments, the amplifying step (b) further includes amplifying, in the reaction container, the second oligonucleotide to produce a second primer including the reverse complement of the second consensus region.

In some embodiments, (i) the amplification sequence of the first oligonucleotide includes a first adapter sequence and the second amplification sequence includes a second adapter sequence or

(ii) the amplification sequence includes a second adapter sequence and the amplification sequence includes the first adapter sequence.

In some embodiments, (i) the adapter sequence of the first set of oligonucleotide includes a P5 adapter sequence, and the adapter sequence of the second set of oligonucleotide includes a P7 adapter sequence or (ii) the adapter sequence of the first set of oligonucleotide includes a P7 adapter sequences, and the adapter sequence of the second set of oligonucleotide includes a P5 adapter sequences.

In some embodiments, the oligonucleotide, the second oligonucleotide, or both is linear.

In some embodiments, the oligonucleotide, the second oligonucleotide, or both, further include a nick endonuclease recognition site or a reverse complement of a nick endonuclease recognition site.

In some embodiments, the oligonucleotide, the second oligonucleotide, or both, further includes at least one barcode.

In some embodiments, the first oligonucleotide, the second oligonucleotide, or both, include from 5′ to 3′: (a) a consensus region, a barcode, an amplification sequence, and a nick endonuclease recognition sequence, or any combination or orientation thereof; or (b) a consensus region, a barcode, an amplification sequence, and a reverse complement of a nick endonuclease recognition sequence, or any combination or orientation thereof.

In some embodiments, the oligonucleotide, second oligonucleotide, or both, further include a stem loop sequence.

In some embodiments, the oligonucleotide, the second oligonucleotide, or both, further includes at least one barcode.

In some embodiments, the oligonucleotide, second oligonucleotide, or both, further include a nick endonuclease recognition sequence, a reverse complement of a nick endonuclease recognition sequence, or both.

In some embodiments, the oligonucleotide, second oligonucleotide, or both include from 5′ to 3′: (a) a consensus region, a barcode, an amplification sequence, a nick endonuclease recognition sequence, and a stem loop sequence, or any combination or orientation thereof; or (b) a consensus region, a barcode, an amplification sequence, a nick endonuclease recognition site, a stem loop sequence, and a reverse complement of a nick endonuclease recognition sequence, or any combination or orientation thereof.

In some embodiments, the amplification primer, the second amplification primer, or both, further include a nick endonuclease recognition site.

In some embodiments, the amplification primer includes from 5′ to 3′: a nick endonuclease recognition site and a nucleotide sequence that is complementary to the amplification sequence on the oligonucleotide.

In some embodiments, the second amplification primer, includes from 5′ to 3′: a nick endonuclease recognition site and a nucleotide sequence that is complementary to the second amplification sequence on the second oligonucleotide.

In some embodiments, the oligonucleotide and the amplification primer are annealed prior to introducing into the reaction container.

In some embodiments, the oligonucleotide and the amplification primer are not annealed prior to introducing into the reaction container.

In some embodiments, the second oligonucleotide and the second amplification primer are annealed prior to introducing into the reaction container.

In some embodiments, the second oligonucleotide and the second amplification primer are not annealed prior to introducing into the reaction container.

In some embodiments, the amplifying step (b) includes amplifying via isothermal amplification, the oligonucleotides to produce the primers.

In some embodiments, the isothermal amplification is performed using an isothermal polymerase.

In some embodiments, the isothermal polymerase is selected from Klenow Fragment (Exo−), Bsu Large Fragment, Bst DNA polymerase, Bst2.0, Sequenase, Bsm DNA Polymerase, EquiPhi29, and Phi29 DNA polymerase.

In some embodiments, the amplifying in step (b) is performed under conditions that allow for primer invasion.

In some embodiments, the amplifying in step (b) further includes a nick endonuclease.

In some embodiments, the nick endonuclease is selected from nt.BspQI, nt.CviPII, nt.BstNBI, nb.BsrDI, nb.BtsI, nt.AlwI, nb.BbvcI, nt.BbvcI, nb.BsmI, nb.BssSI, nt.BsmAI, nb.Mva1269I, nb.Bpu10I, and nt.Bpu10I.

In some embodiments, the amplifying in step (b) is performed under conditions that allow for both nicking via the nick endonuclease binding to the nick endonuclease recognition site (and nicking) and amplification to generate the primers.

In some embodiments, the amplifying in step (b) includes amplifying via a thermostable polymerase and temperature cycling, the first oligonucleotides, second oligonucleotides, or both, to generate the primers.

In some embodiments, the thermostable polymerase is selected from a DNA polymerase, a RNA polymerase, an RNA-dependent DNA polymerase, or a DNA-dependent RNA polymerase.

In some embodiments, the method further including:

(c) contacting nucleic acid fragments with the first primer including the consensus region, the second primer including the second consensus region, or both; and

(d) amplifying the nucleic acid fragments with first primer, second primer, or both, to produce a set of amplicon products, where the set of amplicon products include:

(i) the amplification sequence or the reverse complement thereof, the targeting sequence or the reverse complement thereof, and all or a portion of the nucleic acid fragment,

(ii) the second amplification sequence or the reverse complement thereof, the second targeting sequence or the reverse complement thereof, and all or a portion of the nucleic acid fragment, or

(iii) the amplification sequence or the reverse complement thereof, the targeting sequence or the reverse complement thereof, all or a portion of the nucleic acid fragment, the second targeting sequence or a reverse complement thereof, the second amplification sequence or the reverse complement thereof.

In some embodiments, the method further including prior to step (c) the nucleic acid fragment is labeled with one or more adapter sequences.

In some embodiments, the targeting sequence of the first primer is complementary to the one or more adapter sequences.

In some embodiments, the targeting sequence of the second primer is complementary to the one or more adapter sequences.

In some embodiments, the targeting sequence of the first primer is complementary to a first strand of a nucleic acid fragment.

In some embodiments, the second targeting sequence is complementary to a second strand of the same nucleic acid fragment.

In some embodiments, the second targeting sequence is complementary to a first strand of a different nucleic acid fragment.

In some embodiments, the targeting sequence of the first primer, the second targeting sequence, or both, are complementary to an R1 adapter sequence or an R2 adapter sequence.

In some embodiments, the targeting sequence of the first primer, the second targeting sequence, or both, are complementary to a DNA fragment.

In some embodiments, the DNA fragment is selected from a DNA amplicon product, a DNA product of tagmentation, a DNA product of a ligation, and genomic DNA.

In some embodiments, the nucleic acid fragments in step (a) includes: a 5′ consensus read region; a 3′ consensus read region; and a target region.

In some embodiments, the reaction container is selected from a cell (in situ), a subcellular compartment (e.g., nucleus, cytoplasm), a tube, a well, a partition, a solution, and a droplet. In some embodiments, the reaction container is a pool of cells. In some embodiments, the reaction container is a cell. In some embodiments, the reaction container is a partition.

In some embodiments, the method further includes, after contacting the amplicon product with a set of indexing primers, and performing an amplification reaction to produce a second set of amplicon products.

In another aspect, this disclosure features a cell barcoding kit including:

(a) a first set of barcoding oligonucleotides, each barcoding oligonucleotide including:

a first barcode;

two consensus regions, where the two consensus regions of each barcoding primer includes:

one of the two consensus regions includes a nucleotide sequence that is complementary to a 5′ read region of a first strand of one of the DNA or RNA fragments, and

the second of the two consensus regions includes a first adapter sequence;

(b) a second set of barcoding oligonucleotides, each barcoding oligonucleotide including:

a second barcode;

two consensus regions, where the two consensus regions of each barcoding primer includes:

one of the two consensus regions includes a nucleotide sequence that is complementary to a 5′ read region of a second strand of one of the DNA or RNA fragments, and

the second of the two consensus regions includes a second adapter sequence.

In some embodiments, each of the first barcoding oligonucleotides is annealed to a first primer including a nucleotide sequence that is complementary to the first adapter sequence of the first barcoding oligonucleotide.

In some embodiments, each of the second barcoding oligonucleotides is annealed to a second primer including a nucleotide sequence that is complementary to the second adapter sequence of the second barcoding oligonucleotide.

In some embodiments, the first and second barcoding oligonucleotides are hairpin oligonucleotides.

In some embodiments, the first barcoding oligonucleotides each further include a first cleavage site, and where the second barcoding oligonucleotides each further include a second cleavage site.

In some embodiments, the first primer further includes a third cleavage site that is complementary to the first cleavage site of the first barcoding oligonucleotides, and where the second primer further includes a fourth cleavage site that is complementary to the second cleavage site of the second barcoding oligonucleotides.

In some embodiments, the one or more enzymes is selected from one or more of: DNA polymerase, RNA polymerase, nicking enzyme, a Bst2.0 polymerase, a Phi29 polymerase, an enzymatic fragmentation enzyme, an End Repair A-tail enzyme, a DNA ligase, or a combination thereof.

In some embodiments, the kit further includes one or more buffers selected from: a lysis buffer, an enzyme fragmentation buffer, an End Repair A-tail buffer, a ligation buffer, buffer 3.0, buffer 3.1, PCR amplification buffer, isothermal amplification buffer, and a combination there.

In some embodiments, the barcode includes a degenerate nucleotide sequence.

In some embodiments, the barcode includes 8-50 nucleotides.

In another aspect, this disclosure features a cell barcoding composition including:

(a) cell suspension or tissue slices including nucleic acid fragments;

(b) a first primer set including barcoding primers configured to bridge and extend from the 5′ region of the nucleic acid fragments;

where each first barcoding primer includes:

a first barcode or a reverse complement thereof;

a first consensus region or a reverse complement thereof including a nucleotide sequence that is complementary to a 5′ read region of a first strand of one of the nucleic acid fragments, and

a second consensus region or a reverse complement thereof including a first adapter sequence;

(c) a second primer set including barcoding primers configured to bridge and extend from the 5′ region of the opposite strand of the nucleic acid fragments,

where each second barcoding primer includes:

a second barcode or a reverse complement thereof;

a second consensus region or a reverse complement thereof including a nucleotide sequence that is complementary to a 5′ read region of a second strand of one of the nucleic acid fragments, and

a second consensus region or a reverse complement thereof including a second adapter sequence;

where the first and second barcoding primer sets do not amplify a target region of the nucleic acid sequences;

(d) a third primer set including nucleotide sequences that are complementary to the first adapter sequence of the first primer set; and

(e) a fourth primer set including nucleotide sequences that are complementary to the second adapter sequence of the second primer set.

In some embodiments, the barcode includes a degenerate nucleotide sequence.

In some embodiments, the barcode includes 8-50 nucleotides.

In some embodiments, the DNA sequence is a DNA amplicon product.

In some embodiments, the nucleic acid sequence is a DNA product of ligation.

In some embodiments, the DNA sequence is selected from: a Y-adapter nucleotide sequence, a hairpin nucleotide sequence, and a duplex nucleotide sequence.

In some embodiments, the nucleic acid sequence is a product of tagmentation.

In some embodiments, the DNA sequence includes genomic DNA (gDNA).

In some embodiments, the nucleic acid sequence includes: a 5′ consensus read region;

a 3′ consensus read region; and a target region.

In another aspect, this disclosure features a composition including an amplification primer, an oligonucleotide, and a primer, where the primer is a capable of hybridizing to a consensus region of a nucleic acid fragment.

In another aspect, this disclosure features a kit including: (a) an oligonucleotide, where the oligonucleotide includes: an amplification sequence, and a consensus region that is complementary to a target sequence of a nucleic acid fragment.

In some embodiments, the method further including an amplification primer including a nucleotide sequence that is complementary to the amplification sequence on the oligonucleotide.

In another aspect, this disclosure features a kit including:

(a) an oligonucleotide, where the oligonucleotide includes:

an amplification sequence, and

a consensus region that is complementary to a target sequence of a nucleic acid fragment; and

(b) a second oligonucleotide, where the second oligonucleotide includes:

a second amplification sequence, and

a second consensus region that complementary to a target sequence of a nucleic acid fragment.

In some embodiments, the method further including:

(c) a first amplification primer including a nucleotide sequence that is complementary to the amplification sequence on the oligonucleotide

(d) a second amplification primer including a nucleotide sequence that is complementary to the second amplification sequence on the second oligonucleotide.

In some embodiments, the oligonucleotide, the second oligonucleotide, or both is linear.

In some embodiments, the oligonucleotide, the second oligonucleotide, or both, further include a nick endonuclease recognition site or a reverse complement of a nick endonuclease recognition site

In some embodiments, the oligonucleotide, the second oligonucleotide, or both, further includes at least one molecular cellular label.

In some embodiments, the first oligonucleotide, the second oligonucleotide, or both, include from 5′ to 3′:

(a) a consensus region, a barcode, an amplification sequence, and a nick endonuclease recognition sequence, or any combination or orientation thereof; or

(b) a consensus region, a barcode, an amplification sequence, and a reverse complement of a nick endonuclease recognition sequence, or any combination or orientation thereof.

In some embodiments, the oligonucleotide, second oligonucleotide, or both, further include a stem loop sequence.

In some embodiments, the oligonucleotide, the second oligonucleotide, or both, further includes at least one barcode.

In some embodiments, the oligonucleotide, second oligonucleotide, or both, further include a nick endonuclease recognition sequence, a reverse complement of a nick endonuclease recognition sequence, or both.

In some embodiments, the oligonucleotide, second oligonucleotide, or both include from 5′ to 3′:

(a) a consensus region, a barcode, an amplification sequence, a nick endonuclease recognition sequence, and a stem loop sequence, or any combination or orientation thereof; or

(b) a consensus region, a barcode, an amplification sequence, a nick endonuclease recognition site, a stem loop sequence, and a reverse complement of a nick endonuclease recognition sequence, or any combination or orientation thereof.

In some embodiments, the first and second oligonucleotides are hairpin oligonucleotides.

In some embodiments, the amplification primer, the second amplification primer, or both, further include a nick endonuclease recognition site.

In some embodiments, the amplification primer, the second amplification primer, or both include from 5′ to 3′: a nick endonuclease recognition site and a nucleotide sequence that is complementary to the amplification sequence on the oligonucleotide.

In some embodiments, the kit further includes one or more enzymes.

In some embodiments, the one or more enzymes is selected from one or more of: DNA polymerase, RNA polymerase, nicking enzyme, a Bst2.0 polymerase, a Phi29 polymerase, an enzymatic fragmentation enzyme, an End Repair A-tail enzyme, a DNA ligase, or a combination thereof.

In some embodiments, the kit further includes one or more buffers selected from: a lysis buffer, an enzyme fragmentation buffer, an End Repair A-tail buffer, a ligation buffer, buffer 3.0, buffer 3.1, PCR amplification buffer, isothermal amplification buffer, and a combination there.

In some embodiments, the kit further includes a polymerase chain reaction (PCR) buffer.

In some embodiments, the kit further includes a deoxynucleotide triphosphates (dNTPs) buffer.

In another aspect, this disclosure features a composition including a first oligonucleotide and a second oligonucleotide, where:

the first oligonucleotide includes, from 5′ to 3′: (i) the reverse complement of the 5′ terminus of a sequence to be amplified; (ii) a barcode sequence; and (iii) an adapter sequence;

and

the second oligonucleotide includes the reverse complement of (iii).

In another aspect, this disclosure features a composition composition including a first oligonucleotide and a second oligonucleotide, where:

the first oligonucleotide includes, from 5′ to 3′: (i) the reverse complement of the 5′ terminus of a sequence to be amplified; (ii) a barcode sequence; and (iii) an adapter sequence; and

the second oligonucleotide includes, from 5′ to 3′: (iv) ERS′; and (v) the reverse complement of (iii).

In some embodiments, the first and second oligonucleotides are hybridized to each other.

In another aspect, this disclosure features a composition including a first hairpin oligonucleotide and a second hairpin oligonucleotide, where:

the first hairpin oligonucleotide includes, from 5′ to 3′: (i) the reverse complement of the 5′ terminus of the sense strand of a double-stranded DNA sequence to be amplified; (ii) a barcode sequence; (iii) an adapter sequence, (iv) a hairpin structure which includes the reverse complement of a nickase recognition sequence, a linker sequence, and the nickase recognition sequence, where the 3′ end of the hairpin structure can act as a primer for generating the reverse complement copies of (iii), (ii), and (i);

the second hairpin oligonucleotide includes, from 5′ to 3′: (v) the reverse complement of the 5′ terminus of the antisense strand of a double-stranded DNA sequence to be amplified; (vi) a barcode sequence; (vii) an adapter sequence; (viii) a hairpin structure which includes the reverse complement of a nickase recognition sequence, a linker sequence, and the nickase recognition sequence, where the 3′ end of the hairpin structure can act as a primer for generating the reverse complement copies of (vii), (vi), and (v).

In another aspect, this disclosure features a composition including a first, second, third and fourth oligonucleotide, where:

the first oligonucleotide includes, from 5′ to 3′: (i) the reverse complement of the 5′ terminus of the sense strand of a double-stranded DNA sequence to be amplified; (ii) a barcode sequence; and (iii) an adapter sequence; and the second oligonucleotide includes the reverse complement of (iii);

the third oligonucleotide includes, from 5′ to 3′: (iv) the reverse complement of the 5′ terminus of the antisense strand of a double-stranded DNA sequence to be amplified; (v) a barcode sequence; and (vi) an adapter sequence; and the fourth oligonucleotide includes the reverse complement of (vi).

In some embodiments, (a) the first and second oligonucleotides are hybridized to each other; and/or (b) the third and fourth oligonucleotides are hybridized to each other.

### BRIEF DESCRIPTION OF THE DRAWINGS

FIG. 1 provides an overview of the PCR amplification workflow for cellular barcoding in situ using linear barcoding oligonucleotides. Inputs into the PCR reaction include: A: In Situ Insert Library with Consensus regions (CR1 and CR2) appended to DNA; B. Barcode oligonucleotides 5′-CR1′-DS (degenerate sequence)-CR3′-3′ (provided in restricted amounts) and barcode amplification primer 5′-CR3-3′ (provided in excess); and C. Barcode oligonucleotides 5′-CR2′-DS-CR4′-3′ (provided in restricted amounts) and barcode amplification primer 5′-CR4-3′ (provided in excess). The products from the PCR reaction include: D: in situ insert library containing two DS regions each surrounded by two consensus regions. Barcoding primers are generated in the first round of amplification as well as in subsequent rounds. Barcoding primers are used in the second round of amplification to bind and amplify the in situ insert library, thereby producing D. Production of this in situ insert library may require multiple cycles of PCR, and some side products containing one or both of the barcoding oligo sequences may be possible.

FIG. 2A shows the workflow of the isothermal amplification and PCR workflow for cellular barcoding in situ using linear barcoding oligonucleotides. Inputs of the Isothermal amplification reaction include: A. In Situ Insert Library with Consensus regions (CR1 and CR2) appended to DNA; B. Annealed isothermal amplification primer set 1, that includes a barcode oligonucleotide 5′-CR1′-DS (degenerate sequence)-CR3′-3′ and barcode amplification primer 5′-ERS (endonuclease recognition site)-CR3-3′; C. Annealed isothermal amplification primer set 2, that includes barcode oligonucleotide 5′-CR2′-DS-CR4′-3′ and barcode amplification primer 5′-ERS-CR4-3′; and the nicking enzyme and isothermal DNA polymerase. The products that come out of the isothermal amplification reaction include: D. In Situ Insert Library with Consensus regions appended to DNA, exactly same as A; E Amplified barcode oligonucleotide set 1, generated via isothermal amplification of the annealed isothermal amplification primer set 1 (B), where the barcode oligonucleotide extends through the ERS and the barcode amplification primer extends through the DS and CR1 regions. The nicking enzyme can cleave (repeatedly) the top strand of the ERS and allow the isothermal amplification enzyme to extend the ERS over the barcode oligo; F Amplified barcode oligonucleotide set 2, generated via isothermal amplification of the annealed isothermal amplification primer set 2 (C), where the barcode oligonucleotide extends through the ERS and the barcode amplification primer extends through the DS and CR2 regions. The nicking enzyme can cleave (repeatedly) the top strand of the ERS and allow the isothermal amplification enzyme to extend the ERS over the barcode oligo. FIG. 2A describes the next step requiring PCR Amplification on the cells that have undergone isothermal amplification of the barcoding oligonucleotides. The inputs include cells containing the products from FIG. 2A, and the outputs include complete libraries with two sets of degenerate sequences, both surrounded by consensus regions.

FIG. 2B shows barcoding oligonucleotides provided as hairpin oligonucleotides that are used in the workflow of the isothermal amplification and PCR workflow for cellular barcoding in situ. In a non-limiting example, the hairpin barcoding oligonucleotides B and C are used as alternative to B and C from FIG. 2A. Hairpin B (left panel) includes from 5′ to 3′: CR1′-DS-CR3′-ERS' (reverse complement of the endonuclease recognition sequence)-stem loop-ERS-3′. Hairpin C (right panel) includes from 5′ to 3′: CR2′-DS-CR4′-ERS′-stem loop-ERS-3′.

FIG. 3A-3C provides tables of barcoding sequence input concentrations and lengths and how the input amount and length of barcode play together to limit multiple copies of a unique degenerate sequence (DS) from getting into the overall PCR reaction and thus multiple cells.

FIG. 4 provides two types of pruning to create cell clusters, depending on sequencing depth of sample.

FIG. 5A shows amplified libraries run on a Tapestation HSD1000 (Agilent). Left two lanes show replicates of gDNA controls (i.e., not amplified using barcoding primers) (“gDNA SOP”). Right two lanes show replicates of amplification products from the second PCR amplification using barcoding primer-mediated amplification of the genomic DNA amplicons from PCR1 (“gDNA BA”).

FIG. 5B shows quantification of the Tapestation run from FIG. 5A, plotting Sample Intensity (Normalized FU) for the indicated sizes (bp).

FIG. 6 shows a gel of in vitro amplification of barcode oligonucleotides for the different conditions denoted as lanes 1-10 and described in the figure.

FIG. 7A shows amplified libraries run on a Tapestation HSD1000 (Agilent). Left two lanes show two replicates of “in situ control(s)” from a first in situ amplification using targeting primers and a second amplification using P5/P7 amplification. Right two lanes show amplification products from a first in situ amplification using targeting primers followed by a second amplification using barcoding primers generated from barcode oligonucleotides.

FIG. 7B shows quantification of Tapestation run from FIG. 7A, plotting Sample Intensity (Normalized FU) for the indicated sizes.

FIG. 8A shows a gel image from an in situ cell barcoding sample (Agilient Tapestation HSd5000).

FIG. 8B shows an electrophoretogram of the same sample of FIG. 8A.

FIG. 8C shows the base composition of index 1; low complexity bases at base 6, 7, 13, 14, 20, 21, 27, 28, 29, and 30 correspond to non-degenerate bases in the P7 cell barcoding oligo. This shows the correct formation of cell barcodes after sequencing.

FIG. 8D shows the base composition of index 2; low complexity bases at 1, 2, 8, 9, 15, 16, 22, 23, 29, and 30 correspond to non-degenerate bases in the P5 cell barcoding oligonucleotide. This shows the correct formation of cell barcodes after sequencing.

### DEFINITIONS

All publications, patents and patent applications cited herein, whether supra or infra, are hereby incorporated by reference in their entireties.

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a primer” includes a mixture of two or more such primers, and the like. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The terms “cytometry” and “flow cytometry” are also used consistent with their customary meanings in the art. In particular, the term “cytometry” can refer to a technique for identifying and/or sorting or otherwise analyzing cells. The term “flow cytometry” can refer to a cytometric technique in which cells present in a fluid flow can be identified, and/or sorted, or otherwise analyzed, e.g., by labeling them with fluorescent markers and detecting the fluorescent markers via radiative excitation. The terms “about” and “substantially” as used herein to denote a maximum variation of 10%, or 5%, with respect to a property including numerical values.

The practice of the present disclosure will employ, unless otherwise indicated, conventional methods of medicine, chemistry, biochemistry, immunology, cell biology, molecular biology and recombinant DNA techniques, within the skill of the art. Such techniques are explained fully in the literature. See, e.g., T Cell Protocols (Methods in Molecular Biology, G. De Libero ed., Humana Press; 2.sup.nd edition, 2009); C. W. Dieffenbach and G. S. Dveksler, PCR Primer: A Laboratory Manual (Cold Spring Harbor Laboratory Press; 2.sup.nd Lab edition, 2003); Next Generation Sequencing: Translation to Clinical Diagnostics (L. C. Wong ed., Springer, 2013); Deep Sequencing Data Analysis (Methods in Molecular Biology, N. Shomron ed., Humana Press, 2013); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current addition); Sambrook et al., Molecular Cloning: A Laboratory Manual (3.sup.rd Edition, 2001); Methods In Enzymology (S. Colowick and N. Kaplan eds., Academic Press, Inc.).

“Substantially purified” generally refers to isolation of a substance (compound, polynucleotide, oligonucleotide, protein, or polypeptide) such that the substance comprises the majority percent of the sample in which it resides. Typically, in a sample, a substantially purified component comprises 50%, 80%-85%, or 90-95% of the sample. Techniques for purifying polynucleotides, oligonucleotides, and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide or oligonucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50% sequence identity, at least about 75% sequence identity, at least about 80%-85% sequence identity, at least about 90% sequence identity, or at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST®, used with default parameters. For example, BLAST®N and BLAST®P can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank®+EMBL®+DDBJ+PDB+GenBank® CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA, cDNA, or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

A polynucleotide “derived from” a designated sequence refers to a polynucleotide sequence which comprises a contiguous sequence of approximately at least about 6 nucleotides, at least about 8 nucleotides, at least about 10-12 nucleotides, or at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

“Recombinant” as used herein to describe a nucleic acid molecule means a polynucleotide of genomic, cDNA, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

As used herein, a “solid support” refers to a solid surface such as a magnetic bead, latex bead, microtiter plate well, glass plate, nylon, agarose, acrylamide, and the like.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and antisense strands).

The terms “genomic loci,” “genomic location,” “genomic region,” and “genomic target” are used interchangeably and denote a nucleic acid molecule (i.e., genomic DNA) with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The nucleic acid molecule can be DNA or RNA.

The term “primer,” “amplification primer,” “barcoding primer,” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA, cDNA, or RNA polymerase and at suitable temperature, pH, metal concentration, and salt concentration. The primer is generally single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA, cDNA, or RNA synthesis.

The term “binding” as used herein, refers to any form of attaching or coupling two or more components, entities, or objects. For example, two or more components may be bound to each other via chemical bonds, covalent bonds, ionic bonds, hydrogen bonds, electrostatic forces, Watson-Crick hybridization, etc.

The terms “Polymerase chain reaction” or “PCR” as used herein, refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. PCR reaction volumes typically range from a few hundred nanoliters, e.g. 200 nL, to a few hundred μL L, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g., Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” or “first set of primers” in reference to a nested amplification reaction mean the primers used to generate a first amplicon, and “secondary primers” or “second set of primers” mean the one or more primers used to generate a second, or nested, amplicon. In some embodiments, “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

The term “amplicon” or “amplified product” or “amplicon product” refers to the amplified nucleic acid product of a PCR reaction or other nucleic acid amplification process. The “amplicon product” refers to a segment of nucleic acid generated by an amplification process such as the PCR process or other nucleic acid amplification process such as ligation (e.g., ligase chain reaction). The terms are also used in reference to RNA segments produced by amplification methods that employ RNA polymerases, such as NASBA, TMA, etc. (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al., (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis. It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The “melting temperature” or “Tₘ” of double-stranded DNA is defined as the temperature at which half of the helical structure of DNA is lost due to heating or other dissociation of the hydrogen bonding between base pairs, for example, by acid or alkali treatment, or the like. The T.sub.m of a DNA molecule depends on its length and on its base composition. DNA molecules rich in GC base pairs have a higher T.sub.m than those having an abundance of AT base pairs. Separated complementary strands of DNA spontaneously reassociate or anneal to form duplex DNA when the temperature is lowered below the T.sub.m. The highest rate of nucleic acid hybridization occurs approximately 25 degrees C. below the T.sub.m. The T.sub.m may be estimated using the following relationship: T.sub.m=69.3+0.41(GC) % (Marmur et al. (1962) J. Mol. Biol. 5:109-118).

The term “barcode” refers to a nucleic acid sequence that is used to identify a single cell, subpopulation of cells, or sample. Barcode sequences can be linked to a target nucleic acid of interest during NGS library preparation and used to trace back the starting DNA, cDNA, or RNA fragment (starting insert) (e.g., products of PCR, tagmentation, ligation, or the like) to the cell or population from which the target nucleic acid originated. A barcode sequence can be added to a target nucleic acid of interest during amplification by carrying out PCR with a barcoding primer that contains a region comprising the barcode sequence and a region that is complementary to the target nucleic acid such that the barcode sequence is incorporated into the final amplified target nucleic acid product (i.e., amplicon). Barcodes can be included in either the forward primer or the reverse primer or both primers used in PCR to amplify a target nucleic acid. A barcode sequence can alternatively be added using a ligation-based technique. A barcode sequence can consist of specific nucleotides, degenerate nucleotides, or partially degenerate nucleotides, or a combination of the above.

The term “barcoding oligonucleotide” refers to a nucleic acid sequence that includes any one or more of the barcodes (e.g., cellular label(s), sample barcode(s), molecular label(s)) provided herein or known in the art or the reverse complement of any of the barcode (e.g., cellular label(s), sample barcode(s), molecular label(s)) provided herein or known in the art. The barcoding oligonucleotide are amplified using any of the methods described herein to produce one more of a set of barcoding products, including one or more barcoding primers.

The term “cell barcoding oligonucleotide” as used herein refers to a barcoding oligo intended to identify specific cells on their own or in combination with other “cell barcoding oligonucleotides.”

The term “non-barcoding oligonucleotide” as used herein refers an oligonucleotide that does not include a barcode sequence and that is amplified using any of the methods described herein to product one or more primers or one or more sets of primers.

The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; birds; and laboratory animals, including rodents such as mice, rats and guinea pigs, and the like. The term does not denote a particular age. Thus, both adult and newborn individuals are intended to be covered.

The term “Encode,” as used herein reference to a nucleotide sequence of nucleic acid encoding a gene product, e.g., a protein, of interest, is meant to include instances in which a nucleic acid contains a nucleotide sequence that is the same as the endogenous sequence, or a portion thereof, of a nucleic acid found in a cell or genome that, when transcribed and/or translated into a polypeptide, produces the gene product.

“Target nucleic acid” or “target nucleotide sequence,” as used herein, refers to any nucleic acid or nucleotide sequence that is of interest for which the presence and/or expression level in a single cell is sought using a method of the present disclosure. A target nucleic acid may include a nucleic acid having a defined nucleotide sequence (e.g., a nucleotide sequence encoding a cytokine), or may encompass one or more nucleotide sequences encoding a class of proteins.

“Originate,” as used in reference to a source of an amplified piece of nucleic acid, refers to the nucleic acid being derived either directly or indirectly from the source, e.g., a well in which a single T cell is sorted. Thus in some cases, the origin of a nucleic acid obtained as a result of a sequential amplification of an original nucleic acid may be determined by reading barcode sequences that were incorporated into the nucleic acid during an amplification step performed in a location that can in turn be physically traced back to the single T cell source based on the series of sample transfers that was performed between the sequential amplification steps.

The term “population”, e.g., “cell population” or “population of cells”, as used herein means a grouping (i.e., a population) of two or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. The cells of a cell population can be, but need not be, derived from more than one cell, i.e. non-clonal. The cells from which a non-clonal cell population may be derived may be related or unrelated and include but are not limited to, e.g., cells of a particular tissue, cells of a particular sample, cells of a particular lineage, cells having a particular morphological, physical, behavioral, or other characteristic, etc. A cell population can be any desired size and contain any number of cells greater than one cell. For example, a cell population can be 2 or more, 10 or more, 100 or more, 1,000 or more, 5,000 or more, 10⁴ or more, 10⁵ or more, 10⁶ or more, 10⁷ or more, 10⁸ or more, 10⁹ or more, 10¹⁰ or more, 10¹¹ or more, 10¹² or more, 10¹³ or more, 10¹⁴ or more, 10¹⁵ or more, 10¹⁶ or more, 10¹⁷ or more, 10¹⁸ or more, 10¹⁹ or more, or 10²⁰ or more cells.

A “heterogeneous” cell population may include one or more distinct cell populations, where each cell population contains cells that are phenotypically distinct from other cell populations.

As used herein, the term “reaction container” as used herein refers to the physical location of a reaction or where the reaction products are located following completion of the reaction. Non-limiting examples of reaction containers include: a tube, a well, a partition, a solution, a droplet, a cell (in situ), or a subcellular compartment (e.g., cytoplasm).

As used herein, the term “precursor library” refers to a library of nucleic acid sequences that undergoes further processing prior to next generation sequencing. Further processing includes, but is not limited to, amplification, fragmentation, tagmentation, ligation, barcoding-primer-mediated amplification, or any combination thereof. Typically precursor libraries have had one set of consensus regions appended to the flanking ends.

As used herein, the term “in situ library” refers to a library of nucleic acid sequences where preparation of the library occurred within a cell. A non-limiting example of in situ library preparation is described in PCT/US2021/046025 (WO2022/036273), which is herein incorporated by reference in its entirety.

As used herein, the term “rolling circle amplification” (RCA) refers to a polymerization reaction carried out using a single-stranded circular DNA (e.g., a circularized oligonucleotide) as a template and an amplification primer that is substantially complementary to the single-stranded circular DNA (e.g., the circularized oligonucleotide) to synthesize multiple continuous single-stranded copies of the template (e.g., multiple single strand copies of barcoding primers or a product thereof). RCA can include hybridizing one or more amplification primers to the circularized padlock oligonucleotide and amplifying the circularized padlock oligonucleotide using a DNA polymerase with strand displacement activity, for example Phi29 DNA polymerase.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the primer” includes reference to one or more primers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DETAILED DESCRIPTION

Aspects of the present disclosure relate generally to methods, compositions, and kits for barcoding individual cells within a cell population and identifying disease-associated genetic alterations of cell populations within the sample or individual cells. Aspects of the present disclosure also include a computer readable-medium and a processor to carry out the steps of the method described herein.

Aspects of the present methods include preparation of the sample and/or fixation of the cells of the sample performed in such a manner that the prepared cells of the sample maintain characteristics of the unprepared cells, including characteristics of unprepared cells in situ, i.e., prior to collection, and/or unfixed cells following collection but prior to fixation and/or permeabilization and/or labeling. Keeping cells intact during library preparation using the methods described herein preserves the natural structure of the cells during library preparation. In a non-limiting example, the present disclosure provides methods of performing whole cell barcoding where the method includes: (a) contacting nucleic acid fragments within a permeabilized cell suspension or tissue slices with: (i) a first set of barcoding oligonucleotides, each barcoding oligonucleotide including: a first barcode; two consensus regions, wherein the two consensus regions of each barcoding primer includes: one of the two consensus regions includes a nucleotide sequence that is complementary to a 5′ read region of a first strand of one of the DNA, cDNA, or RNA fragments, and the second of the two consensus regions includes a first adapter sequence; (ii) a second set of barcoding oligonucleotides, each barcoding oligonucleotides including: a second barcode; two consensus regions, wherein the two consensus regions of each barcoding primer includes: one of the two consensus regions includes a nucleotide sequence that is complementary to a 5′ read region of a second strand of one of the DNA, cDNA, or RNA fragments, and the second of the two consensus regions includes a second adapter sequence; (b) amplifying: the first set of barcoding oligonucleotides to produce a first set of barcoding primers; and the second set of barcoding oligonucleotides to produce a second set of barcoding primers; (c) amplifying the nucleic acid fragments with first and second set of barcoding primers to produce a set of amplicon products, wherein the set of amplicon products include the first barcoding primer bridging from the 5′ end of the nucleic acid fragments and the second barcoding primer bridging from the 5′ end of the opposite strand of the nucleic acid fragments.

Aspects of the present disclosure also relate to methods, compositions, and kits for amplifying primers from oligonucleotides using linear amplification in a reaction container (e.g., any of the reaction container described herein such as droplets, partitions, and wells). The amplified primers can then be used in downstream applications, including, but not limited to amplification of a nucleic acid sequence. In a non-limiting example, the present disclosure provides methods of generating primers from oligonucleotides using linear amplification where the method includes (a) introducing to a reaction container: (i) an oligonucleotide, wherein the oligonucleotide includes: an amplification sequence, and a consensus region that is at least partially complementary to a target sequence of a nucleic acid fragment; and (b) amplifying, in the reaction container, the oligonucleotides to produce a primer including the reverse complement of the consensus region.

Interrogating the genetic diversity of a tissue or organ (such as a heterogenous tissue or organ) is an emerging field, population “bulk” sequencing involving sampling a large group of cells from the population, extracting DNA, and whole-genome sequencing the entire pool to deep coverage cannot provide single cell detail. Methods are emerging that provide single cell resolution, however they rely on mechanically separating single cells to perform individual amplification reactions, or barcoding populations of cells using time intensive split and pool methods. The cellular barcoding method that described herein obviates these technologies and will allow genotypic tracking of cells for clonal fate mapping, lineage tracing, and high throughput screening. The cellular barcoding method that is described herein does not rely on or need physical isolation of individual cells for labeling single cell with sets of unique cell identifiers, instead it relies on the natural structure of each cell to provide barriers against the intermingling of nucleic acids (DNA, RNA, cDNA) or intracellular proteins from different cells. This method can be performed by splitting an individual population of cells into separate sub-populations of cells (containing 1 or more cells) and then re-combining the pools after cell barcoding is performed, however, it does not require splitting and re-combining to achieve single cell resolution. In fact, one advantage is that it can label DNA/RNA within the cells in a single reaction such that the DNA/RNA can grouped together based on which cell they are from.

Aspects of the present disclosure include methods for preparing barcoding sequences, such as for cellular barcoding in situ, methods for performing barcoding, such as whole cell barcoding of a cellular population (e.g. heterogeneous cell population) in situ, and methods of detecting disease-associated genetic alterations, such as of single cells within a population that were prepared in situ and sequenced.

The methods of the present disclosure include contacting a population, such as a heterogeneous population comprising nucleic acid sequences such as DNA, cDNA, or RNA sequences (e.g., a DNA, cDNA, or RNA insert), with barcoding sequences, for the purpose of extending or bridging cell specific barcoding primers to the ends of the target DNA or RNA sequences within each cell.

Thus, the starting sample for which the barcoding sequences come in contact with include DNA, cDNA, or RNA inserts within the cells which are previously prepared in situ (see e.g., section titled “Preparation of the cellular sample prior to cellular barcoding”). For example, DNA inserts can be prepared using a library prep method that maintains cell integrity during the NGS library preparation, and could be performed by amplifying adapter sequence to DNA, RNA or cDNA (generated by reverse transcription of RNA), ligation of adapters to the nucleic acids, or tagmentation to nucleic acids.

In the process of performing in situ cell barcoding, the following are non-limiting examples of products that may be created:
1. A collection of cells containing precursor libraries and barcoding oligonucleotides, which have the ability to hybridize to each other due to complementary sequences on their 5′ ends, but that cannot amplify each other because the hybridization product creates 3′ overhangs.
2. A collection of cells in which adapters containing one or more universal sequences (e.g., read1 sequence, read2 sequence, P5 sequence, and/or P7 sequence) and a barcode sequence (degenerate/partially degenerate, or set of defined sequences) are added to (e.g., both sides) of genomic fragments/amplicons/RNA/cDNA.
3. An NGS library including fragments with sequencing adapters (e.g., P5 and/or P7 sequences) in which the progeny of each unique molecule may or may not have the same pair of cellular barcodes.
   DNA and RNA Inserts within the Intact Cells

In some embodiments, the nucleic acid inserts (e.g., DNA, cDNA, or RNA inserts) within the cells can be products of PCR amplification (e.g., amplicons), products of ligation, for example, where single stranded DNA, Y-adapters, hairpins, or duplex DNA is ligated on products of tagmentation, reverse transcription, or other methods where genomic DNA (gDNA) or RNA is tagged with consensus read sequences extending from each end of the nucleic acid, and the like. These nucleic acid inserts (e.g., DNA, cDNA, or RNA insert) will contain a target nucleotide sequence region of interest. In some embodiments, the DNA is a double-stranded DNA (dsDNA) insert, a single stranded DNA (ssDNA) insert, and the like. In certain embodiments, the RNA insert is a reverse transcribed RNA fragment, a messenger RNA (mRNA), transfer RNA (tRNA), and ribosomal RNA (rRNA), guide RNA (gRNA), or a trans-activating crispr RNA (tracrRNA).

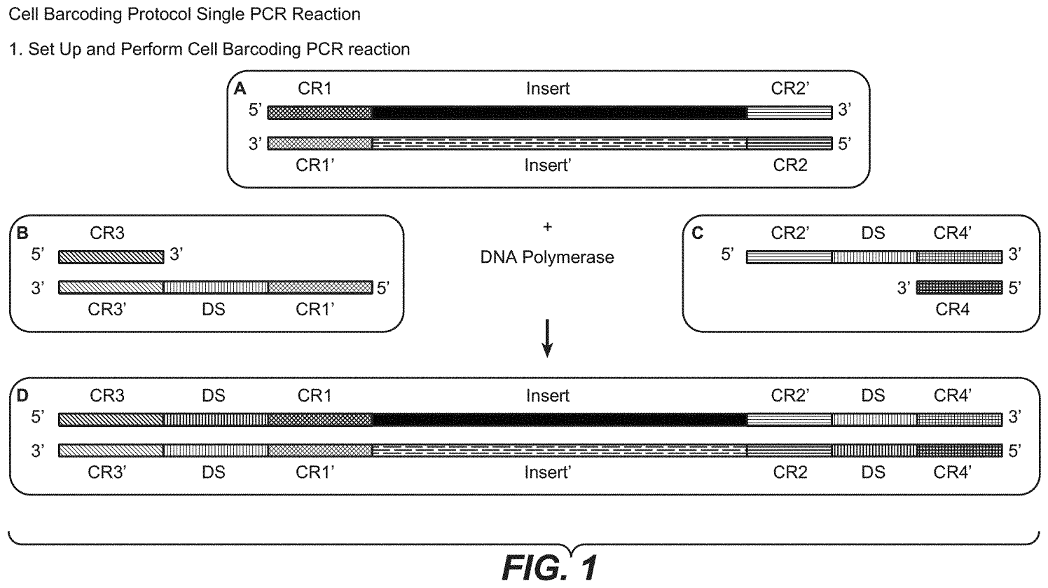

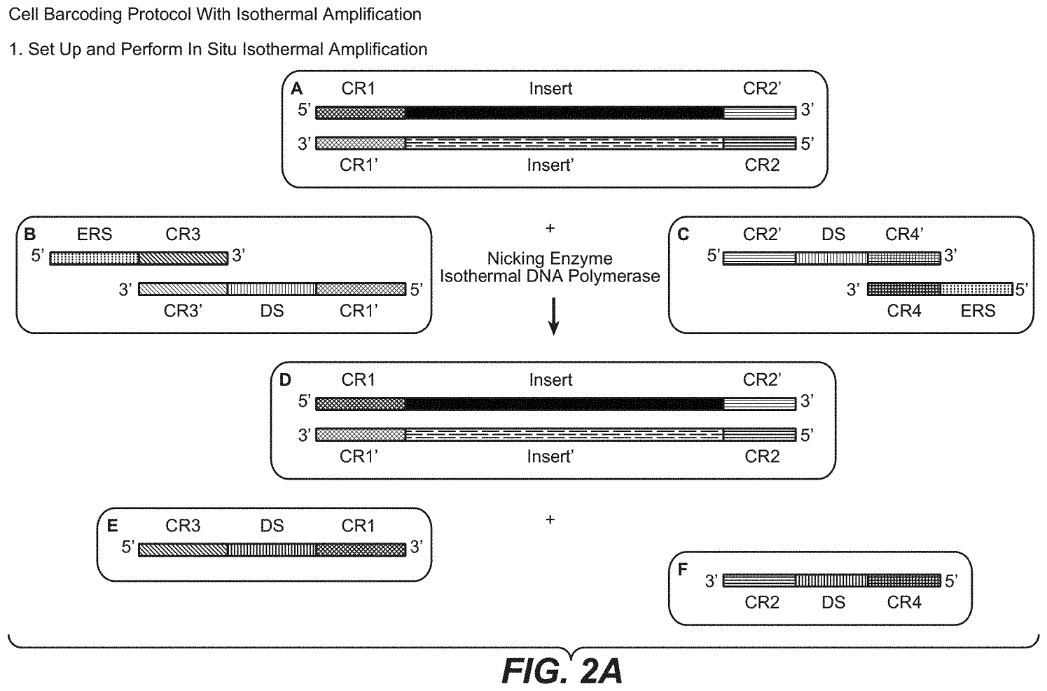

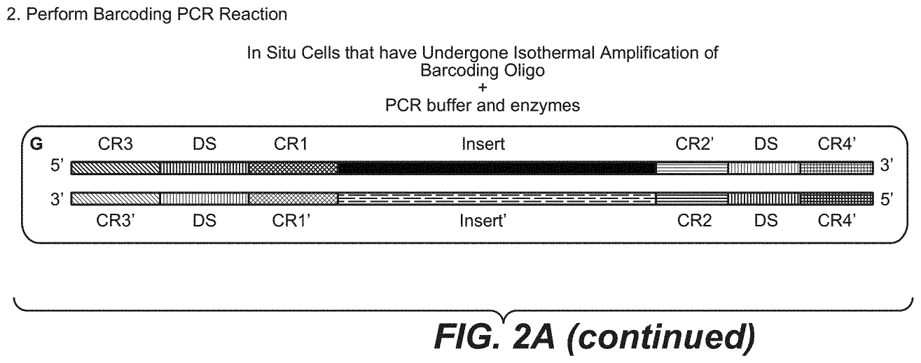

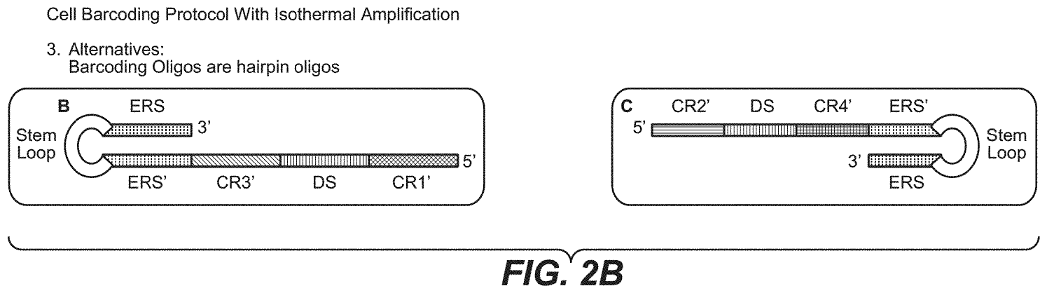

In some embodiments, nucleic acid fragments (e.g., DNA, cDNA, or RNA fragments) are prepared within the cell in situ using target amplification-based methods, or ligation-based methods, described herein, under the section “Preparation of the cellular sample prior to cellular barcoding”. The prepared nucleic acid inserts (e.g., DNA, cDNA, or RNA inserts (now, “DNA or RNA fragments”) will contain a consensus read (CR) sequence at each end of the DNA, cDNA, or RNA sequence, and a target nucleotide region (see e.g., insert of the input library of FIG. 1 OR FIG. 2A) positioned between the two consensus read regions (see e.g., CR1 and CR2′ of the input library of FIG. 1 or FIG. 2A). Thus, the consensus read regions flank the target region (insert). See, for example, part “A” in FIG. 1 or 2A. These consensus read regions are non-native to the genomic DNA, cDNA, or RNA sequence within the cell and are added prior to contacting the cells with the cell barcoding sequences.

If the starting fragment is a DNA fragment, the DNA fragment may be a double stranded DNA (dsDNA) fragment (e.g., within the cell) as shown in the example of FIG. 1 and FIG. 2. In such cases where a dsDNA insert (e.g., within the cell) is used as the starting sample, the dsDNA fragment can have a 5′ strand (e.g., first strand) of DNA with two consensus read regions (CR1 and CR2′) flanking the target nucleotide region (insert), and a 3′ strand (e.g., second strand) of DNA containing two consensus regions (CR1′ and CR2) flanking the target nucleotide region (insert′), which is complementary to the 5′ strand of DNA.

The consensus regions are added to the nucleic acid inserts (e.g., DNA inserts, cDNA inserts, or RNA inserts) using ligation based- and/or amplification-based techniques as described herein in “Preparation of the cellular sample prior to cellular barcoding.” In some embodiments, the consensus regions on the nucleic acid fragments (e.g., DNA, cDNA, or RNA fragments) can be sequencing primer sites that are binding sites for general sequencing primers. In some embodiments, the consensus regions on the nucleic acid fragments include a read/(R1) sequence or a read2 (R2) sequence.

After the nucleic acid fragments (e.g., DNA, cDNA, or RNA fragments) have been prepared within the cells in situ, the method of the present disclosure includes contacting the nucleic acid sequence fragments (e.g., DNA, cDNA, or RNA nucleotide sequence fragments) within the cells with sets of barcoding oligonucleotides.

### Barcoding Oligonucleotides and Non-Barcoding Oligonucleotides

In some embodiments, the barcoding oligonucleotides of the present disclosure include a first set of barcoding oligonucleotides, a second set of barcoding oligonucleotides, or both.

In some embodiments, for the first set of barcoding oligonucleotides, each oligonucleotide includes at least a first barcode (e.g., molecular cellular label (e.g., a degenerate sequence labeled as “DS” of FIGS. 1 and 2, part “B”)), and a consensus read region (e.g., CR1′ in part “B”) that is complementary to a consensus read region (e.g., CR1 in part “A”) of the nucleic acid fragment (e.g., DNA, cDNA, or RNA fragment). In some embodiments, each of the first barcoding oligonucleotides comprise two or more consensus regions (e.g., three or more, four or more, five or more, six or more, or seven or more). In certain embodiments, each oligonucleotide comprises at least two consensus regions (e.g., CR3′ and CR1′ of part “B” of FIGS. 1 and 2).

Similarly, for the second set of barcoding oligonucleotides, each oligonucleotide includes at least a second barcode (e.g., a molecular cellular label (e.g., a degenerate sequence labeled as “DS” of FIGS. 1 and 2, part “C”)), and a consensus read region (CR2′ in part “C”) that is complementary to a consensus read region of the nucleic acid fragment (e.g., DNA, cDNA, or RNA fragment). In some embodiments, each of the second barcoding oligonucleotides comprise two or more consensus regions (e.g., three or more, four or more, five or more, six or more, or seven or more). In certain embodiments, each oligonucleotide comprises at least two consensus region (e.g., CR2′ and CR4′ of part “C” of FIGS. 1 and 2).

In some embodiments, the total length of each of the barcoding oligonucleotides can range from, for example, 50-300 nucleotides. In some embodiments, the length of each barcoding oligonucleotide ranges from 50-300 nucleotides, such as 50-100 nucleotides 90-120 nucleotides, 50-150 nucleotides, 50-200 nucleotides, 50-250 nucleotides, 100-150 nucleotides, 90-150 nucleotides, 90-100 nucleotides, 90-110 nucleotides, 100-200 nucleotides, or 100-300 nucleotides. In certain embodiments, the length of each of the barcoding oligonucleotides is about 30 nucleotides, about 35 nucleotides, about 40 nucleotides about 45 nucleotides, about 50 nucleotides, the 55 nucleotides, about 60 nucleotides, about 65 nucleotides, about 70 nucleotides, about 75 nucleotides, about 80 nucleotides, about 85 nucleotides, about 95 nucleotides, about 100 nucleotides, about 105 nucleotides, about 110 nucleotides, about 115 nucleotides, about 120 nucleotides, about 125 nucleotides about 130 nucleotides, about 135 nucleotides, about 140 nucleotides, about 145 nucleotides, about 150 nucleotides, about 155 nucleotides, about 160 nucleotides, about 165 nucleotides, about 170 nucleotides, about 175 nucleotides, about 180 nucleotides, about 185 nucleotides, about 190 nucleotides, about 195 nucleotides, about 200 nucleotides, about 205 nucleotides, about 210 nucleotides, about 215 nucleotides, about 220 nucleotides, about 225 nucleotides, about 230 nucleotides, about 235 nucleotides, about 240 nucleotides, about 245 nucleotides, about 250 nucleotides, about 255 nucleotides, about 260 nucleotides, about 265 nucleotides, about 270 nucleotides, about 275 nucleotides, about 280 nucleotides, about 285 nucleotides, about 290 nucleotides, about 295 nucleotides, or about 300 nucleotides.

In certain embodiments, the length of each of the first set of barcoding oligonucleotides can range from, for example, 50-300 nucleotides. In some embodiments, the length of each of the first set of barcoding oligonucleotides ranges from 50-300 nucleotides, such as 50-100 nucleotides 90-120 nucleotides, 50-150 nucleotides, 50-200 nucleotides, 50-250 nucleotides, 100-150 nucleotides, 90-150 nucleotides, 90-100 nucleotides, 90-110 nucleotides, 100-200 nucleotides, or 100-300 nucleotides. In certain embodiments, the length of each of the first set of barcoding oligonucleotides is about 20 nucleotide, 25 nucleotides, 30 nucleotides, about 35 nucleotides, about 40 nucleotides about 45 nucleotides, about 50 nucleotides, the 55 nucleotides, about 60 nucleotides, about 65 nucleotides, about 70 nucleotides, about 75 nucleotides, about 80 nucleotides, about 85 nucleotides, about 95 nucleotides, about 100 nucleotides, about 105 nucleotides, about 110 nucleotides, about 115 nucleotides, about 120 nucleotides, about 125 nucleotides about 130 nucleotides, about 135 nucleotides, about 140 nucleotides, about 145 nucleotides, about 150 nucleotides, about 155 nucleotides, about 160 nucleotides, about 165 nucleotides, about 170 nucleotides, about 175 nucleotides, about 180 nucleotides, about 185 nucleotides, about 190 nucleotides, about 195 nucleotides, about 200 nucleotides, about 205 nucleotides, about 210 nucleotides, about 215 nucleotides, about 220 nucleotides, about 225 nucleotides, about 230 nucleotides, about 235 nucleotides, about 240 nucleotides, about 245 nucleotides, about 250 nucleotides, about 255 nucleotides, about 260 nucleotides, about 265 nucleotides, about 270 nucleotides, about 275 nucleotides, about 280 nucleotides, about 285 nucleotides, about 290 nucleotides, about 295 nucleotides, or about 300 nucleotides.

In certain embodiments, the length of each of the second set of barcoding oligonucleotides can range from, for example, 50-300 nucleotides. In some embodiments, the length of each of the second set of barcoding oligonucleotides ranges from 50-300 nucleotides, such as 50-100 nucleotides 90-120 nucleotides, 50-150 nucleotides, 50-200 nucleotides, 50-250 nucleotides, 100-150 nucleotides, 90-150 nucleotides, 90-100 nucleotides, 90-110 nucleotides, 100-200 nucleotides, or 100-300 nucleotides. In certain embodiments, the length of each of the second set of barcoding oligonucleotides is about 30 nucleotides, about 35 nucleotides, about 40 nucleotides about 45 nucleotides, about 50 nucleotides, the 55 nucleotides, about 60 nucleotides, about 65 nucleotides, about 70 nucleotides, about 75 nucleotides, about 80 nucleotides, about 85 nucleotides, about 95 nucleotides, about 100 nucleotides, about 105 nucleotides, about 110 nucleotides, about 115 nucleotides, about 120 nucleotides, about 125 nucleotides about 130 nucleotides, about 135 nucleotides, about 140 nucleotides, about 145 nucleotides, about 150 nucleotides, about 155 nucleotides, about 160 nucleotides, about 165 nucleotides, about 170 nucleotides, about 175 nucleotides, about 180 nucleotides, about 185 nucleotides, about 190 nucleotides, about 195 nucleotides, about 200 nucleotides, about 205 nucleotides, about 210 nucleotides, about 215 nucleotides, about 220 nucleotides, about 225 nucleotides, about 230 nucleotides, about 235 nucleotides, about 240 nucleotides, about 245 nucleotides, about 250 nucleotides, about 255 nucleotides, about 260 nucleotides, about 265 nucleotides, about 270 nucleotides, about 275 nucleotides, about 280 nucleotides, about 285 nucleotides, about 290 nucleotides, about 295 nucleotides, or about 300 nucleotides.

In some embodiments, the first and second set of barcoding oligonucleotides are single stranded oligonucleotides. In some embodiments, the first and second set of barcoding oligonucleotides are duplex oligonucleotides. In some embodiments, the first and second set of barcoding oligonucleotides are duplex oligonucleotides with overhangs. In some embodiments, the first and second set of barcoding oligonucleotides are single stranded oligonucleotides that can form a hairpin structure. In some embodiments the first set of barcoding oligonucleotides comprise circular ssDNA. In some embodiments, the first and second set of barcoding oligonucleotides are contacted with a first and second set of amplification primers to form barcoding primers before contacting the DNA, cDNA, or RNA fragments.

In certain embodiments, the first and second barcoding oligonucleotides can be amplified without addition of an amplification primer. In certain embodiments the duplex or partially duplex oligonucleotide (e.g., hairpin oligonucleotide) acts as its own amplification primer.

Non-limiting examples of the methods of the present disclosure are shown in FIG. 1 and FIG. 2.

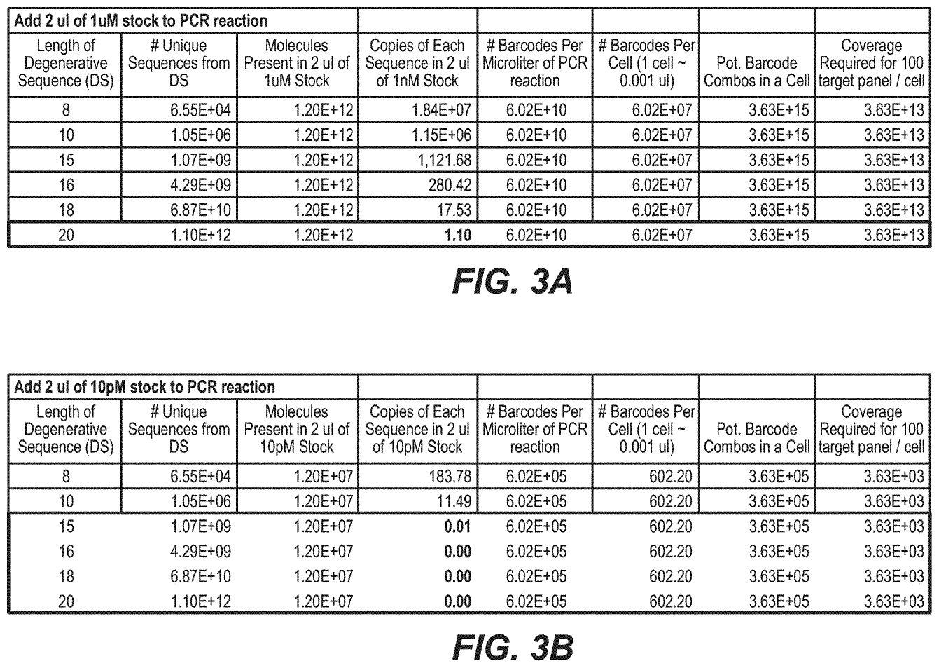

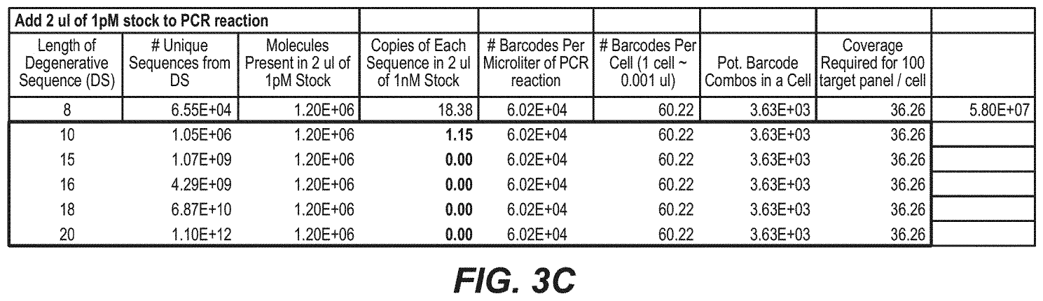

The concentration, volume, and sequence diversity of the first and second set of oligonucleotides are controlled such that there is a low probability that the same first barcoding sequence enters more than one cell and same second barcoding sequence enters more than one cell. For example, the tables of FIGS. 3A-3C shows how the combination of input amount and length of the barcodes, together, can limit multiple copies of a unique cellular label (e.g., degenerate sequence) from getting into the overall PCR reaction and thus multiple cells. Therefore, based on length of unique cellular label and volume and/or concentration of barcoding oligonucleotides used in the reaction as shown in FIGS. 3A-3C, it can be statistically unlikely that duplicates occur.

For example, 2 μl of a 1 μM barcoding oligonucleotide stock where the degenerate sequence is 20 bases, would have 1.1 copies of each barcode sequence. Therefore, it would be unlikely for two different cells in the same reaction to receive the same barcode sequence. However, if 2 μl of a 1 μM barcoding oligonucleotide stock with a degenerate sequence of 15 bases is used, then 1121.7 copies of each barcode sequence would be present in the reaction. In this case, some cells would likely have the same barcode sequence, resulting in reads from two different cells having the same barcode sequence.

Notably, the amplification of barcoding oligonucleotides will work even when the representation of each barcoding sequence is greater than 1.

In some embodiments, the methods provided herein include a non-barcoding oligonucleotide (e.g., an oligonucleotide that does not contain a barcode). In such cases, the primers produced following amplification of the oligonucleotides do not include a barcode sequence or a reverse complement thereof. In some embodiments where the oligonucleotide does not include a barcode, the oligonucleotide includes an amplification sequence and one or more consensus regions.

In some embodiments where the methods include a non-barcoding oligonucleotide, the first oligonucleotide includes an amplification sequence, and a consensus region that is complementary to a target sequence of a nucleic acid fragment; and a second oligonucleotide, wherein the second oligonucleotide comprises: a second amplification sequence (e.g., a primer binding sequence), and a second target sequence that is complementary to a second consensus region of a nucleic acid fragment. In some embodiments, the amplification sequence is at least partially complementary to all or part of an amplification primer. In some embodiments, a target sequence of a nucleic acid fragment includes a consensus region or reverse complement thereof. In some embodiments, the first target sequence is an antisense strand of a dsDNA and a second target sequence is a sense strand of dsDNA. In some embodiments, the amplification sequence is complementary to all or part of an amplification primer.

In some embodiments where the methods include a non-barcoding oligonucleotide, the length of the first non-barcoding oligonucleotide, non-barcoding second oligonucleotide or both is about 20 nucleotide, 25 nucleotides, 30 nucleotides, about 35 nucleotides, about 40 nucleotides about 45 nucleotides, about 50 nucleotides, the 55 nucleotides, about 60 nucleotides, about 65 nucleotides, about 70 nucleotides, about 75 nucleotides, about 80 nucleotides, about 85 nucleotides, about 95 nucleotides, about 100 nucleotides, about 105 nucleotides, about 110 nucleotides, about 115 nucleotides, about 120 nucleotides, about 125 nucleotides about 130 nucleotides, about 135 nucleotides, about 140 nucleotides, about 145 nucleotides, about 150 nucleotides, about 155 nucleotides, about 160 nucleotides, about 165 nucleotides, about 170 nucleotides, about 175 nucleotides, about 180 nucleotides, about 185 nucleotides, about 190 nucleotides, about 195 nucleotides, about 200 nucleotides, about 205 nucleotides, about 210 nucleotides, about 215 nucleotides, about 220 nucleotides, about 225 nucleotides, about 230 nucleotides, about 235 nucleotides, about 240 nucleotides, about 245 nucleotides, about 250 nucleotides, about 255 nucleotides, about 260 nucleotides, about 265 nucleotides, about 270 nucleotides, about 275 nucleotides, about 280 nucleotides, about 285 nucleotides, about 290 nucleotides, about 295 nucleotides, or about 300 nucleotides.

In some embodiments where the methods include a non-barcoding oligonucleotide, the first non-barcoding oligonucleotide, non-barcoding second oligonucleotide or both include an amplification sequence. In such cases, the amplification sequence is at least partially complementary to all or part of an amplification primer. The amplification primer can bind to the amplification sequence in the oligonucleotide and be used in a nucleic acid extension reaction (e.g., PCR or isothermal amplification) to produce an amplicon. In such cases, the resulting amplicon produced comprises the amplification primer and the reverse complement of the consensus region of the oligonucleotide. In some embodiments, the amplicon is a primer that is used to amplify a nucleic acid sequence (see, e.g., FIG. 1). In some embodiments, the amplification sequence comprises an adapter sequence (e.g., a P5 sequence or P7 sequence) or a reverse complement thereof. In some embodiments, the amplification sequence is CR3, CR3′, or a variation thereof. For example, the amplification sequence CR3′ is at least partially complementary to CR3 of an amplification primer (see, e.g., FIG. 1 except the oligonucleotide of B does not comprise a barcode (“DS”)). In some embodiments, the amplification sequence is CR4, CR4′, or a variation thereof. For example, the amplification sequence CR4′ is at least partially complementary to CR4 of an amplification primer (see, e.g., FIG. 1 except the oligonucleotide of C does not comprises a barcode (“DS”)).

In some embodiments where the methods include a non-barcoding oligonucleotide, the amplification sequence of the first oligonucleotide comprises a first adapter sequence and the second amplification sequence comprises a second adapter sequence or (the amplification sequence comprises a second adapter sequence and the amplification sequence comprises the first adapter sequence.

In some embodiments where the methods include a non-barcoding oligonucleotide, the first non-barcoding oligonucleotide, non-barcoding second oligonucleotide or both include one or more consensus regions. In such cases, the one or more consensus regions can include a nucleic acid sequence or a reverse complement thereof that is at least partially complementary to a 5′ consensus read region or a 3′ consensus read region on a nucleic acid sequence (e.g., a nucleic acid fragment). As described herein, upon amplification of the oligonucleotide using an amplification primer and a nucleic acid extension reaction (e.g., PCR or isothermal amplification), the resulting amplicon comprises the amplification primer and the reverse complement of the consensus region of the oligonucleotide. The reverse complement of the consensus region of the oligonucleotide enables hybridization to the 5′ consensus read region of 3′ consensus read region on the nucleic acid sequence (e.g., the nucleic acid fragment). In some embodiments, the one or more consensus regions includes an adapter sequence. In such cases, the adapter sequence of the first set of oligonucleotide comprises a P5 adapter sequence, and the adapter sequence of the second set of oligonucleotide comprises a P7 adapter sequence or the adapter sequence of the first set of oligonucleotide comprises a P7 adapter sequences, and the adapter sequence of the second set of oligonucleotide comprises a P5 adapter sequences.

In some embodiments where the methods include a non-barcoding oligonucleotide, the first non-barcoding oligonucleotide, non-barcoding second oligonucleotide or both are linear.

In some embodiments where the first oligonucleotide, second oligonucleotide, or both does not include a barcode, the oligonucleotide, the second oligonucleotide, or both, further comprise a nick endonuclease recognition site (ERS) or a reverse complement of a nick endonuclease recognition site.

In some embodiments where the methods include a non-barcoding oligonucleotide, the first non-barcoding oligonucleotide, non-barcoding second oligonucleotide or both, comprise from 5′ to 3′: (a) a consensus region, a barcode, an amplification sequence, and a nick endonuclease recognition sequence, or any combination or orientation thereof; or (b) a consensus region, a barcode, an amplification sequence, and a reverse complement of a nick endonuclease recognition sequence, or any combination or orientation thereof.

In some embodiments where the methods include a non-barcoding oligonucleotide, the first non-barcoding oligonucleotide, non-barcoding second oligonucleotide or both, further comprise a stem loop sequence (e.g., any of the stem loop sequences provided herein or known in the art).

In some embodiments where the first oligonucleotide, second oligonucleotide, or both does not include a barcode, the first non-barcoding oligonucleotide, non-barcoding second oligonucleotide or both further comprise a nick endonuclease recognition sequence, a reverse complement of a nick endonuclease recognition site (e.g., any of the ERS described herein or known in the art).

In some embodiments where the first oligonucleotide, second oligonucleotide, or both do not include a barcode, the first non-barcoding oligonucleotide, non-barcoding second oligonucleotide or both comprise from 5′ to 3′: (a) a consensus region, a barcode, an amplification sequence, a nick endonuclease recognition sequence, and a stem loop sequence, or any combination or orientation thereof; or (b) a consensus region, a barcode, an amplification sequence, a nick endonuclease recognition site, a stem loop sequence, and a reverse complement of a nick endonuclease recognition sequence, or any combination or orientation thereof.

### Barcodes

In some embodiments, the first and second barcoding oligonucleotides each include barcode (“DS” of FIGS. 1 and 2). In some embodiments, the barcode is selected from a sample barcode, a molecular barcode, a cellular barcode, a molecular cellular barcode, and a population barcode. In some embodiments, the barcodes include a designed sequence. In some embodiments, the barcode is a designed sequence similar to sample barcodes (e.g., present 1 version in a set). In some embodiments, the barcode is a designed sequence pooled together such that greater than 1 barcode sequence is in a set to greater than 1E6 to greater than 2E20 or more. In some embodiments, the barcode is a designed sequence that can be adjusted for hamming distances. In some embodiments, the barcode is a degenerate sequence. In some embodiments, the barcode is a partially degenerate sequence. In such cases, the partially degenerate sequence is interrupted at specific positions with designed bases. In some embodiments, the barcode is a partially degenerate sequence using degenerate bases that only include a subset of ACGT in a position. The barcode (e.g., a molecular cellular label) can include a degenerate sequence, repeat sequence, variable sequence, or a combination of degenerate, repeat, and/or variable sequences that serve as short nucleotide sequences used to tag each molecule from a single cell with one to hundreds to thousands of unique cellular labels. In some embodiments, the first barcode (e.g., molecular cellular label) includes 1-50 nucleotides (e.g., such as 1-10, 2-10, 3-10, 4-10, 5-10, 6-10, 7-10, 8-10, 8-20, 10-15, 15-20, 20-25, 25-30, 30-35, 35-40, 40-45, or 45-50). In some embodiments, the first barcode (e.g., molecular cellular label) includes 8-50 nucleotides (e.g., such as 8-10, 8-20, 10-15, 15-20, 20-25, 25-30, 30-35, 35-40, 40-45, or 45-50). In certain embodiments, the first barcode (e.g., molecular cellular label) includes a length of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more nucleotides. In certain embodiments, the first barcode (e.g., molecular cellular label) includes 8 nucleotides. The barcode (e.g., molecular cellular label) of the first barcoding oligonucleotide is distinguishable (e.g., has different nucleotide sequences) from the barcode (e.g., molecular cellular label) of the second barcoding oligonucleotide. In some embodiments, the second barcode (e.g., molecular cellular label) includes 1-50 nucleotides (e.g., such as 1-10, 2-10, 3-10, 4-10, 5-10, 6-10, 7-10, 8-10, 8-20, 10-15, 15-20, 20-25, 25-30, 30-35, 35-40, 40-45, or 45-50). In some embodiments, the second barcode (e.g., molecular cellular label) includes 8-50 nucleotides (e.g., such as 8-10, 8-20, 10-15, 15-20, 20-25, 25-30, 30-35, 35-40, 40-45, or 45-50). In certain embodiments, the second barcode (e.g., molecular cellular label) includes a length of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more nucleotides. In certain embodiments, the second barcode (e.g., molecular cellular label) includes 8 nucleotides. The barcoding oligonucleotides of the present methods can include degenerate or mismatch bases within its central region to alter the sequence of the DNA, cDNA, or RNA fragment. Non-limiting examples of barcoding oligonucleotides can be found in U.S. Pat. No. 10,155,944, which is hereby incorporated by reference in its entirety.

In some embodiments, each cell within the heterogeneous cell population of the sample includes less than 10%, less than 8%, less than 7%, less than 6%, less than 5%, less than 4%, less than 3%, less than 2%, or less than 1% of barcoding oligonucleotides with the same first and second barcodes (e.g., molecular cellular label) as a different cell within the heterogeneous cell population. For example, there are distinct first barcoding oligonucleotide and second barcoding oligonucleotide combinations for each sequence within a cell based on the first and second barcodes (e.g., molecular cellular labels). Combinations of the first barcoding oligonucleotide and second barcoding oligonucleotides are then identified and grouped together in a way to identify what combinations of barcodes existed in each cell. In other words, the unique combination of cellular labels within a cell can act as a unique sample index for that cell.

### Consensus Regions

In some embodiments, the first and second barcoding oligonucleotides each include at least one consensus region. In some embodiments, the first and second oligonucleotides that do not include a barcode include at least one consensus region.

In some embodiments, the first and second barcoding oligonucleotides each include at least two consensus regions, at least three consensus regions, at least four consensus regions, at least five consensus regions, at least six consensus region, at least seven consensus regions, at least eight consensus regions, at least nine consensus regions, or at least ten consensus regions.

In some embodiments, the first and second oligonucleotides each include at least one consensus region, at least two consensus regions, at least three consensus regions, at least four consensus regions, at least five consensus regions, at least six consensus regions, at least seven consensus regions, at least eight consensus regions, at least nine consensus regions, or at least ten consensus regions.

In some embodiments, a consensus region comprises a nucleotide sequence length ranging from 15-50 nucleotides, such as 15-20 nucleotides, 20-35 nucleotides, 15-35 nucleotides, 30-35 nucleotides, 40-50 nucleotides, 30-50 nucleotides, 15-40 nucleotides, and the like). In certain embodiments, at least one consensus region comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In some embodiments, a barcode (e.g., a molecular cellular label (“DS” in FIGS. 1-2)) is positioned between two consensus regions. For example, the first consensus region, shown as “CR1′” of the first set of barcoding oligonucleotides (part “B” of FIG. 1 and FIG. 2A) and the first consensus region “CR2′” of the second set of barcoding oligonucleotides (part “B” of FIG. 1 and FIG. 2A) of FIGS. 1 and 2, include nucleotide sequences that are complementary to consensus read regions “CR1” and“CR2” of the nucleic acid fragment (e.g., DNA, cDNA, or RNA fragments (part “A” of FIGS. 1-2)). For example, when a dsDNA fragment (insert) is present, during amplification, the first set of barcoding oligonucleotides and the second set of barcoding oligonucleotides are amplified to generate barcoding primers comprising a barcode flanked by consensus regions (see, e.g., part “E” and “F” of FIG. 1) where one of the consensus region that is complementary to the CR1′ or CR2′ regions of the dsDNA fragment. In another example, when a dsDNA fragment (insert) is present, during amplification, the first set of oligonucleotides (i.e., first set of oligonucleotides without a barcode) and the second set of oligonucleotides (i.e., second set oligonucleotides without a barcode) are amplified to generate primers comprising a consensus region that is complementary to a consensus read region on the nucleic acid fragment.

In some embodiments, the first and second barcoding oligonucleotides include an adapter sequence or a reverse complement thereof (see e.g., “CR3′”, “CR4′” of FIGS. 1 and 2).

In some embodiments, the first and second non-barcoding oligonucleotide includes an adapter sequence.

The adapter sequence can be nucleotide sequences that allow high-throughput sequencing of amplified nucleic acids. These adapter sequences can include, as a non-limiting example, flow cell binding sequences that are platform-specific sequences for library binding to the sequencing instrument and/or a consensus region to allow further amplification and barcoding steps. For example, the adapter sequence of the first set of oligonucleotides or barcoding oligonucleotide can include P5 adapter sequences (or a reverse complement thereof), and the adapter sequence of the second set of oligonucleotides or barcoding oligonucleotides can include P7 adapter sequences (or a reverse complement thereof). In some embodiments, the first and second set of oligonucleotides or barcoding oligonucleotides include at least one adapter sequence, at least two adapter sequences, at least three adapter sequences, at least four adapter sequences, at least five adapter sequences, at least six adapter sequences or at least seven adapter sequences. In certain embodiments, the first and second set of oligonucleotides or barcoding oligonucleotides include one or more adapter sequences, two or more adapter sequences, three or more adapter sequences, four or more adapter sequences, five or more adapter sequences, six or more adapter sequences, seven or more adapter sequences, eight or more adapter sequences, nine or more adapter sequences, or ten or more adapter sequences.

In certain embodiments, the first and second oligonucleotides or barcoding nucleotide sequences each include a consensus region and an adapter sequence that flank the barcode. In certain embodiments, the first or second barcode is positioned between the consensus region and the adapter sequence.

Amplification of each set of barcoding oligonucleotides produces a product (e.g. barcoding primer) that will attach or bridge to either end of the tagged nucleic acid fragment (e.g., DNA or RNA fragment) within the cell, but the barcoding oligonucleotide on its own cannot amplify the tagged nucleic acid fragment (e.g., DNA, cDNA, or RNA fragment). Thus, in some embodiments, the nucleic acid fragment (e.g., DNA, cDNA, or RNA fragment) is not amplified during the first amplification step (see e.g., part “D” of FIG. 2A). For example, each of the first and second barcoding oligonucleotides contains a consensus region that is complementary to one strand of the dsDNA, however due to oligonucleotide orientation there are 3′ overhangs of the hybridization product which cannot be amplified Amplification of the barcode oligonucleotides however produces a set of molecules, that, when hybridized, generate 5′ overhangs that can be amplified. This shows the need for an initial hybridization and amplification reaction of the barcoding oligonucleotides before amplification of the DNA, cDNA, or RNA fragment of interest.

The methods of the present disclosure, in some embodiments, also include contacting the DNA or RNA fragments with an amplification primer and/or first set of amplification primers and a second amplification primer and/or second set of amplification primers. Amplification primers can be added separately, or preligated to molecule of interest, such as barcoding oligonucleotides, or be part of the same oligonucleotide, such as a hairpin oligonucleotide.

In some embodiments, the amplification primer is provided at the same concentration as the barcoding oligo (i.e., pre-ligated to the barcoding oligo). In some cases it is provided in excess of the barcoding oligo.

In some embodiments, the amplification primer and/or first set of amplification primers can include a consensus region or a reverse complement thereof (e.g., Amplification primer 1 CR3 of “B” FIG. 1) which is complementary to CR3′ of the first set of barcoding oligonucleotides. In some embodiments, the amplification primer or first set of amplification primers includes a reverse complement of a consensus read region. In some embodiments, the second set of amplification primers can include a consensus read region (e.g., Amplification primer CR4 of “C” of FIG. 1) which is complementary to CR4′ of the second set of barcoding oligonucleotides. In some embodiments, the second amplification primer or second set of amplification primers includes a reverse complement of a consensus read region (see, e.g., “E” and “F” of FIGS. 1 and 2A).

In some embodiments, for example where barcoding oligonucleotides or non-barcoding oligonucleotides are used to generate primers using linear amplification, the barcoding oligonucleotide or oligonucleotide without a barcode comprise an amplification sequence and one or more consensus regions. In some embodiments, the amplification sequence comprises an adapter sequence or a reverse complement thereof. In some embodiments, the amplification sequence is CR3, CR3′, or a variation thereof. For example, the amplification sequence CR3′ is at least partially complementary to CR3 of an amplification primer. In some embodiments, the amplification sequence is CR4, CR4′, or a variation thereof. In such cases, the amplification sequence CR4′ is at least partially complementary to CR4 of an amplification primer.

In some embodiments, for example where isothermal amplification is performed, the first and second amplification primers may include a cleavage site, such as a nicking endonuclease recognition site (ERS). In such cases, the ERS comprises an ERS and additional nucleic acid sequence to improve cleavage (e.g., by improve efficiency of cleavage on the primers of the present disclosure). In some embodiments, the ERS is located adjacent to a consensus region (e.g., CR3 and CR4) and the additional nucleic acid sequence is located 5′ to the ERS. In some embodiments, the ERS is flanked by additional nucleic acid sequence, where one or both of the additional nucleic acid sequences improve cleavage. In a non-limiting example, FIG. 2A shows a first and second set of amplification primers with an ERS site at the 5′ end of the first and second primer. In some embodiment, the first set of amplification primers can comprise, in 5′ to 3′ order: an ERS site (e.g., an ERS site and additional nucleic acid sequences to improve cleavage) and a consensus read region (e.g., ERS and CR3 of “B” of FIG. 2A) which is complementary to CR3′ of the first set of barcoding oligonucleotides. In some embodiments, the first set of amplification primers can comprise, in 5′ to 3′ order: an ERS site (e.g., an ERS site and additional nucleic acid sequences to improve cleavage) and a reverse complement of a consensus read region (e.g., ERS and CR3 of “B” of FIG. 2A) which is complementary to CR3 of the first set of barcoding oligonucleotides. In some embodiments where an ERS site is present, the second set of amplification primers can comprise, in 5′ to 3′ order: an ERS site (e.g., an ERS site and additional nucleic acid sequences to improve cleavage) and a consensus read region (e.g., ERS and CR4 of “C” of FIG. 2A) which is complementary to CR4′ of the second set of barcoding oligonucleotides. In some embodiments where an ERS site is present, the second set of amplification primers can comprise, in 5′ to 3′ order: an ERS site (e.g., an ERS site and additional nucleic acid sequences to improve cleavage) and a consensus read region (e.g., ERS and CR4 of “C” of FIG. 2A) which is complementary to CR4′ of the second set of barcoding oligonucleotides. The barcode amplification primers and barcode oligonucleotides hybridize to form molecules with 5′ overhangs, which can then be amplified (e.g. using PCR or nick-mediated isothermal amplification). In some embodiments, the first set of barcoding oligonucleotides are annealed to the first set of amplification primers, prior to amplification. In other embodiments, the first set of barcoding oligonucleotides are not annealed to the first set of amplification primers, prior to amplification. In some embodiments, the second set of barcoding oligonucleotides are annealed to the second set of amplification primers, prior to amplification. In some embodiments, the second set of barcoding oligonucleotides are not annealed to the second set of amplification primers, prior to amplification.

In some embodiments, the ERS and the additional nucleic acid sequence can be referred to as a “cleavage site.” In such cases, the additional nucleotide sequences improve efficiency of cleavage on the primers of the present disclosure. In some embodiments, the additional nucleotide sequences of the cleavage site comprises 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides, 11 or more nucleotides, 12 or more nucleotides, 13 or more nucleotides, 14 or more nucleotides, 15 or more nucleotides, 16 or more nucleotides, 17 or more nucleotides, 18 or more nucleotides, 19 or more nucleotides, 20 or more nucleotides, 21 or more nucleotides, 22 or more nucleotides, 23 or more nucleotides, 24 or more nucleotides, 25 or more nucleotides, 40 or more nucleotides, 45 or more nucleotides, or 50 or more nucleotides. In certain embodiments, the cleavage site comprise an ERS site comprising 4-8 nucleotides and an additional nucleotide sequence comprises 4-50 nucleotides. In some embodiments, this additional nucleotide can be referred to a padding sequence. In such cases, the padding sequence improves efficiency of cleavage.

In some embodiments, the cleavage site comprises a nucleotide length ranging from 2 to 50 nucleotides, such as 2-4 nucleotides, 4-8 nucleotides, 2-10 nucleotides, 2-20 nucleotides, 4-20 nucleotides, 4-10 nucleotides, 10-20 nucleotides, 20-50 nucleotides, 25-50 nucleotides, 30-40 nucleotides, 40-50 nucleotides, 30-50 nucleotides, 5-10 nucleotides, 15-20 nucleotides, or 5-50 nucleotides. In certain embodiments, the cleavage site comprises a length of about 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 40 nucleotides, 45 nucleotides, or 50 nucleotides.

In some embodiments, before contacting the prepared nucleic acid fragment (e.g., DNA, cDNA, or RNA fragments) with the barcoding oligonucleotides, the first set of amplification primers are annealed to the complementary consensus region of the first set of oligonucleotides; and the second set of amplification primers are annealed to the complementary consensus region of the second set of oligonucleotides. For example, the methods described herein can include mixing the first and second set of barcoding oligonucleotides with the first and second sets of amplification primers at a molar ratio sufficient to result in annealed oligonucleotides, where the first set of barcoding oligonucleotides are annealed to the first set of amplification primers, and the second set of barcoding oligonucleotides are annealed to the second set of amplification primers. These annealed oligonucleotides are then contacted with the DNA or RNA fragments.

### Barcoding Products

Next, the resulting first and second set of barcoding oligonucleotides are amplified during a PCR amplification reaction, rolling circle amplification reaction, or an isothermal amplification reaction to produce a set of barcoding products (“E” and “F” of FIG. 2A). In some embodiments, for example where oligonucleotides without barcode are used to generate primers using linear amplification, the oligonucleotides are amplified during a PCR amplification reaction, rolling circle amplification reaction, or an isothermal amplification reaction to produce a primer or set of primers, whereby the primer or set of primers do not include a barcode. In some embodiments, the oligonucleotides include a barcode. In such cases, the amplification of the oligonucleotide results in a primer that includes a barcode.

In some embodiments, the barcoding products comprise a first set of barcoding primers and a second set of barcoding primers.

The first set of barcoding primers include, a 5′ oligonucleotide strand, from 5′ to 3′ order: a consensus region (e.g., a first adapter sequence) (CR3 in “E” of FIG. 2A), the first barcode, (DS′), and a consensus region (e.g., sequence complementary to consensus region on insert) (CR1 in “E” of FIG. 2A). The second set of barcoding primers include, from 5′ to 3′ order: a consensus region (e.g., a second adapter sequence) (CR4 in “F” of FIG. 2A) the second barcode (DS of FIG. 2A), and the consensus read region (CR2 in “F” of FIG. 2A).

In some embodiments, for example where oligonucleotides (i.e., without a molecular cellular label) are used to generate primers using linear amplification, the resulting primer includes a 5′ oligonucleotide strand, comprising form 5′ to 3′ order: a reverse complement of the amplification sequence and a reverse complement of the consensus region.

In some embodiments each barcoding primer or primer has a length ranging from 20-120 nucleotides, such as 50-80 nucleotides, 20-50 nucleotides, 20-60 nucleotides, 50-80 nucleotides, 20-60 nucleotides, 20-70 nucleotides, 30-60 nucleotides, 40-80 nucleotides, or 60-80 nucleotides. In certain embodiments, the length of each of the barcoding primers or primers is about 30 nucleotides, about 35 nucleotides, about 40 nucleotides about 45 nucleotides, about 50 nucleotides, the 55 nucleotides, about 60 nucleotides, about 65 nucleotides, about 70 nucleotides, about 75 nucleotides, about 80 nucleotides, about 85 nucleotides, about 95 nucleotides, about 100 nucleotides, about 105 nucleotides, about 110 nucleotides, about 115 nucleotides, or about 120 nucleotides.

In certain embodiments, each barcoding primer or primer in the first set of barcoding primers has a length ranging from 20-120 nucleotides, such as 50-80 nucleotides, 20-50 nucleotides, 20-60 nucleotides, 50-80 nucleotides, 20-60 nucleotides, 20-70 nucleotides, 30-60 nucleotides, 40-80 nucleotides, or 60-80 nucleotides. In certain embodiments, the length of each of the barcoding primers or primers in the first set of barcoding primers is about 30 nucleotides, about 35 nucleotides, about 40 nucleotides about 45 nucleotides, about 50 nucleotides, the 55 nucleotides, about 60 nucleotides, about 65 nucleotides, about 70 nucleotides, about 75 nucleotides, about 80 nucleotides, about 85 nucleotides, about 95 nucleotides, about 100 nucleotides, about 105 nucleotides, about 110 nucleotides, about 115 nucleotides, or about 120 nucleotides.

In certain embodiments, each barcoding primer or primer in the second set of barcoding primers has a length ranging from 20-120 nucleotides, such as 50-80 nucleotides, 20-50 nucleotides, 20-60 nucleotides, 50-80 nucleotides, 20-60 nucleotides, 20-70 nucleotides, 30-60 nucleotides, 40-80 nucleotides, or 60-80 nucleotides. In certain embodiments, the length of each of the barcoding primers or primers in the second set of barcoding primers is about 30 nucleotides, about 35 nucleotides, about 40 nucleotides about 45 nucleotides, about 50 nucleotides, the 55 nucleotides, about 60 nucleotides, about 65 nucleotides, about 70 nucleotides, about 75 nucleotides, about 80 nucleotides, about 85 nucleotides, about 95 nucleotides, about 100 nucleotides, about 105 nucleotides, about 110 nucleotides, about 115 nucleotides, or about 120 nucleotides.

In some embodiments, the first set of barcoding primers and the second set of barcoding primers include a cleavage or endonuclease recognition site (ERS). In some embodiments, the first set of barcoding primers and the second set of barcoding primers do not include a cleavage or endonuclease recognition site (ERS).

In some embodiments, for example where oligonucleotides (i.e., without a molecular cellular label) are used to generate primers using linear amplification, the first primers and the second primers include a cleavage or endonuclease recognition site (ERS). In some embodiments, for example where oligonucleotides (i.e., without a molecular cellular label) are used to generate primers using linear amplification, the first primers and the second primers do not include a cleavage or endonuclease recognition site (ERS).

### PCR Amplification Reactions

Aspects of the present methods include performing PCR amplification to amplify the prepared DNA fragments (e.g., prepared according the methods provided herein, e.g., as described in PCT/US2021/046025 (WO2022/036273), which is herein incorporated by reference in its entirety) and produce a DNA library containing a first barcoding primer bridging from the 5′ end of a first strand of the DNA fragments and the second barcoding primer bridging from the 5′ end of the opposite strand of DNA fragments (see, e.g., FIG. 1).

In some embodiments, production of the DNA, cDNA, or RNA library comprises multiple cycles of PCR. For example, in certain embodiments, the method comprises performing at least one cycle of PCR, at least two cycles of PCR, at least three cycles of PCR, at least four cycles of PCR, at least five cycles of PCR, at least six cycles of PCR, at least seven cycles of PCR, at least eight cycles or PCR, at least nine cycles of PCR, or at least ten cycles of PCR. In certain embodiments, the method comprises at least 3 cycles of PCR. In certain embodiments, the method comprises at least 2 cycles of PCR. In certain embodiments, the method comprises at least 1 cycle of PCR.

For example, a PCR reaction is set up with inputs into the PCR reaction containing the prepared DNA, cDNA, or RNA fragment (in cells), the sets barcode oligonucleotides and barcode oligonucleotide primers. In certain embodiments, the PCR input also contains DNA polymerases and buffers for the various cycles of the PCR reaction. In the first and all subsequent cycles of the PCR reaction, the barcoding oligonucleotides are amplified using the amplification primers to produce barcoding primers (e.g. barcoding products). In the first PCR cycle, amplification of the prepared DNA, cDNA, or RNA fragments does not occur. In the second and all subsequent products, the amplified barcoding oligonucleotide primers can amplify the prepared DNA, cDNA, or RNA fragments. In certain embodiments, it is not until the 3′ and all subsequent PCR cycles that a complete duplex product containing 5′-CR3-DS-CR1-insert-CR2′-DS-CR3′-3′ is formed. In certain embodiments, 3 or more PCR cycles is needed to amplify the DNA or RNA target fragments.

After amplification the cells can be lysed as cellular context is now encoded in the DNA fragments. PCR purification is performed prior to sequencing.

In some embodiments, an additional round of PCR amplification can be included if the CR3 and CR4 adapter sequences used are not sufficient for cluster amplification on a sequencing instrument. If such embodiments, addition sample barcodes could be added.

After the first PCR step of amplifying the barcoding oligonucleotides to produce barcoding primers further amplification reactions are performed. Inputs into the PCR reaction can include one or more enzymes, such as DNA polymerases, buffers, and/or primers needed for amplifying the barcoding oligonucleotides, and amplifying the DNA or RNA fragments to produce a DNA or RNA library containing the one or more molecular cellular labels.

A non-limiting example of the PCR amplification workflow for cellular barcoding in situ is shown in FIG. 1. Inputs into the PCR reaction include: A: In Situ Insert Library with Consensus regions appended to DNA; B. Barcode oligonucleotide 5′-CR1′-DS-CR3′-3′ (provided in restricted amounts) and barcode amplification primer 5′-CR3-3′ (provided in excess); and C. Barcode oligonucleotide 5′-CR2′-DS-CR4′-3′ (provided in restricted amounts) and barcode amplification primer 5′-CR4-3′ (provided in excess). The products from the PCR reaction include D. A library containing two DS regions each surrounded by two consensus regions. Production of this library may require multiple cycles of PCR, and some side products containing one or both degenerate sequences may be possible.

In some embodiments, the aim of the PCR amplification workflow in FIG. 1 is to amplify the 5′-CR1′-DS-CR3′-3′ barcoding oligonucleotides to generate a sufficient number of 5′-CR3-DS-CR1-3′ barcoding primers that enables amplification of the nucleic acid sequence in the system (e.g., DNA, cDNA, or RNA fragments). In some embodiments, the amplification primer (e.g., 5′-CR3-3′) gets used up in the process of amplifying the barcode oligonucleotide (e.g., 5′-CR1′-DS-CR3′-3′). In such cases, providing excess amount of the amplification primer allows for multiple copies of the barcoding primer to be made.

In some embodiments, the aim of the PCR amplification workflow in FIG. 1 is to amplify the 5′-CR2′-DS-CR4′-3′ barcoding oligonucleotides to generate a sufficient number of 5′-CR4-DS-CR2-3′ barcoding primers that enables amplification of the nucleic acid sequence in the system (e.g., DNA, cDNA, or RNA fragments). In some embodiments, the amplification primer (e.g., 5′-CR4-3′) gets used up in the process of amplifying the barcode oligonucleotide (e.g., 5′-CR2′-DS-CR4′-3′). In such cases, providing excess amount of the amplification primer allows for multiple copies of the barcoding primer to be made.

In a non-limiting example, in the workflow of FIG. 2A the barcode oligos 5′-CR1′-DS-CR3′-3′ and 5′-CR2′-DS-CR4′-3′ are provided in restricted amounts but barcode amplification primer 5′-ERS-CR3-3′ and 5′-ERS-CR4-3′ are provided in excess.

In some embodiments, for example where a nick endonuclease site is included in the barcode amplification primer or a nick endonuclease is in the barcoding oligos, providing an excess amount of amplification primer is optional.

In some embodiments, the barcoding oligonucleotides are provided in amounts sufficient to enable unique combinations of barcoding oligonucleotides to be present in a cell. In such cases, having unique combinations of barcoding oligonucleotides enables deconvolving. For example, the concentration of barcoding oligonucleotides are provided at a concentration range from 100 fM to 1 μM (or any of the subranges therein). In another example, the concentration of barcoding oligonucleotides are provided at a concentration range from 1 pM-10 pM (or any of the subranges therein).

In some embodiments, the amplification primer are provided in amounts sufficient to enable amplification of the barcoding oligonucleotides to produce barcoding primers. In some embodiments, the amplification primer is provided at a concentration of about 1 μM to about 100 μM (e.g., about 1 μM to about 90 μM, about 1 μM to about 80 μM, about 1 μM to about 70 μM, about 1 μM to about 60 μM, about 1 μM to about 50 μM, about 1 μM to about 40 μM, about 1 μM to about 30 μM, about 1 μM to about 20 μM, about 1 μM to about 10 μM, about 1 μM to about 5 μM, about 5 μM to about 100 μM, about 5 μM to about 90 μM, about 5 μM to about 80 μM, about 5 μM to about 70 μM, about 5 μM to about 60 μM, about 5 μM to about 50 μM, about 5 μM to about 40 μM, about 5 μM to about 30 μM, about 5 to about 20 μM, about 5 to about 10 μM, about 10 μM to about 100 μM, about 10 μM to about 90 μM, about 10 μM to about 80 μM, about 10 μM to about 70 μM, about 10 μM to about 60 μM, about 10 μM to about 50 μM, about 10 μM to about 40 μM, about 10 μM to about 30 μM, about 10 to about 20 μM, about 20 μM to about 100 μM, about 20 μM to about 90 μM, about 20 μM to about 80 μM, about 20 μM to about 70 μM, about 20 μM to about 60 μM, about 20 μM to about 50 μM, about 20 μM to about 40 μM, about 20 μM to about 30 μM, about 30 μM to about 100 μM, about 30 μM to about 90 μM, about 30 μM to about 80 μM, about 30 μM to about 70 μM, about 30 μM to about 60 μM, about 30 μM to about 50 μM, about 30 μM to about 40 μM, about 40 μM to about 100 μM, about 40 μM to about 90 μM, about 40 μM to about 80 μM, about 40 μM to about 70 μM, about 40 μM to about 60 μM, about 40 μM to about 50 μM, about 50 μM to about 100 μM, about 50 μM to about 90 μM, about 50 μM to about 80 μM, about 50 μM to about 70 μM, about 50 μM to about 60 μM, about 60 μM to about 100 μM, about 60 μM to about 90 μM, about 60 μM to about 80 μM, about 60 μM to about 70 μM, about 70 μM to about 100 μM, about 70 μM to about 90 μM, about 70 μM to about 80 μM, about 80 μM to about 100 μM, about 80 μM to about 90 μM, or about 90 μM to about 100 μM).

In some embodiments, a thermostable polymerase and temperature cycling (e.g., PCR) are used to produce the primers and/or barcoding primers. In some embodiments, a thermostable polymerase and temperature cycling are used to produce the set of primers or barcoding primers before amplifying the prepared DNA, cDNA, or RNA fragments within the cell populations using PCR and the primers or barcoding primers. In some embodiments, production of the primers or barcoding primers comprises multiple cycles of PCR. For example, in certain embodiments, the method comprises performing at least one cycle of PCR, at least two cycles of PCR, at least three cycles of PCR, at least four cycles of PCR, at least five cycles of PCR, at least six cycles of PCR, at least seven cycles of PCR, at least eight cycles or PCR, at least nine cycles of PCR, or at least ten cycles of PCR. In certain embodiments, the method comprises at least 3 cycles of PCR. In certain embodiments, the method comprises at least 2 cycles of PCR. In certain embodiments, the method comprises at least 1 cycle of PCR.

In some embodiments, a PCR reaction is set up with inputs into the PCR reaction containing an oligonucleotide without a barcode, an oligonucleotide with a barcode, a second oligonucleotide with a barcode, and a second oligonucleotide without a barcode, or any combination thereof. In certain embodiments, the PCR input also contains DNA polymerases and buffers for the various cycles of the PCR reaction. In the first and all subsequent cycles of the PCR reaction, the oligonucleotides (e.g., the oligonucleotide without a barcode, the oligonucleotide with a barcode, the second oligonucleotide with a barcode, and the second oligonucleotide without a barcode) are amplified using the amplification primers to produce primers and/or barcoding primers.

After amplification the primers and/or barcoding primers can be used to amplify DNA, cDNA, or RNA fragments (e.g., including prepared and unprepared DNA, cDNA, or RNA fragments). Non-limiting examples of additional uses of the primers and/or barcoding primers following amplification include being used in a ligation reaction, in a capture reaction whereby the primer and/or barcoding primer capture a DNA, cDNA, or RNA fragment that includes the consensus region, or as a standalone label (e.g., barcode).

### Isothermal Amplification and PCR Amplification Reactions

In some embodiments, isothermal amplification is performed to produce the set of amplified barcode oligonucleotide primers (FIGS. 2A-2B) before using PCR to amplify the prepared DNA, cDNA, or RNA fragments within the cell populations. In some embodiments, a nicking enzyme, an isothermal polymerase, first set of annealed cellular barcoding oligonucleotides (e.g. annealed to the first set of amplification primers), and the second set of annealed barcoding oligonucleotides (e.g., annealed to the second set of amplification primers) are added to cells with prepared DNA, cDNA, or RNA fragments.

In some embodiments, the first and second set of barcoding oligonucleotides and the first and second set of amplification primer are added separately.

In alternative embodiments, the first and second set of barcoding oligonucleotides comprise hairpin oligonucleotides that contains both the barcoding oligonucleotides and amplification primers in addition to a hairpin sequence (e.g., a stem loop sequence) in a single molecule. In some embodiments, a first set of hairpin barcoding oligonucleotides comprise a first barcode (e.g., molecular cellular label); and a consensus region comprising a nucleotide sequence that is complementary to a 5′ read region of a first strand of the DNA, cDNA or RNA fragments. In some embodiments, the second set of hairpin barcoding oligonucleotides comprises a second barcode (e.g., molecular cellular label); and a consensus region comprising a nucleotide sequence that is complementary to a 5′ read region of a second strand of the DNA, cDNA, or RNA fragments.

In some embodiments, the hairpin barcoding oligonucleotides in the first set of hairpin barcoding oligonucleotides optionally includes a first adapter sequence (e.g., a P5 or P7 sequence), and the hairpin barcoding oligonucleotides in the second set of hairpin barcoding oligonucleotides optionally includes a second adapter sequence (e.g., a P5 or P7 sequence). The first and second set of hairpin barcoding oligonucleotides optionally include cleavage sites. In some embodiments, the hairpin oligonucleotides comprise a hairpin sequence at the 5′ or 3′ end of the barcoding oligonucleotide (e.g. stem loop). Such embodiments with hairpin oligonucleotides may be alternatives to annealed cellular barcoding oligonucleotides/amplification primers.

For example, during an isothermal amplification reaction, the isothermal polymerase amplifies the barcoding oligonucleotides and the nicking enzyme recognizes the ERS cleaving only one of the strands of the dsDNA and allowing priming for subsequent amplification of the barcode oligonucleotide and release of amplified barcoding oligonucleotide. The resulting barcoding products (barcoding primers) is the reverse complement of the barcoding oligonucleotide without the ERS site, and comprises: 5′-CR3-DS′-CR1-3′ (“E” of FIG. 2A″ and 5′-CR4-DS′-CR2-3′ (“F” of FIG. 2A).

After the isothermal amplification reaction is performed in situ, and the isothermal amplification enzyme and nicking enzymes are heat inactivated, if required, a PCR amplification reaction is performed on the cells. The PCR template (prepared DNA) and PCR barcoding primers (isothermally amplified barcode oligonucleotides) are already present in the cells, so only buffer and enzymes need to be added. During PCR amplification, the dsDNA fragments are denatured or displaced. Following denaturing or displacement, the isothermally amplified barcode primers are annealed and extended in 5′-3′ direction along the DNA fragments. In some embodiments, this process is repeated, via one or more, two or more, three or more, three or more, four or more, five or more, six or more, seven or more, eight or more, nine or more, or ten or more PCR cycles, to ensure that the amplicons contain cell barcode sequences on both sides of the insert. The annealing and extending steps result in a set of amplicon products, containing a duplex molecule where the first strand contains 5′-CR3-DS′-CR1-Insert-CR2′-DS-CR4′-3′ (FIG. 2A) and second strand contains 5′-CR4-DS-CR2-Insert′-CR1′-DS-CR3′-3′ (FIG. 2A).

After the PCR amplification step, the DNA fragments contain all of the required information to associate the sequence read back to the cell it originated from and therefore can be lysed, immediately or after a sorting step. If CR3 and CR4 adapter sequences contained all of the required sequences for amplifying on the flow cell the material can be sequenced or further processed in any ways that adapter sequence-labeled DNA fragment would be used (i.e., can undergo hybrid capture target enrichment protocols, and the like.)

If CR3 and CR4 are not sufficient for amplifying on the flow cell, another PCR amplification reaction may be performed, for example, in vitro. This step can add indexing primers to the amplicons and then the material can be sequenced or further processed in any ways that adapter labeled DNA fragment would be used (i.e., can undergo hybrid capture target enrichment protocols, and the like).

A non-limiting example of the workflow of the isothermal amplification and PCR workflow for cellular barcoding in situ is shown in FIGS. 2A and 2B. Inputs of the Isothermal amplification reaction include: A. In Situ Insert Library with Consensus regions (CR1 and CR2) appended to DNA; B. Annealed isothermal amplification primer set 1, that includes a barcode oligonucleotide 5′-CR1′-DS (degenerate sequence)-CR3′-3′ and barcode amplification primer 5′-ERS-CR3-3′; C. Annealed isothermal amplification primer set 2, that includes barcode oligonucleotide 5′-CR2′-DS-CR4′-3′ and barcode amplification primer 5′-ERS-CR4-3′; and the nicking enzyme and isothermal DNA polymerase. The products that come out of the isothermal amplification reaction include: D. In Situ Insert Library with Consensus regions appended to DNA, exactly same as A; E Amplified Barcode Oligo Set 1, generated via isothermal amplification of the annealed isothermal amplification primer set 1 (B), where the Barcode oligo extends through the ERS and the barcode amplification primer extends through the DS and CR1 regions. The nicking enzyme can cleave (repeatedly) the top strand of the ERS and allow the isothermal amplification enzyme to extend the ERS over the barcode oligo; F. Amplified Barcode Oligo Set 2, generated via isothermal amplification of the annealed isothermal amplification primer set 2 (C), where the Barcode oligo extends through the ERS and the barcode amplification primer extends through the DS and CR2 regions. The nicking enzyme can cleave (repeatedly) the top strand of the ERS and allow the isothermal amplification enzyme to extend the ERS over the barcode oligo. FIG. 2A describes the next step requiring PCR Amplification on the cells that have undergone isothermal amplification of the barcoding oligonucleotides. The inputs include cells containing the products from FIG. 2A, and the outputs include complete libraries with two sets of degenerate sequences, both surrounded by consensus regions.

In some embodiments, isothermal amplification is performed to produce amplified primers (e.g., a first primer and a second primer) where the primers do not include barcode sequences. In some embodiments, a nicking enzyme; an isothermal polymerase; an oligonucleotide comprising an amplification sequence and a consensus region; and an amplification primer comprising a nick endonuclease recognition site or reverse complement thereof and a nucleotide sequence that is at least partially complementary to the amplification sequence on the oligonucleotide are added to a reaction container (e.g., any of the reaction containers provided herein or known in the art). An isothermal amplification reaction generates the primer comprising the reverse complement of the consensus region.

In some embodiments, a nicking enzyme; an isothermal polymerase; an oligonucleotide comprising an amplification sequence and a consensus region; an amplification primer comprising a nick endonuclease recognition site or reverse complement thereof and a nucleotide sequence that is at least partially complementary to the amplification sequence on the oligonucleotide; a second oligonucleotide comprising a second nick endonuclease recognition site or reverse complement thereof; and a second amplification primer comprising a second nick endonuclease recognition site or reverse complement thereof and a nucleotide sequence that is at least partially complementary to the second amplification sequence on the second oligonucleotide are added to a reaction container (e.g., any of the reaction containers provided herein or known in the art). An isothermal amplification reaction generates the primer comprising the reverse complement of the consensus region and the second primer comprising the reverse complement of the second consensus region.

In some embodiments, the first and second set oligonucleotides and the first and second amplification primers are added separately to the reaction container.

In alternative embodiments, the first and second oligonucleotides comprise hairpin oligonucleotides. In some embodiments, the hairpin oligonucleotides include an amplification sequence and a consensus region comprising a nucleotide sequence that is complementary to a target sequence of a DNA or RNA fragment in addition to a hairpin sequence (e.g., a stem loop sequence) in a single molecule. In some embodiments, the second hairpin oligonucleotide comprises a second amplification sequence; and a consensus region comprising a nucleotide sequence that is complementary to a target sequence of a DNA or RNA fragment.

In some embodiments, the hairpin oligonucleotides in the first hairpin oligonucleotides optionally include a first adapter sequence, and the hairpin oligonucleotides in the second s hairpin oligonucleotides optionally include a second adapter sequence. The first and second of hairpin oligonucleotides optionally include cleavage sites (e.g., endonuclease recognition sites). In some embodiments, the hairpin oligonucleotides comprise a hairpin sequence at the 5′ or 3′ end of the barcoding oligonucleotide (e.g. stem loop). Such embodiments with hairpin oligonucleotides may be used an alternative to amplification of primers using thermal stable polymerases and thermal cycling.

In some embodiments, during an isothermal amplification reaction, the isothermal polymerase amplifies the oligonucleotides and the nicking enzyme recognizes the ERS (endonuclease recognition site) cleaving only one of the strands of the dsDNA and allowing priming for subsequent amplification of the oligonucleotide and release of amplified primer. The resulting amplified primer is all or part of the reverse complement of the oligonucleotide without the ERS site. For example, the amplified primer includes all or part of the reverse complement of the amplification sequence and all or part of the reverse complement of the consensus region, where the consensus region includes a sequence that is at least partially complementary to a target sequence of a DNA or RNA fragment.

After the isothermal amplification reaction is performed in the reaction container, the isothermal amplification enzyme and nicking enzymes are heat inactivated. The amplified primers can then be used for downstream applications, including PCR amplification reaction of a DNA or RNA fragment. In such cases, the amplification of the DNA or RNA fragment is performed using the methods described herein.

In some embodiments where the method of amplifying the barcoding oligonucleotide or non-barcoding oligonucleotides include isothermal amplification, the isothermal amplification is performed using an isothermal polymerase. Non-limiting examples of isothermal polymerases include Klenow Fragment (Exo−), Bsu Large Fragment, Bst DNA polymerase, Bst2.0, Sequenase, Bsm DNA Polymerase, EquiPhi29, and Phi29 DNA polymerase.

In some embodiments where the method of amplifying the barcoding oligonucleotide or non-barcoding oligonucleotides include isothermal amplification, the amplification is performed under conditions that allow for primer invasion.

In some embodiments where the method of amplifying the barcoding oligonucleotide or oligonucleotides that do not include a barcode include isothermal amplification, the amplification is in the presence of a nick endonuclease. Non-limiting examples of nick endonuclease include nt.BspQI, nt.CviPII, nt.BstNBI, nb.BsrDI, nb.BtsI, nt.AlwI, nb.BbvcI, nt.BbvcI, nb.BsmI, nb.BssSI, nt.BsmAI, nb.Mva1269I, nb.Bpu10I, and nt.Bpu10I.

In some embodiments where the method of amplifying the barcoding oligonucleotide or oligonucleotides that do not include a barcode include isothermal amplification, the amplification is performed under conditions that allow for both nicking via the nick endonuclease binding to the nick endonuclease recognition site (and nicking) and amplification to generate the primers.

### Concentration of Barcoding Oligonucleotides

The number of barcode oligonucleotides required to uniquely enter any cell in the sample depends on barcode oligonucleotide concentration, amount (e.g, concentration and/or volume) and length of degenerate sequence. In some embodiments, the concentration of the first and second set of barcoding oligonucleotides at which the cell is contacted with ranges from 1 femtoMolar (fM) to 5 microMolar (μM). In certain embodiments, the concentration of the first and second set of barcoding oligonucleotides at which the cell is contacted with ranges from 0.005 μM to 5 μM, such as 0.05 μM to 5 μM, 0.5 μM to 1 μM, 1 μM to 2 fM, 2 μM to 3 μM, 3 μM to 4 μM, or 4 μM to 5 μM. In certain embodiments, the concentration of the first and second set of barcoding oligonucleotides at which the cell is contacted with ranges from 1 nanoMolar (nM) to 1000 nM, such as 1 nM to 500 nM, 1 nM to 250 nM, 1 nM to 100 nM, 1 nM to 10 nM, 1 nM to 5 nM, or 1-2 nM. In certain embodiments, the concentration of the first and second set of barcoding oligonucleotides at which the cell is contacted with ranges from 1 picoMolar (pM) to 1000 pM, such as 1 pM to 100 pM, 1 pM to 50 pM, 50 pM to 100 pM, 1 pM to 10 pM, 1 pM to 5 pM, or 1-2 pM. In certain embodiments, the concentration of the first and second set of barcoding oligonucleotides at which the cell is contacted with ranges from 1 fM to 100 fM, such as 1 fM to 100 fM, 50 fM to 100 fM, 1 fM to 10 fM, 1 fM to 5 fM, or 1 fM to 2 fM.

The number of barcoding oligonucleotides in the first set of barcoding oligonucleotides and the second set of barcoding oligonucleotide entering each cell may depend on the reaction concentration of the barcode oligonucleotide and size of the cell. For example, in certain embodiments, assuming a cell volume is 0.001 μl, about 60 first barcoding oligonucleotides and about 60 second barcoding oligonucleotides may enter each of the cells within the sample when using 2 μl of 1 pM barcoding oligo in a 20 μl reaction (FIG. 3C). However, in certain embodiments, the cell volume could be lower as is the case for B-lymphocytes (130 μm³) and then less than 1 barcode would enter each cell. Therefore stock and reaction concentrations of barcoding oligos may need to be adjusted based on cell volume. In some embodiments, the number of barcoding oligonucleotides in the first set of barcoding oligonucleotides ranges from 1-10,000 barcoding oligonucleotides, such as 1-5000 barcoding oligonucleotides, 5000-10,000 barcoding oligonucleotides, 1-1000 barcoding oligonucleotides, 1-500 barcoding oligonucleotides, 500-1000 barcoding oligonucleotides, 1-10 barcoding oligonucleotides, 1-20 barcoding oligonucleotides, 10-20 barcoding oligonucleotides, 5-100 barcoding oligonucleotides, 100-200 barcoding oligonucleotides, 200-300 barcoding oligonucleotides, 300-400 barcoding oligonucleotides, 400-500 barcoding oligonucleotides, 500-600 barcoding oligonucleotides, 600-700 barcoding oligonucleotides, 700-800 barcoding oligonucleotides, 800-900 barcoding oligonucleotides, or 900-1000 barcoding oligonucleotides. In some embodiments, the number of barcoding oligonucleotides in the first set of barcoding oligonucleotides is 1 or more, 5 or more, 6 or more, 10 or more, 25 or more, 50 or more, 75 or more, 100 or more, 200 or more, 300 or more, 400 or more, 500 or more, 600 or more, 700 or more, 800 or more, 900 or more, or 1000 or more. In some embodiments, the number of barcoding oligonucleotides in the second set of barcoding oligonucleotides ranges from 1-10,000 barcoding oligonucleotides, such as 1-5000 barcoding oligonucleotides, 5000-10,000 barcoding oligonucleotides, 1-1000 barcoding oligonucleotides, 1-500 barcoding oligonucleotides, 500-1000 barcoding oligonucleotides, 1-10 barcoding oligonucleotides, 1-20 barcoding oligonucleotides, 10-20 barcoding oligonucleotides, 5-100 barcoding oligonucleotides, 100-200 barcoding oligonucleotides, 200-300 barcoding oligonucleotides, 300-400 barcoding oligonucleotides, 400-500 barcoding oligonucleotides, 500-600 barcoding oligonucleotides, 600-700 barcoding oligonucleotides, 700-800 barcoding oligonucleotides, 800-900 barcoding oligonucleotides, or 900-1000 barcoding oligonucleotides. In some embodiments, the number of barcoding oligonucleotides in the second set of barcoding oligonucleotides is 1 or more, 5 or more, 6 or more, 10 or more, 25 or more, 50 or more, 75 or more, 100 or more, 200 or more, 300 or more, 400 or more, 500 or more, 600 or more, 700 or more, 800 or more, 900 or more, or 1000 or more.

Indexing Primers

In some embodiments, the set of indexing primers include nucleotide sequences that allow identification of sequence reads during high-throughput sequencing of amplified nucleic acids. In some embodiments, the indexing primers include indexing sequences for pair-end sequencing. Indexing sequences can be used in an amplification reaction of the disclosed method for the desired sequencing method used. For example, if an Illumina sequencing platform is used, the software on the platform is able to identify these indexes on each sequence read, and since the user can input which pair of index primers were added to each sample, the platform then knows which samples to associate that read to, allowing the user to separate the reads for each different sample. In some embodiments, the method includes attaching indexing sequences to amplified nucleic acid from these sub-populations of live cells using a multiplexed PCR-based approach or ligation-based approach.

### Cell Barcoding Compositions

Provided herein are cell barcode (or cell barcoding) compositions. In some embodiments, the cell barcode composition comprises a collection of individual cells. In some embodiments, the cell barcode composition comprises a pool of cells. In some embodiments, the cell barcode composition comprises a single pool or multiple pools of cells. In some embodiments, the cell barcoding composition comprises one or more cells were a cell of the one or more cells comprises nucleic acid or genomic fragments (DNA or RNA fragments or inserts), and each nucleic acid fragment or insert comprises a barcode (e.g., FIGS. 1 and 2, labels B and C). In one example, the cell barcoding composition comprises a nucleic acid or genomic fragment that includes a barcode comprising one or more degenerate sequences, partially degenerate sequences, or set of defined sequences (FIGS. 1 and 2, “DS”). For example, the nucleic acid or genomic fragment includes a degenerate sequence on each end of the nucleic acid fragment. In another embodiment, the cell barcoding composition comprises one or more cells where a cell of the one or more cells comprises a consensus region (e.g., “CR3′”, “CR4′” of FIGS. 1 and 2). In such cases, the consensus regions include sequences that enable sequencing (e.g., a P5 adapter sequence or a P7 adapter sequence).

Further embodiments include a composition comprising a collection of cells including nucleic acid precursor libraries (e.g., FIG. 1, molecule labeled A is an example precursor library) and barcoding oligonucleotides (e.g., FIG. 1, label B, lower molecule including CR1′; label C), upper molecule including CR2′). These are capable of hybridizing to each other (e.g., barcoding oligonucleotides in B and C hybridize with precursor library A) due to complementary sequences on 5′ ends of the precursor libraries (CR1 and CR2), to create a hybridization product (e.g., FIG. 1, molecule labeled G). The hybridization product is not capable of amplification because of the 3′ overhangs on the barcoding oligonucleotides. Additional embodiments include a composition comprising a collection of intact cells, each cell comprising precursor libraries and barcoding oligonucleotides, wherein each precursor library is capable of hybridizing to one or more barcoding oligonucleotides. In fact, a cell or collection of cells at any stage in the steps illustrated by FIG. 1 may comprise a novel composition. The same is true with regard to FIGS. 2A-2B. For example, a novel composition may exist in a cell or collection of cells with precursor libraries only (or with one or more components of precursor libraries, the insert or CRs), with one or more barcoding oligonucleotides only (or with one or more components of barcoding oligonucleotides), or with both precursor libraries and barcoding oligonucleotides, and whether or not partial or full hybridization has occurred, or they are still separate unhybridized components. At any stage, an individual intact cell with any of these components, or a pool of such individual intact cells, may comprise a novel composition.

In another embodiment, the composition comprises a collection of individual cells where each cell comprises a sequencing library including genomic fragments with universal barcodes comprising degenerate sequences attached to the genomic fragments. In some embodiments, the composition comprises a next generation sequencing library made up of nucleic acid fragments with sequencing adaptors, wherein barcoding reactions involving the nucleic acid fragments result in products that include the same nucleic acid fragment with different cellular barcodes on either end of the nucleic acid fragment. In another embodiment, the invention comprises using randomly paired barcodes comprising degenerate sequences to label each end of a nucleic acid fragment in a cell. A further embodiment is a cell or collection of cells comprising a sequencing library including nucleic acid fragments with sequencing adaptors, where the progeny of those components may or may not have the same set of cell barcodes. There may be different, potentially random combinations of degenerate sequences on the same original insert molecule (e.g., two of the same insert may have the same degenerate sequence on one end or on both ends, or different degenerate sequences on both ends).

### Cell Sorting for Phenotypically Distinguishing Cell Populations

In some aspects, preparing the heterogeneous cell population prior to sequencing includes sorting the one or more cell populations.

Cell sorting may be applied before or after any of the steps described herein. Moreover, two or more sorting steps may be applied to a population of microdroplets, e.g., about 2 or more sorting steps, about 3 or more, about 4 or more, or about 5 or more, etc. When a plurality of sorting steps is applied, the steps may be substantially identical or different in one or more ways (e.g., sorting based upon a different property, sorting based upon different phenotypes, sorting using a different technique, and the like). Antibody staining and cell sorting are configured to identify specific populations of cells.

In some embodiments, sorting occurs after receiving the sample containing the heterogeneous cell population before producing the DNA or RNA fragments. Alternatively, in some embodiments, sorting the one or more cell populations occurs after the first step of amplifying nucleic acids from the cell populations using the first primer pool set to produce the first set of amplicon products (e.g., DNA or RNA fragments). In other words, in such embodiments, cell sorting occurs after producing the DNA or RNA fragments. In other alternative embodiments where hybridization capture is performed, sorting occurs after adapter ligation or after population barcoding.

In some embodiments, cell sorting and/or detectable labels facilitates the differentiation of cells by cell size, granularity, DNA content, morphology, differential protein expression (e.g., presence or absence of protein expression, or an amount of protein expression), calcium flux, and the like.

In some embodiments, cell sorting optionally includes antibody staining and sorting the cell population into subpopulations by phenotypes to determine target cells and non-target cells/nuclei.

In some embodiments, sorting the cells or contacting the cells with one or more detectable label provides for sorting protein-expressing cells, cells that secrete proteins, cells expressing an antigen-specific antibody, and the like. In some embodiments, before sorting, the cell population is contacted with an antibody being directed against a distinct cell surface molecule on the cell, under conditions effective to allow antibody binding. In some embodiments, cell sorting and/or contacting the sample with a detectable label provides for differentiating cells by morphology presence or absence of chromatin (e.g., clumped chromatin), or the absence of conspicuous nucleoli.

In some embodiments, the cell population can be prepared to include a detectable label, e.g., aptamers, cell stains, etc. For example, the cell population can be prepared by adding one or more primary and/or secondary antibodies to the sample. Primary antibodies can include antibodies specific for a particular cell type or cell surface molecule on a cell. Secondary antibodies can include detectable labels (e.g., fluorescence label) that bind to the primary antibody. Additional non-limiting examples of detectable labels include: Haematoxylin and Eosin staining, Acid and Basic Fuchsin Stain, Wright's Stain, antibody staining, cell membrane fluorescent dye, carboxyfluorescein succinimidyl ester (CFSE), DNA stains, cell viability dyes such as DAPI, PI, 7-AAD, fixable compatible dyes, amine dyes, and the like.

By sorting the cells after the first amplification step or after the first ligation step of the present methods, the present inventors have found that resolution of variants can be significantly improved from, as a non-limiting example, minimum DNA inputs at 10 ng to single cells.

Non-limiting examples of cell sorting techniques that can be used in the present methods include, but are not limited to, flow cytometry, fluorescence activated cell sorting (FACS), in situ hybridization (ISH), fluorescence in situ hybridization, Ramen flow cytometry, fluorescence microscopy, optical tweezers, micro-pipettes, and microfluidic magnetic separation devices, Magnetic Activated Cell Sorting (MACS) and methods thereof. In some embodiments, the sorting step of the methods of the present disclosure includes FACS techniques, where FACS is used to select cells from the population containing a particular surface marker, or the selection step can include the use of magnetically responsive particles as retrievable supports for target cell capture and/or background removal. For example, a variety FACS systems are known in the art and can be used in the methods of the invention (see e.g., PCT Application Publication No.: WO99/54494, US Application No. 20010006787, U.S. Pat. No. 10,161,007, each expressly incorporated herein by reference in their entirety).

In some embodiments, after sorting, the method further includes pooling two or more distinct cell populations.

### Lysing the Cells

Aspects of the present methods include lysing the cells within the one or more cell populations, including to collect ligated and/or amplified DNA or RNA fragment. In certain embodiments, lysing the cells includes contacting the cells with a cell lysing agent. The lysing step can be accomplished by contacting the DNA or RNA fragments within the cell with a cell lysing agent or physically disrupting the cell structure. In some embodiments, said lysing occurs after the ligation step.

In some embodiments, lysing occurs after one or more PCR steps. In some embodiments, lysing occurs after a sorting step. Lysing the cells with a cell lysing agent facilitates purification and isolation of the DNA or RNA fragments for each cell population.

In some embodiments, the lysing step of the present methods occurs after cellular barcoding and thus on the final amplicon products such as the second or third set of amplicon products. In some embodiments, lysing the cells purifies the amplicon products for each cell population.

In some embodiments, the lysing step of the present methods occurs after producing the second set of amplicon products (e.g., DNA or RNA fragments) or for hybridization capture methods, after amplification used for population cell barcoding. In some embodiments, lysing the cells purifies the second set of amplicon products for each cell population.

In some embodiments, lysing the cell includes contacting the cells with a cell lysing agent.

Non-limiting examples of cell lysing agents include, but are not limited to, an enzyme solution. In some embodiments, the enzyme solution includes a proteases or proteinase K, phenol and guanidine isothiocyanate, RNase inhibitors, SDS, sodium hydroxide, potassium acetate, and the like. However, any known cell lysis buffer may be used to lyse the cells within the one or more cell populations.

Non-limiting examples of cell lysing methods include, but are not limited to, an enzyme solution-based method, mechanical based methods, physical manipulation, or chemical methods. In some embodiments, the lysis solution includes a proteases or proteinase K, phenol and guanidine isothiocyanate, RNase inhibitors, SDS, sodium hydroxide, potassium acetate, and the like. However, any known cell lysis buffer may be used to lyse the cells within the one or more cell populations. Mechanical lysis methods include breaking down cell membranes using shear force. Examples of mechanical lysis methods include, but are not limited to, using a High Pressure Homogenizer (HPH) or a bead mill (also known as the bead beating method). Physical methods include thermal lysis, such as repeated freeze thaws, cavitation, or osmotic shock. Chemical denaturation includes use of detergents, chaotropic solutions, alkaline lysis, or hypotonic solutions. Detergents for cell lysis can be ionic (anionic or cationic) or non-ionic detergents, or mixtures thereof. Examples of non-ionic detergents used for lysis include, but are not limited to, 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), and Triton X-100. A non-limiting example of an ionic detergent used for lysis includes, sodium dodecyl sulfate (SDS). Examples of chaotropic agents include, but are not limited to, ethylenediaminetetraacetic acid (EDTA), and urea.

In some embodiments, lysing includes heating the cells for a period of time sufficient to lyse the cells. In certain embodiments, the cells can be heated to a temperature of about 25° C. or more, 30° C. or more, 35° C. or more, 37° C. or more, 40° C. or more, 45° C. or more, 50° C. or more, 55° C. or more, 60° C. or more, 65° C. or more, 70° C. or more, 80° C. or more, 85° C. or more, 90° C. or more, 96° C. or more, 97° C. or more, 98° C. or more, or 99° C. or more. In certain embodiments, the cells can be heated to a temperature of about 90° C., 95° C., 96° C., 97° C., 98° C., or 99° C.

### Heterogeneous Cell Population

The heterogeneous cell population can be isolated from a tumor sample, such as a tumor sample from the breast, ovarian, lung, prostate, colon, renal, liver, skin blood, bone marrow, lymph nodes, spleen, thymus, etc. In some embodiments, cancer cells that can be detected by the methods of the present disclosure include, but are not limited to, cancer cells from hematological cancers, including leukemia, lymphoma and myeloma, and solid cancers, including for example tumors of the brain (glioblastomas, medulloblastoma, astrocytoma, oligodendroglioma, ependymomas), carcinomas, e.g. carcinoma of the lung, liver, thyroid, bone, adrenal, spleen, kidney, lymph node, small intestine, pancreas, colon, stomach, breast, endometrium, prostate, testicle, ovary, skin, head and neck, and esophagus.

Tumor microenvironments contain a heterogeneous population of cells. Characterizing the composition and the interaction, dynamics, and function of a heterogeneous population of cells at the single-cell resolution are important for fully understanding the biology of tumor heterogeneity, under both normal and diseased conditions. For example, cancer, a disease caused by somatic mutations conferring uncontrolled proliferation and invasiveness, can benefit from advances in single-cell analysis. Cancer cells can manifest resistance to various therapeutic drugs through cellular heterogeneity and plasticity. The tumor microenvironment includes an environment containing tumor cells that cooperate with other tumor cells and host cells in their microenvironment and can adapt and evolve to changing conditions.

The heterogeneous population of cells can include, but are not limited to, inflammatory cells, cells that secret cytokines and/or chemokines, cytotoxic immune cells (e.g., natural killer and/or CD8⁺ T cells), immune cells, macrophages (e.g., immunosuppressive macrophages or tumor-associated macrophages), antigen-presenting cells, cancer cells, tumor-associated neutrophils, erythrocytes, dendritic cells (e.g., myeloid dendritic cells and/or plasmacytoid dendritic cells), B cells, tumor-infiltrated T cells, fibroblasts, endothelial cells, PD1⁺ T cells, and the like.

Additional non-limiting examples of the sample can include cell lines such as ovarian cancer (A4, OVCAR3), teratocarcinoma (NT2), colon cancer (HT29), prostate (PC3, DU145), cervical cancer (ME180), kidney cancer (ACHN), lung cancer (A549), skin cancer (A431), glioma (C6), but are not limited to only these lines.

The cell populations within the sample can be from mutated/malignant tissue, normal or abnormal blood, normal tissue, cell culture cells, or cells isolated from any one of saliva, urine, synovial fluid, liquid biopsies, cerebral spinal fluid, and the like. In some embodiments, the methods of the present disclosure steps are also performed on cell populations within the sample that are from a reference, control sample, such as, but not limited to: mutated/malignant tissue, non-mutated/benign tissue, abnormal or normal blood, normal tissue, cell culture cells, saliva, urine, synovial fluid, cerebral spinal fluid, and the like, which serve as a controls sample. In some embodiments, the cell populations within the sample are from both non-mutated tissue or normal blood, normal tissue, cell culture cells, saliva, urine, synovial fluid, cerebral spinal fluid, and the like can serve as a “tumor-normal” control sample, and mutated/malignant tissue and abnormal blood, abnormal tissue, cell culture cells, saliva, urine, synovial fluid, and the like can serve as a “target” sample. For example, aspects of the present methods also include performing tumor normal analysis from normal cells within a biopsy where the “target” sample came from. Such methods allow for detecting and diagnosing cell populations from non-mutated tissue or normal blood to determine if mutations are found in familial germlines that may also develop in other places of the body, or if the mutations are somatic to provide for better treatment options.

In some embodiments, the one or more cell populations within the sample includes one cell population. In some embodiments, the one or more cell population within the sample includes two or more, three or more, four or more, five or more, six or more, seven or more, eight or more, nine or more, or ten or more, eleven or more, twelve or more, thirteen or more, fourteen or more, fifteen or more, sixteen or more, seventeen or more, eighteen or more, nineteen or more, or twenty or more cell populations.

In some embodiments, the one or more cell populations is a single cell.

In some embodiments the one or more cell populations is in suspension. In some embodiments, the cell suspension comprises a single cell. In some embodiments, the cell suspension comprises a plurality of cells. In some embodiments, the cell population comprises a plurality of cells.

In some embodiments, the cell population is diluted to a volume of about 0.5 μl, about 1 μl, about 1.5 μl, about 2 μl, about 2.5 μl, about 3 μl, about 3.5 μl, about 4 μl, about 4.5 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, or about 20 μl. In some embodiments, the one or more cell populations is diluted to contain about 5 to about 200 ng of DNA. In some embodiments, the one or more cell populations is diluted to contain about 1 to about 100 ng of DNA. In some embodiments, the one or more cell populations is diluted to contain about 1 to about 200 ng of DNA (e.g., about 1 to 25 ng of DNA, about 25 to 50 ng of DNA, about 50 to 75 ng of DNA, about 75 to 100 ng of DNA, about 100 to 125 ng of DNA, about 125 to 150 ng of DNA, about 150 to 175 ng of DNA, or about 175 to 200 ng of DNA). In some embodiments, the one or more cell population is diluted to about 100 ng or less, 75 ng or less, 50 ng or less, 25 ng or less 10 ng or less, 5 ng or less, 2 ng or less, or 1 ng or less of DNA. In some embodiments, the one or more cell populations is diluted to contain about 5 to about 100 ng of DNA. In some embodiments, the one or more cell populations is diluted to contain 5 to 10 ng of DNA, 10 to 15 ng of DNA, 15 to 20 ng of DNA, 20 to 25 ng of DNA, 25 to 30 ng of DNA, 30 to 35 ng of DNA, 35 to 40 ng of DNA, 40 to 45 ng of DNA, 45 to 50 ng of DNA, 50 to 55 ng of DNA, 55 to 60 ng of DNA, 60 to 65 ng of DNA, 65 to 70 ng of DNA, 70 to 75 ng of DNA, 75 to 80 ng of DNA, 80 to 85 ng of DNA, 85 to 90 ng of DNA, 90 to 95 ng of DNA, 95 to 100 ng of DNA, 100 to 105 ng of DNA, 105 to 110 ng of DNA, 110 to 115 ng of DNA, 1150 to 120 ng of DNA, 120 to 125 ng of DNA, 125 to 130 ng of DNA, 130 to 135 ng of DNA, 135 to 140 ng of DNA, 140 to 145 ng of DNA, 145 to 150 ng of DNA, 150 to 155 ng of DNA, 155 to 160 ng of DNA, 160 to 165 ng of DNA, 165 to 170 ng of DNA, 170 to 175 ng of DNA, 180 to 185 ng of DNA, 185 to 190 ng of DNA, 195 to 195 ng of DNA, or 195 to 200 ng of DNA. In some embodiments, the one or more cell populations is diluted to contain 200 to 500,000 ng of DNA, such as 200-500 ng, 500-1000 ng, 1000-1500 ng, 1500-2000 ng, 2000-5000 ng, 5000-10,000 ng, 10,000-15,000 ng, 15,000-20,000 ng, 20,000 to 25,000 ng, 25,000 to 30,000 ng, 30,000 to 35,000 ng, 35,000 to 40,000 ng, 40,000 to 45,000 ng, or 45,000 to 50,000 ng of DNA.

In some embodiments, the one or more cell populations is diluted to contain 1 to 500,000 cells. In some embodiments, the one or more cell populations is diluted to contain 1 to 400,000 cells. In some embodiments, the one or more cell populations is diluted to contain 1 to 300,000 cells. In some embodiments, the one or more cell populations is diluted to contain 1 to 200,000 cells. In some embodiments, the one or more cell populations is diluted to contain 1 to 100,000 cells. In some embodiments, the one or more cell populations is diluted to contain 1 to 50,000 cells. In some embodiments, the one or more cell populations is diluted to contain 1 to 40,000 cells. In some embodiments, the one or more cell populations is diluted to contain 1 to 30,000 cells. In some embodiments, the one or more cell populations is diluted to contain 1 to 30,000 cells. In some embodiments, the one or more cell populations is diluted to contain 1 to 20,000 cells. In some embodiments, the one or more cell populations is diluted to contain 1 to 15,000 cells. In some embodiments, the one or more cell populations is diluted to contain 1 to 16,000 cells. In some embodiments, the one or more cell populations is diluted to contain 1 to 15,000 cells. In some embodiments, the one or more cell populations is diluted to contain 1 to 10,000 cells. In some embodiments, the one or more cell populations is diluted to contain 1 to 100 cells, 100 to 200 cells, 200 to 300 cells, 300 to 400 cells 400 to 500 cells, 500 to 600 cells, 600 to 700 cells, 700 to 800 cells, 800 to 900 cells, 900 to 1000 cells, 1000 to 1100 cells, 1100 to 1200 cells, 1200 to 1300 cells, 1300 to 1400 cells, or 1400 to 1500 cells. In some embodiments, the one or more cell populations is diluted to contain 20,000 cells or less, 19,000 cells or less, 18,000 cells or less, 17,000 cells or less, 16,000 cells or less, 15,000 cells or less, 14,000 cells or less, 13,000 cells or less, 12,000 cells or less, 11,000 cells or less, 10,000 cells or less, 9,000 cells or less, 8,000 cells or less, 7,000 cells or less, 6,000 cells or less, 5,000 cells or less, 4,000 cells or less, 3,000 cells or less, 2,000 cells or less, 1,500 cells or less, 1,000 cells or less, 500 cells, 250 cells or less, 100 cells or less, 50 cells or less, 25 cells or less, 10 cells or less, 5 cells or less, or 2 cells or less. In some embodiments, the one or more cell populations is diluted to contain 1 cell. In some embodiments, the one or more cell populations is diluted to contain 1 to 15,000 cells.

### Preparation of the Cellular Sample Prior to Cellular Barcoding

The steps described in this section occur prior to cellular barcoding to produce DNA or RNA inserts for which cellular barcoding of the present methods is performed on.

### Fixing and Permeabilizing Cells Prior to Barcoding

Before the heterogeneous cell population comes into contact with phenotypic barcodes, the heterogeneous population is fixed and permeabilized.

For example, in some embodiments, the sample is fixed and permeabilized one of more cell populations of the sample. Fixing and permeabilizing cells from one or more cell populations can be performed upon collection of the sample.

In some embodiments, the method includes suspending one or more cells within one or more cell populations in a liquid. In some embodiments, the cellular sample in suspension are fixed and permeabilized as desired.

Fixing and permeabilizing the cellular sample can be performed by any convenient method as desired. For example, in some embodiments, the cellular sample is fixed according to fixing and permeabilization techniques described in U.S. Pat. No. 10,627,389, which is hereby incorporated by reference in its entirety.

In some embodiments, fixing the cellular sample includes contacting the sample with a fixation reagent. Fixation reagents of interest are those that fix the cells at a desired time-point. Any convenient fixation reagent may be employed, where suitable fixation reagents include, but are not limited to: formaldehyde, paraformaldehyde, formaldehyde/acetone, methanol/acetone, IncellFP (IncellDx, Inc), and the like. In some embodiments, the cellular sample is Formalin-Fixed Paraffin-Embedded (FFPE). For example, paraformaldehyde used at a final concentration of about 1 to 2% has been found to be a good cross-linking fixative.

In some embodiments, the cells in the sample are permeabilized by contacting the cells with a permeabilizing reagent. Permeabilizing reagents of interest are reagents that allow the labeled biomarker probes, e.g., as described in greater detail below, to access to the intracellular environment. Any convenient permeabilizing reagent may be employed, where suitable reagents include, but are not limited to: mild detergents, such as EDTA, Tris, IDTE (10 mM Tris, 0.1 mM EDTA), Triton X-100, NP-40, saponin, Tween-20, etc.; methanol, and the like.

In some embodiments, a collected liquid sample, e.g., as obtained from fine needle aspirations (FNA) or a pipette that results in dissociation of the cells, is immediately contacted with solution intended to prepare the cells of the sample for further processing, e.g., fixation solution, permeabilization solution, staining solution, labeling solution, or combinations thereof, so to minimize degradation of the cells of the sample that may occur prior to preparation of the cells or prior to analysis of the cells. By “immediately contacted” used herein and in its conventional sense, the cells of the sample or the sample itself is contacted with the subject agent or solution without unnecessary delay from the time the sample is collected. In some embodiments, a sample is immediately contacted with a preparative agent or solution in 6 or less hours from the time the sample is collected, including but not limited to, e.g., 5 hours or less, 4 hours or less, 3 hours or less, 2 hours or less, 1 hours or less, 30 min. or less, 20 min. or less, 15 min. or less, 10 min. or less, 5 min. or less, 4 min. or less, 3 min. or less, 2 min. or less, 1 min. or less, etc., optionally including a lower limit of the minimum amount of time necessary to physically contact the sample with the preparative agent or solution, which may, in some instances be on the order of 1 sec. to 30 sec or more.

Preparation of the sample and/or fixation of the cells of the sample is performed in such a manner that the prepared cells of the sample maintain several characteristics of the unprepared cells, including, but not limited to, characteristics of unprepared cells in situ, i.e., prior to collection, and/or unfixed cells following collection but prior to fixation and/or permeabilization and/or labeling. Such characteristics that may be maintained include but are not limited to, e.g., cell morphological characteristics including but not limited to, e.g., cell size, cell volume, cell shape, etc. The preservation of cellular characteristics through sample preparation may be evaluated by any convenient means including, e.g., the comparison of prepared to cells to one or more control samples of cells such as unprepared or unfixed or unlabeled samples. Comparison of cells of a prepared sample to cells of an unprepared sample of a particular measured characteristic may provide a percent preservation of the characteristic that will vary depending on the particular characteristic evaluated. The percent preservation of cellular characteristics of cells prepared according to the methods described herein will vary and may range from 50% maintenance or more including but not limited to, e.g., 60% maintenance or more, 65% maintenance or more, 70% maintenance or more, 75% maintenance or more, 80% maintenance or more, 85% maintenance or more, 90% maintenance or more, etc., and optionally with a maximum of 100% maintenance. In some instances, preservation of a particular cellular characteristic may be evaluated based on comparison to a reference value of the characteristic (e.g., from a predetermined measurement of one or more control cells, from a known reference standard based on unprepared cells, etc.). In some embodiments, the cells may be evaluated using a hemocytometer, microscope, and/or any other known cell counting method.

In some embodiments, the method of fixing and permeabilizing the cells include spinning the cells down, contained within a tube, with a centrifuge (e.g., 1,000 G at 5 min) to separate the supernatant from the cells. In some embodiments, the method includes adding 500 μl freezing media after spinning the cells. In some embodiments, the cells in the freezing media are placed in a refrigerator at a temperature of about −20° C.±5° C. In some embodiments, the cells in the freezing media are placed in a refrigerator at a temperature of about −20° C.±10° C.

In such embodiments, the method includes removing the first supernatant without disturbing the cell pellet. In some embodiments, the method includes adding 100 μl IDTE buffer after removing the first supernatant.

In such embodiments, the method includes adding phosphate buffered saline (PBS) to the cells contained within the tube after removing the first supernatant. In some embodiments, the method includes adding 500 μl freezing media after adding PBS to the cells. In some embodiments, the cells in the freezing media are placed in a refrigerator at a temperature of about −20° C.±5° C. In some embodiments, the cells in the freezing media are placed in a refrigerator at a temperature of about −20° C.±10° C.

In such embodiments, the method includes gently mixing the cells after adding PBS by pipetting to re-suspend the cell pellet. In such embodiments, the method includes spinning the cells down (e.g., 1,000 G at 5 min). In such embodiments, the method includes removing the second supernatant without disturbing the cell pellet. In such embodiments, the method includes adding IDTE or any known permeabilizing buffer to the cells. In some embodiments, about 11 μl of IDTE is added to about 16,000 cells.

### Library Prep—Amplification Methods to Produce DNA or RNA Inserts In Situ

In some embodiments, after the heterogeneous cell population is permeabilized and fixed, the one or more cell populations of the sample is contacted with a first primer pool set, and the DNA or RNA nucleic acids from the cell populations are amplified using the first primer pool set-to produce a first set of amplicon products.

In some embodiments, the primers in the first primer pool set are DNA primers. In some embodiments, the primers in the first primer pool set are RNA primers.

Amplification of Nucleic Acids from Cells of a Heterogeneous Sample

Primer Sets

In some embodiments, the one or more cell populations of the sample with a first primer pool set. In some embodiments, the first primer pool set of the present disclosure is designed to amplify multiple targets with the use of multiple primer pairs in a single PCR experiment. In some embodiments, the number of targets include 1 or more target, 2 or more targets, 3 or more targets, 4 or more targets, 5 or more targets, 6 or more targets, 7 or more targets, 8 or more targets, 9 or more targets, or 10 or more targets. In some embodiments, the number of targets include 15 or more, 20 or more, 25 or more, 30 or more, 35 or more, 40 or more, 45 or more, 50 or more, 55 or more, 60 or more, 70 or more, 80 or more, 90 or more, or 100 or more forward and reverse primers. In some embodiments, the first primer pool set comprises 100 or more, 125 or more, 150 or more, 175 or more, 200 or more, 225 or more, 250 or more, 275 or more, 300 or more, 325 or more, 350 or more, 375 or more, 400 or more, 425 or more, 450 or more, 475 or more, or 500 or more targets. In some embodiments, the number of targets includes a range of 5-25, 25 to 50, 50 to 75, 75 to 100, 100 to 150, 150 to 200, 200 to 250, 250 to 300, 300 to 350, 350 to 400, 400 to 450, 450 to 500, 500 to 550, 550 to 600, 600 to 650, 650 to 700, 700 to 750, 750 to 800, 800 to 850, 850 to 900, 900 to 950, or 950 to 1000 targets. In some embodiments, the number of targets includes 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10,000 or more, 10,500 or more, 11,000 or more, 11,500 or more, 12,000 or more, 12,500 or more, 13,000 or more, 13,500 or more, 14,000 or more, 14,500 or more, 15,000 or more, 15,500 or more, 20,000 or more, 20,500 or more, 21,500 or more, 22,000 or more, 22,500 or more, 23,000 or more, 24,500 or more, 25,000 or more, 25,500 or more, 26,000 or more, 26,500 or more, 27,000 or more, 27,500 or more, 28,000 or more, 28,500 or more, or 30,000 or more targets. In some embodiments, the number of targets includes 25,000 or more, 30,000 or more, 35,000 or more, 40,000 or more, 45,000 or more, 50,000 or more, 55,000 or more, 60,000 or more, or 65,000 or more targets. In some embodiments, the number of targets ranges from 1-30,000 targets, 1-25,000 targets, 1-26,000 targets, 1-1000 targets, 1000-2000 targets, 2000-3000 targets, 3000-4000 targets, 4000-5000 targets, 5000-6000 targets, 6000-7000 targets, 7000-8000 targets, 8000-9000 targets, 9000 to 10,000 targets, 10,000 to 11,000 targets, 11,000 to 12,000 targets, 12,000 to 13,000 targets, 13,000 to 14,000 targets, 14,000 to 15,000 targets, 15,000 to 16,000 targets, 16,000 to 17,000 targets, 17,000 to 18,000 targets, 18,000 to 19,000 targets, 19,000 to 20,000 targets, 20,000 to 21,000 targets, 21,000 to 22,000 targets, 22,000 to 23,000 targets, 23,000 to 24,000 targets, 24,000 to 25,000 targets, 25,000 to 26,000 targets, 26,000 to 27,000 targets, 27,000 to 28,000 targets, 28,000 to 29,000 targets, or 29,000 to 30,000 targets.

In some embodiments the first primer pool set comprises a first forward primer pool. In some embodiments, the first primer pool set comprises a first reverse primer pool. In some embodiments the first primer pool set comprises a first forward primer pool and a reverse primer pool. In some embodiments, the first primer pool set comprises 5 or more, 10 or more, 15 or more, 20 or more, 25 or more, 30 or more, 35 or more, 40 or more, 45 or more, 50 or more, 55 or more, 60 or more, 70 or more, 80 or more, 90 or more, or 100 or more forward and reverse primers. In some embodiments, the first primer pool set comprises 100 or more, 125 or more, 150 or more, 175 or more, 200 or more, 225 or more, 250 or more, 275 or more, 300 or more, 325 or more, 350 or more, 375 or more, 400 or more, 425 or more, 450 or more, 475 or more, or 500 or more forward and reverse primers. In some embodiments, the first primer pool set includes a range of 5-1000 forward and reverse primers. In some embodiments, the first primer pool set includes a range of 5-25, 25 to 50, 50 to 75, 75 to 100, 100 to 150, 150 to 200, 200 to 250, 250 to 300, 300 to 350, 350 to 400, 400 to 450, 450 to 500, 500 to 550, 550 to 600, 600 to 650, 650 to 700, 700 to 750, 750 to 800, 800 to 850, 850 to 900, 900 to 950, or 950 to 1000 forward and reverse primers. In some embodiments, the first primer pool set includes 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10,000 or more, 10,500 or more, 11,000 or more, 11,500 or more, 12,000 or more, 12,500 or more, 13,000 or more, 13,500 or more, 14,000 or more, 14,500 or more, 15,000 or more, 15,500 or more, 20,000 or more, 20,500 or more, 21,500 or more, 22,000 or more, 22,500 or more, 23,000 or more, 24,500 or more, 25,000 or more, 25,500 or more, 26,000 or more, 26,500 or more, 27,000 or more, 27,500 or more, 28,000 or more, 28,500 or more, or 30,000 or more forward and reverse primers. In some embodiments, the first primer pool set includes 25,000 or more, 30,000 or more, 35,000 or more, 40,000 or more, 45,000 or more, 50,000 or more, 55,000 or more, 60,000 or more, or 65,000 or more forward and reverse primers. In some embodiments, the first primer pool set ranges from 1-30,000 forward and reverse primers, 1-60,000 forward and reverse primers, 1-50,000 forward and reverse primers, 1-25,000 forward and reverse primers, 1-26,000 forward and reverse primers, 1-1000 forward and reverse primers, 1000-2000 forward and reverse primers, 2000-3000 forward and reverse primers, 3000-4000 forward and reverse primers, 4000-5000 forward and reverse primers, 5000-6000 forward and reverse primers, 6000-7000 forward and reverse primers, 7000-8000 forward and reverse primers, 8000-9000 forward and reverse primers, 9000 to 10,000 forward and reverse primers, 10,000 to 11,000 forward and reverse primers, 11,000 to 12,000 forward and reverse primers, 12,000 to 13,000 forward and reverse primers, 13,000 to 14,000 forward and reverse primers, 14,000 to 15,000 forward and reverse primers, 15,000 to 16,000 forward and reverse primers, 16,000 to 17,000 forward and reverse primers, 17,000 to 18,000 forward and reverse primers, 18,000 to 19,000 forward and reverse primers, 19,000 to 20,000 forward and reverse primers, 20,000 to 21,000 forward and reverse primers, 21,000 to 22,000 forward and reverse primers, 22,000 to 23,000 forward and reverse primers, 23,000 to 24,000 forward and reverse primers, 24,000 to 25,000 forward and reverse primers, 25,000 to 26,000 forward and reverse primers, 26,000 to 27,000 forward and reverse primers, 27,000 to 28,000 forward and reverse primers, 28,000 to 29,000 forward and reverse primers, 29,000 to 30,000 forward and reverse primers, 30,000 to 40,000 forward and reverse primers, 40,000 to 50,000 forward and reverse primers, or 50,000 to 60,000 forward and reverse primers.

In some embodiments, the forward primer pool comprises 5 or more, 10 or more, 15 or more, 20 or more, 25 or more, 30 or more, 35 or more, 40 or more, 45 or more, 50 or more, 55 or more, 60 or more, 70 or more, 80 or more, 90 or more, or 100 or more forward primers. In some embodiments, the first primer pool comprises 100 or more, 125 or more, 150 or more, 175 or more, 200 or more, 225 or more, 250 or more, 275 or more, 300 or more, 325 or more, 350 or more, 375 or more, 400 or more, 425 or more, 450 or more, 475 or more, or 500 or more forward primers. In some embodiments, the forward primer pool includes a range of 5-1000 forward primers. In some embodiments, the forward primer pool includes a range of 5-25, 25 to 50, 50 to 75, 75 to 100, 100 to 150, 150 to 200, 200 to 250, 250 to 300, 300 to 350, 350 to 400, 400 to 450, 450 to 500, 500 to 550, 550 to 600, 600 to 650, 650 to 700, 700 to 750, 750 to 800, 800 to 850, 850 to 900, 900 to 950, or 950 to 1000 forward primers. In some embodiments, the forward primer pool includes 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10,000 or more, 10,500 or more, 11,000 or more, 11,500 or more, 12,000 or more, 12,500 or more, 13,000 or more, 13,500 or more, 14,000 or more, 14,500 or more, 15,000 or more, 15,500 or more, 20,000 or more, 20,500 or more, 21,500 or more, 22,000 or more, 22,500 or more, 23,000 or more, 24,500 or more, 25,000 or more, 25,500 or more, 26,000 or more, 26,500 or more, 27,000 or more, 27,500 or more, 28,000 or more, 28,500 or more, or 30,000 or more forward primers. In some embodiments, the forward primer pool ranges from 1-30,000 forward primers, 1-60,000 forward primers, 1-50,000 forward primers, 1-25,000 forward primers, 1-26,000 forward primers, 1-1000 forward primers, 1000-2000 forward primers, 2000-3000 forward primers, 3000-4000 forward primers, 4000-5000 forward primers, 5000-6000 forward primers, 6000-7000 forward primers, 7000-8000 forward primers, 8000-9000 forward primers, 9000 to 10,000 forward primers, 10,000 to 11,000 forward primers, 11,000 to 12,000 forward primers, 12,000 to 13,000 forward primers, 13,000 to 14,000 forward primers, 14,000 to 15,000 forward primers, 15,000 to 16,000 forward primers, 16,000 to 17,000 forward primers, 17,000 to 18,000 forward primers, 18,000 to 19,000 forward primers, 19,000 to 20,000 forward primers, 20,000 to 21,000 forward primers, 21,000 to 22,000 forward primers, 22,000 to 23,000 forward primers, 23,000 to 24,000 forward primers, 24,000 to 25,000 forward primers, 25,000 to 26,000 forward primers, 26,000 to 27,000 forward primers, 27,000 to 28,000 forward primers, 28,000 to 29,000 forward primers, or 29,000 to 30,000 forward primers. In some embodiments, each forward primer includes a nucleotide sequence having a length ranging from 10 to 200 nucleotides; such as, 10 to 20 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, 140 to 150 nucleotides, 150 to 160 nucleotides, 160 to 170 nucleotides, 170 to 180 nucleotides, 180 to 190 nucleotides, or 190 to 200 nucleotides. In some embodiments, each forward primer includes a nucleotide sequence having a length ranging from 10 to 50 nucleotides, such as 10 to 30, 20 to 40, or 30 to 50 nucleotides. In some embodiments, each forward primer includes a nucleotide sequence having a length ranging from 10 to 20 nucleotides, such as 10 to 12, 12 to 14, 10 to 15, 14 to 16, 16 to 18, or 18 to 20 nucleotides. In some embodiments, each forward primer includes a nucleotide sequence having a length of 10, 11, 12, 13, 14, 15, 16, 17, 18, 19, 20, 21, 22, 23, 24, 25, 26, 27, 28, 29, or 30 nucleotides.

In some embodiments, each forward primer comprises a nucleotide sequence that hybridize to an anti-sense strand of a nucleotide sequence encoding a target region of one or more cells. In some embodiments, each primer comprises a unique nucleotide sequence that hybridizes to an anti-sense strand of a nucleotide sequence encoding a different target region of one or more cells. Thus, a forward primer pool can include a plurality of forward primers, where each forward primer hybridizes to a distinct target nucleic acid.

In some embodiments, the reverse primer pool comprises 5 or more, 10 or more, 15 or more, 20 or more, 25 or more, 30 or more, 35 or more, 40 or more, 45 or more, 50 or more, 55 or more, 60 or more, 70 or more, 80 or more, 90 or more, or 100 or more reverse primers. In some embodiments, the first primer pool comprises 100 or more, 125 or more, 150 or more, 175 or more, 200 or more, 225 or more, 250 or more, 275 or more, 300 or more, 325 or more, 350 or more, 375 or more, 400 or more, 425 or more, 450 or more, 475 or more, or 500 or more reverse primers. In some embodiments, the reverse primer pool includes a range of 5-1000 reverse primers. In some embodiments, the reverse primer pool includes a range of 5-25, 25 to 50, 50 to 75, 75 to 100, 100 to 150, 150 to 200, 200 to 250, 250 to 300, 300 to 350, 350 to 400, 400 to 450, 450 to 500, 500 to 550, 550 to 600, 600 to 650, 650 to 700, 700 to 750, 750 to 800, 800 to 850, 850 to 900, 900 to 950, or 950 to 1000 reverse primers. In some embodiments, the reverse primer pool includes 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10,000 or more, 10,500 or more, 11,000 or more, 11,500 or more, 12,000 or more, 12,500 or more, 13,000 or more, 13,500 or more, 14,000 or more, 14,500 or more, 15,000 or more, 15,500 or more, 20,000 or more, 20,500 or more, 25,000 or more, 25,500 or more, 26,000 or more, 26,500 or more, 27,000 or more, 27,500 or more, 28,000 or more, 28,500 or more, or 30,000 or more reverse primers. In some embodiments, the reverse primer pool ranges from 1-30,000 reverse primers, 1-60,000 reverse primers, 1-50,000 reverse primers, 1-25,000 reverse primers, 1-26,000 reverse primers, 1-1000 reverse primers, 1000-2000 reverse primers, 2000-3000 reverse primers, 3000-4000 reverse primers, 4000-5000 reverse primers, 5000-6000 reverse primers, 6000-7000 reverse primers, 7000-8000 reverse primers, 8000-9000 reverse primers, 9000 to 10,000 reverse primers, 10,000 to 11,000 reverse primers, 11,000 to 12,000 reverse primers, 12,000 to 13,000 reverse primers, 13,000 to 14,000 reverse primers, 14,000 to 15,000 reverse primers, 15,000 to 16,000 reverse primers, 16,000 to 17,000 reverse primers, 17,000 to 18,000 reverse primers, 18,000 to 19,000 reverse primers, 19,000 to 20,000 reverse primers, 20,000 to 21,000 reverse primers, 21,000 to 22,000 reverse primers, 22,000 to 23,000 reverse primers, 23,000 to 24,000 reverse primers, 24,000 to 25,000 reverse primers, 25,000 to 26,000 reverse primers, 26,000 to 27,000 reverse primers, 27,000 to 28,000 reverse primers, 28,000 to 29,000 reverse primers, or 29,000 to 30,000 reverse primers.

In some embodiments, each reverse primer includes a nucleotide sequence having a length ranging from 10 to 200 nucleotides; such as, 10 to 20 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, 140 to 150 nucleotides, 150 to 160 nucleotides, 160 to 170 nucleotides, 170 to 180 nucleotides, 180 to 190 nucleotides, or 190 to 200 nucleotides. In some embodiments, each reverse primer includes a nucleotide sequence having a length ranging from 10 to 50 nucleotides, such as 10 to 30, 20 to 40, or 30 to 50 nucleotides. In some embodiments, each reverse primer includes a nucleotide sequence having a length ranging from 10 to 20 nucleotides, such as 10 to 12, 12 to 14, 10 to 15, 14 to 16, 16 to 18, or 18 to 20 nucleotides. In some embodiments, each reverse primer includes a nucleotide sequence having a length of 10, 11, 12, 13, 14, 15, 16, 17, 18, 19, 20, 21, 22, 23, 24, 25, 26, 27, 28, 29, or 30 nucleotides.

In some embodiments, each reverse primer comprises a nucleotide sequence that hybridize to a sense strand of a nucleotide sequence encoding a target region of one or more cells. In some embodiments, each primer comprises a unique nucleotide sequence that hybridizes to an anti-sense strand of a nucleotide sequence encoding a different target region of one or more cells. Thus, a reverse primer pool can include a plurality of reverse primers, where each reverse primer hybridizes to a distinct target nucleic acid.

As described herein, a first primer pool set can include publicly available primer pool sets of known nucleic target regions of interest. In some embodiments, a forward primer pool includes primers of a rhAmp PCR Panel. In some embodiments, a reverse primer pool includes primers of a rhAmp PCR Panel.

Aspects of the present disclosure include amplifying nucleic acids from the cell population using the first primer pool set to produce a first set of amplicon products. In some embodiments, the nucleic acids of the one or more cell populations are amplified in situ.

The term “amplicon”, as used herein and in its conventional sense, refers to the amplified nucleic acid product of a PCR reaction or other nucleic acid amplification process (e.g., ligase chain reaction (LGR), nucleic acid sequence based amplification (NASBA), transcription-mediated amplification (TMA), Q-beta amplification, strand displacement amplification, target mediated amplification, and the like) Amplicons may comprise RNA or DNA depending on the technique used for amplification. For example, DNA amplicons may be generated by RT-PCR, whereas RNA amplicons may be generated by TMA/NASBA.

Multiplexed Polymerase Chain Reaction

As explained above, the primer sets described herein is used in multiplexed PCR-based techniques, such as RT-PCR or in situ PCR, for amplification of target nucleic acids in a sample containing a heterogeneous cell population to produce amplicon products. PCR is a technique for amplifying desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, N Y 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

Aspects of the present methods include using multiplexed PCR for amplification of multiple targets in a single PCR experiment. As a non-limiting example, in a multiplexing assay, more than one target sequence can be amplified by using multiple primer pairs in a reaction mixture.

In particular, PCR uses relatively short oligonucleotide primers which flank the target nucleotide sequence to be amplified, oriented such that their 3′ ends face each other, each primer extending toward the other. The polynucleotide sample is extracted and denatured, e.g., by heat, and hybridized with first and second primers that are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. In some cases, PCR is carried out with a commercially available thermal cycler, e.g., Perkin Elmer.

RNA may be amplified by reverse transcribing the RNA into cDNA, and then performing PCR (RT-PCR), as described above. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall et al. (1994) PCR Meth. App. 4:80-84. Suitable DNA polymerases include reverse transcriptases, such as avian myeloblastosis virus (AMV) reverse transcriptase (available from, e.g., Seikagaku America, Inc.) and Moloney murine leukemia virus (MMLV) reverse transcriptase (available from, e.g., Bethesda Research Laboratories).

Any PCR reaction mixture and heat-resistant DNA polymerase may be used to produce amplicon products. For example, those contained in a commercially available PCR kit can be used. As the reaction mixture, any buffer known to be usually used for PCR can be used. Examples include IDTE (10 mM Tris, 0.1 mM EDTA; Integrated DNA Technologies), Tris-HCl buffer, a Tris-sulfuric acid buffer, a tricine buffer, and the like. Examples of heat-resistant polymerases include Taq DNA polymerase (e.g., FastStart Taq DNA Polymerase (Roche), Ex Taq (registered trademark) (Takara), Z-Taq, AccuPrime Taq DNA Polymerase, M-PCR kit (QIAGEN), KOD DNA polymerase, and the like.

The amounts of the primer and template DNA used, etc., in the present disclosure can be adjusted according to the PCR kit and device used. In some embodiments, about 0.1 to 1 μl of the first primer pool set is added to the PCR reaction mixture. In some embodiments, a forward primer pool of about 0.5 μl, about 1 μl, about 1.5 μl, about 2 μl, about 2.5 μl, about 3 μl, about 3.5 μl, about 4 μl, about 4.5 μl, or about 5 μl is added to the PCR reaction mixture. In some embodiments, a reverse primer pool of about 0.5 μl, about 1 μl, about 1.5 μl, about 2 μl, about 2.5 μl, about 3 μl, about 3.5 μl, about 4 μl, about 4.5 μl, or about 5 μl is added to the PCR reaction mixture.

In some embodiments, the PCR reaction mixture includes the first primer pool set, the population of cells, and a PCR library mix. In some embodiments, the library mix is a rhAmpSeq Library Mix. In some embodiments, a forward primer pool of the first primer pool set includes forward primers of a rhAmp PCR Panel. In some embodiments, a reverse primer pool of the first primer pool set includes reverse primers of a rhAmp PCR Panel.

In some embodiments, about 0.1 to 10 μl of the PCR library mix is added to the PCR reaction mixture. In some embodiments, a PCR library mix of about 0.5 μl, about 1 μl, about 1.5 μl, about 2 μl, about 2.5 μl, about 3 μl, about 3.5 μl, about 4 μl, about 4.5 μl, about 5 μl, about 6 μl, about 7 μl, about 8 μl, about 9 μl, or about 10 μl, is added to the PCR reaction mixture.

The PCR reaction mixture of the present disclosure includes one or more cell populations. In some embodiments, the cell population is diluted to a volume of about 0.5 μl, about 1 μl, about 1.5 μl, about 2 μl, about 2.5 μl, about 3 μl, about 3.5 μl, about 4 μl, about 4.5 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, or about 20 μl. In some embodiments, the one or more cell populations is diluted to contain about 5 to about 200 ng of DNA. In some embodiments, the one or more cell populations is diluted to contain about 1 to about 100 ng of DNA. In some embodiments, the one or more cell populations is diluted to contain about 1 to about 200 ng of DNA (e.g., about 1 to 25 ng of DNA, about 25 to 50 ng of DNA, about 50 to 75 ng of DNA, about 75 to 100 ng of DNA, about 100 to 125 ng of DNA, about 125 to 150 ng of DNA, about 150 to 175 ng of DNA, or about 175 to 200 ng of DNA). In some embodiments, the one or more cell population is diluted to about 100 ng or less, 75 ng or less, 50 ng or less, 25 ng or less 10 ng or less, 5 ng or less, 2 ng or less, or 1 ng or less of DNA. In some embodiments, the one or more cell populations is diluted to contain about 5 to about 100 ng of DNA. In some embodiments, the one or more cell populations is diluted to contain 5 to 10 ng of DNA, 10 to 15 ng of DNA, 15 to 20 ng of DNA, 20 to 25 ng of DNA, 25 to 30 ng of DNA, 30 to 35 ng of DNA, 35 to 40 ng of DNA, 40 to 45 ng of DNA, 45 to 50 ng of DNA, 50 to 55 ng of DNA, 55 to 60 ng of DNA, 60 to 65 ng of DNA, 65 to 70 ng of DNA, 70 to 75 ng of DNA, 75 to 80 ng of DNA, 80 to 85 ng of DNA, 85 to 90 ng of DNA, 90 to 95 ng of DNA, 95 to 100 ng of DNA, 100 to 105 ng of DNA, 105 to 110 ng of DNA, 110 to 115 ng of DNA, 1150 to 120 ng of DNA, 120 to 125 ng of DNA, 125 to 130 ng of DNA, 130 to 135 ng of DNA, 135 to 140 ng of DNA, 140 to 145 ng of DNA, 145 to 150 ng of DNA, 150 to 155 ng of DNA, 155 to 160 ng of DNA, 160 to 165 ng of DNA, 165 to 170 ng of DNA, 170 to 175 ng of DNA, 180 to 185 ng of DNA, 185 to 190 ng of DNA, 195 to 195 ng of DNA, or 195 to 200 ng of DNA. In some embodiments, the one or more cell populations is diluted to contain 200 to 500,000 ng of DNA, such as 200-500 ng, 500-1000 ng, 1000-1500 ng, 1500-2000 ng, 2000-5000 ng, 5000-10,000 ng, 10,000-15,000 ng, 15,000-20,000 ng, 20,000 to 25,000 ng, 25,000 to 30,000 ng, 30,000 to 35,000 ng, 35,000 to 40,000 ng, 40,000 to 45,000 ng, or 45,000 to 50,000 ng of DNA.

In some embodiments, the one or more cell populations is diluted to contain 1 to 500,000 cells. In some embodiments, the one or more cell populations is diluted to contain 1 to 400,000 cells. In some embodiments, the one or more cell populations is diluted to contain 1 to 300,000 cells. In some embodiments, the one or more cell populations is diluted to contain 1 to 200,000 cells. In some embodiments, the one or more cell populations is diluted to contain 1 to 100,000 cells. In some embodiments, the one or more cell populations is diluted to contain 1 to 50,000 cells. In some embodiments, the one or more cell populations is diluted to contain 1 to 40,000 cells. In some embodiments, the one or more cell populations is diluted to contain 1 to 30,000 cells. In some embodiments, the one or more cell populations is diluted to contain 1 to 30,000 cells. In some embodiments, the one or more cell populations is diluted to contain 1 to 20,000 cells. In some embodiments, the one or more cell populations is diluted to contain 1 to 15,000 cells. In some embodiments, the one or more cell populations is diluted to contain 1 to 16,000 cells. In some embodiments, the one or more cell populations is diluted to contain 1 to 15,000 cells. In some embodiments, the one or more cell populations is diluted to contain 1 to 10,000 cells. In some embodiments, the one or more cell populations is diluted to contain 1 to 100 cells, 100 to 200 cells, 200 to 300 cells, 300 to 400 cells 400 to 500 cells, 500 to 600 cells, 600 to 700 cells, 700 to 800 cells, 800 to 900 cells, 900 to 1000 cells, 1000 to 1100 cells, 1100 to 1200 cells, 1200 to 1300 cells, 1300 to 1400 cells, or 1400 to 1500 cells. In some embodiments, the one or more cell populations is diluted to contain 20,000 cells or less, 19,000 cells or less, 18,000 cells or less, 17,000 cells or less, 16,000 cells or less, 15,000 cells or less, 14,000 cells or less, 13,000 cells or less, 12,000 cells or less, 11,000 cells or less, 10,000 cells or less, 9,000 cells or less, 8,000 cells or less, 7,000 cells or less, 6,000 cells or less, 5,000 cells or less, 4,000 cells or less, 3,000 cells or less, 2,000 cells or less, 1,500 cells or less, 1,000 cells or less, 500 cells, 250 cells or less, 100 cells or less, 50 cells or less, 25 cells or less, 10 cells or less, 5 cells or less, or 2 cells or less. In some embodiments, the one or more cell populations is diluted to contain 1 cell. In some embodiments, the one or more cell populations is diluted to contain 1 to 15,000 cells.

As described herein, the PCR cycling conditions are not particularly limited as long as the desired target genes can be amplified. For example, the thermal denaturation temperature can be set to 92 to 100° C., e.g., 94 to 98° C. The thermal denaturation time can be set to, for example, 5 to 180 seconds, e.g., 10 to 130 seconds. The annealing temperature for hybridizing primers can be set to, for example, 55 to 80° C., e.g., 60 to 70° C. The annealing time can be set to, for example, 10 to 60 seconds, e.g., 10 to 20 seconds. The extension reaction temperature can be set to, for example, 55 to 80° C., e.g., 60 to 70° C. The elongation reaction time can be set to, for example, 4 to 15 minutes, e.g., 10 to 20 minutes. In some embodiments, the annealing and extension reaction can be performed under the same conditions. In some embodiments, the operation of combining thermal denaturation, annealing, and an elongation reaction is defined as one cycle. This cycle can be repeated until the required amounts of amplification products are obtained. For example, the number of cycles can be set to 30 to 40 times, e.g., about 30 to 35 times. In some embodiments, the number of cycles can be set to 5 to 10 cycles, 10 to 15 cycles, 15 to 20 cycles, 20 to 25 cycles, 25 to 30 cycles, 35 to 40 cycles, 45 to 50 cycles, or 55 to 60 cycles.

In the present disclosure, the “PCR cycling conditions” may include one of, any combination of, or all of the conditions with respect to the temperature and time of each thermal denaturation, annealing, and elongation reaction of PCR and the number of cycles. When PCR cycling conditions are set, the touchdown PCR method can be used in terms of inhibiting non-specific amplification. Touchdown PCR is a technique in which the first annealing temperature is set to a relatively high temperature and the annealing temperature is gradually reduced for each cycle, and, midway and thereafter, PCR is performed in the same manner as general PCR. Shuttle PCR may also be used in terms of inhibiting non-specific amplification. Shuttle PCR is a PCR in which annealing, and extension reaction are performed at the same temperature.

Although different PCR cycling conditions can be used for each primer pair, it is preferable from the viewpoint of operation and efficiency that PCR cycling conditions are set in such a manner that the same PCR cycling conditions can be used for different primer pairs and the variation of PCR cycling conditions used to obtain necessary amplification products is minimized. The number of variations of PCR cycling conditions is preferably 10 or less, 5 or less, more preferably 4 or less, still more preferably 3 or less, even more preferably 2 or less, and even still more preferably 1. When the number of variations of PCR cycling conditions used to obtain all the necessary amplification products is reduced, PCRs using the same PCR cycling conditions can be simultaneously performed using one PCR device. Accordingly, the desired amplification products can be obtained in a short time using smaller amounts of resources.

In some embodiments, the method of the present disclosure includes, after producing the first set of amplicon products, purifying the first set of amplicon products. Techniques for purifying amplicon products are well-known in the art and include, for example, using magnetic bead purification reagent, passing through a column, use of ampure beads, and the like.

Ligation

Aspects of the present disclosure include amplifying or ligating the first set of amplicon products to produce a second set of amplicon products comprising indexed libraries.

In some embodiments, amplifying the first set of amplicon products includes performing PCR.

In some embodiments, amplifying the first set of amplicon products includes performing ligation. For example, adapters that contain one or more primer sequences (e.g., read/and read2 sequences), and/or barcoding sequences that contain one or more primers can be ligated to the ends of a target nucleic acid, where one type of adapter or multiple types of adapters and/or barcodes can be used in the ligation reaction. Such methods enable one or more target nucleic acid molecules to be amplified in a single amplification reaction, including, for example, target nucleic acids of known and unknown sequence, as well as multiple target nucleic acids of identical or different sequences. Such reformatted target nucleic acids and/or libraries thereof can be readily subjected to various qualitative and quantitative analyses.

In some embodiments, ligating includes performing ligase chain reaction (LCR). The ligase chain reaction (LCR) is an amplification process that involves a thermostable ligase to join two probes or other molecules together. In some embodiments, the ligated product is then amplified to produce a second amplicon product. In some embodiments, LCR can be used as an alternative approach to PCR. In other embodiments, PCR can be performed after LCR.

In some embodiments, the thermostable ligase can include, but is not limited to Pfu ligase, or a Taq ligase.

In some embodiments, after producing the second set of amplicon products, the method includes purifying the second set of amplicon products according to the methods described herein. As described above, techniques for purifying amplicon products are well-known in the art and include, for example, using magnetic bead purification reagent, passing through a column, use of ampure beads, and the like.

In some embodiments, purifying the amplicon product of the present methods creates an enriched library for sequencing. The term “enriched” as used herein and in its conventional sense, refers to isolated nucleotide sequences containing the genomic regions of interest (e.g., target regions) using known purification techniques (e.g., hybridization capture, magnetic bead purification techniques, and the like). In some embodiments, the enriched library includes adapter (e.g., “indexed library”). In some embodiments, the enriched library includes adapter and barcoding sequences (e.g., “barcoded indexed library”). The enriched libraries described in the methods herein includes the final purified library before sequencing.

### Library Preparation—Hybridization Capture Methods to Produce DNA or RNA Inserts In Situ

Aspects of the present methods include receiving a sample comprising a heterogeneous cell population from a sample; contacting one or more cell populations with a set of indexing primers; ligating nucleic acids in one or more cell populations with a set of indexing primers to produce an indexed library; performing hybridization capture on the indexed library to produce an enriched library; sequencing the enriched library; and analyzing the sequenced enriched library to determine the presence or absence of disease-associated genetic alterations within the cell populations.

In some embodiments, performing hybridization capture is an alternative method to amplification techniques for producing DNA or RNA inserts in situ. In some embodiments, performing hybridization capture can be combined with amplification techniques

In certain aspects where hybridization capture is used, in order to create DNA or RNA inserts in situ for cellular barcoding, the following steps can be performed: (a) receiving a sample comprising a heterogeneous cell population; (b) contacting one or more cell populations with a fragmentation buffer and a fragmentation enzyme to form a mixture; (c) performing an enzymatic fragmentation reaction on the mixture to form fragmented DNA or RNA within the one or more cell populations; (d) contacting the one or more cell populations comprising fragmented DNA or RNA with a set of adapter sequences (e.g., R1, R2); (e) ligating the fragmented DNA or RNA to the adapter (e.g., an adapter that includes a R1 sequence or a R2 sequence) to produce an indexed library; (f) performing hybridization capture on the indexed library to produce an enriched indexed library; and (g) analyzing the enriched indexed library to determine the presence or absence of disease-associated genetic alterations within the cell populations.

Non-limiting examples of general hybridization techniques used on gDNA of lysed cells can be found at www(dot)idtdna(dot)com/pages/technology/next-generation-sequencing/library-preparation/ligation-based-library-prep, which is hereby incorporated by reference in its entirety.

Enzymatic Fragmentation

In some embodiments, the method includes contacting the cell population with a fragmentation buffer and a fragmentation enzyme to form an enzymatic fragmentation mixture. Performing an enzymatic fragmentation reaction in the present ligation-based method provides for generating smaller sized DNA or RNA fragments containing the target region of interest. Methods for fragmenting DNA or RNA can include mechanical, chemical, or enzyme-based fragmenting. Mechanical shearing methods include acoustic shearing, sonication, hydrodynamic shearing and nebulization. Chemical fragmentation methods include the use of agents which generate hydroxyl radicals for random DNA cleavage or the use of heat with divalent metal cations, while enzyme-based methods include transposases, restriction enzymes (e.g., mung bean nucleases, nuclease P1, or micrococcal nuclease), DNase I, non-specific nucleases, and nicking enzymes, or a mixture thereof. In some embodiments, enzyme-based DNA/RNA fragmentation methods include using a mixture of at least two different enzymes e.g., two or more of the enzymes mentioned in the preceding sentence e.g. two or more nucleases. Any standard enzymatic fragmentation buffer and enzymatic fragmentation enzyme can be used for fragmenting the DNA or RNA.

In some embodiments, the one or more cell populations, the fragmentation buffer, and fragmentation enzyme are pipetted into a test tube. In some embodiments, the test tube is on ice.

In certain embodiments, this method optionally includes denaturing, by heat, prior to enzymatic fragmentation to improve fragmentation, likely by opening the chromatin structure of DNA or RNA in the one or more cell populations. In alternative embodiments, the heat denaturation step is not performed prior to enzymatic fragmentation.

In some embodiments, the cell population within the enzymatic fragmentation mixture is diluted to a volume of about 0.5 μl or more, about 1 μl or more, about 1.5 μl or more, about 2 μl or more, about 2.5 μl or more, about 3 μl or more, about 3.5 μl or more, about 4 μl or more, about 4.5 μl or more, about 5 μl or more, about 6 μl or more, about 7 μl or more, about 8 μl or more, about 9 μl or more, about 10 μl or more, about 11 μl or more, about 12 μl or more, about 13 μl or more, about 14 μl or more, about 15 μl or more, about 16 μl or more, about 17 μl or more, about 18 μl or more, about 19 μl or more, about 20 μl or more, about 25 μl or more, about 30 μl or more, about 35 μl or more, about 40 μl or more, about 45 μl or more, about 50 μl or more, about 55 μl or more, about 60 μl or more, or about 65 μl or more, or about 70 μl or more, or about 75 μl or more, or about 80 μl or more, or about 85 μl or more, or about 90 μl or more, or about 95 μl or more, or about 100 μl or more.

In some embodiments, the enzymatic fragmentation mixture is adjusted to a volume of about 10 μl to about 200 μl. In some embodiments, the enzymatic fragmentation mixture is adjusted to a volume of about 10 μl to about 100 μl. In some embodiments, the enzymatic fragmentation mixture is adjusted to a volume of about 65 μl to about 200 μl. In some embodiments, the enzymatic fragmentation mixture is adjusted to a volume of about 65 μl to about 100 μl.

In some embodiments, the one or more cell populations in the enzymatic fragmentation mixture is diluted to contain 1 to 1,000,000 cells. In some embodiments, the cell population in the enzymatic fragmentation mixture is diluted to contain 1 to 1,000,000 cells. In some embodiments, the cell population is diluted to contain 1 to 100,000 cells. In some embodiments, the cell population is diluted to contain 1 to 90,000 cells. In some embodiments, the cell population is diluted to contain 1 to 80,000 cells. In some embodiments, the cell population is diluted to contain 1 to 70,000 cells. In some embodiments, the cell population is diluted to contain 1 to 60,000 cells. In some embodiments, the cell population is diluted to contain 1 to 50,000 cells.

In some embodiments, the one or more cell populations is diluted to contain 1 to 500,000 cells. In some embodiments, the one or more cell populations is diluted to contain 1 to 400,000 cells. In some embodiments, the one or more cell populations is diluted to contain 1 to 300,000 cells. In some embodiments, the one or more cell populations is diluted to contain 1 to 200,000 cells. In some embodiments, the one or more cell populations is diluted to contain 1 to 100,000 cells. In some embodiments, the one or more cell populations is diluted to contain 1 to 50,000 cells. In some embodiments, the one or more cell populations is diluted to contain 1 to 40,000 cells. In some embodiments, the one or more cell populations is diluted to contain 1 to 30,000 cells. In some embodiments, the one or more cell populations in the enzymatic fragmentation mixture is diluted to contain 1 to 30,000 cells. In some embodiments, the one or more cell populations is diluted to contain 1 to 20,000 cells. In some embodiments, the one or more cell populations is diluted to contain 1 to 15,000 cells. In some embodiments, the one or more cell populations is diluted to contain 1 to 16,000 cells. In some embodiments, the one or more cell populations is diluted to contain 1 to 15,000 cells. In some embodiments, the one or more cell populations is diluted to contain 1 to 10,000 cells. In some embodiments, the one or more cell populations is diluted to contain 1 to 100 cells, 100 to 200 cells, 200 to 300 cells, 300 to 400 cells 400 to 500 cells, 500 to 600 cells, 600 to 700 cells, 700 to 800 cells, 800 to 900 cells, 900 to 1000 cells, 1000 to 1100 cells, 1100 to 1200 cells, 1200 to 1300 cells, 1300 to 1400 cells, or 1400 to 1500 cells. In some embodiments, the one or more cell populations is diluted to contain 1 to 300 cells, 1 to 10 cells, 3 to 10 cells, 10 to 20 cells, 1 to 5 cells, 1 to 15 cells, 1 to 25 cells, 1 to 75 cells, and the like. In some embodiments, the one or more cell populations is diluted to contain 20,000 cells or less, 19,000 cells or less, 18,000 cells or less, 17,000 cells or less, 16,000 cells or less, 15,000 cells or less, 14,000 cells or less, 13,000 cells or less, 12,000 cells or less, 11,000 cells or less, 10,000 cells or less, 9,000 cells or less, 8,000 cells or less, 7,000 cells or less, 6,000 cells or less, 5,000 cells or less, 4,000 cells or less, 3,000 cells or less, 2,000 cells or less, 1,500 cells or less, 1,000 cells or less, 500 cells, 250 cells or less, 100 cells or less, 50 cells or less, 25 cells or less, 10 cells or less, 5 cells or less, or 2 cells or less. In some embodiments, the one or more cell populations is diluted to contain 1 cell. In some embodiments, the one or more cell populations is diluted to contain 1 to 15,000 cells.

In certain embodiments, the enzymatic fragmentation mixture does not include EDTA. In certain embodiments, the enzymatic fragmentation mixture includes EDTA.

In some embodiments, the fragmentation enzyme is a KAPA fragmentation enzyme, TaKara fragmentation enzyme, NEBNext Ultra enzymatic fragmentation enzyme, biodynamic DNA Fragmentation Enzyme Mix, KAPA Fragmentation Kit for Enzymatic Fragmentation, SureSelect Fragmentation enzyme, Ion Shear™ Plus Enzyme, and the like. In some embodiments, the fragmentation enzyme is a Caspase-Activated DNase (CAD). In some embodiments, a fragmentation enzyme and fragmentation buffer are contacted with one or more cell populations in an amount sufficient to perform a fragmentation reaction. In some embodiments, the volume of fragmentation enzyme added to the sample containing one or more cell populations ranges from 10 μl to 100 μl. In some embodiments, the volume of fragmentation enzyme added to the sample containing one or more cell populations ranges from 1 μl to 20 μl, 1 μl to 5 μl, 5 μl μl to 10 μl, 5 μl to 15 or 8 μl to 12 μl. In certain embodiments, the volume of fragmentation enzyme added to the sample containing one or more cell populations is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, or 20 μl or more.

In some embodiments, the fragmentation buffer is selected from a KAPA fragmentation buffer, TaKara fragmentation buffer, NEBNext Ultra enzymatic fragmentation buffer, biodynamic DNA Fragmentation buffer, KAPA Fragmentation buffer, SureSelect Fragmentation Buffer, Ion Shear™ Plus Reaction Buffer, and the like. However, any commercially available enzymatic fragmentation buffer can be used for fragmenting the DNA or RNA of the cell.

In some embodiments, the final enzymatic fragmentation mixture comprises a volume ranging from 10 μl to 100 μl. In some embodiments, the fragmentation buffer is a KAPA fragmentation buffer. In some embodiments, the volume of fragmentation buffer added to the sample containing one or more cell populations ranges from 10 μl to 100 μl. In some embodiments, the volume of fragmentation buffer added to the sample containing one or more cell populations ranges from 1 μl to 20 μl, 1 μl to 5 μl, 5 μl to 10 μl, 5 μl to 15 μl, or 8 μl to 12 μl. In certain embodiments, the volume of fragmentation buffer added to the sample containing one or more cell populations is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In some embodiments, the final volume of the enzymatic fragmentation mixture containing one or more cells, a fragmentation buffer, and a fragmentation enzyme ranges from 5 μl to 100 μl. In some embodiments, the final volume of the enzymatic fragmentation mixture containing one or more cells, a fragmentation buffer, and a fragmentation enzyme is 10 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the enzymatic fragmentation mixture comprises a conditioning solution. In some embodiments, the volume of conditioning solution added to the enzymatic fragmentation mixture ranges from 1 μl to 20 μl. In some embodiments, the volume of 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, or 20 μl or more. In some embodiments, the conditioning solution is a solution that adjusts the enzymatic fragmentation buffer to handle highly sensitive reagent compositions, and in some cases sequesters EDTA (or other chelators) in the sample. In some embodiments, the conditioning solution contains a reagent that binds EDTA in the sample. In some embodiments, the conditioning solution contains Magnesium or other cations to bind to EDTA in the cell population. In some embodiments, the conditioning solution is a solution that binds to magnesium in the sample. In some embodiments, the conditioning solution contains a divalent cation chelator to bind to excess magnesium in the sample.

In some embodiments, the method includes performing enzymatic fragmentation of the nucleic acids (e.g., DNA or RNA) within the one or more cell populations to form an enzymatic fragmentation reaction mixture. In some embodiments, performing an enzymatic fragmentation reaction on the mixture comprises loading the enzymatic fragmentation mixture into a suitable temperature-control device (although, in some such embodiments: (a) the mixture contains fewer than 15,000 fixed cells, or from 17,000-79,000 fixed cells, or more than 81,000 fixed cells; and/or (b) the temperature-control device maintains the temperature at from 15-36° C. or from 38-45° C. during the fragmentation reaction; and/or (c) for fewer than 59 minutes). In some embodiments, performing an enzymatic fragmentation reaction on the mixture comprises loading the enzymatic fragmentation mixture onto a thermocycler. In some embodiments, performing an enzymatic fragmentation reaction on the mixture comprises loading the enzymatic fragmentation mixture onto a heat block. In some embodiments, performing an enzymatic fragmentation reaction on the mixture comprises loading the enzymatic fragmentation mixture into a water bath. In some embodiments, performing an enzymatic fragmentation reaction on the mixture comprises loading the enzymatic fragmentation mixture into an incubator.

In some embodiments, the method includes incubating the enzymatic fragmentation mixture in the temperature control device (e.g., thermocycler for a duration/time period ranging from 1 minute to 120 minutes, 1 minute to 50 minutes, 3 minutes to 10 minutes, 5 minutes to 20 minutes, 10 minutes to 25 minutes, or 20 minutes to 40 minutes. In certain embodiments, the duration is 1 minute or more, 2 minutes or more, 3 minutes or more, 4 minutes or more, 5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, or 60 minutes or more.

In some embodiments, performing an enzymatic fragmentation reaction on the mixture comprises loading the mixture onto a thermocycler and incubating the mixture at a temperature ranging from 2° C. to 50° C., such as 4° C. to 37° C., 4° C. to 50° C., or 5° C. to 40° C. In some embodiments, the method includes incubating the mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, or 50° C., or more, 55° C. or more, 60° C. or more, 65° C. or more, 70° C. or more, 75° C. or more, or 80° C. or more. In some embodiments, performing an enzymatic fragmentation reaction on the mixture comprises loading the mixture onto a temperature-control device (e.g. thermocycler or heat-block) and incubating the mixture at a temperature of 14-20° C. In some embodiments, performing an enzymatic fragmentation reaction on the mixture comprises loading the mixture onto a temperature-control device (e.g. thermocycler or heat-block) and incubating the mixture at a temperature of 20-30° C. In some embodiments, performing an enzymatic fragmentation reaction on the mixture comprises loading the mixture onto a temperature-control device (e.g. thermocycler or heat-block) and incubating the mixture at a temperature 35-38° C.

In some embodiments, before the ligating step (c) of the ligation-based method, the method includes performing an end-repair and/or A-tailing reaction on the one or more DNA or RNA fragments. In some embodiments the enzymatic fragmentation enzyme is heat inactivated before end repair and A (ERA) tailing (described below) at a known temperature for inactivating the specific enzyme 65-99.5° C. for 5-60 minutes. In some embodiments the End repair and A tailing incubation step also acts as the heat inactivation step for enzymatic fragmentation enzymes.

In some embodiments, the End-repair and A-tailing reaction and the enzymatic fragmentation reaction occurs in a single reaction, with multiple temperature incubations. For example, the End repair and/or A-tailing reaction can occur during the enzymatic fragmentation reaction in a single reaction. In some embodiments the End repair reaction can occur at a certain temperature. Subsequently, A-tailing reaction can occur at a different temperature following a temperature change. In other embodiments, the End repair and/or A-tailing reaction can occur in different, separate reactions. In some embodiments, the End-repair and A-tailing reaction and the enzymatic fragmentation reaction are separate reactions.

End Repair and A-Tailing

In some embodiments, the method includes performing an end-repair and/or A-tailing reaction on the one or more fragmented DNA or RNA within the one or more cell populations. End Repair and A-Tailing are two enzymatic steps configured to blunt the DNA or RNA fragments and add an overhanging A nucleotide to the end of the DNA or RNA fragments, for example, to improve ligation efficiency. The end-repair and/or A-tailing reaction may be performed before ligating the DNA or RNA fragments. In some embodiments, the End Repair and/or A-tailing can occur in the same reaction as the enzymatic fragmentation reaction described above.

In some embodiments, performing an end-repair and A-tailing reaction comprises contacting the fragmented DNA or RNA within the one or more cell populations with an End Repair A-tail buffer and an End Repair A-tail enzyme to form an End Repair A-tail mixture. In some embodiments, performing an end-repair and A-tailing reaction comprises contacting the fragmented DNA or RNA within the one or more cell populations in the enzymatic fragmentation reaction mixture with an End Repair A-tail buffer and an End Repair A-tail enzyme to form an End Repair A-tail mixture. In some embodiments, contacting the fragmented DNA or RNA within the one or more cell populations in the enzymatic fragmentation reaction mixture with an End Repair A-tail buffer and an End Repair A-tail enzyme occurs a temperature ranging from 1° C. to 10° C. In some embodiments, contacting the fragmented DNA or RNA within the cell population in the enzymatic fragmentation reaction mixture with an End Repair A-tail buffer and an End Repair A-tail enzyme occurs on ice. The temperature may then be increased for enzymatic reactions to occur e.g., to from 25-40° C.

In some embodiments, the fragmented DNA (e.g., double stranded DNA or single stranded DNA) or RNA within the End Repair A-tail mixture is diluted to a volume of about 0.5 or more, about 1 μl or more, about 1.5 μl or more, about 2 μl or more, about 2.5 μl or more, about 3 μl or more, about 3.5 μl or more, about 4 μl or more, about 4.5 μl or more, about 5 μl or more, about 6 μl or more, about 7 μl or more, about 8 μl or more, about 9 μl or more, about 10 or more, about 11 μl or more, about 12 μl or more, about 13 μl or more, about 14 μl or more, about 15 μl or more, about 16 μl or more, about 17 μl or more, about 18 μl or more, about 19 or more, about 20 μl or more, about 25 μl or more, about 30 μl or more, about 35 μl or more, about 40 μl or more, about 45 μl or more, about 50 μl or more, about 55 μl or more, about 60 or more, about 65 μl or more, about 70 μl or more, about 75 μl or more, about 80 μl or more, about 85 μl or more, about 90 μl or more, about 95 μl or more, or about 100 μl or more.

In some embodiments, the volume of end Repair A-tail enzyme added to the enzymatic fragmentation reaction mixture (e.g., containing the fragmented DNA or RNA inserts) ranges from 1 μl to 20 μl, 1 μl to 5 μl, 5 μl to 10 μl, 5 μl to 15 or 8 μl to 12 μl. In certain embodiments, the volume of fragmentation enzyme added to the sample containing one or more cell populations is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, or 20 μl or more.

In some embodiments, the volume of End Repair A-tail buffer added to the enzymatic fragmentation reaction mixture (e.g., containing the fragmented DNA or RNA inserts) ranges from 10 μl to 100 μl. In some embodiments, the volume of fragmentation buffer added to the sample containing one or more cell populations ranges from 1 μl to 20 μl, 1 μl to 5 μl, 5 μl to 10 μl, 5 μl to 15 μl, or 8 μl to 12 μl. In certain embodiments, the volume of End Repair A-tail buffer added to the sample containing one or more cell populations is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In some embodiments, the final volume of the End Repair A-tail mixture containing one or more cells, a End Repair A-tail buffer, and a End Repair A-tail enzyme ranges from 5 μl to 100 In some embodiments, the final volume of the End Repair A-tail mixture containing one or more cells, a End Repair A-tail buffer, and a End Repair A-tail enzyme is 10 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the method further comprises running the End Repair A-tail mixture in a thermocycler to form an End Repair A-tail reaction mixture.

In some embodiments, the End Repair A-tail mixture is incubated in the thermocycler at a temperature ranging from 2° C. to 90° C. In some embodiments, performing an End Repair A-tail reaction on the End Repair A-tail mixture comprises loading the End Repair A-tail mixture onto a thermocycler and incubating the End Repair A-tail mixture at a temperature ranging from 2° C. to 50° C., such as 4° C. to 37° C., 4° C. to 50° C., or 5° C. to 40° C. In some embodiments, the step includes incubating the End Repair A-tail mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, 50° C. or more, 55° C. or more, 60° C. or more, 65° C. or more, 70° C. or more, 75° C. or more, 85° C. or more, 85° C. or more, 90° C. or more, 95° C. or more, or 100° C. or more.

In some embodiments, the End Repair A-tail mixture is incubated for a duration ranging from 5 minutes to 50 minutes. In some embodiments, the step includes incubating the End Repair A-tail mixture in the thermocycler for a duration/time period ranging from 1 minute to 50 minutes, 3 minutes to 10 minutes, 5 minutes to 20 minutes, 10 minutes to 25 minutes, or 20 minutes to 40 minutes. In certain embodiments, the duration is 1 minute or more, 2 minutes or more, 3 minutes or more, 4 minutes or more, 5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, or 60 minutes or more. In some embodiments the End repair and A tail enzymes are heat inactivated before proceeding to ligation at 65-100° C. for 5-60 minutes or more. A-tail enzymes are heat inactivated before proceeding to ligation at 65° C. or more, 70° C. or more, 75° C. or more, 80° C. or more, 85° C. or more, 90° C. or more, 95° C. or more, or 100° C. or more (but below 180° C.). A-tail enzymes are heat inactivated before proceeding to ligation for 5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, or 60 minutes or more (but for shorter than 180 minutes).

Adapter-Indexing Ligation

The present ligation-based method includes ligating, in each cell, the DNA or RNA fragments to one or more adapters in situ to create a ligated library comprising ligated DNA or RNA fragments.

Ligation adapter sequences may include modifications such as: methylation, capping, 3′-deoxy-2′,5′-DNA, N3′ P5′ phosphoramidates, 2′-O-alkyl-substituted DNA, 2′-O-methyl DNA, 2′ Fluoro DNA, Locked Nucleic Acids (LNAs) with 2′-O-4′-C methylene bridge, inverted T modifications (e.g. 5′ and 3′), or PNA (with such modifications at one or more nucleotide positions). Ligation adapter sequences may also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters).

In some embodiments, ligating includes performing ligase chain reaction (LCR). The ligase chain reaction (LCR) is an amplification process that involves a thermostable ligase to join two probes or other molecules together. In some embodiments, the thermostable ligase can include, but is not limited to, Pfu ligase, Taq ligase, HiFi Taq DNA ligase, 9° N DNA ligase, Thermostable 5′ AppDNA/RNA ligase, Ampligase® ligase, or a T4 RNA ligase (e.g. T4 RNA ligase 2). In some embodiments, the ligated product is then amplified to produce an amplicon product. In some embodiments, LCR can be used as an alternative approach to PCR. In other embodiments, PCR can be performed after LCR.

Ligating the DNA fragments to the adapter (e.g., an adapter that includes a R1 sequence or a R2 sequence) comprises running the DNA fragments and adapters in a thermocycler at a temperature and duration sufficient to ligate the DNA fragmented to the adapter sequences. Ligation reagents and/or enzymes can be used for ligating the DNA or RNA fragments. In some embodiments, ligation chain reaction (LCR) can be used for ligating the DNA or RNA fragments.

Ligation of fragments to adapters (e.g., adapters that includes a R1 sequence or a R2 sequence) sequences can also be performed using ligation without LCR (e.g. without the use of thermal cycling). Adapters can be ligated enzymatically, using any suitable DNA/RNA ligase. For instance, ligation can use Pfu ligase from Pyrococcus furiosus, Taq ligase from Thermus aquaticus (e.g. HiFi Taq DNA ligase), DNA ligase from Cholorella virus (e.g. PBCV-1 DNA ligase), T4 DNA ligase, Quick ligase, Blunt/TA ligase, T3 bacteriophage DNA ligase, T7 bacteriophage DNA ligase, a DNA ligase from Thermococcus (e.g. 9° N DNA ligase), Thermostable 5′ AppDNA/RNA ligase, Ampligase® ligase, Instant Sticky End ligase, T4 RNA ligases (e.g. T4 RNA ligase 1, T4 RNA ligase 2 truncated, T4 RNA ligase truncated K227Q, and T4 RNA ligase 2 truncated KQ), or a RtcB ligase. Ligases which are able to be heat-inactivated are preferred. For example, ligases which can be heat inactivated through heating to 65° C. for 10 minutes are preferred.

The fragmented DNA or RNA are contacted with adapter (e.g., an adapter that includes a R1 sequence or a R2 sequence) to form a ligated library/ligation mixture containing the ligated DNA or RNA fragments. In some embodiments, the ligation mixture can include a Ligation Master Mix. In some embodiments, the ligation mixture can include a Blunt/TA Ligase Master Mix, or an Instant Sticky End Ligase Master Mix.

Adapter Ligation enzymatically combines (e.g., ligates) adapters provided in the reaction to the prepared DNA or RNA fragments. Non-limiting examples of adapter sequences include, but are not limited to, adapter nucleotide sequences that allow high-throughput sequencing of amplified or ligated nucleic acids. In some embodiments, the adapter sequences are selected from one or more of: a Y-adapter nucleotide sequence, a hairpin nucleotide sequence, a duplex nucleotide sequence, and the like. In some embodiments, the adapter sequences (e.g., P5 and P7 sequences) are included for pair-end sequencing. Adapter sequences (e.g., P5 and P7 sequences) can be used in a ligation reaction of the disclosed method for the desired sequencing method used. In some embodiments, the method includes attaching sequence adapters to amplified nucleic acid from these sub-populations of live cells using a ligation-based approach.

In some embodiments, the ligation mixture or enzymatic fragmentation reaction mixture, includes the End-repair A-tail reaction mixture, a set of adapter, and a ligation master mix. In certain embodiments, ligation mixture includes the End-repair A-tail reaction mixture, a set of indexed nucleotide sequences, nuclease free H2O, and a ligation master mix. In certain embodiments, the ligation mixture includes a final volume ranging from 10 μl to 200 such as 10 μl to 100 μl, 10 μl to 150 μl, 50 μl to 150 μl, 50 μl to 120 μl, 70 μl to 115 μl, or 90 μl to 110 μl. In certain embodiments, the ligation mixture includes a final volume of 35 μl or more, 40 μl, or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, 100 μl or more, 105 μl or more, 110 μl or more, 115 μl or more, 120 μl or more, 125 μl or more, 130 μl or more, 135 μl or more, 140 μl or more, 145 μl or more, 150 μl or more, 155 μl or more, 160 μl or more, 165 μl or more, 170 μl or more, 175 μl or more, 180 μl or more, 185 μl or more, 190 μl or more, 195 μl or more, or 200 μl or more.

In some embodiments, the ligation mixture includes the End-repair A-tail reaction mixture in a volume ranging from 1 μl to 100 In some embodiments, the ligation mixture includes the End-repair A-tail reaction mixture in a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the ligation mixture includes the set of adapters (e.g., adapters that includes a R1 sequence or a R2 sequence) in a volume ranging from 1 μl to 20 μl, 1 μl to 5 μl, or 1 μl to 10 μl. In some embodiments, the ligation mixture includes the set of adapters (e.g., adapters that includes a R1 sequence or a R2 sequence) in a volume of 1 μl or more, 1.5 μl or more, 2 μl or more, 2.5 μl or more, 3 μl or more, 3.5 μl or more, 4 μl or more, 4.5 μl or more, 5 μl or more, 5.5 μl or more, 6 μl or more, 6.5 μl or more, 7 μl or more, 7.5 μl or more, 8 μl or more, 8.5 μl or more, 9 μl or more, 9.5 μl or more, 10 μl or more, 11 μl or more, 12 μl or more, 13 μl or more, 14 μl or more, 15 μl or more, or 20 μl or more.

In some embodiments, the nuclease free H2O in the ligation mixture comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, or 15 μl or more. In some embodiments the nuclease free H₂O is replaced with a buffered solution (e.g., such as PBS).

In some embodiments, the ligation master mix comprises nuclease free H2O, a ligation buffer, and a DNA ligase. In some embodiments, the ligation master mix includes a final volume ranging from 5 μl to 100 such as 10 μl to 50 μl, 25 μl to 50 or 30 μl to 60. In some embodiments, the ligation master mix includes a final volume of 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the nuclease free H2O in the ligation master mix comprises a volume of 1 μl or more, 2 μl or more, 3 μl or more, 4 μl or more, 5 μl or more, 6 μl or more, 7 or more, 8 μl or more, 9 μl or more, 10 μl or more, 11 μl or more, 12 μl or more, 13 μl or more, 14 μl or more, or 15 μl or more.

In some embodiments, the ligation buffer in the ligation master mix comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In some embodiments, the DNA ligase in the ligation master mix comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In certain embodiments, the method comprises preparing the ligation master mix to a final volume ranging from 10 μl to 100 μl. In some embodiments, the final volume of the ligation master mix ranges from 1 μl to 20 μl, 1 μl to 5 μl, 5 μl to 10 μl, 5 μl to 15 μl, or 8 μl to 12 μl. In certain embodiments, the final volume of the ligation master mix is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the method includes ligating the fragmented DNA or RNA to the adapter (e.g., an adapter that includes a R1 sequence or a R2 sequence). In certain embodiments, ligating the fragmented DNA or RNA to the adapter (e.g., an adapter that includes a R1 sequence or a R2 sequence) comprises running the ligation mixture in the thermocycler at a temperature and duration sufficient to ligate the fragmented DNA or RNA to the adapter (e.g., an adapter that includes a R1 sequence or a R2 sequence).

In some embodiments, the temperature ranges from 4° C. to 90° C. In some embodiments, the method includes incubating the ligation mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, 50° C. or more, 55° C. or more, 60° C. or more, 65° C. or more, 70° C. or more, 75° C. or more, 85° C. or more, 85° C. or more, 90° C. or more, 95° C. or more, or 100° C. or more. In some embodiments, the method includes incubating the ligation mixture at a temperature of 20±5° C. In some embodiments, the method includes incubating the ligation mixture at a temperature of about 20° C.

In some embodiments, the duration ranges from 5 minutes to 4 hours. In some embodiments, the method includes incubating the ligation mixture in the thermocycler for a duration/time period ranging from 1 minute to 5 hours, 1 minute to 4 hours, 1 minute to 50 minutes, 3 minutes to 10 minutes, 5 minutes to 20 minutes, 10 minutes to 25 minutes, or 20 minutes to 40 minutes. In certain embodiments, the duration is 1 minute or more, 2 minutes or more, 3 minutes or more, 4 minutes or more, 5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, or 60 minutes or more. In certain embodiments, the duration is 1 hour or more, 1.5 hours or more, 2 hours or more, 2.5 hours or more, 3 hours or more, 3.5 hours or more, 4 hours or more. 4.5 hours or more, or 5 hours or more.

In some embodiments the ligase enzyme is heat inactivated e.g. at a temperature ranging from 65-99.5° C. for a duration ranging from 5-60 minutes before proceeding to the next steps. In some embodiments, ligase enzymes do not need to be heat inactivated.

Examples of Additional Amplification of Ligated Library

creating more copies of the DNA or RNA fragments, reducing the likelihood of region drop out due to in efficiencies in purification and/or hybridization capture protocols. Additionally, the method allows for adding additional sequences such as adapter sequences, read sequences, full primer sequences with sample barcodes, and the like during amplification. In some embodiments, amplifying the ligated DNA or RNA fragments to form amplicon products comprises contacting the ligated DNA or RNA fragments with amplification primers (e.g., primers used to hybridize with sample DNA or RNA that define the region to be amplified, but can also include, barcoding primers, P5/P7 primers, R1/R2 primers, other sequencing primers, and the like).

Additionally, multiple PCR reactions may be performed, for example, after ligation but before sequencing the ligated DNA or RNA fragments of the cells. Some, none, or all of these additional PCR steps could occur before cell lysis, while some, none, or all of these additional PCR steps could occur after cell lysis. Additional PCR steps can include adding additional components to a PCR reaction, with each addition defined as a “PCR step”. For example, adding targeting primers, followed by adding amplification primers can take place in two PCR reactions, e.g. two PCR steps or one PCR reaction, e.g., one PCR step. In some embodiments, one or more, two or more, three or more, four or more, five or more, six or more, seven or more, eight or more, nine or more, or ten or more distinct PCR reactions can be performed. In certain embodiments, two PCR reactions are performed between ligation and sequencing steps (e.g., after ligation, but before lysing). In certain embodiments, three PCR reactions are performed between ligation and sequencing steps (e.g., after ligation, but before lysing). In certain embodiments, four PCR reactions are performed between ligation and sequencing steps (e.g., after ligation, but before lysing). In certain embodiments, the PCR reactions are performed after ligation but before the lysing step. In certain embodiments, the PCR reactions are performed after ligation but before the lysing step.

When performing amplification after the ligation step, the method includes contacting the ligated library (e.g., adapter ligated DNA or RNA fragments) with primers. In some embodiments, the method includes amplifying the ligated library with primers containing minimal sequences (e.g., read 1, read 2 sequences, P5 and/or P7 sequences, etc.). In some embodiments, the method includes amplifying the ligated library with primers including sample barcodes. In some embodiments, the method includes amplifying the ligated library with primers including the adapter sequences, such as P5 and P7.

Primers may include modifications such as: methylation, capping, 3′-deoxy-2′,5′-DNA, N3′ P5′ phosphoramidates, 2′-O-alkyl-substituted DNA, 2′-O-methyl DNA, 2′ Fluoro DNA, Locked Nucleic Acids (LNAs) with 2′-O-4′-C methylene bridge, inverted T modifications (e.g. 5′ and 3′), or PNA (with such modifications at one or more nucleotide positions). Ligation adapter sequences may also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters),In some embodiments, the method includes amplifying the adapter-ligated fragments (e.g., ligated library) to create more copies before going through hybridization capture and/or sequencing. In some embodiments, the method includes amplifying the adapter-ligated fragments to add full length adapter sequences onto the adapter-ligated fragments, if necessary.

In some embodiments, after the ligating step to produce the ligated library but before sequencing, the method includes contacting the ligated library with an amplification mixture. In some embodiments, the amplification mixture comprises any readily available, standard amplification library mix or one or more components thereof, a set of amplification primers, and the adapter-ligated library. In some embodiments, the amplification mixture comprises a KAPA HiFi Hotstart Ready Mix (2×) or one or more components from the ready mix thereof, a set of amplification primers, and the adapter-ligated library. In some embodiments, the amplification mixture comprises a xGen Library Amplification Primer Mix or one or more components from the primer mix thereof, a set of amplification primers, and the adapter-ligated library. In other embodiments, the amplification mixture includes a Library Amplification Hot Start Master Mix and a xGen UDI primer Mix (IDT).

In some embodiments, the amplification mixture comprises a total volume ranging from 10 to 100 In some embodiments, the final volume of the amplification mixture ranges from 1 μl to 20 μl, 1 μl to 5 μl, 5 μl to 10 μl, 5 μl to 15 or 8 μl to 12 μl. In certain embodiments, the final volume of the amplification mixture is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the amplification library mix (e.g., KAPA HiFi Hotstart Ready Mix (2×), xGen Library Amplification Primer Mix, or Amplification Hot Start Master Mix) within the amplification mixture comprises a volume ranging from 10 to 100 μl. In some embodiments, the KAPA HiFi Hotstart Ready Mix (2×) within the amplification mixture ranges from 1 μl to 20 μl, 1 μl to 5 μl, 5 μl μl to 10 μl, 5 μl to 15 or 8 μl to 12 μl. In certain embodiments, the KAPA HiFi Hotstart Ready Mix (2×) within the amplification mixture comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the set of amplification primers within the amplification mixture comprises a volume ranging from 10 to 100 μl. In some embodiments, the set of amplification primers within the amplification mixture ranges from 1 μl to 20 μl, 1 μl to 5 μl, 5 μl to 10 μl, 5 μl to 15 μl, or 8 μl to 12 In certain embodiments, the set of amplification primers within the amplification mixture comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the Library Amplification Hot Start Master Mix within the amplification mixture comprises a volume ranging from 1-100 In some embodiments, the Library Amplification Hot Start Master Mix within the amplification mixture comprises a volume of about 10 μl, 15 μl, 20 μl, 25 μl, 30 μl, 35 μl, 40 μl, 45 μl, 50 μl, 55 μl, 60 μl, 65 μl, 70 μl, 75 μl, 80 μl, 85 μl, 90 μl, 95 μl, or 100 μl.

In some embodiments, the primer Mix within the amplification mixture comprises a volume ranging from 1-10 In some embodiments, the primer Mix (IDT) within the amplification mixture comprises a volume of about 1 μl, 2 μl, 3 μl, 4 μl, 5 μl, 6 μl, 7 μl, 8 μl, 9 μl, or about 10 μl.

In some embodiments, the ligated library within the amplification mixture comprises a volume ranging from 10 to 100 μl. In some embodiments, the indexed library within the amplification mixture ranges from 1 μl to 20 μl, 1 μl to 5 μl, 5 μl to 10 μl, 5 μl to 15 μl, or 8 μl to 12 μl. In certain embodiments, the ligated library within the amplification mixture comprises a volume of 1 μl or more, 2 μl or more, 3 μl or more, 4 μl or more, 5 μl or more, 6 μl or more, 7 or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the method comprises amplifying the amplification mixture to produce a first set of amplicon products. In some embodiments, amplifying is performed using a thermocycler. In some embodiments, amplifying is performed using polymerase chain reaction (PCR).

In some embodiments, amplifying comprises running the amplification mixture in the thermocycler for a duration ranging from 1 second to 5 minutes. In some embodiments, amplifying comprises running the amplification mixture in the thermocycler for a duration ranging from 1 second to 1 minute. In some embodiments, amplifying comprises running the amplification mixture in the thermocycler for a duration ranging from 30 seconds to 1 minute. In some embodiments, amplifying comprises running the amplification mixture in the thermocycler for a duration ranging from 45 seconds to 1 minute. In some embodiments, amplifying comprises running the amplification mixture in the thermocycler for a duration of 1 second or more, 5 seconds or more, 15 seconds or more, 20 seconds or more, 25 seconds or more, 30 seconds or more, 35 seconds or more, 40 seconds or more, 45 seconds or more, 50 seconds or more, 55 seconds or more, 60 seconds or more, 1 minute or more, or 1.5 minutes or more.

In some embodiments, the temperature of incubation of the amplification mixture in the thermocycler ranges from 4° C. to 110° C. In some embodiments, the method includes incubating the ligation mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, 50° C. or more, 55° C. or more, 60° C. or more, 65° C. or more, 70° C. or more, 75° C. or more, 85° C. or more, 85° C. or more, 90° C. or more, 95° C. or more, 100° C. or more, 105° C. or more, 110° C. or more, 115° C. or more, 120° C. or more, 125° C. or more, 130° C. or more, 140° C. or more, 145° C. or more, or 150° C. or more.

Hybridization Capture

In some embodiments, the ligation-based method includes performing hybridization capture on the purified library. For example, this step can occur before sequencing. This purified library may optionally contain barcoded sequences ligated or amplified onto the DNA or RNA fragments.

Hybridization capture can be performed using any conventionally acceptable hybridization capture technique. For example, in one embodiment, performing hybridization capture comprises contacting the purified library (e.g., purified library with or without barcode sequences) with oligonucleotides configured to hybridize to one or more target DNA or RNA sequences and performing hybridization capture on purified DNA or RNA fragments.

Oligonucleotides may include modifications such as: methylation, capping, 3′-deoxy-2′,5′-DNA, N3′ P5′ phosphoramidates, 2′-O-alkyl-substituted DNA, 2′-O-methyl DNA, 2′ Fluoro DNA, Locked Nucleic Acids (LNAs) with 2′-O-4′-C methylene bridge, inverted T modifications (e.g. 5′ and 3′), or PNA (with such modifications at one or more nucleotide positions). Ligation adapter sequences may also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters).

In some embodiments, performing hybridization capture includes hybridizing the purified DNA or RNA fragments of the purified library with oligonucleotides to produce the enriched nucleic acid library. In some embodiments, performing hybridization capture includes contacting the purified DNA or RNA fragments with a one or more oligonucleotides that hybridize to target In such embodiments, the method further includes hybridizing blocking oligonucleotides in the same hybridization reaction. In certain embodiments, the blocking oligonucleotides are xGen Universal Blockers. In certain embodiments, the blocking oligonucleotides are Twist Universal Blockers. In certain embodiments, the blocking oligonucleotides are NEXTFLEX® Universal Blockers. In certain embodiments, the blocking oligonucleotides are Illumina Free Adapter Blocking Reagent.

In some embodiments, the one or more oligonucleotides comprises a set of 5′ oligonucleotides that are biotinylated.

In some embodiments, hybridization capture further comprises adding magnetic streptavidin beads that bind to the one or more oligonucleotide probes. In some embodiments, after the oligonucleotide probes are captured using magnetic streptavidin bead, the captured/enriched amplicon product is eluted and amplified another time.

In some embodiments, hybridization capture occurs in solution or on a solid support.

A non-limiting example of a hybridization capture method includes hybridizing oligonucleotide probes to the purified DNA or RNA fragments. Oligonucleotide probes can be DNA or RNA, and can be double-stranded, or single-stranded. In some embodiments, the oligonucleotides have biotinylated nucleotides incorporated into the oligonucleotides. Hybridization typically occurs by repeatedly heating and cooling the sample to increase association of the probe to the DNA or RNA. In some embodiments, oligonucleotide blockers are added to reduce likelihood of over-represented genomic sequences from mis-associating with the probes and also prevent the adapters attached to the PCR DNA or RNA fragments from binding to each other or genomic sequences. After hybridization, the probes are captured using magnetic streptavidin bead (via strong association with the biotin on the probe), then the “captured” Pre-Cap PCR product (e.g., indexed amplicon product) is eluted and amplified.

In some embodiments, after hybridization capture, the method includes eluting the purified DNA or RNA fragment. In some embodiments, the method includes amplifying the eluted captured/enriched purified DNA or RNA fragment.

Oligonucleotides for Hybridization Capture

In some embodiments, the oligonucleotides of the present disclosure are designed to hybridize to multiple targets with the use of multiple oligonucleotides in a single hybridization capture experiment.

In some embodiments, the oligonucleotides are DNA oligonucleotides. In some embodiments, the oligonucleotides are RNA oligonucleotides. In some embodiments, the oligonucleotides are single stranded. In some embodiments, the oligonucleotides are double stranded.

In some embodiments, capture oligonucleotides are used during the hybridization capture method. For example, capture oligonucleotides are biotinylated oligonucleotide baits. Oligonucleotide biotinylated baits are designed to hybridize to regions of interest (e.g., target regions). In certain embodiments, after hybridization of oligonucleotide baits to the target regions, contacting the hybridized oligonucleotide baits with streptavidin beads to separate the bait:target nucleic acid complex from other fragments that are not bound to baits.

In some embodiments, each oligonucleotide comprises a nucleotide sequence that hybridize to an anti-sense strand of a nucleotide sequence encoding a target region of one or more cells. In some embodiments, each oligonucleotide comprises a unique nucleotide sequence that hybridizes to an anti-sense strand of a nucleotide sequence encoding a different target region of one or more cells. Thus, a oligonucleotide pool can include a plurality of oligonucleotides, where each oligonucleotide hybridizes to a distinct target nucleic acid. In embodiments where hybrid capture is performed, an oligonucleotide pool includes oligonucleotides of a xGen Lockdown Panel. In certain embodiments where hybrid capture is performed, a oligonucleotide pool includes oligonucleotides of a xGen Probe Pool. In certain embodiments where hybrid capture is performed, a oligonucleotide pool includes oligonucleotides of a xGen lockdown Panels and Probe Pools. In certain embodiments where hybrid capture is performed, a oligonucleotide pool includes oligonucleotides of a xGen lockdown Panels and Probe Pools. In some embodiments, the panels comprise probes to target genes associated with a disease or condition. In some embodiments, the target genes are selected from one or more of: PD-L1, PD-1, HER2, BL1, CCDC6, EIF1AX, HIST1H2BD, MED12, POLE, SMARCB1, UPF3A, ACO1, CCND1, EIF2S2, HIST1H3B, MED23, POT1, SMC1A, VHL, ACVR1, CD1D, ELF3, HIST1H4E, MEN1, POU2AF1, SMC3, WASF3, ACVR1B, CD58, EML4, HLA-A, MET, POU2F2, SMO, WT1, ACVR2A, CD70, EP300, HLA-B, MGA, PPM1D, SMTNL2, XIRP2, ACVR2B, CD79A, EPAS1, HLA-C, MLH1, PPP2R1A, SNX25, XPO1, ADNP, CD79B, EPHA2, HNF1A, MPL, PPP6C, SOCS1, ZBTB20, AJUBA, CDC27, EPS8, HOXB3, MPO, PRDM1, SOX17, ZBTB7B, AKT1, CDC73, ERBB2, HRAS, MSH2, PRKAR1A, SOX9, ZFHX3, ALB, CDH1, ERBB3, IDH1, MSH6, PSG4, SPEN, ZFP36L1, ALK, CDH10, ERCC2, IDH2, MTOR, PSIP1, SPOP, ZFP36L2, ALPK2, CDK12, ERG, IKBKB, MUC17, PTCH1, SPTAN1, ZFX, AMER1, CDK4, ESR1, IKZF1, MUC6, PTEN, SRC, ZMYM3, APC, CDKN1A, ETNK1, IL6ST, MXRA5, PTPN11, SRSF2, ZNF471, APOL2, CDKN1B, EZH2, IL7R, MYD88, PTPRB, STAG2, ZNF620, ARHGAP35, CDKN2A, FAM104A, ING1, MYOCD, QKI, STAT3, ZNF750, ARHGAP5, CDKN2C, FAM166A, INTS12, MYOD1, RAC1, STAT5B, ZNF800, ARID1A, CEBPA, FAM46C, IPO7, NBPF1, RACGAP1, STK11, ZNRF3, ARID1B, CHD4, FAT1, IRF4, NCOR1, RAD21, STK19, ZRSR2, ARID2, CHD8, FBXO11, ITGB7, NF1, RASA1, STX2, ARID5B, CIB3, FBXW7, ITPKB, NF2, RB1, SUFU, ASXL1, CIC, FGFR1, JAK1, NFE2L2, RBM10, TBC1D12, ATM, CMTR2, FGFR2, JAK2, NIPBL, RET, TBL1XR1, ATP1A1, CNBD1, FGFR3, JAK3, NOTCH1, RHEB, TBX3, ATP1B1, CNOT3, FLG, KANSL1, NOTCH2, RHOA, TCEB1, ATP2B3, COL2A1, FLT3, KCNJ5, NPM1, RHOB, TCF12, ATRX, COL5A1, FOSL2, KDM5C, NRAS, RIT1, TCF7L2, AXIN1, COL5A3, FOXA1, KDM6A, NSD1, RNF43, TCP11L2, AXIN2, CREBBP, FOXA2, KDR, NT5C2, RPL10, TDRD10, AZGP1, CRLF2, FOXL2, KEAP1, NTN4, RPL22, TERT, B2M, CSDE1, FOXQ1, KEL, NTRK3, RPL5, TET2, BAP1, CSF1R, FRMD7, KIT, NUP210L, RPS15, TG, BCLAF1, CSF3R, FUBP1, KLF4, OMA1, RPS2, TGFBR2, BCOR, CTCF, GAGE12J, KLF5, OR4A16, RPS6KA3, TGIF1, BHMT2, CTNNA1, GATA1, KLHL8, OR4N2, RREB1, TIMM17A, BIRC3, CTNNB1, GATA2, KMT2A, OR52N1, RUNX1, TNF, BMPR2, CUL3, GATA3, KMT2B, OTUD7A, RXRA, TNFAIP3, BRAF, CUL4B, GNA11, KMT2C, PAPD5, SELP, TNFRSF14, BRCA1, CUX1, GNA13, KMT2D, PAX5, SETBP1, TOP2A, BRCA2, CYLD, GNAQ, KRAS, PBRM1, SETD2, TP53, BRD7, DAXX, GNAS, KRT5, PCBP1, SF3B1, TRAF3, C3orf70, DDX3X, GNB1, LATS2, PDAP1, SGK1, TRAF7, CACNA1D, DDX5, GNPTAB, LCTL, PDGFRA, SH2B3, TRIM23, CALR, DIAPH1, GPS2, LZTR1, PDSS2, SLC1A3, TSC1, CARD11, DICER1, GTF2I, MAP2K1, PDYN, SLC26A3, TSC2, CASP8, DIS3, GUSB, MAP2K2, PHF6, SLC44A3, TSHR, CBFB, DNM2, H3F3A, MAP2K4, PHOX2B, SLC4A5, TTLL9, CBL, DNMT3A, H3F3B, MAP2K7, PIK3CA, SMAD2, TYRO3, CBLB, EEF1A1, HIST1H1C, MAP3K1, PIK3R1, SMAD4, U2AF1, CCDC120, EGFR, HIST1H1E, MAX, PLCG1, SMARCA4, and UBR5. In situ cell barcoding performed in a single pool of cells.

As mentioned in an earlier section, one advantage of the methods and compositions described throughout is that cell barcoding may be performed in a single pool of cells without the need for or without requiring dividing or splitting of the cells into multiple pools (though the cell barcoding can also be performed in protocols where the cells are split into more than one pool). In fact, any of the description throughout can be applied in a single pool of cells. To illustrate, a few specific examples are presented here, though there are many other variations that are possible. As one example, in a single pool of cells, barcoding oligonucleotides may be introduced within a cell suspension, where each barcoding oligonucleotide comprises a molecular cellular label (e.g., “DS” in FIGS. 1 and 20), and a consensus region (“CR”). In this embodiment, the method includes amplifying, within individual cells of the single pool of cells, the barcoding oligonucleotides to produce a set of barcoding primers. The method further includes amplifying, within individual cells of the single pool of cells, the DNA or RNA with the barcoding primers to produce a set of amplicon products that comprise the barcoding primers, resulting in situ barcoded cells in the single pool of cells.

In other example embodiments, in the single pool of cells, the method comprises performing, in each cell, a fragmentation process to form nucleic acid fragments, performing, in each cell, an amplification or ligation of the nucleic acid fragments with universal sequences, and introducing barcoding oligonucleotides to the single pool of cells. The method also includes amplifying, within individual cells of the single pool of cells, the barcoding oligonucleotides to produce a set of barcoding primers. The method additionally includes amplifying, within individual cells of the single pool of cells, the nucleic acid fragments with the barcoding primers to produce a set of amplicon products that comprise the barcoding primers, resulting in situ barcoded cells in the single pool of cells.

Additional embodiments include a method of amplifying an oligonucleotide in situ to generate multiple copies of a reverse complement of the oligonucleotide.

### Buffer Exchange and Cell Washing

Some embodiments of the method include fragmentation and labeling of nucleic acids (e.g., genomic DNA) in a single pool, and using buffer exchanges or cell washing steps. A buffer exchange can advantageously occur, in fact, between any main steps of the process. For example, after an amplification step and cells are spun down, the liquid may be removed and replaced with a different buffer or set of reagents. Instead of performing this buffer exchange to do a simple wash of excess molecules from the cells, this step is performed to provide for a change in the ionic composition of the cells. For example, fragmentation and end repair steps may be performed in a first optimal buffer (or set of reagents) for those processes, and then a buffer or reagent exchange is performed to allow ligation to occur in a second optimal buffer (or set of reagents) for ligation, where the second buffer has a composition different from the first. This allows a sequence of chemical sequencing library preparation reaction to occur that would not otherwise be possible if a buffer exchange were not performed between steps.

To illustrate this, a few specific examples are presented here, though there are many other variations that are possible. As one example, the method includes, in the single pool of cells, performing, in each cell, a fragmentation process to form nucleic acid fragments, and further performing, in each cell, an amplification or ligation of the nucleic acid fragments with universal sequences in a reaction comprising a first buffer. The method then includes conducting a buffer exchange and/or cell washing step, wherein the first buffer is removed and replaced with a second buffer having a different composition specific to performing barcoding of the nucleic acid fragments that have been amplified. The method also includes introducing barcoding oligonucleotides to the single pool of cells, and amplifying, within individual cells of the single pool of cells, the barcoding oligonucleotides to produce a set of barcoding primers. In addition, the method includes amplifying, within individual cells of the single pool of cells, the nucleic acid fragments with the barcoding primers to produce a set of amplicon products that comprise the barcoding primers, resulting in situ barcoded cells in the single pool of cells.

In a further example, the method includes, in a single pool of cells, performing, in each cell, a fragmentation process to form genomic DNA fragments, and performing, in each cell, an amplification or ligation of the genomic DNA fragments with a first set of reagents. The method also includes conducting a cell washing step, wherein the first set of reagents is removed and replaced with a second set of reagents specific to performing barcoding of the genomic DNA fragments that have been amplified. The method additionally includes performing, in each cell, an amplification or ligation of the genomic DNA fragments with barcoding oligonucleotides in the second set of reagents, to create an in situ barcoded library in the single pool of cells.

In an additional example, the method comprises, in a single pool of cells, performing, in each cell, a fragmentation process to form genomic DNA fragments, and performing, in each cell, an amplification or ligation of the genomic DNA fragments involving a first buffer. The method also includes conducting a buffer exchange and cell washing step, wherein a first buffer having a composition designed for the amplification in step is removed and replaced with a second buffer having a different composition optimized for performing barcoding of the genomic DNA fragments that have been amplified. The method also includes performing, in each cell, in situ barcode amplification, and amplification or ligation of the genomic DNA fragments with barcoding products to create an in situ barcoded library in the single pool of cells.

Other embodiments comprise a method in which, in a single pool of cells, the method involves performing, in each cell, a fragmentation process to form genomic DNA fragments, and conducting a buffer exchange and/or cell washing step. In this method, a first buffer is removed from a product resulting from the fragmentation process and replaced with a second buffer having a different composition designed to change ionic composition of the cells to permit additional steps of the method. The method also includes performing, in each cell, in situ barcode amplification and amplification or ligation of the genomic DNA fragments with barcoding products to create an in situ barcoded library in the single pool of cells.

Embodiments of the method also include, in a single pool of cells, performing, in each cell, an amplification of genomic DNA fragments in the cell, and conducting a cell washing step to modify ionic composition of each of the cells. This method also includes amplifying, in each cell with modified ionic composition, barcoding oligonucleotides. Further, the method includes performing, in each cell with modified ionic composition, in situ amplification of the barcoding oligonucleotides, and amplification or ligation of the genomic DNA fragments with barcoding products to create an in situ barcoded library in the single pool of cells.

### Maintaining Intact Cells

A further advantage of the compositions and method described throughout includes that the steps are designed to allow cells to remain intact until it is desired to lyse the cells. For example, multiple PCR steps may be performed, but the protocols are designed such that these can be performed in situ without any of the PCR steps lysing the cells (or with lysis of only a minimal number of cells). This allows for further steps to occur following library preparation and cell barcoding where it is advantageous to have intact cells, including cell sorting steps. In contrast, conventional methods are not carefully designed to avoid lysing the cells, and may simply provide for analyzing libraries from lysed cells afterward without ensuring that most of the cells remain intact or focusing on avoiding cell lysis. In addition, in the present methods, if a limited number of cells are lysed during some steps, the intermediate buffer exchange steps described above allow for removal of any nucleic acids or cell materials from such lysed cells, so that the library preparation and cell barcoding methods can continue to be performed with a focus on the intact cells and continuing to maintain those cells intact.

To illustrate this, a few specific examples are presented here, though there are many other variations that are possible. As one example, the method includes performing, in each cell, an amplification of genomic DNA fragments in the cell, wherein the cells are not lysed by the amplification. The method also includes conducting a cell washing step to modify ionic composition of each of the cells. Additionally, the method includes performing, in each cell, in situ barcode amplification, and amplification or ligation of the genomic DNA fragments with barcoding products to create an in situ barcoded library in the single pool of cells.

Additional embodiments comprise performing, in each cell, an amplification of genomic DNA fragments in the cell, resulting in a cell supernatant, wherein a majority of the cells in the cell supernatant are not lysed by the amplification. This method includes conducting a cell washing step to remove from the cell supernatant cellular materials from cells that were lysed by the amplification. The method also includes performing, in each cell, in situ barcode amplification, and amplification or ligation of the genomic DNA fragments with barcoding products to create an in situ barcoded library in the cells that remain un-lysed.

A further embodiment comprises, in a single cell pool of cells for in situ cell barcoding, use of one or more washing steps in between reactions to replace each set of reagents for each reaction with a different set of reagents specific to a next reaction.

### Sequencing of Nucleic Acids Following Cellular Barcoding

Aspects of the present methods include sequencing the purified libraries. Sequencing occurs after the purification step; after the purification and additional ligation/PCR steps; or after the purification and additional ligation/PCR and hybridization capture steps.

Any high-throughput technique for sequencing can be used in the practice of the methods described herein. For example, DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence target nucleic acids of interest, for example, nucleic acids encoding target genes and other phenotypic markers amplified from the cell/nuclei populations.

In some embodiments, sequencing comprises whole genome sequencing.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLID sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

In some embodiments, sequencing may be performed using a flow cell. DNA/RNA fragments, which contain adapter molecules on either end, are washed across a flow cell (DNA is first denatured into single stranded DNA). This flow cell contains primers which are complementary to the adapter sequences. The bound DNA/RNA is then amplified repeatedly, using unlabelled nucleotides. This forms clusters of DNA/RNA which help produce an amplified signal during sequencing. During sequencing, primers and 4 different fluorescently labelled (reversible) terminator nucleotides are added. Each time a fluorescently labelled nucleotide is incorporated, the label is excited and the fluorescence detected by a camera. The fluorescently labelled terminator can then be removed and the process can continue to sequence the whole fragment. In some embodiments, sequencing is performed on the Illumina® MiSeq platform, (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference in its entirety), NovaSeq, NextSeq, HiSeq, and the like

### Analysis of Sequencing Data

Aspects of the present disclosure include methods of detecting disease-associated genetic alterations of single ells within a heterogeneous population in situ.

The present disclosure prepares NGS sequencing libraries from multiple cells where each cell had multiple first cell barcoding oligos and second cell barcoding oligos each with distinct barcoding sequences within. The barcoding oligos were amplified into multiple copies of barcoding primers such that during in situ amplification of the preliminary libraries different combinations of the first barcoding sequence and second barcoding sequence are combined together. The reaction concentration of the barcoding oligos and cell size work effect the number of each barcode in the each of the size such that 1-thousands of each barcode oligo can be present into a cell.

Sequence analysis aims to cluster the barcode sequences into cell groups based on the observed combinations of the first barcoding sequence and second barcoding sequence. The number of sequencing reads needed to perform this deconvolution is dependent on the number of cells and number of first barcoding sequences and second barcoding sequences per cell.

The present disclosure also provides a method for analyzing multiplexed sequencing data, such as those acquired using the library preparation method described herein. Such methods are implemented by a computer-implemented method, where a user may access a file on a computer system, wherein the file is generated by sequencing multiplexed amplification products from one or more cell populations of a heterogeneous sample by, e.g., a method of analyzing a heterogeneous cell population, as described herein. Thus, the file may include a plurality of sequencing reads for a plurality of nucleic acids derived from the heterogeneous cell population. Each of the sequencing reads may be a sequencing read of a nucleic acid that contains a target nucleic acid nucleotide sequence (e.g., a nucleotide sequence encoding a target region of interest) and one or more barcode sequences that identifies the single cell source (e.g., a single cell in a multi-well plate, a capillary, a microfluidic chamber, a microcentrifuge tube, or any other sample collection device) from which the nucleic acid originated (e.g., after PCR and/or ligation of the target nucleic acid expressed by the one or more cells in the in the well). In some embodiments, the sequencing read is a paired-end sequencing read.

The sequencing reads in the file may be aligned to a target nucleic acid nucleotide sequence by matching the nucleotide sequence comprising the sequencing read to a corresponding target nucleic acid nucleotide sequence, with appropriate sequencing error correction. After the indexed barcoded library is sequenced (barcoded sequenced library), the barcoded sequenced library undergoes a series of bioinformatics processing steps using an algorithm to populate sequencing reads for each cell into a single file.

The present inventors have developed an algorithm to tag sequencing reads from an in situ single-cell sequencing sample with a cell ID and quantifies structural variants within these single cells, from these sequencing reads.

Bioinformatics Pre-Processing of Barcoded Sequenced Library

For a given sample, a graph is created where barcodes are stored as “nodes” and the reads (which each contain 2 cell barcodes) are stored as “edges”. Graph-based algorithms are then used to cluster these barcoded reads into individual cells. In particular, the graph is “pruned” so that reads that appear due to leakage of a barcode from one cell to another cell are removed. What is left is a graph containing clusters of barcoding (†)/sequencing reads, where each cluster is a cell. All of the barcodes and reads associated with that cell are then output to a sequence FASTQ file, one per cell.

The program takes as input zipped R1, R2, I1, and I2 FASTQ files, and creates a Graph containing nodes representing barcodes, and edges representing a read containing those barcodes. Actual read sequences and associated quality scores are stored in a read dictionary. Note that in some cases where sequencing depth is not sufficient, cells may contain several sub-graphs. In these cases, appropriate methods may be used to combine these sub-graphs into a single sub-graph for a given cell. After appropriate pruning, the Graph should contain sub-graphs where each sub-graph is a “cell’. This program then returns individual FASTQ files of reads, one for each “cell”.

The method of processing the barcoded sequenced library that was prepared in situ involves processing with a computer readable medium, comprising instructions, that cause the processor to (a) produce a graphical representation of the sequenced barcoded library, perform a clustering analysis on the sequenced barcoded library, and outputting each cluster of barcoded read sequences into an individual sequence file, where each sequencing file contains barcoded read sequences for a single cell.

The clustering analysis on the sequenced barcoded library is performed to remove any barcoding errors, to cluster the barcoded sequenced library to create clusters of barcoded read sequences, where each cluster of barcoded read sequences is associated with a single cell.

Aspects of the present methods also include analyzing the sequencing file of the sequenced barcoded library for each cell to determine the presence or absence of disease-associated alterations within each cell of the permeabilized cell suspension.

In some embodiments, analyzing includes identifying, in each of the sequenced barcoded libraries, whether the sequenced libraries contain one or more indexing/barcoding/sequencing errors.

In some embodiments, analyzing the sequenced barcoded libraries includes correcting one or more indexing errors if an indexing error is present.

In some embodiments, analyzing the sequenced barcoded libraries includes removing one or more indexed libraries that does not contain an indexed sequence.

In some embodiments, analyzing the sequenced indexed libraries includes demultiplexing each of the sequenced barcoded libraries according to each of their barcode sequence.

In some embodiments, demultiplexing includes separating the reads of different barcoded libraries, as determined by the barcode sequence, into individual files, where each cell will have an individual file containing sequencing reads.

Pruning Algorithm and FASTQ Output

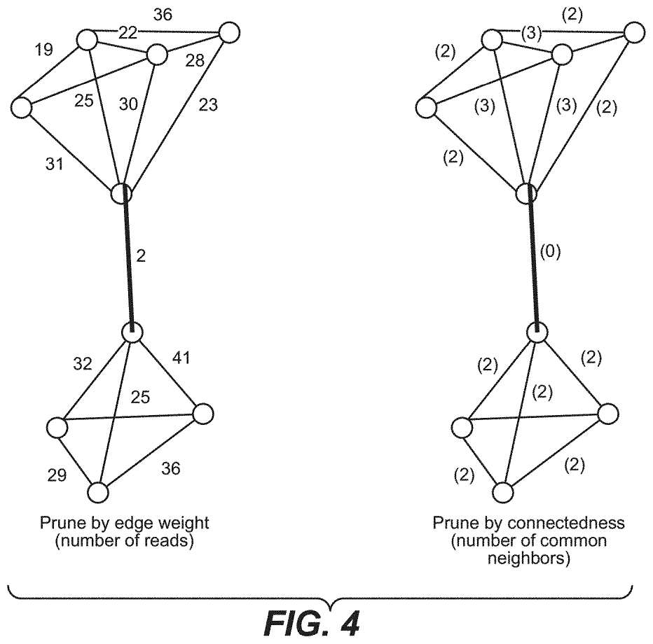

There are two types of graph pruning that can occur, depending on the read depth of the sequenced sample (see e.g., FIG. 4).

In some embodiments, the graphical representation includes nodes representing the first or second molecular cellular labels (e.g., barcode-pair), and edges representing barcoded sequencing reads comprising the sequenced barcoded library with the first and second molecular cellular label.

Graphical representation (1). If the read depth is high enough so that we get on average tens of reads per barcode-pair, this script will prune by edge weight (i.e., number of reads for a given barcode-pair. The pruning algorithm will calculate an empirical read threshold based on the data—any edges with weight less than this read threshold will be pruned. This empirical threshold is modeled based on known average experimental rates of barcode leakage from one cell to another cell, the sequencing error rates, the empirical shapes of the signal and noise distributions in the data (note: for initial testing, a constant read threshold will be used). Any singleton nodes (nodes with no edges) as a result of pruning are removed. Resulting sub-graph clusters are representative of our cells, and so read information is then output for each sub-graph cluster, one cluster per file in FASTQ format. The resultant FASTQs can then be fed into any single cell alignment and/or single cell variant calling programs.

In some embodiments, the computer readable medium causes a processor to, before performing a clustering analysis, calculating an edge weight read threshold based on the average experimental rates of barcode leakage from one cell to another, sequencing error rates, and/or the empirical shapes of the signal and noise distributions in the sequenced barcoded library.

In some embodiments, removing any barcoding errors includes pruning the graphical representation by edge weight, where edge weight is determined by the number of barcoded sequencing reads that include both the first molecular cellular label and the second molecular cellular label as a barcoded pair. In some embodiments, pruning the graphical representation by edge weight includes removing edges with an edge weight less than the edge weight read threshold. Additionally, pruning the graphical representation by edge weight results in singleton nodes that include nodes without edges being removed from the graphical representation.

Graphical representation (2). If the read depth is too low for pruning-by-edge-weight, the script will instead prune by ‘connectedness’ of barcode pairs. Connectedness is defined as follows—given two barcodes A and B of a paired-barcode read (there is an edge A-B representing this read), this algorithm finds all barcode neighbors of A, and separately all barcode neighbors of B. The algorithm then counts how many barcode neighbors A and B share in common versus distinct barcode neighbors, which gives a quantitative measure of how likely barcodes A and B are in the same cluster (same cell). This is calculated for all barcode pairs (so this is an N\hat{ }2 operation), and an empirical threshold is calculated based on the distribution of these fraction of common neighbors, the sequencing error rate, and an initial expected leakage rate based on the experiment (again, for initial testing we will start with fixed thresholds). Any barcode pairs with a fraction of common neighbors less than this threshold are pruned, and any singleton nodes as a result of pruning are removed. Resultant sub-graph clusters are representative of our cells, and so read information is then output for each sub-graph cluster, one cluster per file in FASTQ format. The resultant FASTQs can then be fed into any single cell alignment and/or single cell variant calling programs. In some embodiments, appropriate methods may be used to merge multiple sub-graphs within a cell into a single sub-graph.

In some embodiments, removing any barcoding errors includes pruning the graphical representation by connectedness of the first molecular cellular label and the second molecular cellular label as a barcoded pair. In some embodiments, connectedness of the barcoded pair comprises detecting barcode neighbors of the first molecular cellular label and barcode neighbors of the second molecular cellular label; and counting the number of barcode neighbors the first molecular cellular label and the second molecular cellular label share in common versus distinct barcode neighbors. In some embodiments, detecting barcode neighbors provides a quantitative measure of the probability of the first molecular cellular label and second molecular cellular label to be within the same cluster.

In some embodiments, pruning the graphical representation by the connectedness of the first and second molecular cellular labels comprises removing barcode pairs with a fraction of common barcode neighbors less than a threshold. For example, a threshold can be calculated based on the distribution of the fraction of common barcode neighbors, the sequencing error rate, and/or an initial expected barcode leakage rate. In some embodiments, pruning the graphical representation by connectedness of the first and second molecular cellular labels results in singleton nodes comprising nodes without edges being removed from the graphical representation.

Error Correction

Barcodes

Because cell barcodes are random, there is a chance two distinct barcodes may only be one mismatch apart (Hamming Distance of 1). Thus, we cannot assume that two barcodes with Hamming Distance of 1 arise from sequencing error and correct a priori. Instead, we allow the pruning algorithm to naturally remove edges between two barcodes that are one mismatch apart if either the number of reads with this barcode-pair or the number of common neighbors is less than the empirically-calculated threshold, based on the pruning algorithm used. Note that this empirically-calculated threshold takes into account the sequencing error rate, thus effectively providing sequencing-based error correction within the algorithm.

Aligned Reads

The cell barcodes for each read will be stored in the header of each sequence, and so will carry over into the alignment SAM/BAM files.

### Processing Sequencing Reads for Each FASTQ File

In some embodiments, analyzing the sequenced indexed libraries includes trimming each of the sequenced barcoded libraries to remove at least a portion of the barcode and/or adapter sequence. In some embodiments, analyzing the sequenced barcoded libraries includes trimming each of the barcoding/consensus/adapter sequences to remove the full barcode and/or adapter sequences. The barcode information is kept in the header of the read. Thus, the header information (e.g., barcode) will be carried through to subsequent steps in the bioinformatics analysis. The full barcode and/or adapter sequences is removed before alignment to a reference or target sequence.

In some embodiments, analyzing the sequenced indexed libraries includes aligning each of the indexed libraries to a target or reference sequence and producing an alignment file for each of the indexed libraries. In some embodiments, analyzing the sequenced indexed libraries comprises running each of the alignment files through a variant caller configured to identify and quantify genetic alterations within the indexed libraries. A variant caller, used herein in its conventional sense, is an algorithm that calls structural variants and writes them to an output file. In some embodiments, the variant caller includes additional statistical tests in addition to variant identification. In some embodiments, the variant caller does not include additional statistical tests in addition to variant identification. In some embodiments, a consensus region is first generated that is comprised of all sequencing reads that align to the same target or reference sequence and share the same error-corrected barcode molecular labels.

In some embodiments, the genetic alterations include structural variants. Non-limiting examples of structural variants include, but are not limited to splice variations, somatic mutations, or genetic polymorphisms. In some embodiments, structural variants include genetic variations and mutations associated with cancer. In some embodiments, the structural variants of the one or more populations of cells are compared with cell types with known structural variants using reference samples and variant databases.

In some embodiments, the indexed libraries are aligned to a reference sequence with one or more genome or transcriptome read aligners selected from Burrows Wheeler Aligner (BWA), BWA-MEM, Bowtie2, RNA-STAR, and Salmon. In some embodiments, the reference sequence is a sequence of the human genome. In some embodiments, the reference sequence is a sequence for the target nucleic acid in a reference database, such as GenBank®. Thus, in some embodiments, a target nucleotide sequence in a first sequencing read in a subset of sequencing reads, as described above, is 80% or more, e.g., 85% or more, 90% or more, 95% or more, or up to 100% identical to a reference sequence for the target nucleic acid from a reference database. In some embodiments, the reference sequence is one or more other sequences in sequencing reads of the same subset. Thus, in such cases, a target nucleotide sequence in a first sequencing read in a subset of sequencing reads, as described above, is 80% or more, e.g., 85% or more, 90% or more, 95% or more, or up to 100% identical to a target nucleotide sequence in a second sequencing read in the same subset. In some instances, a target nucleotide sequence in a first sequencing read in a subset is 80% or more, e.g., 85% or more, 90% or more, 95% or more, or up to 100% identical to a target nucleotide sequence in all other sequencing reads in the same subset.

In some embodiments, identifying the genetic alterations within the indexed barcoded library includes extracting structural variants from each of the alignment files of the indexed libraries. In some embodiments, extracting structural variants comprises listing all the structural variants commonly found in the alignment file for each indexed library.

In some embodiments, identifying includes identifying at least one of: the percentage of genome reads in a region of the sequence containing a variant, the quality scores of nucleotides in reads covering a variant, and the total number of reads at a variant position. In some embodiments, the quality score is output by the sequencer and tells the user the quality of that nucleotide call by the sequencer. For example, the quality score can be represented by a Phred quality score which is a unique character representing the error rate of that nucleotide call.

In some embodiments, quantifying the structural variants includes determining statistical significance of each structural variant using one of more statistical algorithms to calculate a statistical score and/or a significance value for each of the structural variants.

In some embodiments, the statistical algorithm is a binomial distribution model, over-dispersed binomial model, beta, normal, exponential, or gamma distribution model.

In some embodiments, the structural variants are selected from one of more of: single nucleotide variants (SNVs), small insertions, deletions, copy number variations (CNVs), and a combination thereof. However, the methods used herein are not limited to such structural variants.

In some embodiments, the genetic variant may be a single nucleotide variant, that is a change from one nucleotide to a different nucleotide in the same position. In some embodiments, the genetic variant may be an insertion or deletion, that adds or removes nucleotides. In some embodiments, the genetic variant may be a combination of multiple events including single nucleotide variants and insertions and/or deletions. In some embodiments, a genetic variant may be composed of multiple genetic variants present in different regions of interest.

Requiring a positive determination for the genetic variant in a plurality of replicate amplification reactions reduces the probability of a false positive determination of the genetic variant being present in a DNA sample. In some embodiments, the method includes requiring multiple positive determinations in replicate amplification reactions.

In some embodiments, the mean frequency and coefficient of variation (CV) at which a given variant is observed (i.e. in sequencing results) as a result of error in the method used to sequence a DNA sample can be used to determine and/or model background levels (i.e. noise) for a genetic variant. These values can be used, for example, to determine cumulative distribution function (CDF) values and/or to calculate z-scores. In turn, measurements and/or models of background noise for a genetic variant can then be used to establish threshold frequencies above which a genetic variant must be observed to be determined as being present in a given amplification reaction (a positive determination). For a positive determination, the frequency of the variant must be higher than the mean frequency at background levels.

In some embodiments, the method includes comparing the frequency of variants to a threshold frequency, wherein the threshold frequency is determined using, for example, a binomial, over-dispersed binomial, Beta, Normal, Exponential or Gamma probability distribution model. In some embodiments, the threshold frequency at which a given genetic variant must be observed at or above to be determined as being present in a replicate amplification reaction is the frequency at which the cumulative distribution function (CDF) value of that genetic variant reaches a predefined threshold value (CDF_thresh) of 0.95, 0.99, 0.995, 0.999, 0.9999, 0.99999 or greater.

In some embodiments of the method of the invention, the threshold frequency is determined using a z-score cut-off. In some embodiments, the background mean frequency and variance of the frequency for the genetic variant determined in step (i) are modelled with a Normal distribution, and the threshold frequency for calling a mutation is the frequency at the z-score which is a number of standard deviations above the background mean frequency. In some embodiments, the threshold frequency is the frequency at z-score of 20. In some embodiments, the threshold frequency is the frequency at z-score of 30.

In some embodiments, establishing a threshold frequency at or above which the genetic variant must be observed in sequencing results of amplification reactions to assign a positive determination for the presence of the genetic variant in a given amplification reaction comprises (a) based on the read count distribution determined for a plurality of genetic variants—which is optionally a normal distribution defined by the mean frequency and variance of the frequency determined for a plurality of genetic variants, establishing a plurality of threshold frequencies at or above which the genetic variants should be observed in sequencing results of amplification reactions to assign a positive determination for the presence of the genetic variant in a given amplification reaction, and (b) based on step (a), establishing an overall threshold frequency at or above which a genetic variant must be observed in sequencing results of a given amplification reaction to assign a positive determination for the presence of the genetic variant in that amplification reaction, which is the threshold frequency at which 90%, 95%, 97.5%, 99% or more of the threshold frequencies determined in step (a) are less than this value. In some embodiments, threshold frequencies need not be determined for each possible base at each position of the region of interest, and an overall threshold based on a plurality of genetic variants can be used in the method of the disclosure.

A computer system for implementing the present computer-implemented method may include any arrangement of components as is commonly used in the art. The computer system may include a memory, a processor, input and output devices, a network interface, storage devices, power sources, and the like. The memory or storage device may be configured to store instructions that enable the processor to implement the present computer-implemented method by processing and executing the instructions stored in the memory or storage device.

The output of the analysis may be provided in any convenient form. In some embodiments, the output is provided on a user interface, a print out, in a database, as a report, etc. and the output may be in the form of a table, graph, raster plot, heat map etc. In some embodiments, the output is further analyzed to determine properties of the single cell from which a target nucleotide sequence was derived. Further analysis may include correlating expression of a plurality of target nucleotide sequences within single cells, principle component analysis, clustering, statistical analyses, and the like.

### Composition and Kits

Aspects of the present disclosure provides a composition for preparing barcoded libraries from a heterogeneous cell population for analyzing a heterogeneous population of cells. The composition may comprise one or more of the primer sets described herein. The composition may also comprise one or more reagents, enzymes, and/or buffers described herein.

The compositions of the present disclosure may include a first set of barcoding oligonucleotides and a second set of barcoding oligonucleotides,

Aspects of the present disclosure provides a kit for preparing barcoded libraries from a heterogeneous cell population for analyzing a heterogeneous population of cells. The kit may comprise one or more primer sets, barcoding oligonucleotides, reagents, enzymes, and/or buffers described herein contained in the compositions. The kit may further comprise written instructions for processing and analyzing a heterogeneous population of cells based on the sequencing of the cells and phenotypic markers. The kit may comprise one or more primer sets, oligonucleotides, reagents, enzymes, and/or buffers described herein contained in the compositions. The kit may further comprise written instructions for generating primers from oligonucleotides using linear amplification. The kit may also comprise reagents for performing amplification techniques (e.g., PCR, isothermal amplification, ligation, tagmentation etc.), hybridization capture, purification, and/or sequencing (e.g., Next Generation Sequencing). In some cases, the kit also includes reagents for fragmentation and ligation of consensus regions to a DNA or RNA fragment.

### Producing DNA or RNA Inserts

Aspects of the present composition and/or kits can include a first primer pool set. In some embodiments, the first primer pool set of the present disclosure is designed to amplify multiple targets with the use of multiple primer pairs in a single PCR experiment.

In some embodiments the first primer pool set comprises a first forward primer pool. In some embodiments, the first primer pool set comprises a first reverse primer pool. In some embodiments the first primer pool set comprises a first forward primer pool and a reverse primer pool.

In some embodiments, each forward primer comprises a nucleotide sequence that hybridize to an anti-sense strand of a nucleotide sequence encoding a target region of DNA or RNA in one or more cells. In some embodiments, each primer comprises a unique nucleotide sequence that hybridizes to an anti-sense strand of a nucleotide sequence encoding a different target region of DNA or RNA in one or more cells. Thus, a forward primer pool can include a plurality of forward primers, where each forward primer hybridizes to a distinct target nucleic acid.

In some embodiments, each reverse primer comprises a nucleotide sequence that hybridize to a sense strand of a nucleotide sequence encoding a target region of DNA or RNA in one or more cells. In some embodiments, each primer comprises a unique nucleotide sequence that hybridizes to an anti-sense strand of a nucleotide sequence encoding a different target region of DNA or RNA of one or more cells. Thus, a reverse primer pool can include a plurality of reverse primers, where each reverse primer hybridizes to a distinct target nucleic acid.

As described herein, a first primer pool set can include publicly available primer pool sets of known nucleic target regions of interest. In some embodiments, a forward primer pool includes primers of a rhAmp PCR Panel (e.g. 10×rhAmp PCR Panel—forward pool). In some embodiments, a reverse primer pool includes primers of a rhAmp PCR Panel (e.g. 10×rhAmp PCR Panel—reverse pool).

Aspects of the present disclosure include amplifying nucleic acids from the cell population using the first primer pool set to produce a first set of amplicon products (e.g. DNA or RNA inserts). In some embodiments, the nucleic acids of the one or more cell populations are amplified in situ. In some embodiments, the compositions and kits may contain the one or more reagents used to produce the first and/or second amplicon products or DNA or RNA inserts.

Aspects of the present disclosure alternatively include hybridization capture of nucleic acids from the cell population to produce an DNA or RNA inserts. In some embodiments, the nucleic acids of the one or more cell populations are enzymatically sheared, ligated, and amplified via in situ, followed by targeted enrichment using hybridization capture methods on lysed cells. Other embodiments may the nucleic acids of the one or more cell populations are “tagged” with consensus regions using transposase mediated transposition (tagmentation) and amplified in situ before performing targeted enrichment using hybridization capture methods on lysed cells. In some embodiments both of these methods can be sorted for population using FACs before the lysing of cells. In some embodiments, the compositions and kits may contain the one or more reagents used to produce the enriched library. In some embodiments, the compositions and kits may contain the one or more reagents used to produce the enriched indexed libraries. One or more reagents can include, but is not limited to, xGen Hybridization and Wash kits, streptavidin beads, KAPA HyperPlus Kit, Agilent SureSelect and/or Agilent SureSelect QXT, and the like. Other, known library preparation kits, such as KAPA library preparation Kits or Twist Library Preparation Kits may be used for facilitating the ligation-based library preparation of the present methods.

Aspects of the present composition and/or kits, where hybridization capture is performed, include a first primer pool set or a set of oligonucleotide probes. In some embodiments, the first primer pool set, or oligonucleotide probes of the present disclosure is designed to hybridize multiple targets with the use of multiple primer pairs or oligonucleotide probes in a single hybridization capture experiment.

In alternative embodiments where hybrid capture is performed, a primer pool includes primers of a xGen Lockdown Panel. In certain embodiments where hybrid capture is performed, a primer pool includes primers of a xGen Probe Pool. In certain embodiments where hybrid capture is performed, a forward primer pool includes primers of a xGen lockdown Panels and Probe Pools. In certain embodiments where hybrid capture is performed, a primer pool includes primers of a xGen lockdown Panels and Probe Pools.

As described herein when hybrid capture is performed, the composition and kits may include blocking oligonucleotides. In certain embodiments, the blocking oligonucleotides include xGen Universal blockers.

### Enzymes and Buffers

In some embodiments, the composition and/or kits may include comprises one or more enzymes. In certain embodiments, one or more enzymes is selected from one or more of: DNA polymerase, RNA polymerase, nicking enzyme, and a Bst2.0 polymerase, Phi29 polymerase, an enzymatic fragmentation enzyme, an End Repair A-tail enzyme, a DNA ligase, or a combination thereof. In some embodiments, the nicking enzyme is selected from one or more of: nt.BstNBI and nt.BspQI, however, any enzyme which cleaves only one strand of the duplex DNA may be used.

In some embodiments, the composition and/or kits may include one or more buffers selected from: a lysis buffer, an enzyme fragmentation buffer, an End Repair A-tail buffer, a ligation buffer, buffer 3.0, buffer 3.1, PCR amplification buffer, isothermal amplification buffer, and a combination thereof.

Multiplexed Polymerase Chain Reaction

In some embodiments, the compositions and/or kit of the present disclosure may include any reagents or reaction mixtures used for amplification reactions to, for example, amplify target regions of DNA or RNA, to add consensus regions to DNA or RNA inserts (to create DNA or RNA fragments), to amplify barcoding oligonucleotides or primers, and/or to amplify DNA or RNA fragments with amplified barcode primers or oligonucleotides.

Any PCR reaction mixture and heat-resistant DNA polymerase may be used for amplification reactions. For example, those contained in a commercially available PCR kit can be used. As the reaction mixture, any buffer known to be usually used for PCR can be used. Examples include IDTE (10 mM Tris, 0.1 mM EDTA; Integrated DNA Technologies), Tris-HCl buffer, a Tris-sulfuric acid buffer, a tricine buffer, and the like. Examples of heat-resistant polymerases include Taq DNA polymerase (e.g., FastStart Taq DNA Polymerase (Roche), Ex Taq (registered trademark) (Takara), Z-Taq, AccuPrime Taq DNA Polymerase, M-PCR kit (QIAGEN), KOD DNA polymerase, and the like.

The amounts of the primer, oligonucleotide and template DNA used, etc., in the present disclosure can be adjusted according to the PCR kit, concentration of the cellular sample, and device used. In some embodiments, about 0.1 to 1 μl of the first primer pool set is added to the PCR reaction mixture. In some embodiments, a forward primer pool of about 0.5 μl or more, about 1 μl or more, about 1.5 μl or more, about 2 μl or more, about 2.5 μl or more, about 3 μl or more, about 3.5 μl or more, about 4 μl or more, about 4.5 μl or more, or about 5 μl or more is added to the PCR reaction mixture. In some embodiments, a reverse primer pool of about 0.5 μl, about 1 μl or more, about 1.5 μl or more, about 2 μl or more, about 2.5 μl or more, about 3 μl or more, about 3.5 μl or more, about 4 μl or more, about 4.5 μl or more, or about 5 μl or more is added to the PCR reaction mixture.

In some embodiments, the PCR reaction mixture includes the first primer pool set, the population of cells, and a PCR library mix. In some embodiments, the library mix is a rhAmpSeq Library Mix (e.g., 4×rhAmpSeq Library Mix 1). In some embodiments, a forward primer pool of the first primer pool set includes forward primers of a rhAmp PCR Panel. In some embodiments, a reverse primer pool of the first primer pool set includes reverse primers of a rhAmp PCR Panel.

In some embodiments, about 0.1 to 10 μl of the PCR library mix is added to the PCR reaction mixture. In some embodiments, a PCR library mix of about 0.5 μl or more, about 1 μl or more, about 1.5 μl or more, about 2 μl or more, about 2.5 μl or more, about 3 μl or more, about 3.5 μl or more, about 4 μl or more, about 4.5 μl or more, about 5 μl or more, about 6 μl or more, about 7 μl or more, about 8 μl or more, about 9 μl or more, or about 10 μl or more, is added to the PCR reaction mixture.

In some embodiments, the composition and/or kits of the present disclosure include one or more diluted cell populations.

Ligation

In some aspects, the composition and/or kits of the present disclosure include ligation reagents and/or enzymes for ligating the first set of amplicon products to produce a second set of amplicon products comprising indexed libraries. In some embodiments, LCR can be used as an alternative approach to PCR. In other embodiments, PCR can be performed after LCR.

In some embodiments, the thermostable ligase can include, but is not limited to Pfu ligase, or a Taq ligase.

In some embodiments, the composition and/or kits of the present disclosure include one or more reagents for purifying amplicon products. As described above, techniques for purifying amplicon products are well-known in the art and include, for example, using magnetic bead purification reagent, passing through a column, use of ampure beads, and the like.

### Cell Barcoding Oligonucleotides

Compositions and/or kits of the present disclosure can include barcoding oligonucleotides such as a first set of barcoding oligonucleotides and a second set of barcoding oligonucleotides.

For the first set of barcoding oligonucleotides, each oligonucleotide includes a first molecular cellular label (e.g., a degenerate sequence of 8 or more nucleotides labeled as “DS” of the “cell barcoding Oligo 1” of FIGS. 1 and 2), and two consensus regions (e.g., “cell barcoding Oligo 1” containing CR3′ and CR1′ of FIGS. 1 and 2) Similarly, for the second set of barcoding oligonucleotides, each oligonucleotide includes a second molecular cellular label (e.g., a degenerate sequence of 8 or more nucleotides labeled as “DS” of the “cell barcoding Oligo 2” of FIGS. 1 and 2), and two consensus regions (e.g., “cell barcoding Oligo 2” containing CR2′ and CR4′ of FIGS. 1 and 2).

Molecular Cellular Labels

The first and second barcoding oligonucleotides each include molecular cellular labels. The molecular cellular labels can include degenerate sequences, repeat sequences, variable sequences, or a combination of degenerate, repeat, and/or variable sequences that serve as short nucleotide sequences used to uniquely tag each molecule in a given sample library. In some embodiments, the first molecular cellular label includes 8-50 nucleotides (e.g., such as 8-10, 8-20, 10-15, 15-20, 20-25, 25-30, 30-35, 35-40, 40-45, or 45-50). In certain embodiments, the first molecular cellular label includes a length of 8 or more, 9 or more, 10 or more, 11 or more, 12 or more, 13 or more, 14 or more, 15 or more, 16 or more, 17 or more, 18 or more, 19 or more, or 20 or more nucleotides. In certain embodiments, the first molecular cellular label includes 8 nucleotides. The molecular cellular label of the first barcoding sequence is distinguishable (e.g., has different nucleotide sequences) from the molecular cellular label of the second barcoding sequence. In some embodiments, the second molecular cellular label includes 8-50 nucleotides (e.g., such as 8-10, 8-20, 10-15, 15-20, 20-25, 25-30, 30-35, 35-40, 40-45, or 45-50). In certain embodiments, the second molecular cellular label includes a length of 8 or more, 9 or more, 10 or more, 11 or more, 12 or more, 13 or more, 14 or more, 15 or more, 16 or more, 17 or more, 18 or more, 19 or more, or 20 or more nucleotides. In certain embodiments, the second molecular cellular label includes 8 nucleotides. The barcoding oligonucleotides of the present methods can include degenerate or mismatch bases within its central region to alter the sequence of the DNA or RNA fragment. Non-limiting examples of barcoding oligonucleotides can be found in U.S. Pat. No. 10,155,944, which is hereby incorporated by reference in its entirety.

In some embodiments, each cell within the heterogeneous cell population of the sample includes less than 10%, less than 8%, less than 7%, less than 6%, less than 5%, less than 4%, less than 3%, less than 2%, or less than 1% of barcoding oligonucleotides with the same first and second molecular cellular label as a different cell within the heterogeneous cell population. For example, there are distinct first barcoding oligonucleotide and second barcoding oligonucleotide combinations for each sequence within a cell based on the first and second molecular cellular labels. Combinations of the first barcoding oligonucleotide and second barcoding oligonucleotides are then identified and grouped together in a way to identify what combinations of barcodes existed in each cell.

In other words, each molecular cellular label contains a unique sample index.

Concentration of Barcoding Oligonucleotides

The concentration and/or number of barcoding oligonucleotides in the first and second set of barcoding oligonucleotides that enter the sample containing the cells may depend on the number of cells in a sample. In some embodiments, the concentration of the first and second set of barcoding oligonucleotides at which the cell is contacted with ranges from 1 femtoMolar (fM) to 5 microMolar (μM). In certain embodiments, the concentration of the first and second set of barcoding oligonucleotides at which the cell is contacted with ranges from 0.005 μM to 5 μM, such as 0.05 μM to 5 μM, 0.5 μM to 1 μM, 1 μM to 2 fM, 2 μM to 3 μM, 3 μM to 4 μM, or 4 μM to 5 μM. In certain embodiments, the concentration of the first and second set of barcoding oligonucleotides at which the cell is contacted with ranges from 1 nanoMolar (nM) to 1000 nM, such as 1 nM to 500 nM, 1 nM to 250 nM, 1 nM to 100 nM, 1 nM to 10 nM, 1 nM to 5 nM, or 1-2 nM. In certain embodiments, the concentration of the first and second set of barcoding oligonucleotides at which the cell is contacted with ranges from 1 picoMolar (pM) to 1000 pM, such as 1 pM to 100 pM, 1 pM to 50 pM, 50 pM to 100 pM, 1 pM to 10 pM, 1 pM to 5 pM, or 1-2 pM. In certain embodiments, the concentration of the first and second set of barcoding oligonucleotides at which the cell is contacted with ranges from 1 fM to 100 fM, such as 1 fM to 100 fM, 50 fM to 100 fM, 1 fM to 10 fM, 1 fM to 5 fM, or 1 fM to 2 fM.

The number of barcoding oligonucleotides in the first set of barcoding oligonucleotides and the second set of barcoding may depend on the concentration of cells within the sample. For example, in certain embodiments, about 60 first barcoding oligonucleotides and about 60 second barcoding oligonucleotides may enter the cells within the sample at a concentration of 1 pM. In certain embodiments, about 600 first barcoding oligonucleotides and about 600 second barcoding oligonucleotides may enter the cells within the sample at 10 pM. In certain embodiments, about 6 first barcoding oligonucleotides and 6 second barcoding oligonucleotides may enter the cells within the sample at a concentration of 100 fM. In some embodiments, the number of barcoding oligonucleotides in the first set of barcoding oligonucleotides ranges from 1-10,000 barcoding oligonucleotides, such as 1-5000 barcoding oligonucleotides, 5000-10,000 barcoding oligonucleotides, 1-1000 barcoding oligonucleotides, 1-500 barcoding oligonucleotides, 500-1000 barcoding oligonucleotides, 1-10 barcoding oligonucleotides, 1-20 barcoding oligonucleotides, 10-20 barcoding oligonucleotides, 5-100 barcoding oligonucleotides, 100-200 barcoding oligonucleotides, 200-300 barcoding oligonucleotides, 300-400 barcoding oligonucleotides, 400-500 barcoding oligonucleotides, 500-600 barcoding oligonucleotides, 600-700 barcoding oligonucleotides, 700-800 barcoding oligonucleotides, 800-900 barcoding oligonucleotides, or 900-1000 barcoding oligonucleotides. In some embodiments, the number of barcoding oligonucleotides in the first set of barcoding oligonucleotides is 1 or more, 5 or more, 6 or more, 10 or more, 25 or more, 50 or more, 75 or more, 100 or more, 200 or more, 300 or more, 400 or more, 500 or more, 600 or more, 700 or more, 800 or more, 900 or more, or 1000 or more. In some embodiments, the number of barcoding oligonucleotides in the second set of barcoding oligonucleotides ranges from 1-10,000 barcoding oligonucleotides, such as 1-5000 barcoding oligonucleotides, 5000-10,000 barcoding oligonucleotides, 1-1000 barcoding oligonucleotides, 1-500 barcoding oligonucleotides, 500-1000 barcoding oligonucleotides, 1-10 barcoding oligonucleotides, 1-20 barcoding oligonucleotides, 10-20 barcoding oligonucleotides, 5-100 barcoding oligonucleotides, 100-200 barcoding oligonucleotides, 200-300 barcoding oligonucleotides, 300-400 barcoding oligonucleotides, 400-500 barcoding oligonucleotides, 500-600 barcoding oligonucleotides, 600-700 barcoding oligonucleotides, 700-800 barcoding oligonucleotides, 800-900 barcoding oligonucleotides, or 900-1000 barcoding oligonucleotides. In some embodiments, the number of barcoding oligonucleotides in the second set of barcoding oligonucleotides is 1 or more, 5 or more, 6 or more, 10 or more, 25 or more, 50 or more, 75 or more, 100 or more, 200 or more, 300 or more, 400 or more, 500 or more, 600 or more, 700 or more, 800 or more, 900 or more, or 1000 or more.

The first and second barcoding oligonucleotides each include two consensus regions with a molecular cellular label positioned between the two consensus regions. The first consensus regions, shown as “CR1” and “CR1′” of the first barcoding oligonucleotides and the first consensus regions “CR2” and “CR2′” of the second set of barcoding oligonucleotide of FIGS. 1 and 2, include nucleotide sequences that are complementary to sequencing primer sites “CR1”, “CR1′”, “CR2”, and “CR2′” of the dsDNA fragments.

The first and second barcoding oligonucleotides also include an adapter sequence (see e.g., “CR3”, “CR3′”, “CR4” and “CR4′” of FIGS. 1 and 2). The adapter sequence can be nucleotide sequences that allow high-throughput sequencing of amplified nucleic acids. These adapter sequences can include, as a non-limiting example, flow cell binding sequences that are platform-specific sequences for library binding to the sequencing instrument. For example, the adapter sequence of the first set of oligonucleotides can include P5 adapter sequences, and the adapter sequence of the second set of oligonucleotides can include P7 adapter sequences.

The first and second barcoding nucleotide sequences each include a consensus read sequence and an adapter sequence that flank the molecular cellular label. Therefore, the first or second molecular sequence is positioned between the consensus read sequence and the adapter sequence.

Each set of barcoding primer will attach or bridge to either end of the DNA or RNA fragment within the cell. For example, each of the first and second barcoding oligonucleotides contains a consensus region that is complementary to one strand of the dsDNA. For example, CR1′ of Cell Barcode Oligo 1 of FIG. 1 is complementary to CR1 of the 5′ strand of the DNA fragment, while CR2′ of Cell Barcode Oligo 2 is complementary to CR2 of the 3′ strand of the DNA fragment. This provides for an initial hybridization reaction of the barcoding oligonucleotide sequences to the DNA fragment of interest.

In some embodiments, the composition and/or kits of the present disclosure can include a first set of amplification primers and a second set of amplification primers for annealing the barcoded oligonucleotides. In some embodiments, the composition and/or kits of the present disclosure can include annealed/duplex barcoding oligonucleotides already prepared and thus the first set and second set of amplification primers are not required.

The first set of amplification primers can include a consensus read region (e.g., Amplification primer 1 CR3 of FIG. 1) which is complementary to CR3′ of the first set of barcoding oligonucleotides. The second set of amplification primers can include a consensus read region (e.g., Amplification primer CR4 of FIG. 1) which is complementary to CR4′ of the second set of barcoding oligonucleotides. In some embodiments, for example where isothermal amplification is performed, the first and second amplification primers may include a cleavage site, such as a nicking endonuclease recognition site (ERS). For example, FIG. 2 shows a first and second set of amplification primers with an ERS site at the 5′ end of the first and second primer. Thus, in embodiments where an ERS site is present, the first set of amplification primers can comprise, in 5′ to 3′ order: an ERS site and a consensus read region (e.g., Amplification primer 1 CR3 of FIG. 1) which is complementary to CR3′ of the first set of barcoding oligonucleotides. In embodiments where an ERS site is present, the second set of amplification primers can comprise, in 3′ to 5′ order: a consensus read region (e.g., Amplification primer 1 CR4 of FIG. 1) which is complementary to CR4′ of the second set of barcoding oligonucleotides, and an ERS site. The barcode amplification primers and barcode oligonucleotides hybridize to form molecules with 5′ overhangs, which can then be amplified using nick-mediated isothermal amplification.

In some embodiments, before contacting the prepared DNA or RNA fragments with the barcoding sequences, the first set of amplification primers are hybridized to the complementary consensus region of the first set of barcoding oligonucleotides; and the second set of amplification primers are hybridized to the complementary consensus region of the second set of barcoding oligonucleotides. For example, the methods described herein can include mixing the first and second set of barcoding oligonucleotides with the first and second sets of amplification primers at a molar ratio sufficient to result in a first oligonucleotide set comprising duplexed double stranded oligonucleotides and a second oligonucleotide set comprising duplexed double stranded oligonucleotides. These duplexed/annealed oligonucleotides can then be contacted with the DNA or RNA fragments. Thus, in some embodiments, the composition and/or kits may include duplexed/annealed oligonucleotides.

Next, the resulting first and second set of duplexed double stranded oligonucleotides are annealed during a PCR amplification reaction or an isothermal amplification reaction to produce a set of annealed/duplexed barcoding products. The set of annealed barcoding products include, a 5′ oligonucleotide strand, from 5′ to 3′ order: a consensus read region (CR3 in FIG. 1), the first molecular cellular label (DS′), and the consensus read region (CR1 of FIG. 1); and a 3′ oligonucleotide strand complementary to the 5′ oligonucleotide strand, from 3′ to 5′ order: a consensus read region (CR3′ of FIG. 1) the first molecular cellular label (DS of FIG. 1), and the consensus read region (CR1′ of FIG. 1). The set of annealed barcoding products also include, a 3′ oligonucleotide strand, from 3′ to 5′ order: a consensus read region (CR2 in FIG. 1), the second molecular cellular label (DS' of FIG. 1), and the consensus read region (CR4 of FIG. 1); and a 5′ oligonucleotide strand complementary to the 3′ strand, from 5′ to 3′ order: a consensus read region (CR2′ of FIG. 1) the second molecular cellular label (DS), and the consensus read region (CR4′ of FIG. 1).

### Indexing Primers

In some embodiments, the composition and/or kits of the present disclosure include a set of indexing primers which include nucleotide sequences that allow identification of sequence reads during high-throughput sequencing of amplified nucleic acids. In some embodiments, the indexing primers include indexing sequences for pair-end sequencing. Indexing sequences can be used in an amplification reaction of the disclosed method for the desired sequencing method used. For example, if an Illumina sequencing platform is used, the software on the platform is able to identify these indexes on each sequence read, and since the user can input which pair of index primers were added to each sample, the platform then knows which samples to associate that read to, allowing the user to separate the reads for each different sample. In some embodiments, the method includes attaching indexing sequences to amplified nucleic acid from these sub-populations of live cells using a multiplexed PCR-based approach or ligation-based approach. In certain embodiments, indexing primers are added to the barcoded library after lysing the cells, and a subsequent PCR reaction is performed to add the indexing primers.

### Cell Sorting for Phenotypically Distinguishing Cell Populations

In certain aspects, the composition and/or kits of the present disclosure may include reagents and/or antibodies used for sorting the one or more cell populations.

### Lysing the Cells

Aspects of the present disclosure include compositions and/or kits for lysing the one or more cells within the one or more cell populations. In some embodiments, the composition and/or kits include one or more lysing agents.

Non-limiting examples of cell lysing agents include, but are not limited to, an enzyme solution. In some embodiments, the enzyme solution includes a proteases or proteinase K, phenol and guanidine isothiocyanate, RNase inhibitors, SDS, sodium hydroxide, potassium acetate, and the like. In some embodiments, lysing includes heating the cells for a period of time sufficient to lyse the cells. In certain embodiments, the cells can be heated to a temperature of about 80° C. or more, 85° C. or more, 90° C. or more, 96° C. or more, 97° C. or more, 98° C. or more, or 99° C. In certain embodiments, the cells can be heated to a temperature of about 90° C., 95° C., 96° C., 97° C., 98° C., or 99° C.

However, any known cell lysis buffer may be used to lyse the cells within the one or more cell populations.

### Methods of Barcode Oligonucleotide Amplification

This disclosure features methods of amplifying barcode oligonucleotides to generate barcoding primers, where the barcoding primers can be used in any of the downstream applications provided herein. This disclosure also features methods of amplifying oligonucleotides without barcodes to generate primers. In one example, in situ amplification of the barcode oligonucleotides provides an in situ source of reagents (i.e., barcoding primers), thereby eliminating a primary hurdle in situ library preparation: delivery of reagents (e.g., enzymes and enzyme substrates (e.g., primers and dNTPs) into the cells. In such cases, the barcoding primers produced from the amplification of the barcode oligonucleotides can be used to amplify input material (e.g., RNA or DNA) within a cell. In another example, in situ amplification of barcode oligonucleotides is combined with in situ library preparation as described in PCT/US2021/046025 (WO2022/036273), which is herein incorporated by reference in its entirety. In such cases, the barcoding primers produced from the amplification of the barcode oligonucleotides can be used to amplify the in situ libraries. Non-limiting examples of methods for barcode oligonucleotide amplification are provided below.

### Method 1

In one aspect, the method includes a hairpin barcode oligonucleotide and uses nick-mediated isothermal amplification to generate barcoding primers. Nick mediated isothermal amplification of the hairpin barcode oligonucleotide allows for the barcoding oligonucleotide to be amplified using an isothermal polymerase. Nickase-mediated nicking of the amplified barcode oligonucleotide at the nick endonuclease recognition site enables additional amplification of the template (i.e., the hairpin barcode oligonucleotide), thereby producing a second barcode primer. Repeated isothermal amplification followed by nickase-mediated nicking enables a plurality of barcode primers to be generated from the hairpin barcode oligonucleotide.

In such cases, a barcode oligonucleotide includes a hairpin (e.g., a hairpin barcode oligonucleotide) and includes from 5′ to 3′: a targeting sequence, a barcode sequence, a amplification sequence, a nick endonuclease sequence, and a stem loop sequence.

In such cases, the hairpin barcode oligonucleotide further comprises a sequence that is the reverse complement of the nick endonuclease recognition site. In some embodiments, the hairpin barcode oligonucleotide includes from 5′ to 3′: the reverse complement of a targeting sequence, the reverse complement of a barcode sequence, the reverse complement of an amplification sequence, the reverse complement of a nick endonuclease recognition site, a stem loop sequence, and the reverse complement of the nick endonuclease recognition site, or any combination thereof.

In such cases, the targeting sequence (or the reverse complement of the targeting sequence) includes an R1 adapter sequence, an R2 adapter sequence, or any other universal or consensus region provided herein.

In such cases, the barcode sequence (or the reverse complement of a barcode sequence) includes a degenerate sequence or partially degenerate sequence.

In such cases, the amplification sequence (or the reverse complement of the amplification sequence) includes a P5 sequence, or a P7 sequence.

In such cases, the nick endonuclease sequence (or the reverse complement of a nick endonuclease sequence) includes a sequence that is complementary to a reverse complement of the nick endonuclease sequence. In some embodiments, the reverse complement of the nick endonuclease sequence is a sequence that is located on the same contiguous oligonucleotide as the nick endonuclease sequence. For example, the nick endonuclease is oriented 5′ to the reverse complement of the nick endonuclease sequence in the barcode oligonucleotide.

In such cases, the stem loop sequence includes a sequence that includes sufficient number of self-complementary nucleotides at positions that enable formation of a stem loop.

In such cases, the barcode oligonucleotide also includes a sequence that is reverse complement to the nick endonuclease sequence.

In such cases, the endonuclease is selected from nt.BstNBI, nt.BbvCI, or nt.BspQI and the nick endonuclease sequence includes a sequence capable of binding to these endonucleases.

Method 1.1. In a non-limiting example, a barcode oligonucleotide is incubated in a reaction buffer with the nick endonuclease and isothermal polymerase (e.g., one of Bst2.0, Sequenase, Bsu Polymerase, EquiPhi29, and Phi29) under conditions (e.g., buffer conditions and temperature) that allow for both nicking and amplification. Amplification is measured via gel electrophoresis or single strand DNA Qubit assays.

Method 1.2. In another non-limiting example, amplification of a barcoding oligonucleotide is tested for Application 8.1 provided herein. A precursor library is prepared as described in PCT/US2021/046025 (WO2022/036273), which is herein incorporated by reference in its entirety, such that genomic fragments are labeled with R1 and R2 sequences. Barcode oligonucleotides are then added to the cell mixture and amplified in situ using optimized conditions from Method 1.1 (provided above) to create barcoding primers. After the nick mediated isothermal amplification, enzymes are heat inactivated. The input material from the cells (Application 8.1) is amplified using PCR with standard polymerases and the barcoding primers.

### Method 2

In one aspect, the barcode oligonucleotide is linear (e.g., a linear barcode oligonucleotide) and nick-mediated isothermal amplification is used to generate barcoding primers. Nick mediated isothermal amplification of a linear barcode oligonucleotide with an amplification primer allows for the barcode oligonucleotide to be amplified using an isothermal polymerase. Nickase-mediated nicking of the amplified barcode oligonucleotide at the nick endonuclease sequence enables additional amplification of the template (i.e., the barcode oligonucleotide or the amplified barcode oligonucleotide), thereby producing a second barcode primer. Repeated isothermal amplification followed by nickase-mediate nicking enables a plurality of barcode primers to be generated from the linear barcode oligonucleotide.

In such cases, a barcode oligonucleotide is linear (e.g., a linear barcode oligonucleotide) and includes from 5′ to 3′: a targeting sequence, a barcode sequence, and an amplification sequence. In some embodiments, the linear barcode oligonucleotide further comprises a nick endonuclease recognition site. In some embodiments, the linear barcode oligonucleotide further comprise an additional sequence. In some embodiments, the linear barcode sequence further comprises a nick endonuclease sequence and an additional sequence. In some embodiments, the linear barcode oligonucleotide includes from 5′ to 3′: the reverse complement of a targeting sequence, the reverse complement of a barcode sequence, the reverse complement of an amplification sequence, the reverse complement of a nick endonuclease sequence, and the reverse complement of an additional sequence, or any combination or orientation thereof.

In such cases, the targeting sequence (or the reverse complement of the targeting sequence) includes an R1 adapter sequence or an R2 adapter sequence, or any other universal or consensus region provided herein.

In such cases, the barcode sequence (or the reverse complement of a barcode sequence) includes a degenerate sequence or a partially degenerate sequence.

In such cases, the amplification sequence (or the reverse complement of the amplification sequence) includes a P5 sequence or a P7 sequence.

In such cases, the nick endonuclease recognition site (or the reverse complement of a nick endonuclease sequence) is at least partially complementary to the nick endonuclease recognition site of an amplification primer.

In such cases, the additional sequence (or the reverse complement of an additional sequence) includes a sequence having 5-10 nucleotides that allow the nick endonuclease sequence to not be at the end of the barcode oligonucleotide.

In such cases, the linear barcode oligonucleotide is amplified using an amplification primer that includes from 5′ to 3′: a nick endonuclease recognition site. In some embodiments, the nick endonuclease recognition site on the amplification primer is at least partially complementary to the nick endonuclease recognition site on the barcode oligonucleotide. In some embodiments, the linear barcode oligonucleotide is amplified using an amplification primer that includes from 5′ to 3′: the reverse complement of a nick endonuclease recognition site. In some embodiments, the amplification primer includes from 5′ to 3′: an additional sequence, a nick endonuclease recognition site, and an amplification sequence, or any combination or orientation thereof.

In such cases where the linear barcode oligonucleotide is amplified with an amplification primer including a nick endonuclease recognition site, the nick endonuclease recognition site on the amplification primer binds to the nick endonuclease sequence on the barcode oligonucleotide, thereby forming a double strand substrate capable of binding to an endonuclease. In some embodiments, where upon binding of the endonuclease to the double strand substrate, the endonuclease induces a single strand break. In some embodiments, the endonuclease is selected from nt.BstNBI, nt.BbvCI, or nt.BspQI and the nick endonuclease sequence includes a sequence capable of binding to these endonucleases.

Method 2.1. In a non-limiting example, a barcode oligonucleotide and an amplification oligo are incubated in a reaction buffer that includes a nick endonuclease (e.g., nt.BstNBI, nt.BbvCI, or nt.BspQI) and an isothermal polymerase (one of Bst2.0, Sequenase, Bsu Polymerase, EquiPhi29, Phi29) under conditions (e.g., buffer conditions and temperature) that allow for both nicking and amplification. Amplification is measured via gel electrophoresis or single strand DNA Qubit assays.

Method 2.2. In another non-limiting example, amplification of a barcoding oligo is tested for Application 2.1 provided herein. A precursor library is prepared using an NGS amplicon protocol (e.g., any of the protocols described herein or known in the art) that add R1 (read1) adapter and R2 (read2) adapter sequences to the amplicons. Barcode oligonucleotides are then added to the mixture and amplified using optimized conditions from Method 2.1 (provided above) to create barcoding primers. After the nick mediated isothermal amplification, enzymes are heat inactivated. The input material from the cells is amplified using PCR with standard polymerases and the barcoding primers.

### Method 3

In one aspect, the method includes a linear barcode oligonucleotide and uses primer invasion based isothermal amplification of a linear barcode oligonucleotide to generate barcoding primers. Primer invasion using an amplification primer allows for the barcoding oligonucleotide to be amplified using an isothermal polymerase. Repeated amplification of the template (i.e., linear barcode oligonucleotide) using primer invasion and an isothermal polymerase enables a plurality of barcoding primers to be generated from the template. Without wishing to be bound by theory, amplification of the template is promoted through natural denaturation of the template and annealing of the amplification primer to denatured template.

In such cases, a barcode oligonucleotide is linear (e.g., a linear barcode oligonucleotide) and includes from 5′ to 3′: a targeting sequence, a barcode sequence, an amplification sequence, and a primer binding site. In some embodiments, the linear barcode oligonucleotide includes from 5′ to 3′: the reverse complement of a targeting sequence, the reverse complement of a barcode sequence, the reverse complement of an amplification sequence, and the reverse complement of a primer binding site.

In such cases, the targeting sequence (or the reverse complement of the targeting sequence) includes an R1 adapter sequence, an R2 adapter sequence, or any other universal or consensus region provided herein.

In such cases, the barcode sequence (or the reverse complement of a barcode sequence) includes a degenerate sequence or a partially degenerate sequence.

In such cases, the amplification sequence (or the reverse complement of the amplification sequence) includes a P5 sequence or a P7 sequence.

In such cases, the linear barcode oligonucleotide is amplified using an amplification primer. In some embodiments, the amplification primer includes a primer binding site. In such cases, the primer binding site of the amplification primer is at least partially complementary to the primer binding site in the linear barcode oligonucleotide. In some embodiments, a primer binding site is a poly T sequence of 20 bp.

Method 3.1. In a non-limiting example, a barcode oligonucleotide and an amplification primer are incubated in reaction buffer with an isothermal polymerase (one of Bst2.0, Sequenase, Bsu Polymerase, EquiPhi29, Phi29) with a buffer condition that allows for isothermal amplification Amplification is measured via gel electrophoresis or single strand DNA Qubit assays.

Method 3.2. In a non-limiting example, amplification of a barcode oligonucleotide is tested for Application 8.1 provided herein. Precursor libraries are prepared as described in PCT/US2021/046025 (WO2022/036273), which is herein incorporated by reference in its entirety, such that genomic fragments were labeled with R1 and R2 sequences. Barcode oligonucleotides are then added to the cells and amplified in situ using optimized conditions from Method 3.1 provided herein to create barcoding primers. After the isothermal amplification of the barcode oligonucleotides to generate the barcoding primers, enzymes are heat inactivated. As described herein in Application 8.1, the input material from the cells can be amplified using barcoding primers to mediate a PCR with standard polymerases.

### Method 4

In one aspect, the method includes a linear barcode oligonucleotide and PCR amplification of the linear barcode using an amplification primer. This method allows amplification of the linear barcode oligonucleotide to occur in the same reaction as amplification of the library. Repeated amplification of the barcode oligonucleotide occurs through temperature cycling. After two or more rounds of amplification, the amplified barcoding primer amplifies the template library (which is simultaneously being amplified in the same reaction).

In some embodiments, a barcode oligonucleotide is linear (e.g., a linear barcode oligonucleotide) and includes from 5′ to 3′: a targeting sequence, a barcode sequence, and an amplification sequence. In some embodiments, a barcode oligonucleotide is linear and includes from 5′ to 3′: the reverse complement of a targeting sequence, the reverse complement of a barcode sequence, and the reverse complement of an amplification sequence. In some embodiments, the targeting sequence (or the reverse complement of the targeting sequence) includes an R1 adapter sequence, an R2 adapter sequence, or any other universal or consensus region provided herein.

In such cases, the barcode sequence (or the reverse complement of a barcode sequence) includes a degenerate sequence or a partially degenerate sequence.

In such cases, the amplification sequence (or the reverse complement of the amplification sequence) includes a P5 sequence or a P7 sequence.

In such cases, the linear barcode oligonucleotide is amplified using an amplification primer. In some embodiments, the amplification primer includes an amplification sequence. In such cases, the amplification sequence of the amplification primer is at least partially complementary to the amplification sequence in the linear barcode oligonucleotide.

Method 4.1. In a non-limiting example, a precursor library is prepared using an NGS amplicon protocol (e.g., any of the protocols described herein or known in the art) that add R1 adapter and R2 adapter sequences to the amplicons. Barcode oligonucleotides and amplification primers are added to the precursor libraries and amplified using PCR according to the methods provided herein or known in the art. Each PCR cycle amplifies the barcode oligonucleotide, thereby producing barcoding primers. The barcoding primers include sequences that are at least partially complementary to the R1 and/or R2 adapter sequences. As described herein in Application 2.1, the barcoding primers are used in in subsequent PCR cycles to bind to the R1 and/or R2 adapter sequences and amplify the precursor library.

### Method 5

In one aspect, the method includes a circularized barcode oligonucleotide and uses rolling circle amplification to generate barcoding primers. Rolling circle amplification (RCA) amplifies a circularized template containing barcode information using an amplification primer as initial primer. RCA creates a concatemer of primers, which can be cleaved into monomers by introducing an additional oligo which binds an endonuclease site and enables endonuclease-mediated cleavage of the concatemer, thereby creating the monomers. The monomers (i.e., barcoding primers)act as primers of template DNA or precursor libraries.

In some embodiments, a barcode oligonucleotide is circularized (i.e., a circularized barcode oligonucleotide). In some embodiments, the circularized barcode oligonucleotide comprises a targeting sequence, a barcode sequence, and an amplification sequence. In some embodiments, the circularized barcode oligonucleotide further comprises a [[first]] restriction endonuclease site. In some embodiments, the circularized barcode oligonucleotide includes the reverse complement (rc) of a targeting sequence, the reverse complement of a barcode sequence, the reverse complement of an amplification sequence. In some embodiments, the circularized barcode oligonucleotide further comprises the reverse complement of a restriction endonuclease site.

In some embodiments, the circularized barcode oligonucleotide is amplified using an amplification primer. In some embodiments, the amplification primer includes an amplification sequence. In such cases, the amplification sequence of the amplification primer is at least partially complementary to the amplification sequence in the circularized barcode oligonucleotide.

In some embodiments, the circularized barcode oligonucleotide is contacted with an additional oligonucleotide. In some embodiments, the additional oligonucleotide includes a second restriction endonuclease site or a reverse complement of a restriction endonuclease site. In such cases, the restriction endonuclease site (or the reverse complement of a restriction endonuclease site) is at least partially complementary to the restriction endonuclease site in the circularized barcode oligonucleotide.

### Application of Barcode Amplification

This disclosure features methods of using the amplified barcode oligonucleotide. In one embodiment, barcoding primers (generated by amplification of the barcode oligonucleotide) are combined with in situ library preparation as described in PCT/US2021/046025 (WO2022/036273), which is herein incorporated by reference in its entirety. In such cases, the barcoding primers can be used to amplify the in situ libraries. In another embodiment, barcoding primers (generated by amplification of the barcode oligonucleotide) are used to amplifying input material (e.g., DNA). In some cases, the input material was previously isolated from cells. In some cases, barcoding primers are designed to include a sequence that targets one or more genomic regions with the DNA and can serve as the basis for an amplification reaction. In some cases, the barcoding primers recognize precursor libraries containing universal sequences.

Non-limiting examples of methods of using amplified barcode oligonucleotides (e.g., barcoding primers) are provided below.

### Application 1

In one aspect, this disclosure features a method of barcode oligonucleotide amplification in a single reaction container before any steps of library preparation are performed. In some embodiments, the amplification of the barcode oligonucleotide produces a barcode oligonucleotide amplicon (also referred as a barcoding primer). The barcoding primer can be used for further amplification.

In such cases, the input material is present in the single reaction container at the time of barcode oligonucleotide amplification is performed. In one embodiment, the input material is present in the single reaction container at the time of barcode oligonucleotide amplification is performed. In another embodiment, the input material is added to a single reaction container after amplification of the barcode oligonucleotide.

In such cases, input material is selected from genomic DNA, RNA, or cDNA from one or more cells.

In such cases, a reaction container is selected from: a single PCR tube, a single well (in a multi-well plate), or any other reaction container provided herein.

In such cases, the barcoding sequence in the barcoding oligo is selected from a defined sequence (i.e., sample id), a set of defined sequences, or a degenerate sequence. In some embodiments, the barcode oligo does not include a barcode sequence.

In such cases, a targeting sequencing in the barcoding oligo is designed to target a genomic region.

Application 1.1. In one embodiment, one or more different barcoding oligonucleotides designed to recognize specific genomic loci are added to a reaction container and amplified to generate barcoding primers from each of the one or more different barcoding oligonucleotides. In such cases, input material (genomic DNA or cDNA) is then added to the reaction container and amplification (e.g., PCR amplification) of the input material is performed using the barcoding primers. In such cases, additional primers are added to the reaction container as required.

Application 1.2. In one embodiment, one or more different barcoding oligonucleotides designed to recognize specific genomic loci are added to a reaction container containing input material (genomic DNA or cDNA). In such cases, the barcoding oligonucleotides are amplified to generate barcoding primers from each of the one or more different barcoding oligonucleotides. Amplification (e.g., PCR amplification) of the input material is performed using the barcoding primers. In such cases, additional primers are added to the reaction container as required.

Application 1.3. In one embodiments, one or more different barcoding oligonucleotides designed to recognize specific genomic loci are added to a reaction container and amplified to generate barcoding primers from each of the one or more different barcode oligonucleotides. In such cases, input material (e.g., RNA) is then added to the reaction container and reverse transcriptase amplification of the input material is performed using the barcoding primers. cDNA synthesis is completed according to standard procedures.

Application 1.4. In one embodiment, one or more different barcoding oligonucleotides designed to recognize specific genomic loci are added to a reaction container containing input material (e.g., RNA) and the barcoding oligonucleotides are amplified to generate barcoding primers from each of the one or more different barcoding oligonucleotides. The barcoding primers are then used to reverse transcribe the input material (e.g., RNA). cDNA synthesis is completed according to standard procedures.

### Application 2

In one aspect, this disclosure features a method of barcode oligonucleotide amplification in a single reaction containing input material that comprise consensus regions. In such cases, the barcode oligo amplification generates barcoding primers that can be used for amplification of the input material comprising universal sequences. In some embodiments, the input material is a precursor library.

In such cases, input material is selected from genomic DNA, RNA, or cDNA from one or more cells.

In such cases, a reaction container is selected from: a single PCR tube, a single well (in a multi-well plate), or any other reaction container provided herein.

In such cases, the barcoding sequence in the barcoding oligo is selected from a defined sequence (i.e., sample id), a set of defined sequences, or a degenerate sequence. In some embodiments, the barcode oligo does not include a barcode sequence.

In such cases, a targeting sequencing in the barcoding oligo is designed to bind to consensus regions (e.g., a read1 (R1) sequence and/or a read2 (R2) sequence).

Application 2.1. In one embodiment, genomic DNA is amplified with targeting primers containing one or more consensus regions (e.g., a R1 sequence and/or a R2 sequence) to generate DNA amplicons comprising the R1 and/or R2 sequences. In such cases, barcoding oligonucleotides including sequences designed to recognize one or both of the R1 and R2 sequences are added to the reaction container and amplified to generate barcoding primers. The barcoding primers are then used to amplify (e.g., using PCR amplification) the DNA amplicons comprising the R1 or R2 sequences. In such cases, additional amplification primers are added to the reaction container as needed.

Application 2.2. In one embodiment, genomic DNA is fragmented and adapters comprising consensus regions R1 and R2 (e.g., CR1, CR1′, CR2 and/or CR2′) are ligated on to the fragmented DNA, thereby generating DNA fragments comprising the R1 or R2 sequences. In such cases, barcoding oligonucleotides designed to recognize one or both of the R1 and R2 sequences are added to the reaction container and amplified to generate barcoding primers. The barcoding primers are then used to amplify (e.g., using PCR amplification) the DNA fragments comprising the R1 or R2 sequences. In such cases, additional amplification primers are added to the reaction as needed.

Application 2.3. In one embodiment, RNA is converted into cDNA using standard methods for reverse transcription such that a cDNA molecule comprising a R1 sequence or a R2 sequence on either end of the cDNA molecule is produced. In such cases, barcoding oligonucleotides designed to recognize one or both of the R1 and R2 sequences are added to the reaction container and amplified to generate barcoding primers. The barcoding primers are then used to amplify (e.g., using PCR amplification) the cDNA molecule comprising the R1 or R2 sequences as primer binding sites. In such cases, additional amplification are added to the reaction container as needed.

### Application 3

In one aspect, this disclosure features a method of using barcode oligonucleotide amplification to generate barcoding primers in a droplet comprising a cell or cell population. In such cases, barcode oligonucleotides are added to the cell population before droplet formation. In some cases, barcode oligonucleotides are merged with cells after droplet formation. Where barcode oligonucleotides are merged with cells after droplet formation, the barcode oligonucleotides are in a liquid phase and the result of the merger is a single droplet. In some embodiments, a first liquid phase comprising a cell or a cell population, a second liquid phase comprising the barcode oligonucleotides (and other amplification reagents), and a third immiscible phase are combined to form a droplet.

Amplification of the barcode oligonucleotides generates barcoding primers that can be used for amplification of the input material from the cell or cell population. Non-limiting examples include using the barcoding primers in a single-plex or multiplex PCR reaction, or a single-plex or multiplex reverse-transcriptase reaction. Adjusting concentrations of barcoding oligonucleotides in the cell population allows for a distribution of barcode sequences in each reaction container (or in the buffer that merges with a droplet) such that the number of barcodes in each reaction container could be ˜1 or more than 1.

In such cases, input material is selected a cell or population of cells.

In such cases, a reaction container is a droplet.

In some embodiments, droplets and methods of making and using the same are as described in U.S. Patent Publication No. 2018/0216162, which is herein incorporated by reference in its entirety.

In such cases, the barcoding sequence in the barcoding oligo is a set of defined sequences or a degenerate sequence.

In such cases, a targeting sequencing in the barcoding oligo is designed to target a genomic region. In some cases, a targeting sequencing the barcoding oligo is designed to target two or more, three or more, four or more, five or more, six or more, seven or more, eight or more, nine or more, or ten or more genomic regions.

Application 3.1. In one embodiment, cells are mixed with barcoding oligonucleotides designed to recognize specific genomic regions. In such cases, droplets form around the cells and the droplet includes the reagents needed for performing amplification ((e.g., barcode oligonucleotide primers and amplification reagents). Barcode oligonucleotides are amplified to produce barcoding primers. The barcoding primers can be used for amplification (e.g., PCR amplification) of genomic DNA or RNA from the cell(s) in the droplet.

Application 3.2. In some embodiments, a droplet comprising a cell is merged with a droplet comprising reagents (e.g., barcode oligonucleotide primers and amplification reagents) to form a single droplet including both the cell and the reagents. In the merged droplets, barcode oligonucleotides designed to recognize genomic targets are amplified to generate barcoding primers, which are then used as amplification primers in an amplification reaction (e.g., PCR amplification) of genomic DNA or RNA.

### Application 4

In one aspect, this disclosure features a method of using barcode oligonucleotide amplification to generate barcoding primers in a droplet comprising a cell or cell population. In such cases, barcode oligonucleotides are added to the cell population before droplet formation. In some cases, barcode oligonucleotides are merged with cells after droplet formation. Where barcode oligonucleotides are merged with cells after droplet formation, the barcode oligonucleotides are in a liquid phase and the result of the merger is a single droplet. In some embodiments, a first liquid phase comprising a cell or a cell population, a second liquid phase comprising the barcode oligonucleotides (and other amplification reagents), and a third immiscible phase are combined to form a droplet.

Amplification of the barcode oligonucleotides generates barcoding primers that can be used for amplification of the input material from the cell population. Non-limiting examples include using the barcoding primers in a single-plex or multiplex PCR reaction, or a single-plex or multiplex reverse-transcriptase reaction. Adjusting concentrations of barcoding oligonucleotides in the cell or cell population allows for a distribution of barcode sequences in each reaction container (or in the buffer that merges with a droplet) such that the number of barcodes in each reaction container could be ˜1 or more than 1.

In such cases, input material is selected a cell or population of cells.

In such cases, a reaction container is a droplet.

In some embodiments, droplets and methods of making and using the same are as described in U.S. Patent Publication No. 2018/0216162, which is herein incorporated by reference in its entirety.

In such cases, the barcoding sequence in the barcoding oligo is a set of defined sequences or a degenerate sequence.

In such cases, a targeting sequencing in the barcoding oligo is designed to target a genomic region.

Application 4.1. In one embodiment, a droplet forms around a cell and precursor libraries are generated with targeting primers (e.g., targeting primers comprising one or more consensus regions (e.g., a R1 sequence and/or a R2 sequence)) within the droplets. In such cases, a droplet comprising a cell is then merged with reagents (e.g., barcode oligonucleotide primers and amplification reagents) to form a single droplet including both the cell and the reagents. In the merged droplets, barcode oligonucleotides designed to recognize consensus regions are amplified to generate barcoding primers, which are then used as amplification primers in an amplification (e.g., PCR amplification) reaction to amplify the precursor libraries.

### Application 5

In one aspect, barcode oligonucleotides are added to a cell or cell population before sorting individual or populations of cells (e.g., two or more cells) into a position in a multi-well plate (e.g., a reaction container). In another aspect, barcode oligonucleotides are added to a cell or cell population after sorting the cell or cells into a specific well (e.g., a reaction container). Amplification of the barcode oligonucleotides generates barcoding primers that can be used for amplification of the input material from the cell or cell populations. Non-limiting examples include using the barcoding primers in a single-plex or multiplex PCR reaction, or a single-plex or multiplex reverse-transcriptase reaction. Adjusting concentrations of barcoding oligonucleotides in the cell population allows for a distribution of barcode sequences in each reaction container, such that the number of barcodes in each reaction container could be ˜1 or more than 1.

In such cases, input material is selected a cell or population of cells.

In such cases, a reaction container is one or more wells, for example, one or more wells in a multi-well plate.

In such cases, the barcoding sequence in the barcoding oligo is a set of defined sequences or a degenerate sequence.

In such cases, a targeting sequencing in the barcoding oligo is designed to target a genomic region.

Application 5.1. In one embodiment, one or more different barcoding oligonucleotides designed to recognize specific genomic loci are added to a reaction container (e.g., a well) and amplified to generate barcoding primers from each of the one or more different barcode oligonucleotides. In such cases, input material (e.g., a single cell or population of cells) are then added to the reaction container (e.g., a well) and amplification (e.g., PCR amplification) is performed using barcoding primers. In such cases, additional primers are added to the reaction container as required.

Application 5.2. In one embodiment, one or more different barcoding oligonucleotides designed to recognize specific genomic loci are added to input material (e.g., from a cell or a population cells) and then separated into specific reaction containers (e.g., a well) before being amplified to generate barcoding primers from each of the one or more different barcode oligonucleotides. The barcoding primers are then used to amplify (e.g., using PCR amplification) the input material from the cell or population of cells. In such cases, additional primers are added to the reaction container as required.

Application 5.3. In one embodiment, one or more different barcoding oligonucleotides designed to recognize specific genomic loci are added to a reaction container containing input material (e.g., a cell or a population of cells) that has already undergone some processing (e.g., cell lysis, Whole Genome Amplification (WGA)) and amplified to generate barcoding primers from each of the one or more different barcoding oligonucleotides. The barcoding primers are then used to amplify (e.g., using PCR amplification) the input material from the cell or population of cells. In such cases, additional primers are added to the reaction container as required.

### Application 6

In another aspect, the method includes barcode oligonucleotides added to a cell or a cell population before sorting the cell or cell population into a position in a multi-well plate (e.g., a reaction container). In another aspect, the method includes barcode oligonucleotides added to the cell or cell population after sorting to specific wells (e.g., specific reaction container). Amplification of the barcode oligonucleotides generates barcoding primers that can be used to amplify the input material from the cell or cell population. Non-limiting examples include using the barcoding primers in a single-plex or multiplex PCR reaction, or a single-plex or multiplex reverse-transcriptase reaction. Adjusting concentrations of barcoding oligonucleotides in the cell or cell population allows for a distribution of barcode sequences in each reaction container, such that the number of barcodes in each reaction container could be ˜1 or more than 1.

In such cases, input material is selected a cell or population of cells.

In such cases, a reaction container is one or more wells, for example, one or more wells in a multi-well plate.

In such cases, the barcoding sequence in the barcoding oligo is a set of defined sequences or a degenerate sequence.

In such cases, a targeting sequencing in the barcoding oligo is designed to bind to consensus regions (e.g., R1 sequence and/or R2 sequence).

Application 6.1. In one embodiment, one or more different barcoding oligonucleotides designed to recognize a consensus region are added to a reaction container (e.g., a well) containing input material (e.g., a cell or population of cells) which has already undergone some processing (e.g., cell lysis, WGA amplification, RT-PCR, or Ligation) where processing produced input material comprising the consensus region. The barcoding primers are then used to amplify (e.g., using PCR amplification) the input material from the cell or population of cells. Here, barcoding primers bind to the consensus region on the input material. This binding serves as the basis for the PCR amplification. In such cases, barcoding primers include sequences that bind to the consequence region.

### Application 7

In one aspect, barcode oligonucleotides are added to a cell or cell population that has been prepared for in situ library prep (as described in PCT/US2021/046025 (WO2022/036273), which is herein incorporated by reference in its entirety) Amplification of the barcode oligonucleotides generates barcoding primers that can be used for amplification of genomic DNA or RNA present within each reaction container (e.g., the cell). Adjusting concentrations of barcoding oligo in the cell population would allow for a distribution of barcode sequences in each reaction container (each cell), such that the number of barcodes in each reaction container could be ˜1 or more than 1.

In such cases, input material is selected a cell or population of cells.

In such cases, a reaction container is a cell.

In such cases, the barcoding sequence in the barcoding oligo is a set of defined sequences or a degenerate sequence.

In such cases, a targeting sequencing in the barcoding oligo is designed to target a genomic region.

Application 7.1. In one embodiment, barcode oligonucleotides can be added to a cell or cell population that has been prepared for in situ library prep (as described in PCT/US2021/046025 (WO2022/036273), which is herein incorporated by reference in its entirety). Barcode oligonucleotides can be amplified in situ to generate barcoding primers. The barcoding primers are then used to amplify (e.g., using PCR amplification) the in situ prepared libraries. In such cases, additional primers are added as required.

Application 7.2. In one embodiment, barcode oligonucleotides can be added to a cell or cell population that has been prepared for in situ library prep (as described in PCT/US2021/046025 (WO2022/036273), which is herein incorporated by reference in its entirety). Barcode primers can be amplified in situ to generate barcoding primers. The barcoding primers are then used to reverse transcribe the in situ prepared libraries. In such cases, additional primers are added to the reaction container as required.

### Application 8

In one aspect, barcode oligonucleotides are added to a cell or cell population that has been prepared for in situ library prep (as described in PCT/US2021/046025 (WO2022/036273), which is herein incorporated by reference in its entirety) and has undergone processing to generate precursor libraries containing consensus regions (e.g., a R1 sequence or a R2sequence). Amplification of the barcode oligonucleotide generates barcoding primers. The barcoding primers are then used to amplify (e.g., using PCR amplification) the precursor libraries within each reaction container (the cell). Adjusting concentrations of barcode oligonucleotides in the cell population allows for a distribution of barcode sequences in each reaction container (each cell), such that the number of barcodes in each reaction container could be ˜1 or more than 1.

In such cases, input material is from a cell or population of cells.

In such cases, a reaction container is a cell.

In such cases, the barcoding sequence in the barcoding oligo is a set of defined sequences or a degenerate sequence.

In such cases, a targeting sequencing in the barcoding oligo is designed to bind to consensus regions (e.g., read1 (R1) sequence and/or read2 (R2) sequence).

In one embodiments, barcode oligonucleotides can be added to a cell or cell population that has been prepared for in situ library prep (as described in PCT/US2021/046025 (WO2022/036273), which is herein incorporated by reference in its entirety) and have undergone processes to add consensus regions to the library. The barcoding primers are then used to amplify the in situ prepared libraries. For example, barcoding primers bind to the consensus region on the precursor libraries. This binding serves as the basis for the PCR amplification. In such cases, additional primers added are added to the reaction container as required.

### Additional Embodiments

Embodiment 1. A method of performing whole cell barcoding, the method comprising:

(a) contacting DNA or RNA fragments within a permeabilized cell suspension or tissue slices with:

(i) a first set of nucleotide sequences comprising:

(ia) a first set of barcoding oligonucleotides, each barcoding oligonucleotide comprising:

a first molecular cellular label comprising 8 or more nucleotides;

two consensus regions, wherein the two consensus regions of each barcoding primer comprises:

a nucleotide sequence that is complementary to a 5′ read region of a first strand of the DNA or RNA fragments, and

a first adapter sequence,

(ib) a first primer set comprising nucleotide sequences that are complementary to the adapter sequence of the first set of barcoding oligonucleotides;

(ii) a second set of nucleotide sequences comprising:

(iia) a second set of barcoding oligonucleotides, each barcoding oligonucleotides comprising:

a second molecular cellular label comprising 8 or more nucleotides;

two consensus regions, wherein the two consensus regions of each barcoding primer comprises:

a nucleotide sequence that is complementary to a 5′ read region of a second strand of the DNA or RNA fragments, and

a second adapter sequence,

(iib) a second primer set comprising nucleotide sequences that are complementary to the adapter sequence of the second set of barcoding oligonucleotides,

(b) amplifying:

the first set of barcoding oligonucleotides with the first primer set to produce a first set of barcoding primers; and

the second set of barcoding oligonucleotides with the second primer set to produce a second set of barcoding primers;

(c) amplifying the DNA or RNA fragments with first and second set of barcoding primers to produce a set of amplicon products, wherein the set of amplicon products comprise the first barcoding primer bridging from the 5′ end the DNA or RNA nucleotide sequences and the second barcoding primer bridging from the 3′ end of the DNA or RNA fragments.

Embodiment 2. The method of embodiment 1, wherein the first set of barcoding oligonucleotides and the first primer set are annealed prior to said contacting to produce a first set of annealed barcoding oligonucleotides.

Embodiment 3. The method of embodiment 2, wherein the said amplifying in step (b) comprises amplifying via polymerase chain reaction, the first and second set of annealed barcoding oligonucleotides to produce the first and second barcoding primers.

Embodiment 4. The method of embodiment 2, wherein the said amplifying in step (b) comprises amplifying via isothermal amplification, the first and second set of annealed barcoding oligonucleotides to produce the first and second barcoding primers.

Embodiment 5. The method of embodiment 2, wherein the first set of barcoding oligonucleotides and the first primer set are not annealed prior to said contacting.

Embodiment 6. The method of any one of Embodiments 1-5, wherein the DNA or RNA fragments are not amplified during step (b).

Embodiment 7. The method of embodiment 1, wherein the first and second barcoding oligonucleotides comprise hairpin barcoding oligonucleotides.

Embodiment 8. The method of any one of embodiments 1-7, wherein the DNA is a double-stranded DNA (dsDNA) fragment.

Embodiment 9. The method of any one of embodiments 1-8, wherein the first and second molecular cellular labels each comprises a degenerate nucleotide sequence.

Embodiment 10. The method of any one of embodiments 1-9, wherein the first and second molecular cellular labels each comprises 8-50 nucleotides.

Embodiment 11. The method of any one of embodiments 1-10, wherein the degenerate sequence comprises 8-50 nucleotides.

Embodiment 12. The method of any one of embodiments 1-11, wherein the degenerate sequence comprises 8-20 nucleotides.

Embodiment 13. The method of any one of embodiments 1-12, wherein the two consensus regions of the first barcoding oligonucleotides flank the first molecular cellular label.

Embodiment 14. The method of any one of embodiments 1-13, wherein the two consensus regions of the second barcoding oligonucleotides flank the second molecular cellular label.

Embodiment 15. The method of any one of embodiments 1-14, wherein the nucleotide sequence of the first or second molecular cellular label is positioned between the nucleotide sequences of the two consensus regions.

Embodiment 16. The method of any one of embodiments 1-15, wherein the degenerate sequence of each first and second molecular cellular label is distinguishable from one another.

Embodiment 17. The method of any one of embodiments 1-16, wherein the first molecular cellular label of the barcoding oligonucleotides within the first set of barcoding oligonucleotides is distinguishable from other first molecular cellular labels of the first set of barcoding oligonucleotides by its nucleotide sequence.

Embodiment 18. The method of any one of embodiments 1-17, wherein the second molecular cellular labels of the barcoding oligonucleotides within the second set of barcoding oligonucleotides is distinguishable from other second molecular cellular labels of the second set of barcoding oligonucleotides by its nucleotide sequence.

Embodiment 19. The method of any one of embodiments 1-18, wherein said contacting comprises contacting the cell suspension or tissue slices with the first and second set of barcoding oligonucleotides at a concentration such that each cell within the cell suspension or tissue slice comprises a first and second barcoding oligonucleotide that is distinguishable from a first and second barcoding oligonucleotide of a different cell.

Embodiment 20. The method of Embodiment 19, wherein the concentration ranges from 100 fM to 1 μM.

Embodiment 21. The method of Embodiment 20, wherein the concentration ranges from 1 pM-10 pM.

Embodiment 22. The method of any one of embodiments 1-21, wherein said contacting comprises contacting the cell suspension or tissue slices with the first and second set of barcoding oligonucleotides at a concentration such that each cell within the cell suspension or tissue slice comprises 2-1000 barcoding oligonucleotides.

Embodiment 23. The method of any one of embodiments 1-22, wherein a cell within the cell suspension or tissue slice comprises less than 5% of barcoding oligonucleotides with the same first and second molecular cellular label as a different cell within the cell suspension.

Embodiment 24. The method of any one of embodiments 1-23, wherein a cell within the cell suspension or tissue slice does not comprise the first and second molecular cellular label that is the same first and second molecular cellular labels of a second cell within the cell suspension or tissue slice.

Embodiment 25. The method of any one of embodiments 1-24, wherein the DNA fragment is a DNA amplicon product.

Embodiment 26. The method of any one of embodiments 1-25, wherein the RNA fragment is an RNA amplicon product.

Embodiment 27. The method of any one of embodiments 1-26, wherein the DNA or RNA fragment is a DNA or RNA product of ligation.

Embodiment 28. The method of any one of embodiments 1-27, wherein the DNA fragment comprises genomic DNA comprising a target region positioned between a first consensus read region and a second consensus read region, each first and second consensus read region selected from: a Y-adapter nucleotide sequence, a hairpin nucleotide sequence, and a duplex nucleotide sequence.

Embodiment 29. The method of any one of embodiments 1-28, wherein the DNA or RNA fragment is a DNA or RNA product of tagmentation.

Embodiment 30. The method of any one of embodiments 1-33, wherein the DNA fragment comprises genomic DNA (gDNA) modified to contain a first consensus read region at the 5′ end of the DNA sequence and a second consensus read region at the 3′ end of the DNA sequence.

Embodiment 31. The method of any one of embodiments 1-33, wherein the RNA sequence is a reverse transcribed RNA sequence comprising a target region, a first consensus read region, and a second consensus read region.

Embodiment 32. The method of Embodiment 31 wherein the first consensus read region is at the 5′ end of the target region, and the second consensus read region is at the 3′ end of the target region.

Embodiment 33. The method of any one of embodiments 1-33, wherein the RNA sequence is selected from: messenger RNA (mRNA), transfer RNA (tRNA), and ribosomal RNA (rRNA), guide RNA (gRNA), and trans-activating crispr RNA (tracrRNA).

Embodiment 34. The method of any one of embodiments 1-33, wherein the DNA or RNA fragments in step (a) comprises: a 5′ consensus read region; a 3′ consensus read region; and a target region.

Embodiment 35. The method of any one of embodiments 1-34, wherein the method further comprises, after step (c) contacting the amplicon product with a set of indexing primers, and performing an amplification reaction to produce a second set of amplicon products.

Embodiment 36. The method of any one of embodiments 1-34, wherein the method comprises lysing the cells containing the set of amplicon products.

Embodiment 37. The method of Embodiment 36, wherein the method comprises lysing the cells containing the second set of amplicon products.

Embodiment 38. The method of Embodiment 37, wherein the method further comprises contacting the second set of amplicon products with a third primer set comprising amplification primers, and performing an amplification reaction to produce a third set of amplicon products.

Embodiment 39. The method of any one of embodiments 1-38, wherein the method further comprises, after step (c), sequencing the DNA or RNA amplicon product to produce a barcoded sequenced library.

Embodiment 40. The method of any one of embodiments 1-39, wherein the cell suspension comprises 1000 cells or less.

Embodiment 41. The method of any one of embodiments 1-39, wherein the cell suspension comprises 50 cells or less.

Embodiment 42. The method of any one of embodiments 1-41, wherein the cell suspension comprises 5 cells or less.

Embodiment 43. The method of any one of embodiments 1-42, wherein the cell suspension comprises a single cell.

Embodiment 44. The method of any one of embodiments 1-43, wherein the method further comprises, sequencing the amplicon products to produce a sequenced barcoded library comprising barcoding sequences for each cell within the cell suspension or tissue slices.

Embodiment 45. A method of performing whole cell barcoding, the method comprising:

(a) contacting DNA or RNA fragments within a permeabilized cell suspension or tissue slices with:

(i) a first set of nucleotide sequences comprising:

(ia) a first set of barcoding oligonucleotides, each barcoding oligonucleotide comprising:

a first molecular cellular label comprising 8 or more nucleotides;

two consensus regions, wherein the two consensus regions of each barcoding oligonucleotide comprises:

a nucleotide sequence that is complementary to a 5′ read region of a first strand of the DNA or RNA fragments, and

a first adapter sequence,

(ib) a first primer set comprising amplification primers, each amplification primer comprising:

a first cleavage site;

(ii) a second set of barcoding nucleotide sequences comprising:

(iia) a second set of barcoding oligonucleotides, each barcoding oligonucleotides comprising:

a second molecular cellular label comprising 8 or more nucleotides;

two consensus regions, wherein the two consensus regions of each barcoding primer comprises:

a nucleotide sequence that is complementary to a 5′ read region of a second strand of the DNA or RNA fragments, and

a second adapter sequence,

(iib) a second primer set comprising amplification primers, each amplification primer comprising:

a second cleavage site,

(b) amplifying:

the first set of barcoding oligonucleotides and the first primer set to produce a first set of barcoding primers; and

the second set of barcoding oligonucleotides and the second primer set to produce a second set of barcoding primers; and

(c) amplifying the DNA or RNA fragments with the first and second barcoding primers to produce a set of amplicon products, wherein the set of amplicon products comprise the first barcoding primer bridging from the 5′ end the DNA or RNA fragments and the second barcoding primer bridging from the 3′ end of the DNA or RNA fragments.

Embodiment 46. The method of embodiment [00808], wherein the first and second barcoding oligonucleotides comprise hairpin barcoding oligonucleotides.

Embodiment 47. The method of any one of embodiments [00808]-[00830], wherein the first primer set further comprises nucleotide sequences that are complementary to the first adapter sequences of the first set of barcoding oligonucleotides, and wherein the second primer set further comprises nucleotide sequences that are complementary to the second adapter sequences of the first set of barcoding oligonucleotides.

Embodiment 48. The method of embodiment [00831], wherein the first set of barcoding oligonucleotides and the first primer set are annealed prior to said contacting to produce a first set of annealed barcoding oligonucleotides.

Embodiment 49. The method of embodiment [00832], wherein the said amplifying in step (b) comprises amplifying via isothermal amplification, the first and second set of annealed barcoding oligonucleotides to produce the first and second barcoding primers.

Embodiment 50. The method of embodiment [00830], wherein the first set of barcoding oligonucleotides and the first primer set are not annealed prior to said contacting.

Embodiment 51. The method of any one of embodiments [00808]-[00834], wherein the DNA or RNA fragments are not amplified during step (b).

Embodiment 52. The method of any one of embodiments [00808]-[00835], wherein each of the first set of barcoding primers comprise, in 5′ to 3′ order: the nucleotide sequence that is complementary to a 5′ read region of a first strand of the DNA or RNA fragments; the first molecular cellular label; and the first adapter sequence.

Embodiment 53. The method of any one of embodiments [00808]-[00836], wherein each of the second set of barcoding primers comprise, in 5′ to 3′ order: the nucleotide sequence that is complementary to a 5′ read region of the second strand of the DNA or RNA fragments; the second molecular cellular label; and the second adapter sequence.

Embodiment 54. The method of any one of embodiments [00808]-[00837], wherein the DNA is a double-stranded DNA (dsDNA) fragment.

Embodiment 55. The method of any one of embodiments [00808]-[00838], wherein the first and second molecular cellular labels each comprises a degenerate nucleotide sequence.

Embodiment 56. The method of any one of embodiments [00808]-[00839], wherein the first and second molecular cellular labels each comprises 8-50 nucleotides.

Embodiment 57. The method of any one of embodiments [00839]-[00840], wherein the degenerate sequence comprises 8-50 nucleotides.

Embodiment 58. The method of any one of embodiments [00839]-[00841], wherein the degenerate sequence comprises 8-20 nucleotides.

Embodiment 59. The method of any one of embodiments [00808]-[00842], wherein the two consensus regions of the first barcoding oligonucleotides flank the first molecular cellular label.

Embodiment 60. The method of any one of embodiments [00808]-[00842], wherein the two consensus regions of the second barcoding oligonucleotides flank the second molecular cellular label.

Embodiment 61. The method of any one of embodiments [00808]-[00844], wherein the nucleotide sequence of the first or second molecular cellular label is positioned between the nucleotide sequences of the two consensus regions.

Embodiment 62. The method of any one of embodiments [00808]-[00845], wherein the degenerate sequence of each first and second molecular cellular label is distinguishable from one another.

Embodiment 63. The method of any one of embodiments [00808]-[00846], wherein the first molecular cellular label of the barcoding oligonucleotides within the first set of barcoding oligonucleotides is distinguishable from other first molecular cellular labels of the first set of barcoding oligonucleotides by its nucleotide sequence.

Embodiment 64. The method of any one of embodiments [00808]-[00847], wherein the second molecular cellular labels of the barcoding oligonucleotides within the second set of barcoding oligonucleotides is distinguishable from other second molecular cellular labels of the second set of barcoding oligonucleotides by its nucleotide sequence.

Embodiment 65. The method of any one of embodiments [00808]-[00848], wherein said contacting comprises contacting the cell suspension or tissue slices with the first and second set of barcoding oligonucleotides at a concentration such that each cell within the cell suspension or tissue slice comprises a first and second barcoding oligonucleotide that is distinguishable from a first and second barcoding oligonucleotide of a different cell.

Embodiment 66. The method of embodiment [00849], wherein the concentration ranges from 100 fM to 1 μM.

Embodiment 67. The method of embodiment [00850], wherein the concentration ranges from 1 pM-10 pM.

Embodiment 68. The method of any one of embodiments [00808]-[00851], wherein said contacting comprises contacting the cell suspension or tissue slices with the first and second set of barcoding oligonucleotides at a concentration such that each cell within the cell suspension or tissue slice comprises 2-1000 barcoding oligonucleotides.

Embodiment 69. The method of any one of embodiments [00808]-68, wherein a cell within the cell suspension or tissue slice comprises less than 5% of barcoding oligonucleotides with the same first and second molecular cellular label as a different cell within the cell suspension.

Embodiment 70. The method of any one of embodiments [00808]-[00853], wherein a cell within the cell suspension or tissue slice does not comprise the first and second molecular cellular label that is the same first and second molecular labels of a second cell within the cell suspension or tissue slice.

Embodiment 71. The method of any one of embodiments [00808]-[00854], wherein the DNA fragment is a DNA amplicon product.

Embodiment 72. The method of any one of embodiments [00808]-[00854], wherein the RNA fragment is an RNA amplicon product.

Embodiment 73. The method of any one of embodiments [00808]-[00854], wherein the DNA or RNA fragment is a DNA or RNA product of ligation.

Embodiment 74. The method of any one of embodiments [00808]-[00854], wherein the DNA fragment comprises genomic DNA comprising a target region positioned between a first consensus read region and a second consensus read region, each first and second consensus read region selected from: a Y-adapter nucleotide sequence, a hairpin nucleotide sequence, and a duplex nucleotide sequence.

Embodiment 75. The method of any one of embodiments [00808]-[00854], wherein the DNA or RNA fragment is a DNA or RNA product of tagmentation.

Embodiment 76. The method of any one of embodiments [00808]-[00854], wherein the DNA fragment comprises genomic DNA (gDNA) modified to contain a first consensus read region at the 5′ end of the DNA sequence and a second consensus read region at the 3′ end of the DNA sequence.

Embodiment 77. The method of any one of embodiments [00808]-[00854], wherein the RNA sequence is a reverse transcribed RNA sequence comprising a target region, a first consensus read region, and a second consensus read region.

Embodiment 78. The method of embodiment [00860], wherein the first consensus read region is at the 5′ end of the target region, and the second consensus read region is at the 3′ end of the target region.

Embodiment 79. The method of any one of embodiments [00808]-[00854], wherein the RNA sequence is selected from: messenger RNA (mRNA), transfer RNA (tRNA), and ribosomal RNA (rRNA), guide RNA (gRNA), and trans-activating crispr RNA (tracrRNA).

Embodiment 80. The method of any one of embodiments [00808]-[00863], wherein the DNA or RNA fragments in step (a) comprises:

a 5′ consensus read region;

a 3′ consensus read region; and

a target region.

Embodiment 81. The method of any one of embodiments [00808]-[00864], wherein the method further comprises, after step (c) contacting the amplicon product with a set of indexing primers, and performing an amplification reaction to produce a second set of amplicon products.

Embodiment 82. The method of embodiment [00808]-[00864], wherein the method comprises lysing the cells containing the set of amplicon products.

Embodiment 83. The method of embodiment [00869], wherein the method comprises lysing the cells containing the second set of amplicon products.

Embodiment 84. The method of embodiment [00870], wherein the method further comprises contacting the second set of amplicon products with a third primer set comprising amplification primers, and performing an amplification reaction to produce a third set of amplicon products.

Embodiment 85. The method of any one of embodiments [00808]-[00871], wherein the method further comprises, after step (c), sequencing the DNA or RNA amplicon product to produce a barcoded sequenced library.

Embodiment 86. The method of any one of embodiments [00808]-[00871], wherein the cell suspension comprises 1000 cells or less.

Embodiment 87. The method of any one of embodiments [00808]-[00871], wherein the cell suspension comprises 50 cells or less.

Embodiment 88. The method of any one of embodiments [00808]-[00871], wherein the cell suspension comprises 5 cells or less.

Embodiment 89. The method of any one of embodiments [00808]-[00871], wherein the cell suspension comprises a single cell.

Embodiment 90. The method of any one of embodiments [00808]-[00876], wherein the method further comprises, sequencing the amplicon products to produce a sequenced barcoded library comprising barcoding sequences for each cell within the cell suspension or tissue slices.

Embodiment 91. A method of performing whole cell barcoding, the method comprising:

(a) contacting DNA or RNA fragments within a permeabilized cell suspension or tissue slices with:

(i) a first set of nucleotide sequences comprising:

(ia) a first set of barcoding oligonucleotides, each barcoding oligonucleotide comprising:

a first molecular cellular label comprising 8 or more nucleotides; and

a consensus region comprising a nucleotide sequence that is complementary to a 5′ read region of a first strand of the DNA or RNA fragments;

(ii) a second set of barcoding nucleotide sequences comprising:

(iia) a second set of barcoding oligonucleotides, each barcoding oligonucleotides comprising:

a second molecular cellular label comprising 8 or more nucleotides; and

a consensus region comprising a nucleotide sequence that is complementary to a 5′ read region of a second strand of the DNA or RNA fragments;

(b) amplifying:

the first set of barcoding oligonucleotides to produce a first set of barcoding primers;

and

the second set of barcoding oligonucleotides to produce a second set of barcoding primers; and

(c) amplifying the DNA or RNA fragments with the first and second barcoding primers to produce a set of amplicon products, wherein the set of amplicon products comprise the first barcoding primer bridging from the 5′ end the DNA or RNA nucleotide sequences and the second barcoding primer bridging from the 3′ end of the DNA or RNA fragments.

Embodiment 92. The method of embodiment [00878], wherein the first set of barcoding oligonucleotides further comprises a first adapter sequence; and the second set of barcoding oligonucleotides further comprises a second adapter sequence.

Embodiment 93. The method of any one of embodiments [00878], wherein the first set of barcoding oligonucleotides comprises a third adapter sequence that is complementary to the first adapter of the first set of barcoding oligonucleotides, and wherein the second set of barcoding oligonucleotides further comprises a fourth adapter sequence that is complementary to the second adapter sequence of the first set of barcoding oligonucleotides.

Embodiment 94. The method of any one of embodiments [00878]-[00893], wherein the first set of barcoding oligonucleotides further comprise a first cleavage site; and the second set of barcoding oligonucleotides further comprises a second cleavage site.

Embodiment 95. The method of any one of embodiment [00878]-[00894], wherein the first and second set of barcoding oligonucleotides are hairpin barcoding oligonucleotides.

Embodiment 96. The method of embodiment [00895], wherein the first set of barcoding oligonucleotides comprises a third cleavage site that is complementary to the first cleavage site of the first set of barcoding oligonucleotides, and wherein the second set of barcoding oligonucleotides further comprises a fourth cleavage site that is complementary to the second cleavage site of the second set of barcoding oligonucleotides.

Embodiment 97. The method of Embodiment any one of embodiments [00878]-[00896], wherein the said amplifying in step (b) comprises amplifying via isothermal amplification, the first and second set of barcoding oligonucleotides to produce the first and second barcoding primers.

Embodiment 98. The method of any one of embodiments [00878]-[00897], wherein the DNA or RNA fragments are not amplified during step (b).

Embodiment 99. The method of any one of embodiments [00878]-[00898], wherein each of the first set of hairpin barcoding oligonucleotides comprise:

the nucleotide sequence that is complementary to a 5′ read region of a first strand of the DNA or RNA fragments;

the first molecular cellular label;

a stem loop;

optionally a first adapter sequence; and

optionally a first cleavage site.

Embodiment 100. The method of any one of embodiments [00878]-[00899], wherein each of the first set of hairpin barcoding oligonucleotides comprise, in 5′ to 3′ order:

the nucleotide sequence that is complementary to a 5′ read region of a first strand of the DNA or RNA fragments;

the first molecular cellular label;

optionally a first adapter sequence;

optionally a first cleavage site; and

a stem loop.

Embodiment 101 The method of any one of embodiments [00878]-[00905], wherein each of the second set of hairpin barcoding oligonucleotides comprise:

the nucleotide sequence that is complementary to a 5′ read region of a second strand of the DNA or RNA fragments;

the second molecular cellular label;

a stem loop;

optionally a second adapter sequence; and

optionally a second cleavage site.

Embodiment 102. The method of any one of embodiments [00878]-[00911], wherein each of the first set of hairpin barcoding oligonucleotides comprise, in 5′ to 3′ order:

the nucleotide sequence that is complementary to a 5′ read region of a second strand of the DNA or RNA fragments;

the second molecular cellular label;

optionally a second adapter sequence;

optionally a second cleavage site; and

a stem loop.

Embodiment 103. The method of any one of embodiments [00878]-[00917], wherein the DNA is a double-stranded DNA (dsDNA) fragment.

Embodiment 104. The method of any one of embodiments [00878]-[00923], wherein the first and second molecular cellular labels each comprises a degenerate nucleotide sequence.

Embodiment 105. The method of any one of embodiments [00878]-[00924], wherein the first and second molecular cellular labels each comprises 8-50 nucleotides.

Embodiment 106. The method of any one of embodiments [00924]-[00925], wherein the degenerate sequence comprises 8-50 nucleotides.

Embodiment 107. The method of any one of embodiments [00924]-[00926], wherein the degenerate sequence comprises 8-20 nucleotides.

Embodiment 108. The method of any one of embodiments [00878]-[00927], wherein the two consensus regions of the first barcoding oligonucleotides flank the first molecular cellular label.

Embodiment 109. The method of any one of embodiments [00878]-[00928], wherein the two consensus regions of the second barcoding oligonucleotides flank the second molecular cellular label.

Embodiment 110. The method of any one of embodiments [00878]-[00929], wherein the nucleotide sequence of the first or second molecular cellular label is positioned between the nucleotide sequences of the two consensus regions.

Embodiment 111. The method of any one of embodiments [00878]-[00930], wherein the degenerate sequence of each first and second molecular cellular label is distinguishable from one another.

Embodiment 112. The method of any one of embodiments [00878]-[00931], wherein the first molecular cellular label of the barcoding oligonucleotides within the first set of barcoding oligonucleotides is distinguishable from other first molecular cellular labels of the first set of barcoding oligonucleotides by its nucleotide sequence.

Embodiment 113. The method of any one of embodiments [00878]-[00932], wherein the second molecular cellular labels of the barcoding oligonucleotides within the second set of barcoding oligonucleotides is distinguishable from other second molecular cellular labels of the second set of barcoding oligonucleotides by its nucleotide sequence.

Embodiment 114. The method of any one of embodiments [00878]-[00933], wherein said contacting comprises contacting the cell suspension or tissue slices with the first and second set of barcoding oligonucleotides at a concentration such that each cell within the cell suspension or tissue slice comprises a first and second barcoding oligonucleotide that is distinguishable from a first and second barcoding oligonucleotide of a different cell.

Embodiment 115. The method of embodiment [00934], wherein the concentration ranges from 100 fM to 1 μM.

Embodiment 116. The method of embodiment [00935], wherein the concentration ranges from 1 pM-10 pM.

Embodiment 117. The method of any one of embodiments [00878]-[00936], wherein said contacting comprises contacting the cell suspension or tissue slices with the first and second set of barcoding oligonucleotides at a concentration such that each cell within the cell suspension or tissue slice comprises 2-1000 barcoding oligonucleotides.

Embodiment 118. The method of any one of embodiments [00878]-[00937], wherein a cell within the cell suspension or tissue slice comprises less than 5% of barcoding oligonucleotides with the same first and second molecular cellular label as a different cell within the cell suspension.

Embodiment 119. The method of any one of embodiments [00878]-[00938], wherein a cell within the cell suspension or tissue slice does not comprise the first and second molecular cellular label that is the same first and second molecular labels of a second cell within the cell suspension or tissue slice.

Embodiment 120. The method of any one of embodiments [00878]-[00939], wherein the DNA fragment is a DNA amplicon product.

Embodiment 121. The method of any one of embodiments [00878]-[00940], wherein the RNA fragment is an RNA amplicon product.

Embodiment 122. The method of any one of embodiments [00878]-[00941], wherein the DNA or RNA fragment is a DNA or RNA product of ligation.

Embodiment 123. The method of any one of embodiments [00878]-[00942], wherein the DNA fragment comprises genomic DNA comprising a target region positioned between a first consensus read region and a second consensus read region, each first and second consensus read region selected from: a Y-adapter nucleotide sequence, a hairpin nucleotide sequence, and a duplex nucleotide sequence.

Embodiment 124. The method of any one of embodiments [00878]-[00943], wherein the DNA or RNA fragment is a DNA or RNA product of tagmentation.

Embodiment 125. The method of any one of embodiments [00878]-[00945], wherein the DNA fragment comprises genomic DNA (gDNA) modified to contain a first consensus read region at the 5′ end of the DNA sequence and a second consensus read region at the 3′ end of the DNA sequence.

Embodiment 126. The method of any one of embodiments [00878]-[00945], wherein the RNA sequence is a reverse transcribed RNA sequence comprising a target region, a first consensus read region, and a second consensus read region.

Embodiment 127. The method of embodiment [00946], wherein the first consensus read region is at the 5′ end of the target region, and the second consensus read region is at the 3′ end of the target region.

Embodiment 128. The method of any one of embodiments [00878]-[00947], wherein the RNA sequence is selected from: messenger RNA (mRNA), transfer RNA (tRNA), and ribosomal RNA (rRNA), guide RNA (gRNA), and trans-activating crispr RNA (tracrRNA).

Embodiment 129. The method of any one of embodiments [00878]-[00948], wherein the DNA or RNA fragments in step (a) comprises:

a 5′ consensus read region;

a 3′ consensus read region; and

a target region.

Embodiment 130. The method of any one of embodiments [00878]-[00949], wherein the method further comprises, after step (c) contacting the amplicon product with a set of indexing primers, and performing an amplification reaction to produce a second set of amplicon products.

Embodiment 131. The method of embodiment [00878]-[00953], wherein the method comprises lysing the cells containing the set of amplicon products.

Embodiment 132. The method of embodiment [00954], wherein the method comprises lysing the cells containing the second set of amplicon products.

Embodiment 133. The method of embodiment [00955], wherein the method further comprises contacting the second set of amplicon products with a third primer set comprising amplification primers, and performing an amplification reaction to produce a third set of amplicon products.

Embodiment 134. The method of any one of embodiments [00878]-[00956], wherein the method further comprises, after step (c), sequencing the DNA or RNA amplicon product to produce a barcoded sequenced library.

Embodiment 135. The method of any one of embodiments [00878]-[00956], wherein the cell suspension comprises 1000 cells or less.

Embodiment 136. The method of any one of embodiments [00878]-[00957], wherein the cell suspension comprises 50 cells or less.

Embodiment 137. The method of any one of embodiments [00878]-[00959], wherein the cell suspension comprises 5 cells or less.

Embodiment 138. The method of any one of embodiments [00878]-[00960], wherein the cell suspension comprises a single cell.

Embodiment 139. The method of any one of embodiments [00878]-[00961], wherein the method further comprises, sequencing the amplicon products to produce a sequenced barcoded library comprising barcoding sequences for each cell within the cell suspension or tissue slices.

Embodiment 140. A method of detecting disease-associated genetic alterations of single cells within a heterogeneous population in situ, by providing data associated with the sequenced barcoded library to a computer system comprising a computer readable storage medium, having instructions, when executed, that cause a processor to: (a) produce a graphical representation of the sequenced barcoded library; (b) perform a clustering analysis on the sequenced barcoded library to: remove any barcoding errors; cluster the barcoded sequenced library to create clusters of barcoded read sequences, wherein each cluster of barcoded read sequences is associated with a single cell; and (c) output each cluster of barcoded read sequences into an individual sequence file, wherein each sequencing file contains barcoded read sequences for a single cell; analyzing the sequencing file of the sequenced barcoded library for each cell to determine the presence or absence of disease-associated genetic alterations within each cell of the permeabilized cell suspension.

Embodiment 141. The method of embodiment [00963], wherein the graphical representation comprises:

nodes representing the first or second molecular cellular labels; and

edges representing barcoded read sequence comprising the sequenced barcoded library with the first and second molecular cellular label.

Embodiment 142. The method of any one of embodiments [00963]-[00964], wherein the computer readable storage medium comprises further instructions that cause the processor to, before step (b), calculate an edge weight read threshold based on the average experimental rates of barcode leakage from one cell to another, sequencing error rates, and the empirical shapes of the signal and noise distributions in the sequenced barcoded library.

Embodiment 143. The method of any one of embodiments [00963]-[00964], wherein removing any barcoding errors comprises pruning the graphical representation by edge weight, wherein edge weight is determined by the number of barcoded sequencing reads that comprise both the first molecular cellular label and the second molecular cellular label as a barcoded pair.

Embodiment 144. The method of embodiment [00967], wherein pruning the graphical representation by edge weight comprises removing edges with an edge weight less than the edge weight read threshold.

Embodiment 145. The method of embodiment [00969], wherein pruning the graphical representation by edge weight results in singleton nodes comprising nodes without edges being removed from the graphical representation.

Embodiment 146. The method of embodiment [00963], wherein removing any barcoding errors comprises pruning the graphical representation by connectedness of the first molecular cellular label and the second molecular cellular label as a barcoded pair.

Embodiment 147. The method of embodiment [00971], wherein connectedness of the barcoded pair comprises detecting barcode neighbors of the first molecular cellular label and barcode neighbors of the second molecular cellular label; and counting the number of barcode neighbors the first molecular cellular label and the second molecular cellular label share in common versus distinct barcode neighbors.

Embodiment 148. The method of embodiment [00972], wherein detecting barcode neighbors provides a quantitative measure of the probability of the first molecular cellular label and second molecular cellular label to be within the same cluster.

Embodiment 149. The method of embodiment [00973], wherein pruning the graphical representation by the connectedness of the first and second molecular cellular labels comprises removing barcode pairs with a fraction of common barcode neighbors less than a threshold.

Embodiment 150. The method of embodiment [00974], wherein the threshold is calculated based on the distribution of the fraction of common barcode neighbors, the sequencing error rate, and an initial expected barcode leakage rate.

Embodiment 151. The method of embodiment [00974], wherein pruning the graphical representation by connectedness of the first and second molecular cellular labels results in singleton nodes comprising nodes without edges being removed from the graphical representation.

Embodiment 152. The method of embodiment [00963], wherein said analyzing comprises trimming the sequencing files to remove at least a portion of the barcode and/or adapter sequence.

Embodiment 153. The method of embodiment [00977], wherein analyzing further comprises aligning each of the sequencing reads to a target sequence of the human genome and producing an alignment file for each of sequencing files.

Embodiment 154. The method of embodiment [00978], wherein analyzing further comprises running each of the alignment files through a variant caller configured to identify and quantify genetic alterations within the sequenced barcoded library.

Embodiment 155. The method of embodiment [00979], wherein the genetic alterations comprise structural variants.

Embodiment 156. The method of embodiment [00980], wherein the structural variant is a is a germline variant or a somatic variant.

Embodiment 157. The method of embodiment [00980], wherein the sequencing reads are aligned to the sequences of the human genome with one or more genome or transcriptome read aligners selected from Burrows Wheeler Aligner (BWA), BWA-MEM, Bowtie2, RNA-STAR, and Salmon.

Embodiment 158. The method of embodiment [00982], wherein identifying the genetic alterations comprises extracting structural variants from each of the alignment files of the sequencing reads.

Embodiment 159. The method of embodiment [00983], wherein extracting structural variants comprises listing all the structural variants commonly found in the alignment file for each sequenced barcoded library.

Embodiment 160. The method of any one of embodiments [00979]-[00984], wherein identifying comprises identifying at least one of: the percentage of genome reads in a region of the sequence containing a variant, the quality scores of nucleotides in reads covering a variant, and the total number of reads at a variant position.

Embodiment 161. The method of embodiment [00979], wherein quantifying the structural variants comprises determining statistical significance of each structural variant using one of more statistical algorithms to calculate a statistical score and/or a significance value for each of the structural variants.

Embodiment 162. The method of embodiment [00986], wherein the statistical algorithm is a binomial distribution model, over-dispersed binomial model, beta, normal, exponential, or gamma distribution model.

Embodiment 163. The method of embodiment [00983], wherein the structural variants are selected from one of more of: single nucleotide variants (SNVs), small insertions, deletions, indels, germline variant, a somatic variant, and a combination thereof.

Embodiment 164. The method of any one of embodiments [00963]-[00988], wherein the method further comprises calculating a tumor mutational burden (TMB) for each individual cell in the sample.

Embodiment 165. The method of any of embodiment [00989], wherein the TMB comprises a percentage of synonymous and/or non-synonymous somatic mutations in targeted regions of the target or reference sequence.

Embodiment 166. A computer readable storage medium comprising instructions for detecting disease-associated genetic alterations of single cells within a heterogenous population in situ, wherein the instructions, when executed, cause a processor to: (a) produce a graphical representation of sequenced barcoded library prepared in situ; (b) perform a clustering analysis on the sequenced barcoded library to: remove any barcoding errors; cluster the barcoded sequenced library to create clusters of barcoded read sequences, wherein each cluster of barcoded read sequences is associated with a single cell; and (c) output each cluster of barcoded read sequences into an individual sequence file, wherein each sequencing file contains barcoded read sequences for a single cell, the sequence file providing information for determining the presence or absence of disease-associated genetic alterations within each cell of the permeabilized cell suspension.

Embodiment 167. A cell barcoding kit comprising:

(a) a first set of barcoding oligonucleotides, each barcoding oligonucleotide comprising:

a first molecular cellular label comprising 8 or more nucleotides;

two consensus regions, wherein the two consensus regions of each barcoding oligonucleotide comprise:

a nucleotide sequence that is complementary to a 5′ read region of a first strand of a DNA fragment, and

a first adapter sequence,

(b) a second set of barcoding oligonucleotides, each barcoding oligonucleotide comprising:

a second molecular cellular label comprising 8 or more nucleotides;

two consensus regions, wherein the two consensus regions of each barcoding oligonucleotide comprise:

a nucleotide sequence that is complementary to a 5′ read region of a second strand of the DNA fragment, and

a second adapter sequence.

Embodiment 168. The kit of embodiment 167, wherein each of the first barcoding oligonucleotides is annealed to a first primer comprising a nucleotide sequence that is complementary to the first adapter sequence of the first barcoding oligonucleotide.

Embodiment 169. The kit of embodiment 167, wherein each of the second barcoding oligonucleotides is annealed to a second primer comprising a nucleotide sequence that is complementary to the second adapter sequence of the second barcoding oligonucleotide.

Embodiment 170. The kit of embodiment 167, wherein the first and second barcoding oligonucleotides are hairpin oligonucleotides.

Embodiment 171. The kit of embodiment 167, wherein the first barcoding oligonucleotides each further comprise a first cleavage site, and wherein the second barcoding oligonucleotides each further comprise a second cleavage site.

Embodiment 172. The kit of any one of embodiments 167-171, wherein the first primer further comprises a third cleavage site that is complementary to the first cleavage site of the first barcoding oligonucleotides, and wherein the second primer further comprises a fourth cleavage site that is complementary to the second cleavage site of the second barcoding oligonucleotides.

Embodiment 173. The kit of embodiment 167-172, wherein the kit further comprises one or more enzymes.

Embodiment 174. The kit of embodiment [001008], wherein the one or more enzymes is selected from one or more of: DNA polymerase, RNA polymerase, nicking enzyme, a Bst2.0 polymerase, a Phi29 polymerase, an enzymatic fragmentation enzyme, an End Repair A-tail enzyme, a DNA ligase, or a combination thereof.

Embodiment 175. The kit of any one of embodiments 167-174, wherein the kit further comprises one or more buffers selected from: a lysis buffer, an enzyme fragmentation buffer, an End Repair A-tail buffer, a ligation buffer, buffer 3.0, buffer 3.1, PCR amplification buffer, isothermal amplification buffer, and a combination there.

Embodiment 176. The kit of any one of embodiments 167-175, wherein the kit further comprises a polymerase chain reaction (PCR) buffer.

Embodiment 177. The kit of any one of embodiments 167-176, wherein the kit further comprises a deoxynucleotide triphosphates (dNTPs) buffer.

Embodiment 178. The kit of any one of embodiments 167-178, wherein the molecular cellular label comprises a degenerate nucleotide sequence.

Embodiment 179. The kit of any one of embodiments 167-178, wherein the molecular cellular label comprises 8-50 nucleotides.

Embodiment 180. A cell barcoding composition comprising:

(a) permeabilized cell suspension or tissue slices comprising DNA or RNA nucleotide sequences;

(b) a first primer set comprising barcoding primers configured to bridge and extend from the 5′ region of the DNA or RNA nucleotide sequences;

(c) a second primer set comprising barcoding primers configured to bridge and extend from the 3′ region of the DNA or RNA nucleotide sequences, each barcoding primer comprising:

a molecular cellular label comprising 8 or more nucleotides;

two consensus regions, wherein the two consensus regions of each barcoding primer comprises:

a nucleotide sequence that is complementary to a 5′ or a 3′ read region of the DNA or

RNA sequences, and

an adapter sequence,

wherein the first and second barcoding primer sets do not amplify a target region of the DNA or RNA sequences;

(d) a third primer set comprising nucleotide sequences that are complementary to the adapter sequence of the first primer set; and

(e) a fourth primer set comprising nucleotide sequences that are complementary to the adapter sequence of the second primer set.

Embodiment 181 The composition of embodiment 180-181, wherein the composition further comprises one or more enzymes.

Embodiment 182. The composition of embodiment [001026], wherein the enzyme is a

DNA polymerase.

Embodiment 183. The composition of embodiment [001026], wherein the enzyme is an RNA polymerase.

Embodiment 184. The composition of any one of embodiments 180-183, wherein the composition further comprises a lysis buffer.

Embodiment 185. The composition of any one of embodiments 180-184, wherein the composition further comprises polymerase chain reaction (PCR) buffer.

Embodiment 186. The composition of any one of embodiments 180-185, wherein the composition further comprises a deoxynucleotide triphosphates (dNTPs) buffer.

Embodiment 187. The composition of any one of embodiments 180-186, wherein the molecular cellular label comprises a degenerate nucleotide sequence.

Embodiment 188. The composition of any one of embodiments 180-188, wherein the molecular cellular label comprises 8-50 nucleotides.

Embodiment 189. The composition of any one of embodiments 180-189, wherein the DNA sequence is a DNA amplicon product.

Embodiment 190. The composition of any one of embodiments 180-190, wherein the RNA sequence is an RNA amplicon product.

Embodiment 191. The composition of any one of embodiments 180-190, wherein the DNA or RNA sequence is a DNA or RNA product of ligation.

Embodiment 192. The composition of any one of embodiments 180-191, wherein the DNA sequence is selected from: a Y-adapter nucleotide sequence, a hairpin nucleotide sequence, and a duplex nucleotide sequence.

Embodiment 193. The composition of any one of embodiments 180-192, wherein the DNA or RNA sequence is a DNA or RNA product of tagmentation.

Embodiment 194. The composition of any one of embodiments 180-193, wherein the DNA sequence comprises genomic DNA (gDNA).

Embodiment 195. The composition of any one of embodiments 180-194, wherein the RNA sequence is a reverse transcribed RNA sequence with known sequence ends.

Embodiment 196. The composition of any one of embodiments 180-195, wherein the RNA sequence is selected from: messenger RNA (mRNA), transfer RNA (tRNA), and ribosomal RNA (rRNA), guide RNA (gRNA), and trans-activating crispr RNA (tracrRNA).

Embodiment 197. The composition of any one of embodiments 180-196, wherein the DNA or RNA sequence comprises: a 5′ consensus read region; a 3′ consensus read region; and a target region.

Embodiment 198. A method of performing in situ cell barcoding in a single pool of cells, without requiring dividing of the cells into multiple pools of cells, the method comprising: in the single pool of cells: (a) introducing, within a cell suspension, a plurality of barcoding oligonucleotides, each barcoding oligonucleotide comprising a molecular cellular label, and a consensus region; (b) amplifying, within individual cells of the single pool of cells, the barcoding oligonucleotides to produce a set of barcoding primers; and (c) amplifying, within individual cells of the single pool of cells, the DNA or RNA with the barcoding primers to produce a set of amplicon products that comprise the barcoding primers, resulting in situ barcoded cells in the single pool of cells.

Embodiment 199. A method of performing in situ cell barcoding in a single pool of cells, without requiring dividing of the cells into multiple pools of cells, the method comprising: in the single pool of cells: performing, in each cell, a fragmentation process to form nucleic acid fragments; performing, in each cell, an amplification or ligation of the nucleic acid fragments with consensus regions; introducing barcoding oligonucleotides to the single pool of cells; amplifying, within individual cells of the single pool of cells, the barcoding oligonucleotides to produce a set of barcoding primers; and amplifying, within individual cells of the single pool of cells, the nucleic acid fragments with the barcoding primers to produce a set of amplicon products that comprise the barcoding primers, resulting in situ barcoded cells in the single pool of cells.

Embodiment 200. A method of amplifying an oligonucleotide in situ in to generate multiple copies of a reverse complement of the oligonucleotide.

Embodiment 201. A cell barcoding composition comprising a collection of cells, each cell in the collection containing nucleic acid fragments, each nucleic acid fragment comprising a universal barcode having a degenerate sequence on each end of the nucleic acid fragment.

Embodiment 202. A composition comprising a collection of individual cells each comprising a sequencing library including genomic fragments with universal barcodes comprising degenerate sequences attached to the genomic fragments.

Embodiment 203. A composition comprising a collection of individual cells each comprising universal adapters (e.g., any of the adapters or adapter sequences described herein, including any of the consensus regions described herein) containing one or more degenerate or partially degenerate sequences added to one or both sides of genomic fragments.

Embodiment 204. Use of randomly paired barcodes comprising degenerate sequences to label each end of a nucleic acid fragment in a cell.

Embodiment 205a. A composition comprising a collection of cells including nucleic acid precursor libraries and barcoding oligonucleotides that are capable of hybridizing to each other due to complementary sequences on 5′ ends of the precursor libraries, to create a hybridization product, wherein the hybridization product is not capable of amplification because of 3′ overhangs on the barcoding oligonucleotides.

Embodiment 205b. A composition comprising a collection of intact cells, each cell comprising precursor libraries and barcoding oligonucleotides, wherein each precursor library is capable of hybridizing to one or more barcoding oligonucleotides.

Embodiment 206. A composition comprising a next generation sequencing library made up of nucleic acid fragments with sequencing adaptors, wherein barcoding reactions involving the nucleic acid fragments result in products that include a same nucleic acid fragment with different cellular barcodes on either end of the nucleic acid fragment.

Embodiment 207. A method of performing in situ cell barcoding in a single pool of cells, the method comprising: in the single pool of cells: performing, in each cell, a fragmentation process to form nucleic acid fragments; performing, in each cell, an amplification or ligation of the nucleic acid fragments with consensus regions in a reaction comprising a first buffer; conducting a buffer exchange and cell washing step, wherein the first buffer is removed and replaced with a second buffer having a different composition specific to performing barcoding of the nucleic acid fragments that have been amplified; introducing barcoding oligonucleotides to the single pool of cells; amplifying, within individual cells of the single pool of cells, the barcoding oligonucleotides to produce a set of barcoding primers; and amplifying, within individual cells of the single pool of cells, the nucleic acid fragments with the barcoding primers to produce a set of amplicon products that comprise the barcoding primers, resulting in situ barcoded cells in the single pool of cells.

Embodiment 208. A method of performing in situ cell barcoding in a single pool of cells, the method comprising: in the single pool of cells: performing, in each cell, a fragmentation process to form genomic DNA fragments; performing, in each cell, an amplification or ligation of the genomic DNA fragments with a first set of reagents (e.g., reagents comprising buffers, enzymes, and nucleic acid sequences comprising consensus regions); conducting a cell washing step, wherein the first set of reagents is removed and replaced with a second set of reagents (e.g., buffers, enzymes, barcoding oligonucleotides, and barcoding primers, or any combination thereof) specific to performing barcoding of the genomic DNA fragments that have been amplified; and performing, in each cell, an amplification or ligation of the genomic DNA fragments with barcoding oligonucleotides in the second set of reagents, to create an in situ barcoded library in the single pool of cells.

Embodiment 209. A method of performing in situ cell barcoding in a single pool of cells, the method comprising: in the single pool of cells: performing, in each cell, a fragmentation process to form genomic DNA fragments; performing, in each cell, an amplification of the genomic DNA fragments involving a first buffer (e.g., buffer comprising enzymes, and nucleic acid sequences comprising consensus regions); conducting a buffer exchange and cell washing step, wherein a first buffer having a composition designed for the amplification in step (b) is removed and replaced with a second buffer having a different composition optimized for performing barcoding of the genomic DNA fragments that have been amplified (e.g., a second buffer comprising enzymes, barcoding oligonucleotides, and barcoding primers, or any combination thereof); and performing, in each cell, in situ barcode amplification, and amplification or ligation of the genomic DNA fragments with barcoding products to create an in situ barcoded library in the single pool of cells.

Embodiment 210. A method of performing in situ cell barcoding in a single pool of cells, the method comprising: in the single pool of cells: performing, in each cell, a fragmentation process to form genomic DNA fragments; conducting a buffer exchange and cell washing step, wherein a first buffer (e.g., buffer comprising enzymes, and nucleic acid sequences comprising consensus regions) is removed from a product resulting from the fragmentation process and replaced with a second buffer having a different composition designed to change ionic composition of the cells to permit additional steps of the method (e.g., a second buffer comprising enzymes, barcoding oligonucleotides, and barcoding primers, or any combination thereof); and performing, in each cell, in situ barcode amplification and amplification or ligation of the genomic DNA fragments with barcoding products to create an in situ barcoded library in the single pool of cells.

Embodiment 211. A method of performing in situ cell barcoding in a single pool of cells, the method comprising: in the single pool of cells: performing, in each cell, an amplification of genomic DNA fragments in the cell; conducting a cell washing step to modify ionic composition of each of the cells; amplifying, in each cell with modified ionic composition, barcoding oligonucleotides; and performing, in each cell with modified ionic composition, in situ amplification of the barcoding oligonucleotides, and amplification or ligation of the genomic DNA fragments with barcoding products to create an in situ barcoded library in the single pool of cells.

Embodiment 212. In a single cell pool of cells for in situ cell barcoding, use of one or more washing steps in between reactions to replace each set of reagents for each reaction with a different set of reagents specific to a next reaction.

Embodiment 213. A method of performing in situ cell barcoding, the method comprising: performing, in each cell, an amplification of genomic DNA fragments in the cell (e.g., as described in Example 1), wherein the cells are not lysed by the amplification; conducting a cell washing step to modify ionic composition of each of the cells; and performing, in each cell, in situ barcode amplification, and amplification or ligation of the genomic DNA fragments with barcoding products (e.g., barcoding oligonucleotides, amplification primers, and barcoding primers, or any combination thereof) to create an in situ barcoded library in the single pool of cells.

Embodiment 214. A method of performing in situ cell barcoding, the method comprising: performing, in each cell, an amplification of genomic DNA fragments in the cell (e.g., as described in Example 1), resulting in a cell supernatant, wherein a majority of the cells in the cell supernatant are not lysed by the amplification; conducting a cell washing step to remove from the cell supernatant cellular materials from cells that were lysed by the amplification; and performing, in each cell, in situ barcode amplification, and amplification or ligation of the genomic DNA fragments with barcoding products (e.g., barcoding oligonucleotides, amplification primers, and barcoding primers, or any combination thereof) to create an in situ barcoded library in the cells that remain un-lysed.

Embodiment 215. A composition comprising a first, second, third and fourth oligonucleotide, wherein:

the first oligonucleotide comprises, from 5′ to 3′: (i) the reverse complement of the 5′ terminus of the sense strand of a double-stranded DNA sequence to be amplified; (ii) a barcode sequence; and (iii) an adapter sequence; and the second oligonucleotide comprises the reverse complement of (iii);

the third oligonucleotide comprises, from 5′ to 3′: (iv) the reverse complement of the 5′ terminus of the antisense strand of a double-stranded DNA sequence to be amplified; (v) a barcode sequence; and (vi) an adapter sequence; and the fourth oligonucleotide comprises the reverse complement of (vi).

Embodiment 216. The composition of embodiment of 215, wherein (a) the first and second oligonucleotides are hybridized to each other; and/or (b) the third and fourth oligonucleotides are hybridized to each other.

Embodiment 217. The composition of embodiment of 215 or 216, wherein the adapter sequence is a nucleotide sequence that allows high-throughput sequencing of amplified nucleic acids.

Embodiment 218. The composition of embodiment of any one of embodiments 215-217, wherein the adapter sequence which permits capture on a flow cell.

Embodiment 219. The composition of embodiment of any one of embodiments 215-218, wherein the adapter sequence is a P5 sequence, a P7 sequence, or the reverse complement of a P5 or P7 sequence.

Embodiment 220. The composition of embodiment of any one of embodiments 215-219, wherein the P5 sequence is SEQ ID NO: 3 or SEQ ID NO: 10 and the P7 sequence is SEQ ID NO: 4 or SEQ ID NO: 11.

Embodiment 221. A method for amplifying a double-stranded DNA fragment of interest, comprising steps of: (a) generating a tagged version of the double-stranded DNA fragment, having a first double-stranded tag at one end and a second double-stranded tag at the other end, the first and second tags flanking the double-stranded DNA fragment of interest; (b) contacting the tagged double-stranded fragment with the composition of embodiment 180 and a DNA polymerase, wherein part (i) of the first oligonucleotide hybridizes to the sense strand of the first double-stranded tag and part (iv) of the third oligonucleotide hybridizes to the antisense strand of the second double-stranded tag; (c) extending the second and fourth oligonucleotides to generate amplification primers from the first and third oligonucleotides; and (d) using the primers to amplify the double-stranded DNA fragment of interest.

Embodiment 222. The method of embodiment of 221, wherein steps (a) to (c) occur in situ within a cell.

Embodiment 223. The method of embodiment of 221 or 222, wherein steps (a) to (d) occur in situ within a cell.

Embodiment 224. The method of any one of embodiments of 221-223, wherein the cells are lysed after step (d).

Embodiment 225. The method of any one of embodiments 221-224, the method further comprising PCR after lysis.

Embodiment 226. The method of any one of embodiments 221-225, wherein step (c) involves thermal cycling and the DNA polymerase is thermostable.

Embodiment 227. The method of any one of embodiments 221-226, wherein step (c) is performed isothermally, and step (b) includes a nickase.

Embodiment 228. A method for sequencing a DNA fragment of interest, comprising steps of: (a) amplifying the DNA fragment of interest by the method of claim X; (b) sequencing the amplified DNA fragment.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: In Situ Amplicon Library Preparation and Barcoding of a Heterogeneous Cell Population

The example provided herein shows in situ library preparation of intact cells.

The first step of library preparation includes Targeted rhAmpSeq PCR 1. PCR 1 adds consensus regions (CR1 and CR2) during amplification. For example, the rhAmpSeq PCR Panel forward primers include a read1 sequence (i.e., CR1) and the reverse primers include a read2 sequence (i.e., CR2). Following amplification, an amplified nucleic acid fragment includes read1 sequence on one end of the amplicon and a read2 sequence on the other end of the amplicon.

### Prepare Reagents:

Thaw at room temperature:
10×rhAmp PCR Panel—Forward Pool
10×rhAmp PCR Panel—Reverse Pool

Thaw on ice:
4×rhAmpSeq Library Mix 1
   Targeted rhAmpPCR 1 Protocol:

1) Dilute 16,000 permeabilized cells to a final volume of 11 ul using IDTE, ph8.0

2) Using PCR Strip Tubes, Add the following to each reaction:

TABLE 1

| Reagent | Volume (Per Rxn) | |
|---|---|---|
| Cell Dilution (16,000 cells) | 11 | ul |
| 4X rhAmpSeq Library Mix 1 | 5 | ul |
| 10X rhAmp PCR Panel -- Forward Pool | 2 | ul |
| 10X rhAmp PCR Panel -- Reverse Pool | 2 | ul |
| Total Volume | 20 | ul |

4) Seal Tubes, Vortex Briefly then Centrifuge

5) Run the Target rhAmp PCR 1 Program on Thermocycler

TABLE 2

| Step | Cycle | Temperature (*C.) | Duration | |
|---|---|---|---|---|
| Activate Enzyme | 1 | 95 | 10 | min |
| Amplify | 14 | 95 | 15 | sec |
| | | 61 | 8 | min |
| Deactivate Enzyme | 1 | 99.5 | 15 | min |

| | | | |
|---|---|---|---|
| Hold | 1 | 4 | Forever |

7) Remove PCR product when Program completes

8) Centrifuge Cell Samples from LOD Dilution of Targeted rhAmpSeq PCR 1 for 5 min at 1,500×g

Remove Supernatant

The next step of the method involves in situ Cell Barcoding during rhAmpSeq PCR 2.

Incubate Cells with Cell Barcode Oligos

Resuspend cell pellet with the following:

TABLE 1

| Reagent | Volume (Per Rxn) | |
|---|---|---|
| PBS | 16 | ul |
| P5 Barcode Oligo at (1 uM, 1 nM, or 1 pM) | 2 | ul |
| P7 Barcode Oligo at (1 uM, 1 nM, or 1 pM) | 2 | ul |
| Total Volume | 20 | ul |

Incubate 5 min

Centrifuge Cells for 5 min at 1,500×g

Remove Supernatant

Resuspend in 11 ul of PBS

### Perform Cell Barcoding PCR2 Protocol

### Prepare Reagents:

1) Thaw at room temperature:
Amplification Primer P5 and P7

2) That on ice:
4×rhAmpSeq Library Mix 2
   Targeted rhAmpSeq PCR 2 Protocol:

1) Briefly vortex the thawed reagents

2) Prepare PCR 2 in a new PCR Strip Tube

TABLE 3

| Component | Volume (per reaction) | |
|---|---|---|
| 4x rhAmpSeq Library Mix 2 | 5 | uL |
| P5 (1 uM) | 2 | uL |
| P7 (1 uM) | 2 | uL |
| Barcode Oligo Incubated Cells | 11 | uL |
| Total Volume | 20 | uL |

3) Seal the indexing PCR reactions

4) Vortex

5) Centrifuge

6) Run the Target rhAmp PCR 2 Program on Thermocycler

7) Use a preheated lid (105° C., if the temperature can be programed)

TABLE 5

| Step | Cycle | Temperature (*C.) | Duration | |
|---|---|---|---|---|
| Activate Enzyme | 1 | 95 | 3 | min |
| Amplify | 29 | 95 | 15 | sec |
| | | 60 | 30 | sec |
| | | 72 | 30 | sec |
| Final Extension | 1 | 72 | 1 | min |

| | | | |
|---|---|---|---|
| Hold | 1 | 4 | Forever |

### Cell Lysis Protocol

1) Add 5 ul PBS to the Cells (final volume 25 ul)

8) Add 5 μl QIAGEN Protease or proteinase K.

9) Add 25 μl Buffer AL.

10) Mix thoroughly by vortexing for 15 s.

11) Incubate at 70° C. for 10 min

12) Briefly centrifuge the tube to remove drops from the lid.

13) Total Volume is 55 ul

AMPure XP PCR Cleanup of rhAmpSeq Library

### Prepare Reagents:

Bring to room temperature:
Agencourt AMPure XP Beads
Prepare Fresh
80% Ethanol—500 uL per sample

### Protocol:

1) Add 55 ul AMPure XP Beads (1×)

2) Thoroughly Pipette mix

3) Incubate 10 minutes at Room Temp

4) Centrifuge

5) Place on Plate Magnet for 5 minutes, or until solution is clear

6) While on Magnet Do Steps 7-11 2×

7) Remove the supernatant, avoiding magnetic pellet

8) Add 200 ul 80% EtOH

9) Incubate at room temp for 30 sec

10) Briefly Spin down strip tube

11) Place back on magnet and let beads separate for 30 secs

12) Keeping on magnet do steps 13-15:

13) Use a fresh pipette tip to remove all traces of ethanol from the tube

14) Allow beads to dry for 3 minutes at room temp

15) Add 22 ul IDTE, pH 8.0 to the library pool

17) Vortex thoroughly

18) Incubate at room Temperature 3 minutes

19) Place on Plate Magnet for 1 minute or until solution is clear

20) Keeping on magnet, Transfer 20 ul to a new PCR strip

A quality control (QC) step was performed using the following protocol (https://www.agilent.com/en/product/automated-electrophoresis/tapestation-systems/tapestation-dna-screentape-reagents/high-sensitivity-dna-screentape-analysis-228262)

Post rhAmpSeq PCR 2 product:

Prepare Reagents:

Bring to room temperature, 30 minutes:
D1000 Sample Buffer
D1000 Ladder
D1000 Tape

### Protocol:

1) Vortex Sample Buffer before use

2) Add 3 ul Sample Buffer to required number number of tubes (# Samples+1 Ladder)

3) Add 1 ul of D1000 Ladder or 1 uL Sample to respective tubes

4) Spin down

5) Vortex using IKA vortexer and adaptor at 2000 rpm for 1 min

6) Spin down to position the sample at the bottom of the tube.

7) Load samples into the 2200 TapeStation instrument.

8) Select the required samples on the 2200 TapeStation Controller Software (must be even number)

### Reagents and Materials Used in Example 1

1) Qiagen QiaAmp DNA Mini Kit

2) Ethanol

3) Nuclease Free Water

4) IDTE, ph 7.5

5) IDTE, ph 8.0

6) Agencourt AMPure XP Beads

7) PCR Strip Tubes (may need 2 types)

8) 1.5 ml Eppendorf Tubes

9) 96 Well Magnet Plate: https://www.thermofisher.com/order/catalog/product/12331D?SID=srch-srp-12331D#/12331D?SID=srch-srp-12331D

12) Agilent tapestation highsensitivity d1000 solutions and tapes

### Example 2: In Situ Cell Barcoding with Nick Mediated Isothermal

### Amplification

The purpose of this study was to test the feasibility of nick-mediated isothermal amplification for use in single cell barcoding.

In Vitro Annealing of Barcode Oligo and Amplification Primer

1. Mix the P5 barcoding oligonucleotide containing an ERS site (100 μM) and its amplification primer (100 μM) at 1:1 molar ratio in a microfuge tube, resulting duplex is at 50 μM.

2. Separately, mix the P7 barcoding oligonucleotide containing an ERS site (100 μM) and its amplification primer (100 μM) at 1:1 molar ratio in a microfuge tube, resulting duplex is at 50 μM.

3. Anneal both in PCR Machine:

| Step | Cycle | Temperature (*C.) | Duration | |
|---|---|---|---|---|
| Ensure Denaturation | 1 | 95 | 5 | min |
| Cool | 70 | 95-1/cycle | 1 | min |

| | | | |
|---|---|---|---|
| Hold | 1 | 4 | Forever |

4. Dilute each annealed primer set to 1 μM, 1 nM, and 1 pM using IDTE

| | | | | | Volume | Volume |
|---|---|---|---|---|---|---|
| Final | | Starting | | | Oligo | IDTE |
| Concentration | | Concentration | | Dilution | (uL) | (uL) |
| 1 | μM | 100 | μM | 1:100 | 10 | 990 |
| 10 | nM | 1 | μM | 1:100 | 10 | 990 |
| 1 | μM | 10 | nM | 1:10 | 100 | 900 |
| 10 | pM | 1 | μM | 1:100 | 10 | 990 |
| 1 | pM | 10 | pM | 1:10 | 100 | 900 |

Targeted rhAmpSeq PCR 1

### Prepare Reagents:

Thaw at room temperature:
10×rhAmp PCR Panel—Forward Pool
10×rhAmp PCR Panel—Reverse Pool

Thaw on ice:
4×rhAmpSeq Library Mix 1
   Targeted rhAmpPCR 1 Protocol:

1) Dilute 16,000 permeabilized cells to a final volume of 11 ul using IDTE, ph8.0

2) Using PCR Strip Tubes, Add the following to each reaction:

TABLE 1

| Reagent | Volume (Per Rxn) | |
|---|---|---|
| Cell Dilution (16,000 cells) | 11 | ul |
| 4X rhAmpSeq Library Mix 1 | 5 | ul |
| 10X rhAmp PCR Panel -- Forward Pool | 2 | ul |
| 10X rhAmp PCR Panel -- Reverse Pool | 2 | ul |
| Total Volume | 20 | ul |

4) Seal Tubes, Vortex Briefly then Centrifuge

5) Run the Target rhAmp PCR 1 Program on Thermocycler

TABLE 2

| Step | Cycle | Temperature (*C.) | Duration | |
|---|---|---|---|---|
| Activate Enzyme | 1 | 95 | 10 | min |
| Amplify | 14 | 95 | 15 | sec |
| | | 61 | 8 | min |
| Deactivate Enzyme | 1 | 99.5 | 15 | min |

| | | | |
|---|---|---|---|
| Hold | 1 | 4 | Forever |

7) Remove PCR product when Program completes

8) Centrifuge Cell Samples from LOD Dilution of Targeted rhAmpSeq PCR 1 for 5 min at 1,500×g

Remove Supernatant

Resuspend in 9 μl of PBS, vortex gently and centrifuge briefly.

Incubate Cells with Cell Barcode Oligos

Resuspend cell pellet with the following:

TABLE 1

| Reagent | Volume (Per Rxn) | |
|---|---|---|
| PBS | 16 | ul |
| Annealed P5 Barcode Oligo at (1 uM, | 2 | ul |
| 1 nM, or 1 pM) | | |
| Annealed P7 Barcode Oligo at (1 uM, | 2 | ul |
| 1 nM, or 1 pM) | | |
| Total Volume | 20 | ul |

Incubate 5 min

Centrifuge Cells for 5 min at 1,500×g

Remove Supernatant

Resuspend in 13 ul of PBS

Nick-Mediated Isothermal Amplification—all Isothermal Protocol Samples

1. Add to the Cells:

| Reagent | Volume | |
|---|---|---|
| Barcode Incubated Cells | 13 | μl |
| 10X Isothermo Amplification Buffer | 2 | uL |
| dNTP | 2 | μl |
| MgSo4 | 1 | μl |
| Bst2.0 (8 units/μl) | 1 | μl |
| Nt. BspQI (10 units/μl) | 1 | μl |

2. Resuspend by vortexing gently, and centrifuge briefly.

3. Perform the following Isothermal Amplification Reaction followed by heat inactivation.

| Step | Cycle | Temperature (*C.) | Duration |
|---|---|---|---|
| Isothermal | 1 | 55 | 2, 5, or 10 min |
| Amplification | | | |
| Heat Inactivation | 1 | 80 | 20 min |
| Hold | 1 | 4 | Forever |

4. Centrifuge Cells for 5 min at 1,500×g

5. Remove Supernatant

6. Resuspend in 15 ul of PBS

Targeted rhAmpSeq PCR 2 Protocol:

1) Briefly vortex the thawed reagents

2) Prepare PCR 2 in a new PCR Strip Tube

TABLE 3

| Component | Volume (per reaction) | |
|---|---|---|
| 4x rhAmpSeq Fibrary Mix 2 | 5 | uL |
| Isothermal Amplified Cells | 15 | uL |
| Total Volume | 20 | uL |

3) Seal the indexing PCR reactions

4) Vortex

5) Centrifuge

6) Run the Target rhAmp PCR 2 Program on Thermocycler

7) Use a preheated lid (105° C., if the temperature can be programed)

TABLE 5

| Step | Cycle | Temperature (*C.) | Duration | |
|---|---|---|---|---|
| Activate Enzyme | 1 | 95 | 3 | min |
| Amplify | 29 | 95 | 15 | sec |
| | | 60 | 30 | sec |
| | | 72 | 30 | sec |
| Final Extension | 1 | 72 | 1 | min |

| | | | |
|---|---|---|---|
| Hold | 1 | 4 | Forever |

### Cell Lysis Protocol

1) Add 5 ul PBS to the Cells (final volume 25 ul)

8) Add 5 μl QIAGEN Protease or proteinase K.

9) Add 25 μl Buffer AL.

10) Mix thoroughly by vortexing for 15 s.

11) Incubate at 70° C. for 10 min

12) Briefly centrifuge the tube to remove drops from the lid.

13) Total Volume is 55 ul

AMPure XP PCR Cleanup of rhAmpSeq Library

Prepare Reagents:

Bring to room temperature:
Agencourt AMPure XP Beads
Prepare Fresh
80% Ethanol—500 uL per sample

### Protocol:

1) Add 55 ul AMPure XP Beads (1×)

2) Thoroughly Pipette mix

3) Incubate 10 minutes at Room Temp

4) Centrifuge

5) Place on Plate Magnet for 5 minutes, or until solution is clear

6) While on Magnet Do Steps 7-11 2×

7) Remove the supernatant, avoiding magnetic pellet

8) Add 200 ul 80% EtOH

9) Incubate at room temp for 30 sec

10) Briefly Spin down strip tube

11) Place back on magnet and let beads separate for 30 secs

12) Keeping on magnet do steps 13-15:

13) Use a fresh pipette tip to remove all traces of ethanol from the tube

14) Allow beads to dry for 3 minutes at room temp

15) Add 22 ul IDTE, pH 8.0 to the library pool

17) Vortex thoroughly

18) Incubate at room Temperature 3 minutes

19) Place on Plate Magnet for 1 minute or until solution is clear

20) Keeping on magnet, Transfer 20 ul to a new PCR strip

A quality control (QC) step was performed using the following protocol (https://www.agilent.com/en/product/automated-electrophoresis/tapestation-systems/tapestation-dna-screentape-reagents/high-sensitivity-dna-screentape-analysis-228262)

Post rhAmpSeq PCR 2 Product:

### Prepare Reagents:

Bring to room temperature, 30 minutes:
D1000 Sample Buffer
D1000 Ladder
D1000 Tape

### Protocol:

1) Vortex Sample Buffer before use

2) Add 3 ul Sample Buffer to required number number of tubes (# Samples+1 Ladder)

3) Add 1 ul of D1000 Ladder or 1 uL Sample to respective tubes

4) Spin down

5) Vortex using IKA vortexer and adaptor at 2000 rpm for 1 min

6) Spin down to position the sample at the bottom of the tube.

7) Load samples into the 2200 TapeStation instrument.

8) Select the required samples on the 2200 TapeStation Controller Software (must be even number)

### Example 3: Bioinformatics Processing Workflow and Analysis

The bioinformatics workflow described herein is used to process sequencing reads from barcoded nucleic acid amplified in situ from sub-populations of live cells within a heterogeneous human biological sample. Within a heterogeneous sample, each amplicon of DNA (or cDNA) isolated from a cellular sub-population will contain a known, unique nucleotide sequence barcode specific to that sub-population. After pooling amplified DNA (or cDNA) from multiple cellular sub-populations and sequencing this pooled nucleic acid sample, the unique barcode is used to identify sequence reads originating from a particular cellular sub-population. Using quality scores for each nucleotide readout of a sequence read, standard error-detection and error-correcting methods are used to correct barcode sequences containing a sequencing error, or to remove reads not containing a known barcode even after error-correction.

After error-correction and removal of reads without a known barcode, all reads for a given sample are demultiplexed according to their sequence barcode, resulting in multiple sequence read files. For example, a standard FASTQ file format is used to store sequence reads containing a given barcode. We will refer to these demultiplexed sequence files as ‘barcoded’ files. Barcode information is saved in the header of each sequence read.

An algorithm was developed in order tag reads from an in situ single-cell sequencing sample with a cell ID and quantify structural variants from these reads.

The Program takes as input zipped R1, R2, I1, and I2 FASTQ files, and creates a Graph containing nodes representing barcodes, and edges representing a read containing those barcodes. Actual read sequences and associated quality scores are stored in a read dictionary. After appropriate pruning, the Graph should contain sub-graphs where each sub-graph is a “cell”. This program then returns individual FASTQ files of reads, one for each “cell”.

The basic idea is that, for a given sample, a graph is created where barcodes are stored as “nodes” and the reads (which each contain 2 cell barcodes) are stored as “edges”. The key is that the graph is “pruned” so that reads that appear due to leakage of a barcode from one cell to another cell are removed. What is left is a graph containing clusters of Vreads, where each cluster is a cell. All of the barcodes and reads associated with that cell are then output to a sequence FASTQ file, one per cell.

### Specifications

### Pruning Algorithm and FASTQ Output:

There are two types of graph pruning that can occur, depending on the read depth of the sequenced sample (also see FIG. 4).

(1) If the read depth is high enough so that we get on average tens of reads per barcode-pair, this script will prune by edge weight (i.e., number of reads for a given barcode-pair. The pruning algorithm will calculate an empirical read threshold based on the data—any edges with weight less than this read threshold will be pruned. This empirical threshold is modeled based on known average experimental rates of barcode leakage from one cell to another cell, the sequencing error rates, the empirical shapes of the signal and noise distributions in the data (note: for initial testing, a constant read threshold will be used). Any singleton nodes (nodes with no edges) as a result of pruning are removed. Resulting sub-graph clusters are representative of our cells, and so read information is then output for each sub-graph cluster, one cluster per file in FASTQ format. The resultant FASTQs can then be fed into any single cell alignment and/or single cell variant calling programs.

### Error Correction

Barcodes

Because cell barcodes are random, there is a chance two distinct barcodes may only be one mismatch apart (Hamming Distance of 1). Thus, we cannot assume that two barcodes with Hamming Distance of 1 arise from sequencing error and correct a priori. Instead, we allow the pruning algorithm to naturally remove edges between two barcodes that are one mismatch apart if either the number of reads with this barcode-pair or the number of common neighbors is less than the empirically-calculated threshold, based on the pruning algorithm used. Note that this empirically-calculated threshold takes into account the sequencing error rate, thus effectively providing sequencing-based error correction within the algorithm.

Aligned Reads

The cell barcodes for each read will be stored in the header of each sequence, and so will carry over into the alignment SAM/BAM files.

(2) If the read depth is too low for pruning-by-edge-weight, the script will instead prune by ‘connectedness’ of barcode pairs. Connectedness is defined as follows—given two barcodes A and B of a paired-barcode read (there is an edge A-B representing this read), this algorithm finds all barcode neighbors of A, and separately all barcode neighbors of B. The algorithm then counts how many barcode neighbors A and B share in common versus distinct barcode neighbors, which gives a quantitative measure of how likely barcodes A and B are in the same cluster (same cell). This is calculated for all barcode pairs (so this is an N\hat{ }2 operation), and an empirical threshold is calculated based on the distribution of these fraction of common neighbors, the sequencing error rate, and an initial expected leakage rate based on the experiment (again, for initial testing we will start with fixed thresholds). Any barcode pairs with a fraction of common neighbors less than this threshold are pruned, and any singleton nodes as a result of pruning are removed. Resultant sub-graph clusters are representative of our cells, and so read information is then output for each sub-graph cluster, one cluster per file in FASTQ format. The resultant FASTQs can then be fed into any single cell alignment and/or single cell variant calling programs

### Development Steps

Graph data structures for storing barcodes and barcode relationships

Read Class

Id: Usually the read header, but could be something else.

Seq: Read sequence. Could compress this if memory is an issue.

Qual: Quality score.

Type: Type of read (e.g., R1, R2, I1, I2).

Read Graph Class

Graph structure—stores barcode nodes and read edges. Contains the following sub-data structures:

Dictionary of Read Objects
{1: [Read R1, Read R2, Read BC1, Read BC2], 2: [ . . . ], . . . }

Nodes
“AAAATTTTT” (node IDs are the barcode strings)

Edges
Contain references to the actual reads for each barcode-pair (each read contains a pair of barcodes, which are the corresponding nodes)
List of integer indexes, where these indexes reference keys in the dictionary of read objects (e.g., [1, 4, 7, 10, . . . ])

NetworkX

Python library for storing graph of nodes and edges. Has a lot of useful graph operations.

### Graph Pruning Functions

prune_by_edge_weight(int):

Prunes all edges with weight less than int (threshold weight).

prune_by_connectedness(float):

Prunes edges for which the two nodes share very few neighbors (or none at all). The cutoff is determined by float, which is the minimum % of shared neighbors, relative to the average number of neighbors for each node.

After the read information is output for each sub-graph cluster, one cluster per file in FASTQ format, the reads are trimmed to remove the barcode sequence as well as any adaptor sequences. Trimmed reads are then sequence aligned to the human genome. For a given barcoded sequence file, we use at least two aligners to minimize the number of variants falsely called due to alignment issues. The alignment programs used depend on the sample—for DNA amplified from genomic DNA, genome read aligners are used (e.g., BWA-MEM or Bowtie2); for cDNA amplified from RNA, transcriptome read aligners are used (e.g., RNA-STAR or Salmon). Aligned reads are stored in uncompressed (SAM) or compressed (BAM) alignment files, with one group of alignment files per barcoded sample.

Next, aligned reads from each barcoded SAM or BAM file are then separately run through a variant caller to find structural variants. This involves a two-step process of first extracting all possible structural variants from a barcoded alignment file (variant identification), and second using statistical methods to quantify structural variants as statistically significant (variant quantification). Variant identification consists of listing all structural variants commonly found in the group of alignment files for each barcoded sample. Identified variants can be written out in any appropriate format—in Example X, the uncompressed Variant Call Format (VCF) and compressed Binary Call Format (BCF) are used. Information including the percentage of reads in a region containing a variant, the quality scores of all nucleotides in reads covering a variant, the total number of reads at a variant position, and the genomic location(s) of the variant are listed within this VCF file. Variant quantification consists of using any of a number of statistical tests to calculate a statistical score and/or a significance value for a given variant. In Example X, for single nucleotide variants (SNVs) and small insertions and deletions (indels), we use a Hypothesis Test where we assume that the presence of a variant follows a Binomial distribution where the probability of a variant is equal to the average nucleotide error rate at that position. The nucleotide error rate is a function of the sequencing error rate as given by the Phred quality score and the average nucleotide misincorporation rate from PCR of the relevant genomic region. Hypothesis testing on binomially distributed populations works well for small sample sizes meaning we can quantify variants from small sub-populations containing only a few reads

The variant-detection bioinformatics workflow described herein finds structural variants specific to a sub-population of live cells within a heterogeneous sample. Our multiplexed data allows us to compare structural variants among cellular sub-populations within this sample.

In addition to finding structural variants, the invention covers the use of targeted DNA amplification panels and exome or transcriptome sequencing to characterize genotypes and deconvolve phenotypes for each of the barcoded cell sub-populations within a heterogeneous sample run through this assay. More specifically, reads from barcoded sequence files are aligned to the human genome.

The entire sequence data processing workflow outlined above was implemented within a custom bioinformatics processing pipeline developed using cloud compute resources. Specifically, each step of the processing pipeline is packaged into a Docker application that is saved as a Container image within an appropriate Container image repository. In this example cloud compute resources was used from Amazon Web Services to run each Docker container, although any cloud or on-premise compute resources with Docker installed could be used. In total, the compute resources used comprise a cloud-based end-to-end bioinformatics data processing pipeline.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 4. Amplification of Barcoding Oligo in a Reaction of Genomic DNA [Method 4.1]

This example provides a method for amplifying barcode oligos to generate barcoding primers that are then used to amplify an amplicon generated from a genomic DNA sample.

In these experiments, a commercially available amplicon sequencing kit was used to perform two PCR reactions (Amplicon Kit). In the first PCR (PCR1), 10Ong of genomic DNA was amplified using a standard amplicon panel for the Amplicon Kit thereby producing a library of genomic DNA amplicons. Amplicons generated with PCR1 include consensus regions (e.g., CR1 and CR2). The consensus regions are at least partially complementary to the reverse complement of the P5 barcoding oligo (i.e., GTCGTGTAGGGAAAGAGTG (5′ nucleotides of SEQ ID NO: 14)) or the reverse complement of the P7 barcoding oligo (i.e., ACACGTCTGAACTCCAGTCA (5′ nucleotides of SEQ ID NO: 15)).

The PCR1 reaction amplicons were subjected to a 99.5° C. incubation for 15 minutes. Following incubation, a second PCR amplification was used to amplify the barcode oligonucleotides (SEQ ID NO: 1 and SEQ ID NO: 2) to generate barcoding primers. The barcoding primers were then used to amplify the genomic DNA in subsequent rounds of amplification in the second PCR. The second PCR reaction (e.g., the barcoding reaction) was performed including 5.5 ul of a 1:10 dilution of the PCR 1 reaction amplicons and final concentration of 0.1 μM each P5 barcoding oligo (SEQ ID NO: 1), P7 barcoding oligo (SEQ ID NO: 2), P5 amplification primer (SEQ ID NO: 3) and P7 amplification primer (SEQ ID NO: 4) and 1× Amplicon Kit PCR2 Master Mix. The second PCR reaction was performed using the standard protocol for the Amplicon Kit with the addition of 5 extra PCR cycles.

P5 barcoding oligo:
(SEQ ID NO: 1)
5′-GTCGTGTAGGGAAAGAGTGTNNNNNNNNNNNNNNNNNNNNGTGT
AGATCTCGGTGGTCGCCGTATCATT-3′
P7 barcoding oligo:
(SEQ ID NO: 2)
5′-ACACGTCTGAACTCCAGTCACNNNNNNNNNNNNNNNNNNNNATC
TCGTATGCCGTCTTCTGCTTG-3′
P5 amplification primer:
(SEQ ID NO: 3)
5′-AATGATACGGCGACCACCGAGATCTACA-3′
P7 amplification primer:
(SEQ ID NO: 4)
5′-CAAGCAGAAGACGGCATACGAGAT-3′

Barcoded Libraries were then purified using a 1×AmpPure/SPRI (beckman coulter) purification and eluted in 20 ul of IDTE (IDT). Control libraries were generated using genomic DNA and amplification using the Amplicon Kit standard amplifying conditions. Libraries were then analyzed on a Tapestation (Agilent) to see if amplification occurred with the cell barcoding strategy.

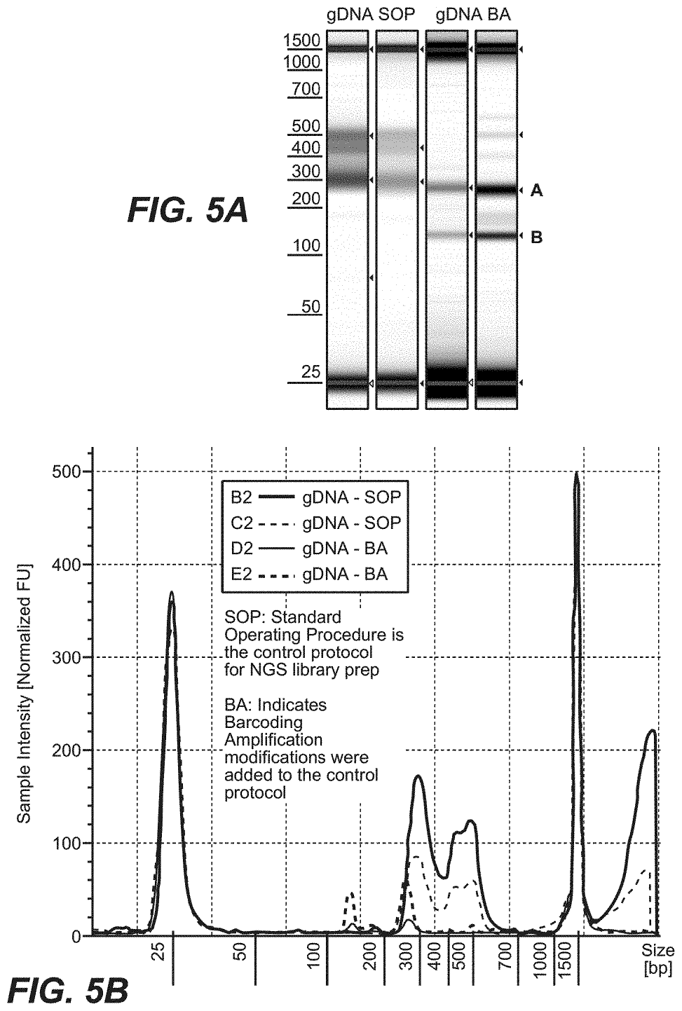

The results of this experiment are provided in FIGS. 5A and 5B. FIG. 5A shows images of the libraries run on a Tapestation and FIG. 5B shows quantification of the bands from the gel in FIG. 5A. The data shows that two main bands (FIG. 5A) or peaks (FIG. 5B) were observed in the barcode amplification samples, A (˜300 bp) and B (˜140 bp). Peak B is putative primer dimer and peak A is the amplicon of one or more target amplicons amplified using the barcoding primers.

This data suggests that amplification of a linear barcode oligonucleotide can occur in the same reaction as amplification of the precursor library.

### Example 5. In Vitro Amplification of Barcoding Oligo [Method 3.1]

This example provides a method for in vitro isothermal amplification of barcode oligonucleotides using an isothermal polymerase and an amplification oligo.

Barcoding oligonucleotides (SEQ ID NO: 5 and SEQ ID NO: 6) and amplification oligos (SEQ ID NO: 7) were incubated at 60° C. in a 1× isothermal amplification buffer (NEB) with warm start Bst2.0 isothermal polymerase (NEB) for 15 minutes Amplification was measured via gel electrophoresis.

P5 barcoding oligo:
(SEQ ID NO: 5)
5′-GTCGTGTAGGGAAAGAGTGTNNNNNNNNNNNNNNNNNNNNGTGTA
GATCTCGGTGGTCGCCGTATCATTAAAAAAAAAAAAAAAAAAAAA-3′
P7 barcoding oligo:
(SEQ ID NO: 6)
5′-ACACGTCTGAACTCCAGTCACNNNNNNNNNNNNNNNNNNNNATCT
CGTATGCCGTCTTCTGCTTGAAAAAAAAAAAAAAAAAAAAA-3′
Amplification oligo:
(SEQ ID NO: 7)
5′-TTTTTTTTTTTTTTTTTTTT-3′

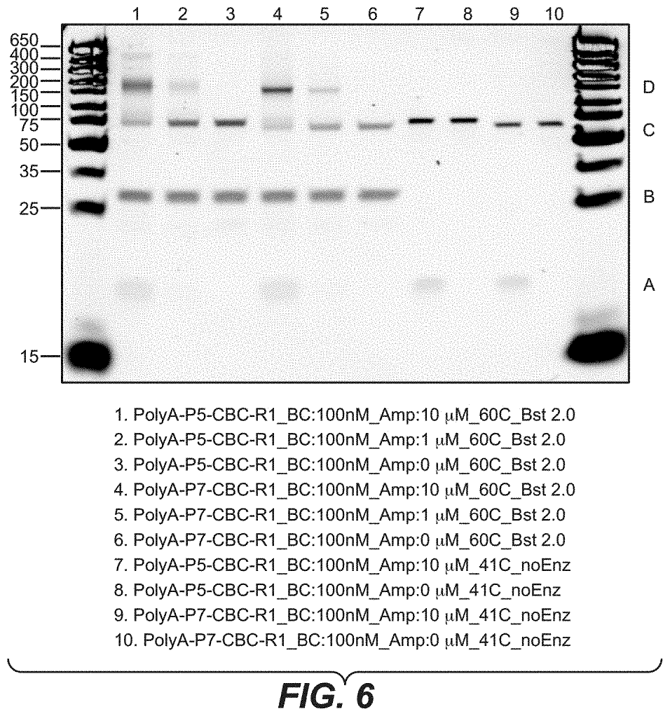

The results of the amplification are shown in the gel in FIG. 6. Band A indicates the amplification oligo. Band B indicates the aptamer used to inhibit Bst2.0 in the warm start isothermal polymerase, and is not present when the Bst2.0 enzyme was not included. Band C indicates the barcoding oligo starting material. Band D indicates amplification product.

These results show successful amplification of barcode oligonucleotides using in vitro isothermal amplification comprising an isothermal polymerase and an amplification oligo.

### Example 6. Amplification of Barcoding Oligo In Situ [Method 3.2]

In this example, barcode oligonucleotides were amplified to generate barcoding primers using isothermal amplification and the barcoding primers were used to amplify an in situ prepared library.

For these experiments, a first in situ step was performed to generate a library. In this first step, targeting oligos were used to amplify input material. A second in situ step was used to amplify barcode oligonucleotides to generate barcoding primers.

Step 1: An in situ library prep was prepared using a method developed in house using multiplexed primers and a high fidelity DNA polymerase. In particular, for the in situ library prep, the PCR master mix included 1× polymerase master mix, 5 nM final concentration of each targeting oligo (e.g., SEQ ID NO: 8 and SEQ ID NO: 9), and an extra unit of high-fidelity DNA polymerase.

Step 2: In situ barcode amplification reaction was performed using 100 nM barcode oligos (SEQ ID NO: 5 and SEQ ID NO: 6), which were incubated with the cells (i.e., in situ prepared libraries from step 1) at 41° C. for 15 minutes followed by introduction of a 1× reaction mix including Bst2.0, an isothermal buffer, and 1 μM of an amplification oligo (SEQ ID NO: 7). The reactions were incubated at 60° C. for 15 minutes.

As a control, in situ prepared libraries from step 1 were not subject to in situ barcode amplification but instead were used directly in step 3. This control was referred to as the “in situ control.”

Step 3: After barcode amplification, a second in situ PCR was performed using the same reaction conditions as earlier for the first in situ library step. The in situ control was amplified using a P5 amplification primer (SEQ ID NO: 10) and a P7 amplification primer (SEQ ID NO: 11). The reaction from step 2 comprising the in situ prepared libraries from step 1 and the barcoding primers from step 2 were subjected to the same thermal cycler conditions as the control with the barcoding primers enabling amplification of the in situ library prep. Libraries were SPRI purified following cell lysis and a third PCR was performed.

R1 Targeting Primer:
(SEQ ID NO: 8)
5′-ACACTCTTTCCCTACACGACACTATTCCGATCT + 15-
25 bp Targeting Sequence-3′
R2 Targeting Primer:
(SEQ ID NO: 9)
5′-TGACTGGAGTTCAGACGTGTACTATTCCGATCT + 15-
25 bp Targeting Sequence-3′
P5 barcoding oligo:
(SEQ ID NO: 5)
5′-GTCGTGTAGGGAAAGAGTGTNNNNNNNNNNNNNNNNNNNNGTGTA
GATCTCGGTGGTCGCCGTATCATTAAAAAAAAAAAAAAAAAAAAA-3′
P7 barcoding oligo:
(SEQ ID NO: 6)
5′-ACACGTCTGAACTCCAGTCACNNNNNNNNNNNNNNNNNNNNATCT
CGTATGCCGTCTTCTGCTTGAAAAAAAAAAAAAAAAAAAAA-3′
Amplification oligo:
(SEQ ID NO: 7)
5′-TTTTTTTTTTTTTTTTTTTT-3′
P5 amplification primer:
(SEQ ID NO: 10)
5′-AATGATACGGCGACCACCGA-3′
P7 amplification primer:
(SEQ ID NO: 11)
5′-CAAGCAGAAGACGGCATACGA-3′

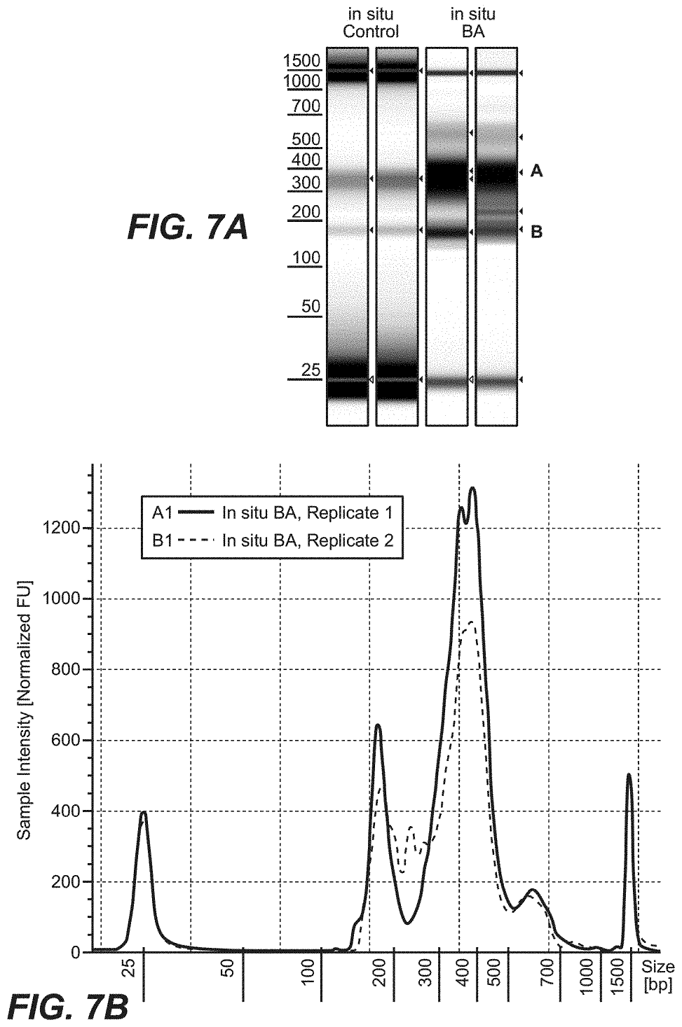

The results are shown in FIGS. 7A and 7B. Each lane in FIG. 7A indicates a biological replicate and each replicate of the in situ BA is indicated by a separate line in FIG. 7B. FIG. 7A shows images of the libraries run on a Tapestation and FIG. 7B shows quantification of the bands from the gel in FIG. 7A. Two main bands (FIG. 7A) or peaks (FIG. 7B) were observed in each sample. In the in situ control samples, Band A denotes the amplified libraries prepared by a first in situ amplification using the targeting primers (SEQ ID NO: 8 and SEQ ID NO: 9) and a second amplification using amplification primers (SEQ ID NO: 10 and SEQ ID NO: 11). In the in situ BA samples, Band A denotes amplified library prepared by a first in situ amplification using targeting primers (SEQ ID NO: 8 and SEQ ID NO: 9) and a second amplification using in situ amplification of barcoding primers (SEQ ID NO: 5 and SEQ ID NO: 6) (i.e., following amplification of the barcode oligonucleotides in step 2 using an amplification oligo (SEQ ID NO: 7)). Band B is a putative primer dimer. FIG. 7B shows quantification of the Tapestation run in FIG. 7A.

Overall, the results show that barcode oligonucleotides can be amplified to generate barcoding primers using isothermal amplification and the barcoding primers can then be used to amplified an in situ prepared library.

### Example 7: In Situ Cell Barcoding with Isothermal Amplification

Cultured B-cells (GM12878, Coriell Institute for Medical Research) were fixed and permeabilized with 1 ml of 1× IncellMax reagent (incellDx) for 1 million cells for 1 hour at room temperature. 16,000 cells were subjected to a 20-minute pre-treatment at 95° C., followed by a one-step enzymatic fragmentation, end-repair and a-tailing reaction using 1× Fragmentation and A-tailing Buffer, and 1.5× Fragmentation and A-tailing Enzyme Cocktail (Watchmaker Genomics). Cells were incubated in this mixture for 20 minutes at 37° C. and 30 minutes at 65° C. Fragmented DNA was ligated in situ to 1 μM Xgen stubby adapters (IDT) in 1× ligation master mix for 15 minutes at 20° C., and then enzymatic inactivation was performed for 15 minutes at 65° C. This step added adapters, which included consensus regions (e.g., CR1 and CR2), to the end of the fragmented DNA.

After ligation and its subsequent inactivation, the cells were washed in dPBS, pelleted at 1,500×g for 5 minutes, and resuspended in dPBS containing 33 nM each of P5 and P7 cell barcoding oligos. Cell barcodes were allowed to equilibrate at 41° C. for 30 minutes. Cells were washed in dPBS, pelleted at 1,500×g for 5 min and then cell barcodes were amplified in situ with 19.2 U Bst2.0 Warm Start polymerase, 1.4 nM dNTPs, 6 mM Mg₂(SO)₄, and 100 nM of amplification oligo at 41° C. for 30 minutes. Cells were once again washed with dPBS followed by centrifugation at 1,500×g for 5 min and amplified in 1×PCR Amplification Mix (Watchmaker Genomics) with an initial denaturation of 95° C. for 45 seconds, and 12 cycles of amplification (denaturation of 95° C. for 15 seconds, annealing of 60° C. for 30 seconds, and extension of 72° C. for 30 seconds). A final wash with dPBS and centrifugation before lysing the cells in 1× lysis buffer (Qiagen) supplemented with 3.3 ug/ul proteinase K for 10 minutes at 70° C. in lysis buffer Barcoded DNA fragments were purified using SPRIselect beads (BeckmanCoulter) at a 1.5× bead to sample ratio. Purified barcoded libraries were subjected to an additional PCR using a 1×P5/P7 amplification primer mix and 1×NEBNext Q5 Hot Starr HiFi PCR Master Mix (Qiagen). Amplified libraries were purified using a 1.2×SPRI to sample volume ratio.

Amplification oligo:
(SEQ ID NO: 7)
5′-TTTTTTTTTTTTTTTTTTTT*-3′
(* indicates a phosphonothioate bond)
P5 barcoding oligo:
(SEQ ID NO: 12)
5′-GTCGTGTAGGGAAAGAGTGTAANNNNNGTNNNNNGTNNNNNGTNNN
NNCCGTGTAGATCTCGGTGGTCGCCGTATCATTAAAAAAAAAAAAAAAA
AAAA-3′
P7 barcoding oligo:
(SEQ ID NO: 13)
5′-ACACGTCTGAACTCCAGTCACNNNNNACNNNNNACNNNNNACNNNN
NATCTCGTATGCCGTCTTCTGCTTGAAAAAAAAAAAAAAAAAAAAA-3′
P5 amplification primer:
(SEQ ID NO: 10)
5′-AATGATACGGCGACCACCGA-3′
P7 amplification primer:
(SEQ ID NO: 11)
5′-CAAGCAGAAGACGGCATACGA-3′

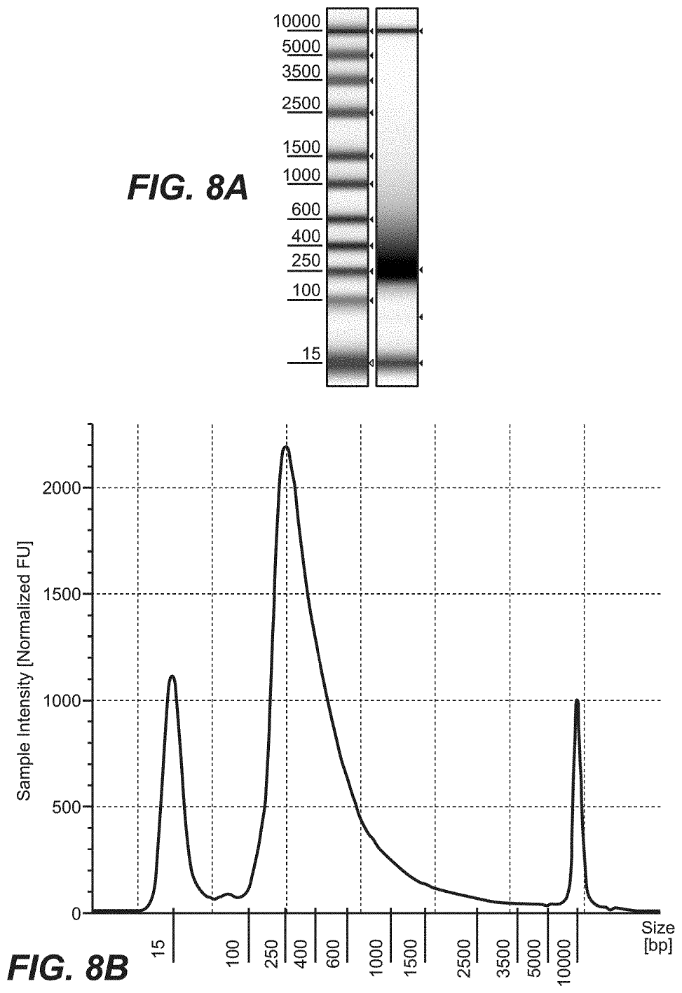

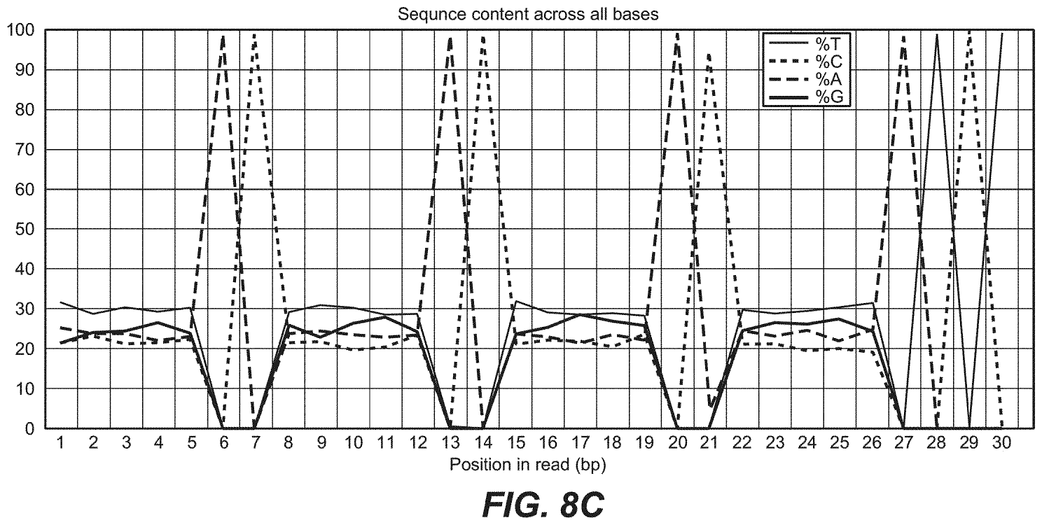

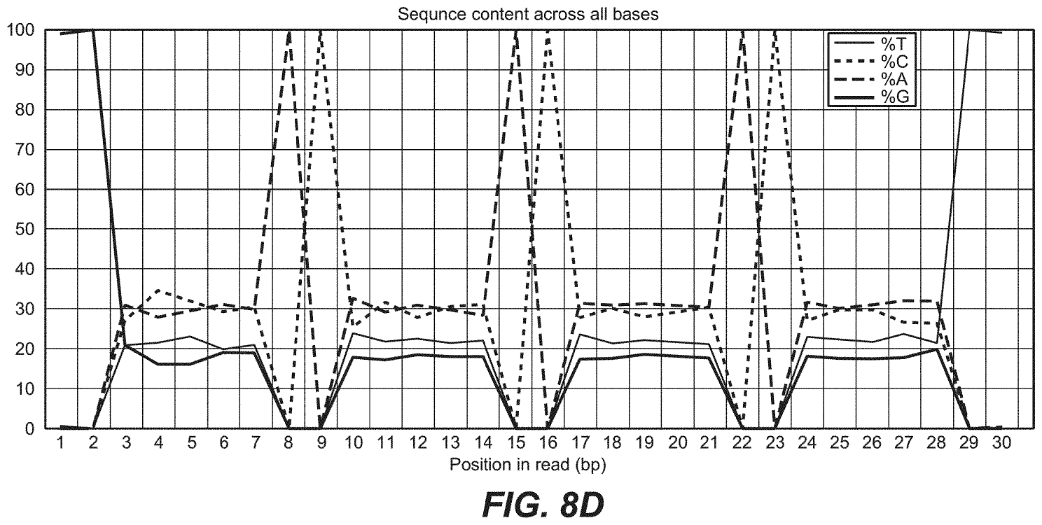

The results are shown in FIGS. 8A through 8D. FIG. 8A shows a gel image from an in situ cell barcoding sample run on a Agilient Tapestation HSd5000. FIG. 8B shows an electrophoretogram of the same sample. FIG. 8C provides the base composition of index 1, where low complexity bases at base 6, 7, 13, 14, 20, 21, 27, 28, 29, and 30 correspond to non-degenerate bases in the P7 cell barcoding oligo. FIG. 8D provides the base composition of index 2, where low complexity bases at 1, 2, 8, 9, 15, 16, 22, 23, 29, and 30 correspond to non-degenerate bases in the P5 cell barcoding oligo. FIGS. 8C and 8D show the correct formation of or cell barcodes after sequencing. Below is a table output from the sequencing run. A majority of reads have the expected cell barcode pair and map to the human genome table output from the of the sequencing run. A vast majority of reads have the expected cell barcode pair and map to the human genome.

TABLE 8

| | | |
|---|---|---|
| Sample | Reads (%) | % Mapped (hg38) |

| | | | |
|---|---|---|---|
| Full Run | 4.8M | (100%) | NA |
| Cell Barcode Reads | 4.3M | (89%) | 98.7% |
| PhiX | 0.17M | (3.6%) | NA |

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

Sequence Appendix

| SEQ | | |
|---|---|---|
| ID | | |
| NO: | Description | Sequence |
| 1 | P5 barcoding | GTCGTGTAGGGAAAGAGTGTNNNNNNNNNNN |
| | oligo | NNNNNNNNNGTGTAGATCTCGGTGGTCGCCG |
| | | TATCATT |
| 2 | P7 barcoding | ACACGTCTGAACTCCAGTCACNNNNNNNNNN |
| | oligo_1 | NNNNNNNNNNATCTCGTATGCCGTCTTCTGC |
| | | TTG |
| 3 | P5 | AATGATACGGCGACCACCGAGATCTACA |
| | amplification | |
| | primer_1 | |
| 4 | P7 | CAAGCAGAAGACGGCATACGAGAT |
| | amplification | |
| | primer_1 | |
| 5 | P5 barcoding | GTCGTGTAGGGAAAGAGTGTNNNNNNNNNNN |
| | oligo_2 | NNNNNNNNNGTGTAGATCTCGGTGGTCGCCG |
| | | TATCATTAAAAAAAAAAAAAAAAAAAAA |
| 6 | P7 barcoding | CACGTCTGAACTCCAGTCACNNNNNNNNNNN |
| | oligo_2 | NNNNNNNNNATCTCGTATGCCGTCTTCTGCT |
| | | TGAAAAAAAAAAAAAAAAAAAAA |
| 7 | Amplification | TTTTTTTTTTTTTTTTTTTT |
| | oligo | |
| 8 | R1 Targeting | ACACTCTTTCCCTACACGACACTATTCCGAT |
| | Primer | CT |
| 9 | R2 Targeting | TGACTGGAGTTCAGACGTGTACTATTCCGAT |
| | Primer | CT |
| 10 | P5 | AATGATACGGCGACCACCGA |
| | amplification | |
| | primer_2 | |
| 11 | P7 | CAAGCAGAAGACGGCATACGA |
| | amplification | |
| | primer_2 | |
| 12 | P5 barcoding | GTCGTGTAGGGAAAGAGTGTAANNNNNGTNN |
| | oligo 3 | NNNGTNNNNNGTNNNNNCCGTGTAGATCTCG |
| | | GTGGTCGCCGTATCATTAAAAAAAAAAAAAA |
| | | AAAAAA |
| 13 | P7 barcoding | ACACGTCTGAACTCCAGTCACNNNNNACNNN |
| | oligo 3 | NNACNNNNNACNNNNNATCTCGTATGCCGTC |
| | | TTCTGCTTGAAAAAAAAAAAAAAAAAAAAA |

1. A method of performing whole cell barcoding, the method comprising:
(a) contacting nucleic acid fragments within a cell suspension or tissue slices with:
(i) a first set of barcoding oligonucleotides, each barcoding oligonucleotide comprising:
a first barcode;
two consensus regions, wherein the two consensus regions of each barcoding primer comprises:
one of the two consensus regions comprises a nucleotide sequence that is complementary to a 5′ read region of a first strand of one of the nucleic acid fragments, and
the second of the two consensus regions comprises a first adapter sequence;
(ii) a second set of barcoding oligonucleotides, each barcoding oligonucleotides comprising:
a second barcode;
two consensus regions, wherein the two consensus regions of each barcoding primer comprises:
one of the two consensus regions comprises a nucleotide sequence that is complementary to a 5′ read region of a second strand of one of the nucleic acid fragments, and
the second of the two consensus regions comprises a second adapter sequence;
(b) amplifying:
the first set of barcoding oligonucleotides to produce a first set of barcoding primers; and
the second set of barcoding oligonucleotides to produce a second set of barcoding primers;
(c) amplifying the nucleic acid fragments with first and second set of barcoding primers to produce a set of amplicon products, wherein the set of amplicon products comprise the first barcoding primer bridging from the 5′ end of the 5′ strand of the nucleic acid fragments and the second barcoding primer bridging from the 5′ end of the opposite strand (3′ strand) of the nucleic acid fragments.

2. (canceled)

3. (canceled)

4. The method of claim 1,
wherein step (i) further comprises:
contacting the first barcoding oligonucleotide with a first primer set comprising nucleotide sequences that is complementary to the amplification sequence, or
contacting the first barcoding oligonucleotide with a first primer set comprising nucleotide sequences that are complementary to the adapter sequence of the first barcoding oligonucleotides.

5. The method of claim 3, wherein step (ii) further comprises:
contacting the second barcoding oligonucleotides with a second primer set comprising a nucleotide sequence that is complementary to the amplification sequence; or
contacting the second barcoding oligonucleotides with a second primer set comprising a nucleotide sequence that is complementary to the second adapter sequence of the second set of barcoding oligonucleotides.

6. (canceled)

7. (canceled)

8. The method of claim 1, wherein the said amplifying in step (b) comprises amplifying via isothermal amplification, the first and second set of barcoding oligonucleotides with the first and second set of primers to produce the first and second barcoding primers.

9. (canceled)

10. (canceled)

11. (canceled)

12. (canceled)

13. The method of claim 1, wherein the first and second barcoding oligonucleotides comprise hairpin barcoding oligonucleotides.

14. (canceled)

15. The method of claim 1, wherein the first and second barcodes each comprises:
a degenerate nucleotide sequence; or
a partially degenerative nucleotide sequence.

16. (canceled)

17. (canceled)

18. (canceled)

19. (canceled)

20. The method of claim 1, wherein the set of first and set of second barcoding oligonucleotides comprise pooled barcoding oligos with multiple different defined sequences.

21. (canceled)

22. (canceled)

23. (canceled)

24. The method of claim 1, wherein the nucleotide sequence of the first or second barcode is positioned between the nucleotide sequences of the two consensus regions.

25. (canceled)

26. The method of claim 1, wherein:
the first barcode of the barcoding oligonucleotides within the first set of barcoding oligonucleotides is distinguishable from other first barcodes of the first set of barcoding oligonucleotides by its nucleotide sequence; or
the second barcode of the barcoding oligonucleotides within the second set of barcoding oligonucleotides is distinguishable from other second barcode of the second set of barcoding oligonucleotides by its nucleotide sequence.

27. (canceled)

28. The method of claim 1, wherein said contacting comprises:
contacting the cell suspension or tissue slices with the first and second set of barcoding oligonucleotides at a concentration such that each cell within the cell suspension or tissue slice comprises a first and second barcoding oligonucleotide that is distinguishable from a first and second barcoding oligonucleotide of a different cell; or
contacting the cell suspension or tissue slices with the first and second set of barcoding oligonucleotides at a concentration such that each cell within the cell suspension or tissue slice comprises 2-1000 barcoding oligonucleotides.

29. (canceled)

30. (canceled)

31. (canceled)

32. The method of claim 28, wherein a cell within the cell suspension or tissue slice:
comprises less than 5% of barcoding oligonucleotides with the same first and second barcode as a different cell within the cell suspension; or
does not comprise the first and second barcode that is the same first and second barcode of a second cell within the cell suspension or tissue slice.

33. (canceled)

34. The method of claim 1, wherein the nucleic acid fragment is a DNA amplicon product or a DNA product of ligation.

35. (canceled)

36. The method of claim 34, wherein the method comprises ligating a consensus read region comprising a first 5′ read region and a consensus read region comprising a second 5′ read region to a DNA fragment using a Y-adapter, a hairpin adapter, or a duplex adapter.

37. (canceled)

38. (canceled)

39. (canceled)

40. (canceled)

41. (canceled)

42. (canceled)

43. The method of claim 42, wherein the method comprises lysing:
the cells containing the set of amplicon products, or
the cells containing the second set of amplicon products.

44. (canceled)

45. (canceled)

46. (canceled)

47. The method of claim 1, wherein the cell suspension comprises: 1000 cells or less, a single cell, or a single pool of cells.

48. (canceled)

49. (canceled)

50. (canceled)

51. (canceled)

52. (canceled)

53. The method of claim 47, wherein the method is performed within individual cells of the single pool of the cells.

54. The method of claim 1, further comprising:
fragmenting nucleic acid within the permeabilized cell suspension or tissue slices to form the nucleic acid fragments; and
ligating a consensus read region to one or both ends of the nucleic acid fragments.

55. (canceled)

56. (canceled)

57. The method of claim 54, wherein the fragmenting and ligating steps are performed in a first buffer and the introducing step (a) and the amplifying steps (b) and (c) are performed in a second buffer.

58. The method of claim 57, wherein the method comprises conducting a buffer exchange and cell washing step, wherein the first buffer is removed and replaced with a second buffer.

59. (canceled)

60. (canceled)

61. (canceled)

62. A method of generating primers from oligonucleotides using linear amplification, the method comprising:
(a) introducing to a reaction container:
(i) an oligonucleotide, wherein the oligonucleotide comprises:
an amplification sequence, and
a consensus region that is complementary to a target sequence of a nucleic acid fragment; and
(b) amplifying, in the reaction container, the oligonucleotides to produce a primer comprising the reverse complement of the consensus region.

63. The method of claim 62, wherein the introducing step (a) further comprises introducing an amplification primer comprising a consensus region that is complementary to the amplification sequence on the oligonucleotide.

64. The method of claim 62,
wherein the introducing step (a) further comprises:
introducing a second oligonucleotide, wherein the second oligonucleotide comprises: a second amplification sequence, and a second consensus region that is complementary to a second target sequence of a nucleic acid fragment, and
introducing a second amplification primer comprising a consensus region that is complementary to the second amplification sequence on the second oligonucleotide; and
wherein the amplifying step (b) further comprises amplifying, in the reaction container, the second oligonucleotide to produce a second primer comprising the reverse complement of the second consensus region.

65. (canceled)

66. (canceled)

67. (canceled)

68. (canceled)

69. (canceled)

70. (canceled)

71. (canceled)

72. The method of claim 62, wherein the first oligonucleotide, the second oligonucleotide, or both, comprise:
from 5′ to 3′:
(a) a consensus region, a barcode, an amplification sequence, and a nick endonuclease recognition sequence, or any combination or orientation thereof; or
(b) a consensus region, a barcode, an amplification sequence, and a reverse complement of a nick endonuclease recognition sequence, or any combination or orientation thereof;
a stem loop sequence; or
a nick endonuclease recognition sequence, a reverse complement of a nick endonuclease recognition sequence, or both.

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. The method of claim 62, wherein the amplifying in step (b) comprises amplifying via isothermal amplification, the oligonucleotides to produce the primers, and
wherein the amplifying in step (b):
is performed under conditions that allow for primer invasion, or
further comprises a nick endonuclease.

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. The method of claim 62, wherein the reaction container is selected from a cell (in situ), a subcellular compartment (e.g., nucleus, cytoplasm), a tube, a well, a partition, a solution, and a droplet.

105. (canceled)

106. (canceled)

107. (canceled)

108. (canceled)

109. A cell barcoding kit comprising:
(a) a first set of barcoding oligonucleotides, each barcoding oligonucleotide comprising:
a first barcode;
two consensus regions, wherein the two consensus regions of each barcoding primer comprises:
one of the two consensus regions comprises a nucleotide sequence that is complementary to a 5′ read region of a first strand of one of DNA or RNA fragments, and
the second of the two consensus regions comprises a first adapter sequence;
(b) a second set of barcoding oligonucleotides, each barcoding oligonucleotide comprising:
a second barcode;
two consensus regions, wherein the two consensus regions of each barcoding primer comprises:
one of the two consensus regions comprises a nucleotide sequence that is complementary to a 5′ read region of a second strand of one DNA or RNA fragments, and
the second of the two consensus regions comprises a second adapter sequence.

110. The kit of claim 109, wherein each of the first barcoding oligonucleotides is annealed to a first primer comprising a nucleotide sequence that is complementary to the first adapter sequence of the first barcoding oligonucleotide or each of the second barcoding oligonucleotides is annealed to a second primer comprising a nucleotide sequence that is complementary to the second adapter sequence of the second barcoding oligonucleotide.

111. (canceled)

112. The kit of claim 109, wherein the first and second barcoding oligonucleotides are hairpin oligonucleotides.

113. (canceled)

114. (canceled)

115. The kit of claim 112, wherein the kit further comprises one or more enzymes selected from one or more of: DNA polymerase, RNA polymerase, nicking enzyme, a Bst2.0 polymerase, a Phi29 polymerase, an enzymatic fragmentation enzyme, an End Repair A-tail enzyme, a DNA ligase, or a combination thereof.

116. (canceled)

117. (canceled)

118. (canceled)

119. A cell barcoding composition comprising:
(a) cell suspension or tissue slices comprising nucleic acid fragments;
(b) a first primer set comprising barcoding primers configured to bridge and extend from the 5′ region of the nucleic acid fragments;
wherein each first barcoding primer comprises:
a first barcode or a reverse complement thereof;
a first consensus region or a reverse complement thereof comprising a nucleotide sequence that is complementary to a 5′ read region of a first strand of one of the nucleic acid fragments, and
a second consensus region or a reverse complement thereof comprising a first adapter sequence;
(c) a second primer set comprising barcoding primers configured to bridge and extend from the 5′ region of the opposite strand of the nucleic acid fragments,
wherein each second barcoding primer comprises:
a second barcode or a reverse complement thereof;
a second consensus region or a reverse complement thereof comprising a nucleotide sequence that is complementary to a 5′ read region of a second strand of one of the nucleic acid fragments, and
a second consensus region or a reverse complement thereof comprising a second adapter sequence;
wherein the first and second barcoding primer sets do not amplify a target region of the nucleic acid sequences;
(d) a third primer set comprising nucleotide sequences that are complementary to the first adapter sequence of the first primer set; and
(e) a fourth primer set comprising nucleotide sequences that are complementary to the second adapter sequence of the second primer set.

120. The composition of claim 119, wherein the barcode comprises a degenerate nucleotide sequence.

121. (canceled)

122. The composition of claim 119, wherein the nucleic acid fragment comprises a DNA sequence, wherein the DNA sequence is a DNA amplicon product or a DNA product of ligation.

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)


SEQUENCE LISTING (15 sequences)

SEQ ID NO: 1 - DNA, 69 bp, Artificial Sequence
  synthetic sequence
  misc_feature (21)..(40): n is a, c, g, or t
gtcgtgtagggaaagagtgtnnnnnnnnnnnnnnnnnnnngtgtagatctcggtggtcgccgtatcatt

SEQ ID NO: 2 - DNA, 65 bp, Artificial Sequence
  synthetic sequence
  misc_feature (22)..(41): n is a, c, g, or t
acacgtctgaactccagtcacnnnnnnnnnnnnnnnnnnnnatctcgtatgccgtcttctgcttg

SEQ ID NO: 3 - DNA, 28 bp, Artificial Sequence
  Synthetic sequence
aatgatacggcgaccaccgagatctaca

SEQ ID NO: 4 - DNA, 24 bp, Artificial Sequence
  synthetic sequence
caagcagaagacggcatacgagat

SEQ ID NO: 5 - DNA, 90 bp, Artificial Sequence
  synthetic sequence
  misc_feature (21)..(40): n is a, c, g, or t
gtcgtgtagggaaagagtgtnnnnnnnnnnnnnnnnnnnngtgtagatctcggtggtcgccgtatcattaaaaaaaaaaaaaaaaaaaaa

SEQ ID NO: 6 - DNA, 86 bp, Artificial Sequence
  synthetic sequence
  misc_feature (22)..(41): n is a, c, g, or t
acacgtctgaactccagtcacnnnnnnnnnnnnnnnnnnnnatctcgtatgccgtcttctgcttgaaaaaaaaaaaaaaaaaaaaa

SEQ ID NO: 7 - DNA, 20 bp, Artificial Sequence
  synthetic sequence
tttttttttttttttttttt

SEQ ID NO: 8 - DNA, 33 bp, Artificial Sequence
  synthetic sequence
acactctttccctacacgacactattccgatct

SEQ ID NO: 9 - DNA, 33 bp, Artificial sequence
  synthetic sequence
tgactggagttcagacgtgtactattccgatct

SEQ ID NO: 10 - DNA, 20 bp, Artificial sequence
  synthetic sequence
aatgatacggcgaccaccga

SEQ ID NO: 11 - DNA, 21 bp, Artificial sequence
  synthetic sequence
caagcagaagacggcatacga

SEQ ID NO: 12 - DNA, 99 bp, Artificial sequence
  synthetic sequence
  misc_feature (23)..(27): n is a, c, g, or t
  misc_feature (30)..(34): n is a, c, g, or t
  misc_feature (37)..(41): n is a, c, g, or t
  misc_feature (44)..(48): n is a, c, g, or t
gtcgtgtagggaaagagtgtaannnnngtnnnnngtnnnnngtnnnnnccgtgtagatctcggtggtcgccgtatcattaaaaaaaaaaaaaaaaaaaa

SEQ ID NO: 13 - DNA, 92 bp, Artificial sequence
  synthetic sequence
  misc_feature (22)..(26): n is a, c, g, or t
  misc_feature (29)..(33): n is a, c, g, or t
  misc_feature (36)..(40): n is a, c, g, or t
  misc_feature (43)..(47): n is a, c, g, or t
acacgtctgaactccagtcacnnnnnacnnnnnacnnnnnacnnnnnatctcgtatgccgtcttctgcttgaaaaaaaaaaaaaaaaaaaaa

SEQ ID NO: 14 - DNA, 19 bp, Artificial sequence
  synthetic sequence
gtcgtgtagggaaagagtg

SEQ ID NO: 15 - DNA, 20 bp, Artificial sequence
  synthetic sequence
acacgtctgaactccagtca